(19) United States
(12) Patent Application Publication
(10) Pub. No.: US 2025/0000965 A1
(43) Pub. Date: Jan. 2, 2025

(54) IDENTIFICATION AND USES OF PEPTIDE SEQUENCES OF SARS-COV-2 T CELL AND B CELL EPITOPES

(71) Applicant: THE HONG KONG UNIVERSITY OF SCIENCE AND TECHNOLOGY, Clear Water Bay, Kowloon (HK)

(72) Inventors: Robert Matthew McKay, Clear Water Bay, Kowloon (HK); Ahmed Abdul Quadeer, Sai Kung N.T. (HK); Syed Faraz Ahmed, Sai Kung N.T. (HK); Muhammad Saqib Sohail, Sai Kung N.T. (HK)

(21) Appl. No.: 18/260,767

(22) PCT Filed: Jan. 10, 2022
(86) PCT No.: PCT/CN2022/070948

§ 371 Date: Jul. 7, 2023

Related U.S. Application Data
(60) Provisional application No. 63/136,175, filed on Jan. 11, 2021.
(60) Provisional application No. 63/229,063, filed on Aug. 3, 2021.

(51) Int. Cl. A61K 39/215; C07K 14/005; G01N 33/569

(52) U.S. Cl. CPC A61K 39/215; C07K 14/005; G01N 33/56972; C12N 2770/20022; C12N 2770/20034; G01N 2333/165; G01N 2333/70539

(57) ABSTRACT

The present disclosure identifies a set of T cell and B cell epitopes derived from Severe Acute Respiratory Syndrome Coronavirus 1 (SARS-CoV-1) and their use in designing vaccines against SARS-CoV-2. A method for eliciting an immune response for prophylactic or therapeutic application using the T cell and/or B cell epitopes is described. Further disclosed are SARS-CoV-2-specific polypeptides, nucleic acids, host cells, and corresponding compositions for eliciting or measuring an immune response to SARS-CoV-2 and/or other coronaviruses.

### CROSS-REFERENCES TO RELATED APPLICATIONS

This application claims priority to U.S. Provisional Patent Application No. 63/229,063, filed Aug. 3, 2021, and U.S. Provisional Patent Application No. 63/136,175, filed Jan. 11, 2021, the contents of both are hereby incorporated by reference in the entirety for all purposes.

### BACKGROUND

COVID-19 is caused by a novel coronavirus, Severe Acute Respiratory Syndrome Coronavirus 2 (SARS-CoV-2). Worldwide collaborative efforts from scientists are working on this disease and SARS-CoV-2 to develop effective interventions for controlling and preventing it [6-9].

Currently, treatment options for COVID-19, which is caused by a novel coronavirus, Severe Acute Respiratory Syndrome Coronavirus 2 (SARS-CoV-2), are limited. The Food and Drug Authorization (FDA) has approved the antiviral drug Veklury (remdesivir) for adults and certain pediatric patients with COVID-19 who are sick enough to need hospitalization. By Emergency Use Authorization (EUA), the FDA may authorize the use of unapproved drugs or unapproved uses of approved drugs under certain conditions. For example, the FDA has issued EUAs for several monoclonal antibody treatments for COVID-19 for the treatment, and in some cases prevention (prophylaxis), of COVID-19 in adults and pediatric patients. Monoclonal antibodies are laboratory-made molecules that act as substitute antibodies. They can help the immune system recognize and respond more effectively to the virus, making it more difficult for the virus to reproduce and cause harm. Like other infectious organisms, SARS-CoV-2 can mutate over time, resulting in genetic variation in the population of circulating viral strains. The risk-benefit assessment for using certain monoclonal antibodies may not be favorable due to the increased frequency of resistant variants. Global efforts to combat COVID-19 have led to the rapid development of multiple vaccines. As the virus continues to circulate worldwide, virus variants have emerged in several regions, raising concerns about their potential to escape vaccine-induced antibody responses.

As such, there is an imminent need to improve efficacy of COVID-19 treatments and provide protection against severe disease and hospitalization. While vaccines have been pursued with success, there is an unmet need for developing vaccines that may induce a robust neutralizing antibody response to combat the COVID-19 pandemic.

### SUMMARY

The summary is provide to introduce a selection of concepts that are further described below in the Detailed Description. This Summary is not intended to identify key or essential features of the claimed subject matter, nor is it intended to be used as an aid in limiting the scope of the claimed subject matter.

This present disclosure provides new compositions and methods useful for eliciting an immune response to SARS-CoV-2 and/or other coronavirus infections, based on the discovery that SARS-CoV-2 and/or other coronavirus infections may elicit T-cell and/or B cell response in a subject. Thus, in one aspect, the present disclosure is related to a peptide of no more than 500 amino acids, comprising (1) at least one T cell epitope set forth in Table 3 or Tables 15-47 or (2) at least one B cell epitope set forth in Table 4, the peptide optionally further comprising at least one heterologous amino acid sequence.

In some embodiments, the peptide comprises at least one T cell epitope set forth in Table 3 and at least one T cell epitope set forth in Tables 15-47. In some embodiments, the peptide comprises at least one T cell epitope set forth in Table 3 and at least one B cell epitope set forth in Table 4. In some embodiments, the peptide comprises at least one T cell epitope set forth in Tables 15-47 and at least one B cell epitope set forth in Table 4. In some embodiments, the peptide comprises at least one T cell epitope set forth in Table 3 and at least one T cell epitope set forth in Tables 15-47 and at least one B cell epitope set forth in Table 4. In any cases, the peptide may optionally further comprise at least one heterologous amino acid sequence. In some embodiments, the peptide comprises or consists of at least one of the T cell epitopes. In some embodiments, the peptide comprises or consists of at least one of the B cell epitopes. In some embodiments, the peptide comprises or consists of at least one of the T cell epitopes and at least one heterologous amino acid sequence. In some embodiments, the peptide comprises or consists of at least one of the B cell epitopes and at least one heterologous amino acid sequence.

In some embodiments, the peptide comprising the one or more T cell epitope set forth in Table 3 and/or Tables 15-47, and/or one or more B cell epitopes set forth in Table 4 comprises of no more than 500, 450, 400, 350, 300, 250, 200, 150, 120, 100, 90, 80, 70, 60, 50, 40, 30, 20, 10, or 5 amino acids. In some embodiments, the peptide comprising the one or more T cell epitope set forth in Table 3 and/or Tables 15-47, and/or one or more B cell epitopes set forth in Table 4 comprises at least 5, 10, 20, 30, 40, 50, 60, 70, 80, 90, 100, 120, 150, 200, 250, 300, 350, 400, 450, or 500 amino acids. In some embodiments, the peptide comprising the one or more T cell epitope set forth in Table 3 and/or Tables 15-47, and/or one or more B cell epitopes set forth in Table 4 comprises between about 5-500, about 20-100, about 40-200, about 50-450, about 80-350, about 100-400, or about 150-350 amino acids. In some embodiments, the peptide comprising the one or more T cell epitope set forth in Table 3 and/or Tables 15-47, and/or one or more B cell epitopes set forth in Table 4 comprises about 20 amino acids. In some embodiments, the peptide comprising the one or more T cell epitope set forth in Table 3 and/or Tables 15-47, and/or one or more B cell epitopes set forth in Table 4 comprises about 100 amino acids. In any cases, the peptide may further comprise at least one heterologous amino acid sequence.

The second aspect of the present disclosure provides a nucleic acid comprising a polynucleotide sequence encoding the peptide of no more than 500 amino acids, comprising (1) at least one T cell epitope set forth in Table 3 or Tables 15-47 or (2) at least one B cell epitope set forth in Table 4, the peptide optionally further comprising at least one heterologous amino acid sequence, or a peptide comprising or consisting of at least one of the T cell epitopes, or a peptide comprising or consisting of at least one of the B cell epitopes, or a peptide comprising or consisting of at least one of the T cell epitopes and at least one heterologous amino acid sequence, or a peptide comprising or consisting of at least one of the B cell epitopes and at least one heterologous amino acid sequence. In some embodiments, the polynucleotides encodes a peptide of at least 5, 10, 20, 30, 40, 50, 60, 70, 80, 90, 100, 120, 150, 200, 250, 300, 350, 400, 450, or 500 amino acids. In some embodiments, the polynucleotides encodes a peptide of between about 5-500, about 20-100, about 40-200, about 50-450, about 80-350, about 100-400, or about 150-350 amino acids. In some embodiments, the polynucleotides encodes a peptide of about 20 amino acids. In some embodiments, the polynucleotides encodes a peptide of about 100 amino acids. In some embodiments, the polynucleotide sequence comprises deoxyribonucleic acid (DNA). In some embodiments, the polynucleotide sequence comprises ribonucleic acid (RNA) such as messenger RNA (mRNA). In some embodiments, the present disclosure provides an expression cassette comprises any of the peptide as described herein and the peptide is operably linked to a promoter. In some embodiments, the present disclosure provides a vector comprising the expression cassette comprising any of the peptides as described herein and the peptide is operably linked to a promoter. In some embodiments, the present disclosure provides a host cell comprising the vector comprising the expression cassette.

In a third aspect, the present disclosure provides a composition comprising (1) a peptide of no more than 500 amino acids, comprising (1) at least one T cell epitope set forth in Table 3 or Tables 15-47 or (2) at least one B cell epitope set forth in Table 4, the peptide optionally further comprising at least one heterologous amino acid sequence, or a peptide comprising or consisting of at least one of the T cell epitopes, or a peptide comprising or consisting of at least one of the B cell epitopes, or a peptide comprising or consisting of at least one of the T cell epitopes and at least one heterologous amino acid sequence, or a peptide comprising or consisting of at least one of the B cell epitopes and at least one heterologous amino acid sequence, the nucleic acid encoding any of the peptides, the expression cassette comprising any of the peptides, the vector comprising the expression cassette, or the host cell; and (2) a pharmaceutically acceptable excipient. In some embodiments, the composition further comprises an adjuvant. In some embodiments, the composition comprises a plurality of peptides each comprising a T cell epitope set forth in Table 3 or Tables 15-47. In some embodiments, the peptide comprising the one or more T cell epitope set forth in Table 3 and/or Tables 15-47, and/or one or more B cell epitopes set forth in Table 4 comprises of no more than 500, 450, 400, 350, 300, 250, 200, 150, 120, 100, 90, 80, 70, 60, 50, 40, 30, 20, 10, or 5 amino acids. In some embodiments, the peptide comprising the one or more T cell epitope set forth in Table 3 and/or Tables 15-47, and/or one or more B cell epitopes set forth in Table 4 comprises at least 5, 10, 20, 30, 40, 50, 60, 70, 80, 90, 100, 120, 150, 200, 250, 300, 350, 400, 450, or 500 amino acids. In some embodiments, the peptide comprising the one or more T cell epitope set forth in Table 3 and/or Tables 15-47, and/or one or more B cell epitopes set forth in Table 4 comprises between about 5-500, about 20-100, about 40-200, about 50-450, about 80-350, about 100-400, or about 150-350 amino acids. In some embodiments, the peptide comprising the one or more T cell epitope set forth in Table 3 and/or Tables 15-47, and/or one or more B cell epitopes set forth in Table 4 comprises about 20 amino acids. In some embodiments, the peptide comprising the one or more T cell epitope set forth in Table 3 and/or Tables 15-47, and/or one or more B cell epitopes set forth in Table 4 comprises about 100 amino acids.

In a fourth aspect, the present disclosure provides a method of eliciting an immune response in a subject in need thereof, the method comprising administering to the subject an effective amount of a composition comprising (1) a peptide of no more than 500 amino acids, comprising (1) at least one T cell epitope set forth in Table 3 or Tables 15-47 or (2) at least one B cell epitope set forth in Table 4, the peptide optionally further comprising at least one heterologous amino acid sequence, or a peptide comprising or consisting of at least one of the T cell epitopes, or a peptide comprising or consisting of at least one of the B cell epitopes, or a peptide comprising or consisting of at least one of the T cell epitopes and at least one heterologous amino acid sequence, or a peptide comprising or consisting of at least one of the B cell epitopes and at least one heterologous amino acid sequence, the nucleic acid encoding any of the peptides, the expression cassette comprising any of the peptides, or the vector comprising the expression cassette. In some embodiments, the method comprises administering the composition to the subject by a route selected from the group consisting of, subcutaneous, intramuscular, and oral. In some embodiments, the subject is at risk of exposure to SARS-CoV, SARS-CoV-2 or other coronavirus infections. In some embodiments, the subject is at risk of exposure to SARS-CoV-2 infections. In some embodiments, the subject is at risk of developing severe illnesses when contracted with SARS-CoV-2 (e.g., COVID-19). In some embodiments, the peptide comprising the one or more T cell epitope set forth in Table 3 and/or Tables 15-47, and/or one or more B cell epitopes set forth in Table 4 comprises of no more than 500, 450, 400, 350, 300, 250, 200, 150, 120, 100, 90, 80, 70, 60, 50, 40, 30, 20, 10, or 5 amino acids. In some embodiments, the peptide comprising the one or more T cell epitope set forth in Table 3 and/or Tables 15-47, and/or one or more B cell epitopes set forth in Table 4 comprises at least 5, 10, 20, 30, 40, 50, 60, 70, 80, 90, 100, 120, 150, 200, 250, 300, 350, 400, 450, or 500 amino acids. In some embodiments, the peptide comprising the one or more T cell epitope set forth in Table 3 and/or Tables 15-47, and/or one or more B cell epitopes set forth in Table 4 comprises between about 5-500, about 20-100, about 40-200, about 50-450, about 80-350, about 100-400, or about 150-350 amino acids. In some embodiments, the peptide comprising the one or more T cell epitope set forth in Table 3 and/or Tables 15-47, and/or one or more B cell epitopes set forth in Table 4 comprises about 20 amino acids. In some embodiments, the peptide comprising the one or more T cell epitope set forth in Table 3 and/or Tables 15-47, and/or one or more B cell epitopes set forth in Table 4 comprises about 100 amino acids.

In a fifth aspect, the present disclosure provides a kit for eliciting an immune response in a subject in need thereof, the kit comprising a first container containing the composition comprising (1) a peptide of no more than 500 amino acids, comprising (1) at least one T cell epitope set forth in Table 3 or Tables 15-47 or (2) at least one B cell epitope set forth in Table 4, the peptide optionally further comprising at least one heterologous amino acid sequence, or a peptide comprising or consisting of at least one of the T cell epitopes, or a peptide comprising or consisting of at least one of the B cell epitopes, or a peptide comprising or consisting of at least one of the T cell epitopes and at least one heterologous amino acid sequence, the nucleic acid encoding any of the peptides, the expression cassette comprising any of the peptides, or the vector comprising the expression cassette. In some embodiments, the kit optionally contains an additional container containing a therapeutic agent against SARS-CoV-2. In some embodiments, the kit further comprises at least a second container each containing at least one different composition. In some embodiments, the peptide comprising the one or more T cell epitope set forth in Table 3 and/or Tables 15-47, and/or one or more B cell epitopes set forth in Table 4 comprises of no more than 500, 450, 400, 350, 300, 250, 200, 150, 120, 100, 90, 80, 70, 60, 50, 40, 30, 20, 10, or 5 amino acids. In some embodiments, the peptide comprising the one or more T cell epitope set forth in Table 3 and/or Tables 15-47, and/or one or more B cell epitopes set forth in Table 4 comprises at least 5, 10, 20, 30, 40, 50, 60, 70, 80, 90, 100, 120, 150, 200, 250, 300, 350, 400, 450, or 500 amino acids. In some embodiments, the peptide comprising the one or more T cell epitope set forth in Table 3 and/or Tables 15-47, and/or one or more B cell epitopes set forth in Table 4 comprises between about 5-500, about 20-100, about 40-200, about 50-450, about 80-350, about 100-400, or about 150-350 amino acids. In some embodiments, the peptide comprising the one or more T cell epitope set forth in Table 3 and/or Tables 15-47, and/or one or more B cell epitopes set forth in Table 4 comprises about 20 amino acids. In some embodiments, the peptide comprising the one or more T cell epitope set forth in Table 3 and/or Tables 15-47, and/or one or more B cell epitopes set forth in Table 4 comprises about 100 amino acids.

In a sixth aspect, the present disclosure provides a method for detecting T cell immunity against SARS-CoV-2 in a subject, comprising: (1) contacting T cells obtained from the subject with a T cell epitope set forth in Table 3 or Tables 15-47 and antigen-presenting cells having an HLA allele associated with the epitope; and (2) detecting activation of the T cells, thereby detecting presence of T cell immunity against SARS-CoV-2 in the subject. In some embodiments, step (2) of the method comprises detection of T cell proliferation or T cell secretion of one or more cytokines. In some embodiments, step (2) of the method comprises T cell proliferation assay, flow cytometry, ELISPOT, or ELISA. In some embodiments, step (1) of the method comprises contacting T cells obtained from the subject with a composition comprising a plurality of peptides each comprising a T cell epitope set forth in Table 3 or Tables 15-47 and antigen-presenting cells having HLA alleles associated with each of the plurality of epitopes.

### BRIEF DESCRIPTION OF THE DRAWINGS

FIGS. 1A-B show comparison of the similarity of structural proteins of SARS-CoV-2 with the corresponding proteins of SARS-CoV and MERS (Middle East Respiratory Syndrome)-CoV. FIG. 1A shows the percentage genetic similarity of the individual structural proteins of SARS-CoV-2 with those of SARS-CoV and MERS-CoV. The reference sequence of each coronavirus (Materials and Methods) was used to calculate the percentage genetic similarity. FIG. 1B is a circular phylogram of the phylogenetic trees of the four structural proteins. All trees were constructed based on the available unique sequences using PASTA and rooted with the outgroup Zaria Bat CoV strain (accession ID: HQ166910.1).

FIGS. 2A-C depict location of SARS-CoV S protein subunits and SARS-CoV-derived B cell epitopes on the protein structure (PDB ID: 5XLR). FIG. 2A shows subunits S1 and S2 are indicated in medium and light grey color, respectively. The receptor binding motif lies within the S1 subunit and is indicated in dark grey color. FIG. 2B shows residues of the linear B cell epitopes, that were identical in SARS-CoV-2 (Table 4), are shown as shaded. The black and grey colors reflect the surface and buried residues, respectively. FIG. 2C shows locations of discontinuous B cell epitopes that share at least one identical residue with corresponding SARS-CoV-2 sites (Table 5). Identical epitope residues are shown in dark grey color, while the remaining epitope residues are shown in medium grey color. Both the side view (left panel) and the top view (right panel) of the structure are shown.

FIG. 3 shows fraction of mutations in the observed sequences of the structural proteins of the three coronaviruses. Mutation is defined here as an amino acid difference from the reference sequence of the respective coronavirus; accession IDs: NC_045512.2 (2019-nCoV), NC_004718.3 (SARS-CoV), and NC_019843.3 (MERS-CoV).

FIG. 4 illustrates location of identified T cell epitopes on the SARS-CoV S protein structure (PDB ID: 5XLR). Residues of the SARS-CoV-derived T cell epitopes (determined using positive MHC binding assays and that were identical in SARS-CoV-2) are shown with filled color. The dark and light shade reflect the surface and buried residues, respectively.

FIG. 5 shows pairwise sequence alignment of the reference sequences of the S proteins of SARS-CoV and SARS-CoV-2 (accession ID: NP_828851.1 and YP_009724390.1, respectively). Identical residues are indicated by *.

FIGS. 6A-C show global diversity of HLA class I alleles and the corresponding T cell responses, and summary of the experimentally-determined SARS-CoV-2 CD8+ T cell epitope data. FIG. 6A shows different HLA class I alleles are prevalent in different geographical regions (left panel); heatmaps depicting the diverse HLA allele distribution in North America, North East Asia, and Australia are shown as examples (middle panel). Each square in the heatmap represents a distinct HLA class I allele, and its color shade represents the frequency of the allele in the geographical region with a dark shade representing high frequency and vice versa; and different HLA alleles present different SARS-CoV-2 peptides, and consequently the T cell responses elicited against SARS-CoV-2 are expected to differ among geographical regions (right panel). Thus, peptide pools to test for SARS-CoV-2 CD8+ T cell responses need to be designed specific to the HLA alleles prevalent in the population being tested. This figure was created with BioRender.com. FIG. 6B shows the number of HLA alleles in each geographical region for which at least one SARS-CoV-2 experimentally-determined CD8+ T cell epitope is known. FIG. 6C shows the number of experimentally-determined epitopes within spike and other SARS-CoV-2 proteins determined so far for class I HLA alleles (left panel) and the frequencies of these HLA alleles in different geographical regions (right panel) (for details of experimentally-determined epitope data, see Methods).

FIGS. 7A-B show an optimized in silico strategy to predict SARS-CoV-2 CD8+ T cell epitopes. FIG. 7A shows a performance comparison of in silico HLA class I epitope prediction methods based on predicting experimentally-determined SARS-CoV-2 CD8+ T cell epitopes. Intersection of the top predictions of the analysed 12 in silico methods showed that MHCflurry2.0P predicts the highest number of epitopes not predicted by any other method. For each method, the set of top-ranked predicted peptides consisted of n=200 predictions, with 20 predictions for each of the 10 HLA alleles having the most experimental data available (see Methods). FIG. 7B shows the set of top-ranked predictions (n=341) obtained from the proposed approach, union of MHCflurry2.0P and NetMHCpan4.1BA (see Methods), comprised of more experimentally-determined epitopes than the sets of top-ranked predictions of the individual methods

FIGS. 8A-B show a schematic of SARS2TPools framework and snapshot of the platform's interface for a use case. FIG. 8A is a schematic showing how the SARS2TPools, based on user-selected options (protein, geographical region, specific HLA alleles), combines experimentally-determined epitope data, in silico predictions, and information of prevalent HLA alleles across regions to obtain optimized peptide pools for assessing vaccine-induced T cell responses. This figure was created with BioRender.com. FIG. 8B is a snapshot of the SARS2TPools interface for the “Region-specific” tab with selected options (Region: Oceania; Protein: NSP12; Pool-size of peptides: small; Threshold for in silico predictions: Default; and Length of peptides: 9).

FIGS. 9A-C are summary of region-specific CD8+ T cell peptide pools provided by SARS2TPools. FIG. 9A shows the number of peptides in region specific pools from experimental studies and in silico predictions. FIG. 9B shows overlap between pairs of region-specific pools. FIG. 9C shows a source of peptides for each region-specific pool. Each circle represents one of the top 30 prevalent HLAs for a specific region and the color of the circle indicates whether the peptides were obtained from only experimental studies, or only from in silico predictions, or from both.

FIG. 10 shows histograms of ranks assigned by different in silico epitope prediction methods to the experimentally-determined SARS-CoV-2 CD8+ T cell epitopes associated with the 10 HLA alleles with the most experimental data (see Methods). Each bin here represents a set of peptides within the specified rank range of all 10 HLA alleles.

FIGS. 11A-D show the observation that predictions of MHCflurry2.0P are distinct from those of other in silico methods (FIG. 12) was robust to the size of the set of top-ranked predictions compared. Distinct sets of predicted epitopes ranked by the considered 12 in silico methods in their (FIG. 11A) top 10, (FIG. 11B) top 15, (FIG. 11C) top 20, and (FIG. 11D) top 25 predictions corresponding to each of the 10 HLA alleles having the most experimental data (see Methods).

FIG. 12 shows the strategy of combining top-ranked predictions of MHCflurry2.0P with any of the other 11 in silico methods considered in this study had a higher hit-rate on experimentally-determined SARS-CoV-2 CD8⁺ epitope data than either of the individual methods. Here, hit-rate represents the fraction of experimentally known epitopes present in the set of top-ranked predicted peptides.

FIG. 13 shows hit-rates of union methods comprising of MHCflurry2.0P and one of the other 11 in silico epitope prediction methods. While none of these clearly outperformed the rest, the 4 union methods comprising of MHCflurry2.0P and either NetMHCpan4.1BA, NetMHCpan4.0BA, NetMHC4.0, or NetMHCpan4.1EL were among the top 4 performing methods (also see Table 13). Here, hit-rate represents the fraction of experimentally known epitopes present in the set of top-ranked predicted peptides.

FIGS. 14A-B illustrate COVIDep providing an up-to-date set of B-cell and T-cell epitopes that can serve as potential vaccine targets for SARS-CoV-2. FIG. 14A shows the identified epitopes are experimentally-derived from SARS-CoV and have a close genetic match with the available SARS-CoV-2 sequences. FIG. 14B is an example of the T-cell epitopes reported by COVIDep (as of 20 May 2020) for the spike protein of SARS-CoV-2. Here, the Search box (in the top right) was used to select only the HLA-A*02:01-restricted epitopes. (An explanation of all interactive COVIDep visualizations is incorporated in the “How to use COVIDep page” of the platform.) Of the 14 epitopes listed in the display, 9 of them (IEDB IDs 36724, 54507, 54725, 69657, 71663, 2801, 54680, 16156, and 37289) overlap with epitopes against which cytotoxic CD8+ T cell responses have been observed in peripheral blood mononuclear cells isolated from COVID-19 patients. T cell responses were also recorded against protein regions overlapping with the epitope with IEDB ID 71663 in a pre-clinical trial of a DNA vaccine candidate.

FIG. 15 is an epitope screening protocol used by COVIDep for providing vaccine target recommendations for SARS-CoV-2. COVIDep periodically pools SARS-CoV-2 sequence data from the GISAID database (www.gisaid.org) and compares with experimentally-determined T cell and B cell epitopes of SARS-CoV, obtained from the ViPR database (www.viprbrc.org). The T cell epitopes were determined based on either positive T cell assays or positive MHC binding assays for SARS-CoV. For the B cell epitopes, both linear and discontinuous epitopes were considered. The system outputs those epitopes that are genetically similar in SARS-CoV-2, based on an epitope screening parameter. This user-defined parameter allows the user to select epitopes based on their conservation in the SARS-CoV-2 sequence data, where conservation is defined as the fraction of SARS-CoV-2 sequences with the exact epitope sequence. The value of this parameter is set to 0.95 as default; however, the user may change this value to adjust the stringency of the screening criterion. For example, reducing the value of the parameter will allow for the consideration of epitopes with greater genetic variation, potentially increasing the set of recommended SARS-CoV-2 vaccine targets. For the identified T cell epitopes, the population coverage analysis tool available at IEDB (www.iedb.org) is used to estimate the percentage of a specified population that can elicit a response against them.

### DEFINITIONS

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 60%, 70%, 80%, 85%, 90%, 95%, or 99% pure, more preferably at least 95% pure, and most preferably at least 99% pure.

In this application, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure different from the general chemical structure of an amino acid, but capable of functioning in a manner similar to a naturally occurring amino acid.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, or mRNA encoded by a gene.

When the relative locations of elements in a polynucleotide sequence are concerned, a “downstream” location is one at the 3′ side of a reference point, and an “upstream” location is one at the 5′ side of a reference point.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds. In this application, the amino acid sequence of a polypeptide is presented from the N-terminus to the C-terminus. In other words, when describing an amino acid sequence of a peptide, the first amino acid from the N-terminus is referred to as the “first amino acid.”

When used in the context of describing partners of a fusion peptide, the term “heterologous” refers to the relationship of one peptide fusion partner to the another peptide fusion partner: the manner in which the fusion partners are present in the fusion peptide is not one that can be found a naturally occurring protein. For instance, a “heterologous polypeptide” fused with a T cell or B cell epitope to form a fusion peptide may be one that is originated from a protein other than the antigen from which the T cell or B cell epitope is derived, such as a granulocyte-macrophage colony-stimulating factor (GM-CSF). On the other hand, a “heterologous polypeptide” may be one derived from another portion of the T cell or B cell protein that is not immediately contiguous to the T cell or B cell epitope. A “heterologous polypeptide” may contain modifications of a naturally occurring protein sequence or a portion thereof, such as deletions, additions, or substitutions of one or more amino acid residues. Regardless of the origin of the “heterologous polypeptide” (i.e., whether it is derived from the antigen from which the T cell or B cell epitope is derived or another protein), the fusion peptide should not contain a subsequence of the human T cell or B cell that encompasses the amino acid sequences in Table 3 and Tables 15-47 and have more than 5, 10, 15, 20, 25, 30, 40, 50, 60, 70, 80, 90, 100, 120, 150, 180, 200, 250, 300, 350, 400, 450, 500, 600, 700, 800, 900, 1000, or more amino acids in length. In some exemplary embodiments, the fusion peptide should not contain a subsequence of the human T cell or B cell that encompasses the amino acid sequences in Table 3 and Tables 15-47 and have more than 500 amino acids in length. In some exemplary embodiments, a “heterologous polypeptide” for use in the present disclosure has no more than 15-20 amino acids in length; in other embodiments, a “heterologous polypeptide” has at least 100 amino acids in length. In some exemplary embodiments, a “heterologous polypeptide” is a recombinant polynucleotide (or a copy or complement of a recombinant polynucleotide) that has been manipulated using well known methods. For example, a “heterologous polypeptide” can comprise a recombinant expression cassette comprising a promoter operably linked to a second polynucleotide (e.g., a coding sequence of the antigen from which the T cell or B cell epitope is derived). In this context, the promoter is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). As another example, the “heterologous polypeptide” may comprise a promoter that is heterologous to a second polynucleotide encoding the polypeptide of interest (e.g., a T cell and/or a B cell epitope) or a fragment thereof, and a third polynucleotide encoding a detecting (e.g., a tag or identifier) molecule. The detecting molecule can be a fluorescent protein, e.g., a green fluorescent protein (GFP) or a variant or the like, such as DsRed and other red fluorescent protein.

The word “fuse” or “fused,” as used in the context of describing a peptide of this disclosure that comprises a T cell or B cell epitope joined with a heterologous polypeptide, refers to a connection between the epitope and the heterologous polypeptide by any covalent bond, including a peptide bond.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform to codon preference in a specific host cell.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “recombinant,” when used with reference, e.g., to a cell, or nucleic acid, protein, or vector, indicates that the cell, nucleic acid, protein or vector, has been modified by the introduction of a nucleic acid or protein from an outside source or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all.

The term “administration” or “administering” refers to various methods of contacting a substance with a mammal, especially a human. Modes of administration may include, but are not limited to, methods that involve contacting the substance intravenously, intraperitoneally, intranasally, transdermally, topically, subcutaneously, parentally, intramuscularly, orally, or systemically, and via injection, ingestion, inhalation, implantation, or adsorption by any other means. One exemplary means of administration of a T cell and/or B cell peptide of this disclosure or a fusion peptide comprising a T cell and/or B cell epitope (e.g., a T cell or B cell epitope derived from the S protein, N protein, or full length of SARS-CoV and/or SARS-CoV-2) and a heterologous polypeptide is via intramuscular delivery, where the peptide or fusion peptide can be formulated as a pharmaceutical composition in the form suitable for intramuscular injection, such as an aqueous solution, a suspension, or an emulsion, etc. Other means for delivering a T cell and/or B cell epitope or a fusion peptide of this disclosure includes intradermal injection, subcutaneous injection, intravenous injection, or transdermal application as with a patch.

An “effective amount” of a certain substance refers to an amount of the substance that is sufficient to effectuate a desired result. For instance, an effective amount of a composition comprising a peptide of this disclosure that is intended to induce an anti-SARS-CoV or SARS-CoV-2 immunity is an amount sufficient to achieve the goal of inducing the immunity when administered to a subject. The effect to be achieved may include the prevention, correction, or inhibition of progression of the symptoms of a disease/condition and related complications to any detectable extent. The exact quantity of an “effective amount” will depend on the purpose of the administration, and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

A “therapeutically effective amount” of a substance/molecule of the disclosure may vary depending on factors such as: the disease state, age, sex and weight of the individual, and the ability of the substance/molecule to induce a desired response in the individual. A therapeutically effective amount is also an amount that has a therapeutically beneficial effect over any toxic or detrimental effect of the substance/molecule.

A “prophylactically effective amount” refers to an amount of dosage and time necessary to effectively achieve the desired prophylactic result. Since prophylactic doses are used in individuals prior to or early in the disease, the prophylactically effective amount is typically (but not necessarily) less than the therapeutically effective amount.

A “physiologically or pharmaceutically acceptable excipient” is an inert ingredient used in the formulation of a composition of this disclosure, which contains the active ingredient(s) of a T cell and/or B cell peptide or a fusion peptide comprising a T cell and/or B cell peptide and a heterologous polypeptide and is suitable for use, e.g., by injection into a patient in need thereof. This inert ingredient may be a substance that, when included in a composition of this disclosure, provides a desired pH, consistency, color, smell, or flavor of the composition.

As used herein, the term “T cell immune response” refers to activation of antigen specific T cells as measured by proliferation or expression of molecules on the cell surface or secretion of proteins such as cytokines.

As used herein, the term “B cell immune response” refers to either a T cell-independent immune response or a T cell-dependent immune response. In a T cell-independent response, B cells respond directly to the antigen. In a T cell-dependent immune response, B cells rely on the assistance from T cells to respond. Activated B cells may express IgA, IgE, IgG or retain IgM expression. Cytokines produced by T cells and others may determine what isotype the B cells express. Several general techniques are commonly used to identify antigen-specific B cells. Non-limiting examples are B cell enzyme linked immunospot (ELISPOT), limiting dilution, flow cytometry, adoptive transfer, microscopy, and B cell receptor (BCR) transgenic mice.

As used herein, the term “immune response” generally refers to the immune system recognizing the antigens, e.g., proteins, on the surface of substances or microorganisms, such as bacteria or virus, and attacks and destroys, or tries to destroy, them. As described herein, the term “immune response” refers to a cell-mediated (T-cell) immune response and/or an antibody (B-cell) immune response.

As described herein, the terms “immunogenic composition,” or “vaccine” are used interchangeably and refer to a composition that elicits an immune response in a subject, especially a human. An immunogenic composition or vaccine can be used prophylactically to prevent COVID-19 (SARS-CoV-2) or other coronavirus diseases (e.g., SARS-CoV).

As described herein, the term “immunity” refers to protection from an infectious disease. For instance, if a subject is immune to a disease, the object can be exposed to it without becoming infected.

As described herein, the term “vaccine” generally refers to a preparation that is used to stimulate the body's immune system to generate immunity for a disease. For instance, it refers to an antigen-containing formulation consisting of whole pathogenic organisms (killed or attenuated) or components of these organisms (such as proteins, peptides or polysaccharides) for conferring immunity against disease caused by these organisms. Vaccine formulations may be natural, synthetic or obtained by recombinant DNA techniques. A vaccine may be administered in any route known in the field.

As described herein, the terms “vaccination” or “immunization” or “inoculation” are used interchangeably. They refer to a process by which a person becomes protected against a disease by introducing a vaccine into the body to produce protection from the specific disease. For example, vaccines are usually administered intramuscularly through needle injections. As another example, vaccines can be administered by mouth (oral) or sprayed into the nose (intranasal).

As described herein, the term “immunogenic” refers to the ability of an immunogen, antigen or vaccine to stimulate an immune response.

As used herein, the term “antigen” is defined as any substance capable of eliciting an immune response. For example, an “antigen” may be a small molecule or a macromolecule such as a protein, a peptide, a polysaccharide, a nucleic acid, a lipid, or a biomolecule.

As used herein, the term “antigen-specific” refers to a property of a population of cells such that the provision of a particular antigen or antigen fragment causes the proliferation of a specific cell.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to fusion proteins can be selected to obtain only those polyclonal antibodies that are specifically immunoreactive with fusion protein and not with individual components of the fusion proteins. This selection may be achieved by subtracting out antibodies that cross-react with the individual antigens. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The terms “antibody” and “immunoglobulin” are used interchangeably in a broad sense and include monoclonal antibodies (e.g., full-length or intact monoclonal antibodies), polyclonal antibodies, multivalent antibodies, multispecific antibodies (e.g., bispecific antibodies, so long as they exhibit the desired biological activity), and may also include certain antibody fragments (as described in more detail herein). The antibody may be a chimeric antibody, a human antibody, a humanized antibody, and/or an affinity matured antibody.

The terms “a,” “an,” and “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, the term “about” denotes a range of +10% of a specified value. For instance, “about 10” denotes a range of 9-11.

The term “subject” or “subject in need of treatment” refers to an individual who seeks medical attention due to risk of, or actual sufferance from, a condition involving undesirable inflammation (e.g., pneumonia or an infection that inflames air sacs) or a condition involving infection of the respiratory tract (e.g., the upper respiratory tract, the lungs). Subjects or individuals in need of treatment include those that demonstrate symptoms of infection of the respiratory tract or those are at risk of later developing the disease or disorder and/or its symptoms. For example, the subject may experience or is at risk of sufferance from a SARS-CoV infection. The subject may have one or more symptoms of SARS-CoV infection as defined by the Centers for Disease Control and Prevention (www.cdc.gov/coronavirus/2019-ncov/symptoms-testing/symptoms.html). The subject may experience or have a wide range of symptoms ranging from mild to severe illness. Exemplary symptoms are fever or chills, cough, shortness of breath or difficulty breathing, fatigue, muscle or body aches, headache, new loss of taste or smell, sore throat, congestion or running nose, nausea or vomiting, or diarrhea, trouble breathing, persistent pain or pressure in the chest, new confusion, inability to wake or stay awake, and/or pale, gray, or blue-colored skin, lips, or nail beds, depending on skin tone, or combinations thereof. Symptoms may appear 0-60 days, 1-30 days, or 2-14 days after exposure to the source of infection (e.g., SARS-CoV or SARS-CoV-2 viruses). The term subject can include both animals, especially mammals, and humans.

### DETAILED DESCRIPTION

### I. Introduction

Efforts have been made to develop vaccines against the novel SARS-CoV-2. Described herein are peptides, their related compositions, and methods of uses thereof. The disclosed peptides comprise at least one T cell epitope and/or at least one B cell epitope for eliciting an immune response in a subject against SARS-CoV-2. Epitopes that provide broad coverage broadly and region-specific are also explored.

We sought to gain insights for vaccine design against SARS-CoV-2 by considering the high genetic similarity between SARS-CoV-2 and SARS-CoV, which caused the outbreak in 2003, and leveraging existing immunological studies of SARS-CoV. By screening the experimentally-determined SARS-CoV-derived B cell and T cell epitopes in the immunogenic structural proteins of SARS-CoV, we identified a set of B cell and T cell epitopes derived from the spike (S) and nucleocapsid (N) proteins that map identically to SARS-CoV-2 proteins. As no mutation has been observed in these identified epitopes among the 120 available SARS-CoV-2 sequences (as of 21 Feb. 2020), immune targeting of these epitopes may potentially offer protection against this novel virus. For the T cell epitopes, we performed a population coverage analysis of the associated MHC alleles and proposed a set of epitopes that is estimated to provide broad coverage globally, as well as in China. The findings provide a screened set of epitopes that can help guide experimental efforts towards the development of vaccines against SARS-CoV-2.

Unless specifically stated, SARS-CoV-2 is referred to the original SARS-CoV-2 (GenBank accession no. NC_045512) or any variants thereof. A variant has one or more mutations that differentiate it from other variants of the SARS-CoV-2 viruses. According to The Center for Disease Control and Prevention (CDC), current known variants include, but are not limited to, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), Delta (B.1.617.2 and AY lineages), and Omicron (B.1.1.529 and BA lineages). In the context of this disclosure, a polynucleotide of the full length SARS-CoV-2 (GenBank accession no. NC_045512), a variant, or a fragment thereof may activates a T cell and/or B cell response. The polynucleotides may have at least 50%, at least 60%, at least 70%, at least 80%, at least 85%, at least 90%, at least 95%, at least 99%, or more sequence identity with the full length SARS-CoV-2 (GenBank accession no. NC_045512), a variant, or a fragment thereof.

### II. Chemical Synthesis of Peptides

The peptides of the present disclosure, particularly those of relatively short length (e.g., no more than 50-100 amino acids, no more than 10-500 amino acids), may be synthesized chemically using conventional peptide synthesis or other protocols well known in the art. In some embodiments, the peptides are of no more than about 10, 20, 30, 40, 50, 60, 70, 80, 90, 100, 150, 200, 250, 300, 350, 400, 450, or 500 amino acids, or in the range of from about 10 to about 50, 100, 200, 300, 400 or 500 amino acids, from about 20 to about 50, 100, 200, 300, 400 or 500 amino acids, from about 50 to about 100, 150, 200, 300, 400 or 500 amino acids, from about 100 to about 150, 200, 250, 300, 400 or 500 amino acids, or from about 200 to about 250, 300, 350, 400 or 500 amino acids.

Peptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, e.g., Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### III. Recombinant Production of Peptides

### A. General Recombinant Technoloy

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

Recombinant production is an effective means to obtain peptides of this disclosure, particularly those of relatively large molecular weight, for example, a fusion peptide of a HER-2/Neu epitope and a GM-CSF. The sequence of a polynucleotide encoding a peptide of this disclosure, and synthetic oligonucleotides can be verified after cloning or subcloning using, e.g., the chain termination method for sequencing double-stranded templates of Wallace et al., Gene 16: 21-26 (1981).

### B. Construction of an Expression Cassette

i. Obtaining a Polynucleotide Sequence Encoding a Peptide

A polynucleotide sequence encoding a peptide of this disclosure can be obtained by chemical synthesis, or can be purchased from a commercial supplier, which may then be further manipulated using standard techniques of molecular cloning.

ii. Modification of Nucleic Acids for Preferred Codon Usage in Host Organism

The polynucleotide sequence encoding a peptide of this disclosure can be optionally altered to coincide with the preferred codon usage of a particular host. For example, the preferred codon usage of one strain of bacterial cells can be used to derive a polynucleotide that encodes a peptide of the disclosure and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the peptides of this disclosure.

Following verification of the coding sequence, the peptide of the present disclosure can be produced using routine techniques in the field of recombinant genetics.

### C. Expression Systems

To obtain high level expression of a nucleic acid encoding a peptide of the present disclosure, one typically subclones a polynucleotide encoding the peptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing a peptide of this disclosure are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available.

In one embodiment, the eukaryotic expression vector is an adenoviral vector, an adeno-associated vector, a retroviral vector, or a yeast artificial chromosome (YAC). In some embodiments, the eukaryotic (e.g., mammalian) expression vector is a pEAK10, pEAK12, pEAK13, pCDNA3.0, pCDNA4.0, pCDM7, pCDM8, pCDM10, pCDM12. In some embodiments, the promoter is CMV, EF1α, or SV40.

For instance, the expression system comprises a promoter operably linked to the peptide nucleic acid (e.g., cDNA) sequence such that it is under control of a promoter. The term “promoter” describes the combination of the promoter (RNA polymerase binding site) and operators. The promoter may function in vivo or in a cell-free system. Any number of promoters may be used depending on the needs and preferences of the practitioner. Promoters for controlling recombinant protein, (e.g., peptide) expression can be any promoter for any DNA dependent RNA polymerase, for example. Generally a promoter (e.g., T7, T3, and SP6 RNA promoters and compatible RNA polymerases) is selected for in vitro expression for producing recombinant protein in a bacterial system, e.g., E. coli, which usually requires the molecular inducer isopropyl-β-D-thiogalactoside (IPTG) for regulating the promoter's transcriptional activity. In some cases, a modified Self-Inducible Expression system that utilizes lactose as an inducer may be used, as described in Briand et al, A self-inducible heterologous protein expression system in Escherichia coli. Sci Rep 6, 33037(2016).

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of a peptide of this disclosure in host cells. A typical expression cassette thus contains a promoter operably linked to the polynucleotide sequence encoding the peptide and signals required for efficient polyadenylation of the transcript, ribosome binding sites, and translation termination. The nucleic acid sequence encoding the peptide is typically linked to a cleavable signal peptide sequence to promote secretion of the peptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene (e.g., encoding the heterologous polypeptide), introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the peptide of this disclosure under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a peptide of the present disclosure) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

### D. Transfection Methods

Standard transfection methods are used to produce bacterial, mammalian, yeast, insect, or plant cell lines that express large quantities of a peptide of this disclosure, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the peptide of this disclosure.

### E. Detection of Recombinant Expression of a Peptide in Host Cells

After the expression vector is introduced into appropriate host cells, the transfected cells are cultured under conditions favoring expression of the peptide of this disclosure. The cells are then screened for the expression of the recombinant peptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a peptide of this disclosure in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a peptide of the present disclosure, particularly one containing a sufficiently large heterolougs polypeptide (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the peptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6: 511-519 (1976).

### F. Purification of Peptides

i. Purification of Chemically Synthesized Peptides

Purification of synthetic peptides is accomplished using various methods of chromatography, such as reverse phase HPLC, gel permeation, ion exchange, size exclusion, affinity, partition, or countercurrent distribution. The choices of appropriate matrices and buffers are well known in the art.

ii. Purification of Chemically Synthesized Peptides
1. Purification of Peptides from Bacterial Inclusion Bodies
a. Solubility Fractionation

Often as an initial step, and if the protein mixture is complex, an initial salt fractionation can separate many of the unwanted host cell proteins (or proteins derived from the cell culture media) from the recombinant protein of interest, e.g., a peptide of the present disclosure. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

b. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a peptide of the present disclosure. The retentate of the ultrafiltration is then ultrafiltered against a membrane with a molecular cut off greater than the molecular weight of the peptide of interest. The recombinant protein will pass through the membrane into the filtrate. The filtrate can then be chromatographed as described below.

c. Column Chromatography

A protein of interest (such as a peptide of the present disclosure) can also be separated from other proteins on the basis of its size, net surface charge, hydrophobicity, or affinity for ligands. In addition, antibodies raised against a peptide of this disclosure can be conjugated to column matrices and the peptide immunopurified. All of these methods are well known in the art.

It will be apparent to one of skill that chromatographic techniques can be performed at any scale and using equipment from many different manufacturers (e.g., Pharmacia Biotech).

When a peptide of the present disclosure is produced recombinantly by transformed bacteria in large amounts, typically after promoter induction, although expression can be constitutive, the peptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, NY). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18:182-190 (2000).

Alternatively, it is possible to purify recombinant polypeptides, e.g., a peptide of this disclosure, from bacterial periplasm. Where the recombinant polypeptide is exported into the periplasm of the bacteria, the periplasmic fraction of the bacteria can be isolated by cold osmotic shock in addition to other methods known to those of skill in the art (see e.g., Ausubel et al., supra). To isolate recombinant peptides from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant peptides present in the supernatant can be separated from the host proteins by standard separation techniques well known to those of skill in the art.

### 2. Standard Protein Separation Techniques for Purification

When a recombinant polypeptide, e.g., a peptide of the present disclosure, is expressed in host cells in a soluble form, its purification can follow the standard protein purification procedure described below. This standard purification procedure is also suitable for purifying peptides obtained from chemical synthesis.

### G. Confirmation of Peptide Sequence

The amino acid sequence of a peptide of this disclosure can be confirmed by a number of well established methods. For example, the conventional method of Edman degradation can be used to determine the amino acid sequence of a peptide. Several variations of sequencing methods based on Edman degradation, including microsequencing, and methods based on mass spectrometry are also frequently used for this purpose.

### H. Modification of Peptides

The peptides of the present disclosure can be modified to achieve more desirable properties. The design of chemically modified peptides and peptide mimics that are resistant to degradation by proteolytic enzymes or have improved solubility or binding ability is well known.

Modified amino acids or chemical derivatives of the T cell or B cell peptides or fusion peptides of this disclosure may contain additional chemical moieties of modified amino acids not normally a part of the T cell or B cell protein. Covalent modifications of the peptides are within the scope of the present disclosure. Such modifications may be introduced into a peptide by reacting targeted amino acid residues of the peptide with an organic derivatizing agent that is capable of reacting with selected side chains or terminal residues. The following examples of chemical derivatives are provided by way of illustration and not by way of limitation.

The design of peptide mimics which are resistant to degradation by proteolytic enzymes is known to those skilled in the art. See e.g., Sawyer, Structure-Based Drug Design, P. Verapandia, Ed., N.Y. (1997); U.S. Pat. Nos. 5,552,534 and 5,550,251. Both peptide backbone and side chain modifications may be used in designing secondary structure mimicry. Possible modifications include substitution of D-amino acids, N^{α}-Me-amino acids, C_{α}-Me-amino acids, and dehydroamino acids. To this date, a variety of secondary structure mimetics have been designed and incorporated in peptides or peptidomimetics.

Other modifications include substitution of a natural amino acid with an unnatural hydroxylated amino acid, substitution of the carboxy groups in acidic amino acids with nitrile derivatives, substitution of the hydroxyl groups in basic amino acids with alkyl groups, or substitution of methionine with methionine sulfoxide. In addition, an amino acid of a HER-2/Neu peptide or a fusion peptide of this disclosure can be replaced by the same amino acid but of the opposite chirality, i.e., a naturally-occurring L-amino acid may be replaced by its D-configuration.

### I. Recombinant Proteins and Synthetic Peptides

Full length recombinant whole or variants thereof of SARS-CoV or SARS-CoV-2 virus and/or other coronavirous, or recombinant functional proteins, e.g., S protein, N protein, M protein, from SARS-CoV or SARS-CoV-2, and/or other coronavirous may be generated and purified using technologies known in the field. For example, full length SARS-CoV-2 can be generated based on “transformation-associated recombination” (TAR) in yeast (Thao et al. (2020), “Rapid reconstruction of SARS-CoV-2 using a synthetic genomics platform,”. bioRxiv, 2020.02.21.959817). As an example, synthetic proteins (e.g., defined as >35 amino acids) and peptides from the SARS-CoV or SARS-CoV-2 recombinant binding domain (RBD) may be synthesized in the Peptide Core at Los Alamos National Laboratory. Positive control rabbit serum (polyclonal, against SARS/SARS-CoV-2 Coronavirus spike protein subunit 1) is Invitrogen PA5-81795. See e.g., Schein et al., 2021. “Synthetic proteins for COVID-19 diagnostics.” Peptides. 143:170583. doi: 10.1016/j.peptides.2021.170583.

In some embodiments, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100, 150, 200, 250, 300, 350, 400, 450, 500 or longer than 500 amino acids. In another example, any protein that includes a stretch of 20, 30, 40, 50, 100, 150, 200, 250, 300, 350, 400, 450, or 500 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, 100, 150, 200, 250, 300, 350, 400, 450, or 500 amino acids that are greater than 40%, 50, 60%, 70%, 80%, 90%, 95%, or 100% identical to any of the sequences described herein, wherein the protein has a stretch of 5, 10, 15, 20, 25, or 30 amino acids that are less than 95%, 90%, 85%, 80%, 75%, 70%, 65% to 60% identical to any of the sequences described herein can be utilized in accordance with the disclosure.

### IV. Fusing T Cell and/or B Cell Epitope with a Heterologous Polypeptide

In one aspect of this disclosure, a peptide corresponding to a SARS-CoV or SARS-CoV-2 promiscuous T cell epitope or B cell epitope is attached to a heterologous polypeptide via a covalent bond to form a fusion peptide, such that the ability of the SARS-CoV or SARS-CoV-2 epitope to induce a T cell or B cell response is enhanced. Usually, this covalent bond is a peptide bond and the SARS-CoV and/or SARS-CoV-2 epitope and the heterologous polypeptide form a new polypeptide. This peptide bond may be a direct peptide bond between the SARS-CoV and/or SARS-CoV-2 epitope and the heterologous polypeptide, or it may be an indirect peptide bond provided by way of a peptide linker between the SARS-CoV or SARS-CoV-2 epitope and the heterologous polypeptide. As an example, the SARS-CoV and/or SARS-CoV-2 epitope may be linked to a detecting molecule such as a GFP, RFP, dsRed, or the like. As another example, the SARS-CoV and/or SARS-CoV-2 epitope may be linked to an affinity tag such as a six histidine (6His) on the N-terminal or the C-terminal, or the like.

Other covalent bonds are also suitable for the purpose of fusing the SARS-CoV or SARS-CoV-2 peptide with the heterologous polypeptide. For instance, a functional group (such as a non-terminal amine group, a non-terminal carboxylic acid group, a hydroxyl group, and a sulfhydryl group) of one peptide may easily react with a functional group of the other peptide and establish a covalent bond, other than a peptide bond, that conjugates the two peptides. A covalent connection between a peptide of a SARS-CoV or SARS-CoV-2 epitope and a heterologous polypeptide can also be provided by way of a linker molecule with suitable functional group(s). Such a linker molecule can be a peptide linker or a non-peptide linker. A linker may be derivatized to expose or to attach additional reactive functional groups prior to conjugation. The derivatization may involve attachment of any of a number of molecules such as those available from Pierce Chemical Company, Rockford, Illinois.

### V. Functional Assays

Adaptive immune response is mediated by the T lymphocytes and B lymphocytes. T lymphocytes express T-cell receptors (TCR) and detect short peptides produced by a proteolytic machinery and presented by major histocompatibility complex (MHC) molecules at the cell surface. (Bercovici et al., 2000). Through antigen processing, T lymphocytes are able to detect foreign peptides synthesized by infected cells. T cells which express CD8 molecules have the capacity to lyse directly the target cells. A subset of CD4+ T lymphocytes is specialized in regulating the immune response via cytokine secretion and activation of the antigen-presenting cells (APC). Chromium release assays and limiting-dilution analyses have been commonly used to measure specific T-cell responses. Additionally, various assays are available for immune monitoring of specific T-cell responses. These various assays are schematically divided into functional assays, which measure the secretion of a particular cytokine (ELISPOT and intracellular cytokines); assays which assess the specificity of the T cells irrespective of their functionality and which are based on structural features of the TCR (tetramers and immunoscope); and assays aimed at detecting T-cell precursors by amplifying cells that proliferate in response to antigenic stimulation.

A SARS-CoV or SARS-CoV-2 epitope of this disclosure (or a fusion peptide comprising a SARS-CoV or SARS-CoV-2 epitope and a heterologous polypeptide) is useful for its capability to induce a T cell immune response specific to a SARS-CoV or SARS-CoV-2 protein, when the epitope is presented by an APC that may have one of a HLA-A, HLA-B, and/or HLA-DR allele. Various functional assays can be used to confirm the ability of a SARS-CoV or SARS-CoV-2 epitope to induce such a SARS-CoV or SARS-CoV-2 specific T cell immune response in a promiscuous manner with regard to antigen presenting cells of different HLA alleles, including proliferation assay and flow cytometry assays detecting the binding between a T cell receptor and a peptide epitope or the production of cytokines by T cells.

As an illustration, the function assay ELISPOT may be used for this purpose. The ELISPOT (enzyme-linked immunospot) technique detects T cells that secrete a given cytokine (e.g., gamma interferon [IFN-γ]) in response to an antigenic stimulation (e.g., an antigen presented by SARS-CoV or SARS-CoV-2). Briefly, T cells are cultured with antigen-presenting cells in wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells specific for a given antigen in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor, and granzyme B-secreting lymphocytes.

As another illustration, the structural assay tetramers may be used for this purpose. T cells recognize short peptides presented by MHC molecules through their clonotypic TCR. Tetramers of MHC class I-peptide complexes have been used in cytometry to enumerate, characterize, and purify peptide-specific CD8 cells. The heavy and light chains of the MHC are produced in Escherichia coli, solubilized in urea, and refolded in vitro in the presence of high concentrations of the antigenic peptide. The refolded complexes are purified by gel filtration, and a single biotin is added at the C-terminal end of the heavy chain using the bacterial BirA enzyme. Incubation with fluorescent streptavidin yields tetramers which can be used like any clonotypic antibody. Tetramers of MHC class II molecules may also be produced and used to analyze CD4⁺ T-cell responses.

B lymphocytes recognize intact proteins and produce immunoglobulins (Ig). (Chaplin, 2010). B cell response is activated in two pathways: T cell-dependent and T cell-independent. In a T-cell dependent manner, antigens that activate T cells as well as B cells establish Ig responses in which T cells provide ‘help’ for the B cells to mature. T cell-independent B cell activation occurs without the assistance of T cell co-stimulatory proteins. In the absence of co-stimulators, monomeric antigens are unable to activate B cells. Polymeric antigens with a repeating structure, in contrast, are able to activate B cells, probably because they can crosslink and cluster Ig molecules on the B cell surface. T cell-independent antigens include bacterial lipopolysaccharide (LPS), certain other polymeric polysaccharides, and certain polymeric proteins.

Several techniques have been used for probing the B cell response in vitro and in vivo by taking advantage of the specificity of B cell receptor (BCR)-associated and secreted antibodies. These include ELISPOT, flow cytometry, mass cytometry, and fluorescence microscopy to identify and/or isolate primary antigen-specific B cells. (Jim Boonyaratanakornkit and Justin Taylor, 2019). The ELISPOT assay is particularly suitable for the purpose of detecting B cell epitope response to SARS-CoV and/or SARS-CoV-2 infection in relation to the disclosure described herein.

Another example for confirming B cell immune response specific to a SARS-CoV or SARS-CoV-2 protein is flow cytometry-based analysis of antigen-specific B cells. This technique is dependent on labeling antigen with a fluorescent tag to allow detection. Fluorochromes can either be attached covalently via chemical conjugation to the antigen, expressed as a recombinant fusion protein, or attached non-covalently by biotinylating the antigen. After biotinylation, fluorochrome-conjugated streptavidin is added to generate a labeled tetramer of the antigen. Biotinylation of the antigen may be set at a ratio <1 biotin to 1 antigen. Alternatively, site directed biotinylation can be accomplished by adding either an AviTag or BioEase tag to the recombinant antigen prior to expression.

### VI. Method for Eliciting an Immune Response in a Subject

The present disclosure further provides a method for eliciting an immune response in a subject in need thereof such as at risk of exposure to SARS-CoV or SARS-CoV-2 infection. According to the CDC, the term exposure refers to contact with infectious agents (bacteria or virus, e.g., SARS-CoV or SARS-CoV-2) in a manner that promotes transmission and increases the likihood of disease. For instance, a subject who has close contact with someone who has COVID-19 is considered at risk of exposure to the disease. The term close contact refers to within 6 feet of someone for a cumulative total of 15 minites or more over a 24-hour period. Subjects who have underlying medical conditions (e.g., immune-compromised due to treatment of immunosuppressants, under chemotherapy, prior heart and cardio diseases, prior pulmonary diseases, cancer, diabetes, obesity, etc.), age (e.g., 65 or over), geneteic predisposition, and/or pregnant or recently pregnant. The subject may be at risk for severe illness when contracted with COVID-19 such that the subject may need hospitalization, intensive care, a ventilator to help them breathe or they may even die. Elicitation an immune response in a subject may provide prophylactic and therapeutic applications. The method includes the following steps: first, lymphocytes including at least a T cell epitope, and/or at least a B cell epitope, and/or optionally an antigen-presenting cell are obtained from a patient. Suitable samples that yield such lymphocytes include blood, and lymph nodes or lymphatic fluids. Second, at least a T cell epitope, and/or at least a B cell epitope, and/or optionally an antigen-presenting cell are exposed to a SARS-CoV or SARS-CoV-2 peptide (or a fusion peptide comprising the SARS-CoV or SARS-CoV-2 peptide and a heterologous peptide) of this disclosure under conditions that would allow, e.g., proper presentation of a T cell epitope by the antigen-presenting cell to the T cell. Third, signs of a T cell response and/or B cell response is measured in vitro by means well known in the art such as ELISPOT, ELISA, proliferation assay, or flow cytometry. When a T cell response and/or B cell response is detected by any of these methods, it can be concluded that there exists a T cell and/or a B cell immune response specific to a SARS-CoV or SARS-CoV-2 protein in the patient.

### A. Vaccines

In certain preferred embodiments of the present disclosure, vaccines are provided. The vaccines will generally comprise a peptide comprising one or more T cell and/or B cell epitopes derived from the S protein, N protein, or full length or a fragment thereof, of a SARS-CoV and/or SARS-CoV-2, such as those discussed above, in combination with an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g., U.S. Pat. No. 4,235,877). Vaccine preparation is generally described in, for example, Powell & Newman, eds., Vaccine Design (the subunit and adjuvant approach) (1995). Pharmaceutical compositions and vaccines within the scope of the present disclosure may also contain other compounds, which may be biologically active or inactive. For example, one or more T cell and/or B cell epitopes derived from immunogenic portions of other coronavirus antigens (e.g., MERS-CoV, HCoV-HKU1, HCoV-229E, HCoV-OC43, and HCoV-NL63) may be present, either incorporated into a fusion polypeptide or as a separate compound, within the composition or vaccine. Accordingly, a pharmaceutical vaccine formulation may comprise the peptide comprising the one or more T cell and/or B cell epitopes derived from the S protein, N protein, or full length or a fragment thereof, of a SARS-CoV and/or SARS-CoV-2, one or more adjuvants, pharmaceutically-acceptable carriers or other ingredients including immunological adjuvants routinely provided in vaccine formulation. Suitable adjuvants are described in detail below.

In some embodiments, the peptide comprising the one or more T cell and/or B cell epitopes derived from the S protein, N protein, or full length or a fragment thereof, of a SARS-CoV and/or SARS-CoV-2 is biologically produced such as using an expression cassette in a host cell. Short to medium length peptides, for example peptides that do no require specific folding, can be chemically synthesized. Large scale production of chemically synthesized peptides may be used for manufacturing large quantities of peptide vaccines. See e.g., Bray, B. L., 2003. Large-scale manufacture of peptide therapeutics by chemical synthesis. Nature Reviews Drug Discovery, 2(7), pp. 587-593.

In some embodiments, the peptide comprising the one or more T cell and/or B cell epitopes derived from the S protein, N protein, or full length or a fragment thereof, of a SARS-CoV and/or SARS-CoV-2 is a cationic peptide. As described herein, a “cationic peptide” refers to a peptide that is positively charged at a pH in the range of 5.0 to 8.0. The net charge on the peptide or peptide cocktails is calculated by assigning a +1 charge for each lysine (K), arginine (R) or histidine (H), a −1 charge for each aspartic acid (D) or glutamic acid (E) and a charge of 0 for the other amino acid within the sequence. The charge contributions from the N-terminal amine (+1) and C-terminal carboxylate (−1) end groups of each peptide effectively cancel each other when unsubstituted. The charges are summed for each peptide and expressed as the net average charge. A suitable peptide has a net average positive charge of +1. Preferably, the peptide has a net positive charge in the range that is larger than +2.

In some embodiments, the peptide comprising the one or more T cell and/or B cell epitopes derived from the S protein, N protein, or full length or a fragment thereof, of a SARS-CoV and/or SARS-CoV-2 is an anionic peptide. As described herein, an “anionic molecule” refers to a molecule that is negatively charged at a pH in the range of 5.0-8.0. The net negative charge on the oligomer or polymer is calculated by assigning a −1 charge for each phosphodiester or phosphorothioate group in the oligomer.

Illustrative vaccines may contain polynucleotides encoding one or more of the polypeptides (e.g., T cell and/or B cell epitopes derived from one or more immunogenic portions of a SARS-CoV, SARS-CoV-2, and/or other coronaviruses) as described above, such that the polypeptide is generated in situ. As noted above, the polynucleotides may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198 (1998), and references cited therein. Appropriate nucleic acid expression systems contain the necessary polynucleotides sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In one embodiment, the polynucleotides may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627 (1988); Rosenfeld et al., Science 252:431-434 (1991); Kolls et al., Proc. Natl. Acad. Sci. USA 91:215-219 (1994); Kass-Eisler et al., Proc. Natl. Acad. Sci. USA 90:11498-11502 (1993); Guzman et al., Circulation 88:2838-2848 (1993); and Guzman et al., Cir. Res. 73:1202-1207 (1993). Techniques for incorporating polynucleotides into such expression systems are well known to those of ordinary skill in the art. The polynucleotides may also be “naked,” as described, for example, in Ulmer et al., Science 259:1745-1749 (1993) and reviewed by Cohen, Science 259:1691-1692 (1993). The uptake of naked polynucleotides may be increased by coating the polynucleotides onto biodegradable beads, which are efficiently transported into the cells. It will be apparent that a vaccine may comprise both a polynucleotide and a polypeptide component. Such vaccines may provide for an enhanced immune response.

In some embodiments, the polynucleotides is formulated in lipid nanoparticles. In some embodiments, the lipid nanoparticle is a mucus penetrating lipid nanoparticle. In some embodiments, the lipid nanoparticle is a solid lipid nanoparticle. Formulations for polynucleotides in lipid nanoparticles are discussed in U.S. patent Ser. No. 10/272,150, which is incorporated herein in its entirety.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In some embodiments the peptide vaccines and/or the polynucleotide vaccines of the disclosure are superior to conventional vaccines by a factor of at least 2 fold, 5 fold, 10 fold, 20 fold, 40 fold, 50 fold, 100 fold, 500 fold or 1,000 fold.

While any suitable carrier known to those of ordinary skill in the art may be employed in the vaccine compositions of this disclosure, the type of carrier will vary depending on the mode of administration. Compositions of the present disclosure may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this disclosure. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present disclosure may be formulated as a lyophilizate. Compounds may also be encapsulated within liposomes using well known technology.

### B. Adjuvants

Any of a variety of immunostimulants may be employed in the vaccines of this disclosure. For example, an adjuvant or an immune potentiator may be included. An adjuvant may act as a co-signal to prime T-cells and/or B-cells and/or NK cells as to the existence of an infection. Adjuvants useful in the present disclosure may include, but are not limited to, natural or synthetic. They may be organic or inorganic. In some embodiments, adjuvants useful in the present disclosure include adjuvants for vaccines (e.g., influenza vaccines) as shown in Table 48. Adjuvants for DNA nucleic acid vaccines (DNA) have been disclosed in, for example, Kobiyama, et al Vaccines, 2013, 1(3), 278-292, the contents of which are incorporated herein by reference in their entirety.

TABLE 48
Adjuvants.

| Adjuvant | |
|---|---|
| category | Adjuvant |
| Salts | Alum |
| Oil-in-water | MF59 (squalene, Span 85, polysorbate 80); AS03 |
| emulsions | (squalene, DL-α-tocopherol, polysorbate 80); AF03 |
| | (squalene, Brij 76); CoVaccine HT (squalene, |
| | polysorbate 80, sucrose fatty acid sulfate esters) |
| Saponins | Iscomatrix; Matrix-M |
| Glycolipids | Alpha-GalCer (alpha-galactosylceramide) |
| Liposomes | CCS/c (cationic liposomes of ceramide carbamoyl- |
| | spermine/cholesterol); CAF01 (cationic liposomes of |
| | DDA/TDB); Vaxfectin (cationic liposomes of GAP- |
| | DMORIE/DPyPE) |
| Bacterial | CTA1-DD (Cholera toxin subunit A); LT patch |
| components | (Escherichia coli enterotoxin); |
| | Salmonella and Escherichia coli flagellins |
| Cytokines | IL-12, IL-23; GM-CSF (Granulocyte-Monocyte |
| | Colony Stimulating Factor); Type 1 IFN (IFNα) |
| TLR agonists/ | GLA (glucopyranosyl lipid A) (TLR4); Bacterial |
| immuno- | flagellins (TLR5); CpG oligodeoxynucleotide (TLR9); |
| modulators | PolyI:C (TLR3); IC31 oligodeoxynucleotide (TLR9); |
| | sLAG-3 (IMP321) (MHC class II ligand); Chitosan; |
| | PCPP ((poly[di(carboxylatophenoxy)phosphazene]); |
| | Advax (delta inulin) |

Most adjuvants contain a substance designed to protect the antigen from rapid catabolism, such as aluminum hydroxide or mineral oil, and a stimulator of immune responses, such as lipid A, Bortadella pertussis or Mycobacterium species or Mycobacterium derived proteins. For example, delipidated, deglycolipidated M. vaccae (“pVac”) can be used. Suitable adjuvants are commercially available as, for example, Freund's Incomplete Adjuvant and Complete Adjuvant (Difco Laboratories, Detroit, MI); Merck Adjuvant 65 (Merck and Company, Inc., Rahway, NJ); AS-2 and derivatives thereof (SmithKline Beecham, Philadelphia, PA); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Selection of appropriate adjuvants will be evident to one of ordinary skill in the art. Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTAI-DD gene fusion protein, DOC/Alum Complex, Gamma Inulin, Gerbu Adjuvant, GM-CSF, GMDP, Recombinant hIFN-gammallnterferon-g, Interleukin-10, Interleukin-2, Interleukin-7, Sclavo peptide, Rehydragel LV, Rehydragel HPA, Loxoribine, M1F59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and/or Matrix-S.

Other adjuvants which may be co-administered with the polypeptides of the disclosure include, but are not limited to interferons, TNF-alpha, TNF-beta, chemokines such as CCL21, eotaxin, HMGB1, SA100-8alpha, GCSF, GMCSF, granulysin, lactoferrin, ovalbumin, CD-40L, CD28 agonists, PD-1, soluble PDI, L1 or L2, or interleukins such as IL-1, IL-2, IL-4, IL-6, IL-7, IL-10, IL-12, IL-13, IL-21, IL-23, IL-15, IL-17, and IL-18.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann & Coffman, Ann. Rev. Immunol. 7:145-173 (1989).

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, WA; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another preferred adjuvant comprises a saponin, such as Quil A, or derivatives thereof, including QS21 and QS7 (Aquila Biopharmaceuticals Inc., Framingham, MA); Escin; Digitonin; or Gypsophila or Chenopodium quinoa saponins. Other preferred formulations include more than one saponin in the adjuvant combinations of the present disclosure, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol® to increase viscosity, or may be formulated in a dry powder form with a powder excipient such as lactose.

In one embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159. Preferably the formulation additionally comprises an oil in water emulsion and tocopherol.

Other adjuvants include Montanide ISA 720 (Seppic, France), SAF-1 (Chiron, California, United States), ISCOMS (CSL), MF-59 (Chiron), the SBAS series of adjuvants (e.g., SBAS-2, AS2′, AS2,″ SBAS-4, or SBAS6, available from SmithKline Beecham, Rixensart, Belgium), Detox (Corixa, Hamilton, MT), RC-529 (Corixa, Hamilton, MT) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other adjuvants include adjuvant molecules of the general formula (I): HO(CH₂CH₂O)ₙ-A-R, wherein, n is 1-50, A is a bond or —C(O)—, R is C₁₋₅₀ alkyl or Phenyl C₁₋₅₀ alkyl.

One embodiment of the present disclosure consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438 (1996)) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. In some embodiments, the vaccine is formulated for induction of systemic or localized mucosal immunity through immunogen entrapment and co-administration with microparticles.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

### VII. Kits

The disclosure provides a variety of kits for conveniently and/or effectively carrying out methods of the present disclosure. Typically kits will comprise sufficient amounts and/or numbers of components to allow a user to perform multiple treatments of a subject(s). The disclosure provides kits for eliciting an immune response in a subject according to the method of the present disclosure. The kits typically include a container that contains a pharmaceutical composition having an effective amount of SARS-CoV-derived or SARS-CoV-2-derived T cell epitope polypeptides or a function fragment thereof, and/or B cell epitope polypeptides or a function fragment thereof), and/or the polypeptides optionally further comprising at least one heterologous polypeptide sequence. The kit may comprise nucleic acid sequences encoding the SARS-CoV-derived or SARS-CoV-2-derived T cell and/or B cell polypeptides, and/or the expression cassettes for expressing the T cell and/or B cell polypeptides, and/or the vectors for expressing the expression cassettes. The kit may optionally comprise an additional container containing a therapeutic agent against SARS-CoV or variants thereof, SARS-CoV-2 or variants thereof, and/or other coronavirus or variants (e.g., MERS-CoV, HCoV-HKU1, HCoV-229E, HCoV-OC43, and HCoV-NL63).

In some embodiments, the present disclosure provides kits comprising the SARS-CoV- and/or SARS-CoV-2-derived T cell and/or B cell epitopes (including any polypeptides, proteins or function fragments thereof) of the disclosure. The T cell and/or B cell epitopes may be derived from one or more functional proteins (e.g., S protein, N protein, or M protein) or full length SARS-CoV or SARS-CoV-2. In some embodiments, the kit further comprises polypeptides comprising one or more T cell and/or B cell epitopes derived from one or more of a SARS-CoV and/or SARS-CoV-2 variants. In some embodiments, the kit further comprises polypeptides comprising one or more T cell and/or B cell epitopes derived from one or more coronaviruses or variants thereof (e.g., MERS-CoV, HCoV-HKU1, HCoV-229E, HCoV-OC43, and HCoV-NL63). The kit may further comprise packaging and instructions and/or a delivery agent to form a formulation composition. The delivery agent may comprise a saline, a buffered solution, a lipidoid or any delivery agent disclosed herein.

The kits can be for protein or polypeptide production, comprising a polynucleotide comprising a translatable region of one or more T cell and/or B cell epitopes derived from a functional protein (e.g., S protein, N protein, or M protein) or full length SARS-CoV or SARS-CoV-2. In some embodiments, the kit further comprises a polynucleotide comprising a translatable region of one or more T cell and/or B cell epitopes derived from one or more coronaviruses (e.g., MERS-CoV, HCoV-HKU1, HCoV-229E, HCoV-OC43, and HCoV-NL63). The kit may further comprise packaging and instructions and/or a delivery agent to form a formulation composition. The delivery agent may comprise a saline, a buffered solution, a lipidoid or any delivery agent disclosed herein.

In some embodiments, the kit further comprises devices for administering the polypeptides or a protein or polypeptide product descried herein. For instance, the kit may comprise syringes, needles, and instructions on how to dispense the pharmaceutical composition, including description of the type of patients who may be treated (e.g., subjects who are at risk of having COVID-19 due to individual health condition, pre-existing condition of any kind, compromised immune system, living condition, age, or occupation; subjects who has close contact with someone who has COVID-19).

All publications and patent applications cited in this specification are herein incorporated by reference as if each individual publication or patent application were specifically and individually indicated to be incorporated by reference.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims.

### EXAMPLES

The following examples are provided by way of illustration only and not by way of limitation. Those of skill in the art will readily recognize a variety of non-critical parameters that could be changed or modified to yield essentially similar results.

### Example 1—Preliminary Identification of Potential Vaccine Targets for the Covid-19 Coronavirus (SARS-COV-2) Based on SARS-COV Immunological Studies

### Introduction

In this study, we sought to gain insights for vaccine design against SARS-CoV-2 by considering the high genetic similarity between SARS-CoV-2 and SARS-CoV, which caused the outbreak in 2003, and leveraging existing immunological studies of SARS-CoV. By screening the experimentally-determined SARS-CoV-derived B cell and T cell epitopes in the immunogenic structural proteins of SARS-CoV, we identified a set of B cell and T cell epitopes derived from the spike (S) and nucleocapsid (N) proteins that map identically to SARS-CoV-2 proteins. As no mutation has been observed in these identified epitopes among the 120 available SARS-CoV-2 sequences (as of 21 Feb. 2020), immune targeting of these epitopes may potentially offer protection against this novel virus. For the T cell epitopes, we performed a population coverage analysis of the associated MHC alleles and proposed a set of epitopes that is estimated to provide broad coverage globally, as well as in China. Our findings provide a screened set of epitopes that can help guide experimental efforts towards the development of vaccines against SARS-CoV-2.

Worldwide collaborative efforts from scientists are working on this disease and SARS-CoV-2 to develop effective interventions for controlling and preventing it [6-9].

Coronaviruses are positive-sense single-stranded RNA viruses belonging to the family Coronaviridae. These viruses mostly infect animals, including birds and mammals. In humans, they generally cause mild respiratory infections, such as those observed in the common cold. However, some recent human coronavirus infections have resulted in lethal endemics, which include the SARS (Severe Acute Respiratory Syndrome) and MERS (Middle East Respiratory Syndrome) endemics. Both of these are caused by zoonotic coronaviruses that belong to the genus Betacoronavirus within Coronaviridae. SARS-CoV originated from Southern China and caused an endemic in 2003. A total of 8098 cases of SARS were reported globally, including 774 associated deaths, and an estimated case-fatality rate of 14%-15% [10]. The first case of MERS occurred in Saudi Arabia in 2012. Since then, a total of 2,494 cases of infection have been reported, including 858 associated deaths, and an estimated high case-fatality rate of 34.4% [11]. While no case of SARS-CoV infection has been reported since 2004, MERS-CoV has been around since 2012 and has caused multiple sporadic outbreaks in different countries.

Like SARS-CoV and MERS-CoV, the recent SARS-CoV-2 belongs to the Betacoronavirus genus [12]. It has a genome size of ˜30 kilobases which, like other coronaviruses, encodes for multiple structural and non-structural proteins. The structural proteins include the spike (S) protein, the envelope (E) protein, the membrane (M) protein, and the nucleocapsid (N) protein. With SARS-CoV-2 being discovered very recently, there is currently a lack of immunological information available about the virus (e.g., information about immunogenic epitopes eliciting antibody or T cell responses). Preliminary studies suggest that SARS-CoV-2 is quite similar to SARS-CoV based on the full-length genome phylogenetic analysis [9,12], and the putatively similar cell entry mechanism and human cell receptor usage [9,13,14]. Due to this apparent similarity between the two viruses, previous research that has provided an understanding of protective immune responses against SARS-CoV may potentially be leveraged to aid vaccine development for SARS-CoV-2.

Various reports related to SARS-CoV suggest a protective role of both humoral and cell-mediated immune responses. For the former case, antibody responses generated against the S protein, the most exposed protein of SARS-CoV, have been shown to protect from infection in mouse models [15-17]. In addition, multiple studies have shown that antibodies generated against the N protein of SARS-CoV, a highly immunogenic and abundantly expressed protein during infection [18], were particularly prevalent in SARS-CoV-infected patients [19,20]. While being effective, the antibody response was found to be short-lived in convalescent SARS-CoV patients [21]. In contrast, T cell responses have been shown to provide long-term protection [21-23], even up to 11 years post-infection [24], and thus have also attracted interest for a prospective vaccine against SARS-CoV [reviewed in [25]]. Among all SARS-CoV proteins, T cell responses against the structural proteins have been found to be the most immunogenic in peripheral blood mononuclear cells of convalescent SARS-CoV patients as compared to the non-structural proteins [26]. Further, of the structural proteins, T cell responses against the S and N proteins have been reported to be the most dominant and long-lasting [27].

Here, by analyzing available experimentally-determined SARS-CoV-derived B cell epitopes (both linear and discontinuous) and T cell epitopes, we identify and report those that are completely identical and comprise no mutation in the available SARS-CoV-2 sequences (as of 21 Feb. 2020). These epitopes have the potential, therefore, to elicit a cross-reactive/effective response against SARS-CoV-2. We focused particularly on the epitopes in the S and N structural proteins due to their dominant and long-lasting immune response previously reported against SARS-CoV. For the identified T cell epitopes, we additionally incorporated the information about the associated MHC alleles to provide a list of epitopes that seek to maximize population coverage globally, as well as in China. Our presented results can potentially narrow down the search for potent targets for an effective vaccine against SARS-CoV-2, and help guide experimental studies focused on vaccine development.

### Materials and Methods

Acquisition and Processing of Sequence Data. A total of 120 whole genome sequences of SARS-CoV-2 were downloaded on 21 Feb. 2020 from the GISAID database (gisaid.org/CoV2020/) (Table 6). We excluded sequences that likely had spurious mutations resulting from sequencing errors, as indicated in the comment field of the GISAID data. These nucleotide sequences were aligned to the GenBank reference sequence (accession ID: NC_045512.2) and then translated into amino acid residues according to the coding sequence positions provided along the reference sequence for SARS-CoV-2 proteins (orf1a, orf1b, S, ORF3a, E, M, ORF6, ORF7a, ORF7b, ORF8, N, and ORF10). These sequences were aligned separately for each protein using the MAFFT multiple sequence alignment program [28]. Reference protein sequences for SARS-CoV and MERS-CoV were obtained following the same procedure from GenBank using the accession IDs NC_004718.3 and NC_019843.3, respectively.

TABLE 6
Whole genome sequences of SARS-CoV-2 downloaded from theGISAID database.

| | | | | |
|---|---|---|---|---|
| EPI_ISL_402119 | EPI_ISL_403930 | EPI_ISL_404228 | EPI_ISL_406533 | EPI_ISL_406597 |
| EPI_ISL_402123 | EPI_ISL_403932 | EPI_ISL_404253 | EPI_ISL_406534 | EPI_ISL_406716 |
| EPI_ISL_402124 | EPI_ISL_403933 | EPI_ISL_404895 | EPI_ISL_406535 | EPI_ISL_406717 |
| EPI_ISL_402125 | EPI_ISL_403934 | EPI_ISL_405839 | EPI_ISL_406536 | EPI_ISL_406798 |
| EPI_ISL_402127 | EPI_ISL_403935 | EPI_ISL_406030 | EPI_ISL_406538 | EPI_ISL_406800 |
| EPI_ISL_402128 | EPI_ISL_403936 | EPI_ISL_406031 | EPI_ISL_406592 | EPI_ISL_406801 |
| EPI_ISL_402129 | EPI_ISL_403937 | EPI_ISL_406034 | EPI_ISL_406593 | EPI_ISL_406844 |
| EPI_ISL_402130 | EPI_ISL_403962 | EPI_ISL_406036 | EPI_ISL_406594 | EPI_ISL_406862 |
| EPI_ISL_402132 | EPI_ISL_403963 | EPI_ISL_406223 | EPI_ISL_406595 | EPI_ISL_406970 |
| EPI_ISL_403929 | EPI_ISL_404227 | EPI_ISL_406531 | EPI_ISL_406596 | EPI_ISL_406973 |

| | | | |
|---|---|---|---|
| EPI_ISL_407071 | EPI_ISL_407896 | EPI_ISL_408479 | EPI_ISL_408665 |
| EPI_ISL_407073 | EPI_ISL_407976 | EPI_ISL_408480 | EPI_ISL_408666 |
| EPI_ISL_407079 | EPI_ISL_407987 | EPI_ISL_408481 | EPI_ISL_408667 |
| EPI_ISL_407084 | EPI_ISL_407988 | EPI_ISL_408482 | EPI_ISL_408668 |
| EPI_ISL_407193 | EPI_ISL_408008 | EPI_ISL_408484 | EPI_ISL_408669 |
| EPI_ISL_407214 | EPI_ISL_408009 | EPI_ISL_408486 | EPI_ISL_408670 |
| EPI_ISL_407215 | EPI_ISL_408010 | EPI_ISL_408488 | EPI_ISL_408976 |
| EPI_ISL_407313 | EPI_ISL_408430 | EPI_ISL_408489 | EPI_ISL_408977 |
| EPI_ISL_407893 | EPI_ISL_408431 | EPI_ISL_408514 | EPI_ISL_409067 |
| EPI_ISL_407894 | EPI_ISL_408478 | EPI_ISL_408515 | EPI_ISL_410044 |

| | | |
|---|---|---|
| EPI_ISL_410045 | EPI_ISL_410546 | EPI_ISL_411060 |
| EPI_ISL_410218 | EPI_ISL_410713 | EPI_ISL_411066 |
| EPI_ISL_410301 | EPI_ISL_410714 | EPI_ISL_411218 |
| EPI_ISL_410486 | EPI_ISL_410715 | EPI_ISL_411219 |
| EPI_ISL_410531 | EPI_ISL_410716 | EPI_ISL_411220 |
| EPI_ISL_410532 | EPI_ISL_410717 | EPI_ISL_411902 |
| EPI_ISL_410535 | EPI_ISL_410718 | EPI_ISL_411915 |
| EPI_ISL_410536 | EPI_ISL_410719 | EPI_ISL_411926 |
| EPI_ISL_410537 | EPI_ISL_410720 | EPI_ISL_411927 |
| EPI_ISL_410545 | EPI_ISL_410984 | EPI_ISL_411929 |

Acquisition and Filtering of Epitope Data. SARS-CoV-derived B cell and T cell epitopes were searched on the NIAID Virus Pathogen Database and Analysis Resource (ViPR) (www.viprbrc.org/; accessed 21 Feb. 2020) [29] by querying for the virus species name: “Severe acute respiratory syndrome-related coronavirus” from “human” hosts. We limited our search to include only the experimentally-determined epitopes that were associated with at least one positive assay: (i) Positive B cell assays (e.g., enzyme-linked immunosorbent assay (ELISA)-based qualitative binding) for B cell epitopes; and (ii) either positive T cell assays (such as enzyme-linked immune absorbent spot (ELISPOT) or intracellular cytokine staining (ICS) IFN-γ release), or positive major histocompatibility complex (MHC) binding assays for T cell epitopes. Strictly speaking, the latter set of epitopes, determined using positive MHC binding assays, are antigens which are candidate epitopes, since a T cell response has not been confirmed experimentally. However, for brevity and to be consistent with the terminology used in the ViPR database, we will not make this qualification, and will simply refer to them as epitopes in this study. The number of B cell and T cell epitopes obtained from the database following the above procedure is listed in Table 1.

TABLE 1
Filtering criteria and corresponding number of Severe
Acute Respiratory Syndrome Coronavirus (SARS-CoV)-
derived epitopes obtained from the Virus Pathogen
Database and Analysis Resource (ViPR) database.

| | | Number of |
|---|---|---|
| Filtering criteria | | epitopes |
| Positive T cell assays | T cell epitopes | 115 |
| Positive major histocompatibility | T cell epitopes | 959 |
| complex (MHC) binding assays | | |
| Positive B cell assays | Linear B cell | 298 |
| | epitopes | |
| | Discontinuous B cell | 6 |
| | epitopes | |

Population-Coverage-Based T Cell Epitope Selection. Population coverages for sets of T cell epitopes were computed using the tool provided by the Immune Epitope Database (IEDB) (tools.iedb.org/population/; accessed 21 Feb. 2020) [30]. This tool uses the distribution of MHC alleles (with at least 4-digit resolution, e.g., A*02:01) within a defined population (obtained from allelefrequencies.net/) to estimate the population coverage for a set of T cell epitopes. The estimated population coverage represents the percentage of individuals within the population that are likely to elicit an immune response to at least one T cell epitope from the set. To identify the set of epitopes associated with MHC alleles that would maximize the population coverage, we adopted a greedy approach: (i) We first identified the MHC allele with the highest individual population coverage and initialized the set with their associated epitopes, then (ii) we progressively added epitopes associated with other MHC alleles that resulted in the largest increase of the accumulated population coverage. We stopped when no increase in the accumulated population coverage was observed by adding epitopes associated with any of the remaining MHC alleles.

Constructing the Phylogenetic Tree. We used the publicly available software PASTA v1.6.4 [31] to construct a maximum-likelihood phylogenetic tree of each structural protein using the unique set of sequences in the available data of SARS-CoV, MERS-CoV, and SARS-CoV-2. We additionally included the Zaria Bat coronavirus strain (accession ID: HQ166910.1) to serve as an outgroup. The appropriate parameters for tree estimation are automatically selected in the software based on the provided sequence data. For visualizing the constructed phylogenetic trees, we used the publicly available software Dendroscope v3.6.3 [32]. Each constructed tree was rooted with the outgroup Zaria Bat coronavirus strain, and circular phylogram layout was used

Data and Code Availability. All sequence and immunological data, and all scripts (written in R) for reproducing the results are available online [33].

### Results

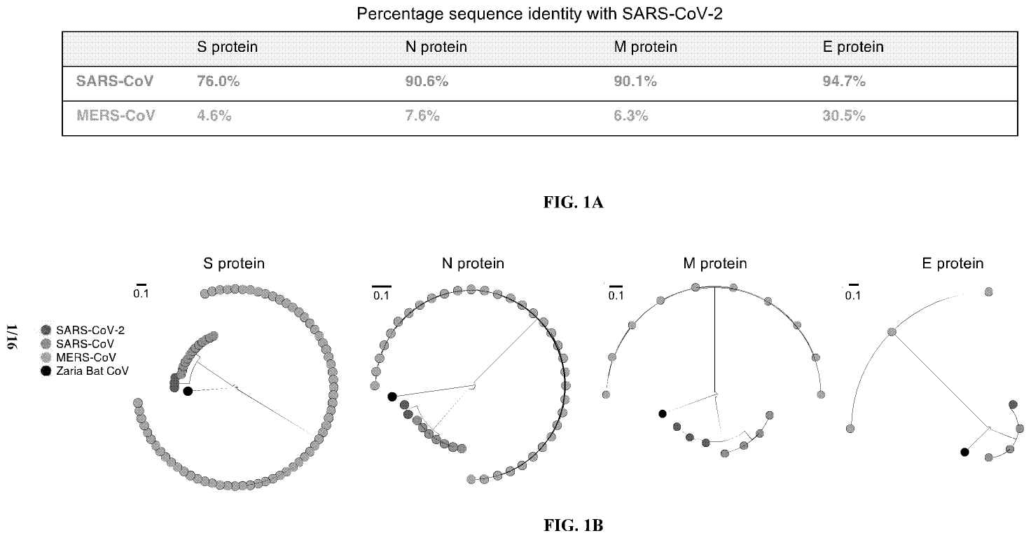

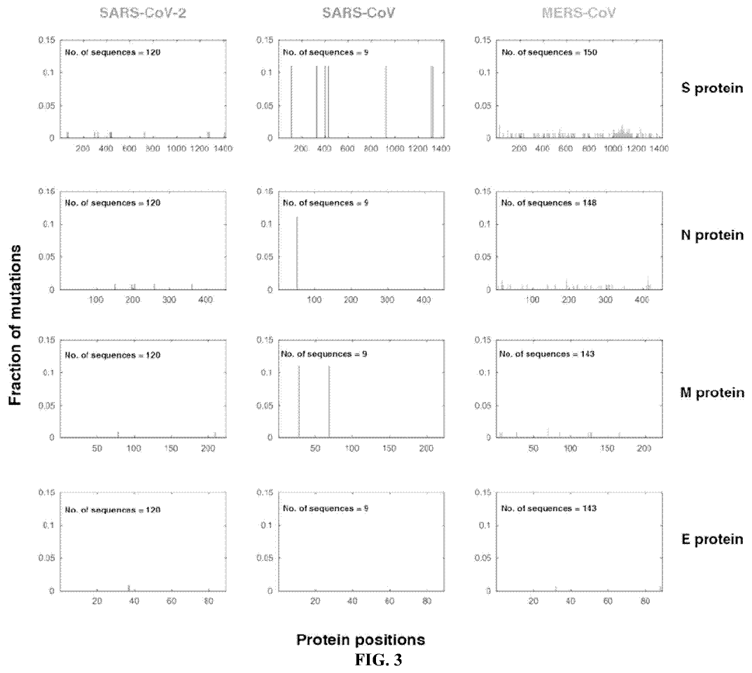

Structural Proteins of SARS-CoV-2 Are Genetically Similar to SARS-CoV, but Not to MERS-CoV. SARS-CoV-2 has been observed to be close to SARS-CoV-much more so than MERS-CoV-based on full-length genome phylogenetic analysis [9,12]. We checked whether this is also true at the level of the individual structural proteins (S, E, M, and N). A straightforward reference-sequence-based comparison indeed confirmed this, showing that the M, N, and E proteins of SARS-CoV-2 and SARS-CoV have over 90% genetic similarity, while that of the S protein was notably reduced (but still high) (FIG. 1A). The similarity between SARS-CoV-2 and MERS-CoV, on the other hand, was substantially lower for all proteins (FIG. 1A); a feature that was also evident from the corresponding phylogenetic trees (FIG. 1B). We note that while the former analysis (FIG. 1A) was based on the reference sequence of each coronavirus, it is indeed a good representative of the virus population, since few amino acid mutations have been observed in the corresponding sequence data (FIG. 3). It is also noteworthy that while MERS-CoV is the more recent coronavirus to have infected humans, and is comparatively more recurrent (causing outbreaks in 2012, 2015, and 2018) (who.int/emergencies/mers-cov/en/), SARS-CoV-2 is closer to SARS-CoV, which has not been observed since 2004.

Given the close genetic similarity between the structural proteins of SARS-CoV and SARS-CoV-2, we attempted to leverage immunological studies of the structural proteins of SARS-CoV to potentially aid vaccine development for SARS-CoV-2. We focused specifically on the S and N proteins as these are known to induce potent and long-lived immune responses in SARS-CoV [15-17,19,20,25,27]. We used the available SARS-CoV-derived experimentally-determined epitope data (see Materials and Methods) and searched to identify T cell and B cell epitopes that were identical- and hence potentially cross-reactive-across SARS-CoV and SARS-CoV-2. We first report the analysis for T cell epitopes, which have been shown to provide a long-lasting immune response against SARS-CoV [27], followed by a discussion of B cell epitopes.

Mapping the SARS-CoV-Derived T Cell Epitopes that ARE identical in SARS-CoV-2, and Determining those with Greatest Estimated Population Coverage. The SARS-CoV-derived T cell epitopes used in this study were experimentally-determined from two different types of assays [29]: (i) Positive T cell assays, which tested for a T cell response against epitopes, and (ii) positive MHC binding assays, which tested for epitope-MHC binding. We aligned these T cell epitopes across the SARS-CoV-2 protein sequences. Among the 115 T cell epitopes that were determined by positive T cell assays (Table 1), we found that 27 epitope-sequences were identical within SARS-CoV-2 proteins and comprised no mutation in the available SARS-CoV-2 sequences (as of 21 Feb. 2020) (Table 2). Interestingly, all of these were present in either the N (16) or S (11) protein. MHC binding assays were performed for 19 of these 27 epitopes, and these were reported to be associated with only five distinct MHC alleles (at 4-digit resolution): HLA-A*02:01, HLA-B*40:01, HLA-DRA*01:01, HLA-DRB1*07:01, and HLA-DRB1*04:01. Consequently, the accumulated population coverage of these epitopes (see Materials and Methods for details) is estimated to not be high for the global population (59.76%), and was quite low for China (32.36%). For the remaining 8 epitopes, since the associated MHC alleles are unknown, they could not be used in the population coverage computation. Additional MHC binding tests to identify the MHC alleles that bind to these 8 epitopes may reveal additional distinct alleles, beyond the five determined so far, that may help to improve population coverage.

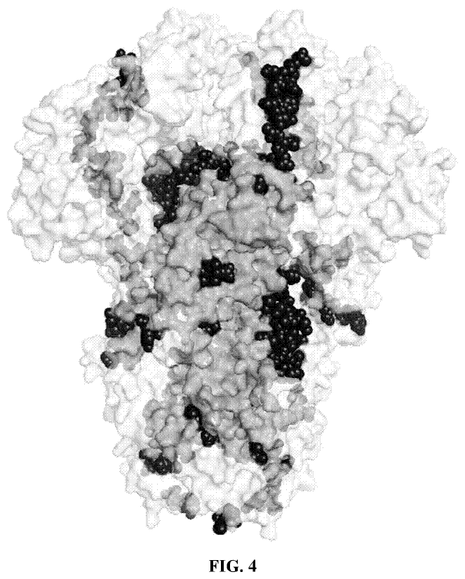

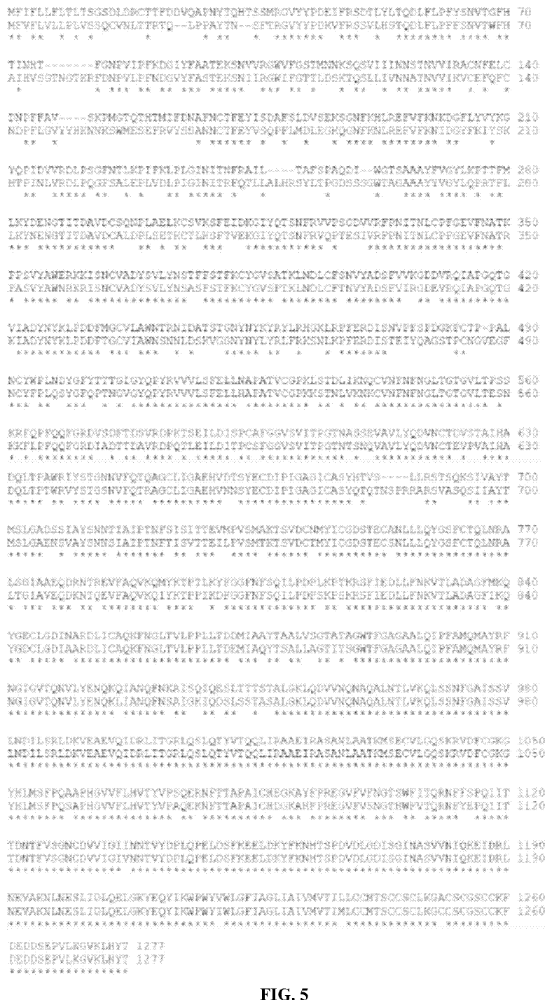

To further expand the search and identify potentially effective T cell targets covering a higher percentage of the population, we next additionally considered the set of T cell epitopes that have been experimentally-determined from positive MHC binding assays (Table 1), but, unlike the previous epitope set, their ability to induce a T cell response against SARS-CoV was not experimentally determined. Nonetheless, they also present promising candidates for inducing a response against SARS-CoV-2. For the expanded set of epitopes, all of which have at least one positive MHC binding assay, we found that 229 epitope-sequences have an identical match in SARS-CoV-2 proteins and have associated MIIHC allele information available (listed in Table 7). Of these 229 epitopes, ˜82% were MHC Class I restricted epitopes (Table 8). Importantly, 102 of the 229 epitopes were derived from either the S (66) or N (36) protein. Mapping all 66 S-derived epitopes onto the resolved crystal structure of the SARS-CoV S protein (FIG. 4) revealed that 3 of these (GYQPYRVVVL, QPYRVVVLSF, and PYRVVVLSF) were located entirely in the SARS-CoV receptor-binding motif (uniprot.org/uniprot/P59594), known to be important for virus cell entry [34].

TABLE 7
List of all SARS-CoV-derived T cell epitopes determined using positive MHC binding
assays (with associated MHC allele information available at 4-digit resolution)
and found to be identical in SARS-CoV-2.

| | | | | MHC |
|---|---|---|---|---|
| | IEDB | | | Allele |
| Protein | ID | Epitope | MHC Allele | Class |
| E | 57395 | SEETGTLIV | HLA-B*45:01/HLA-B*40:01 | I |
| M | 16972 | FLWLLWPVT | HLA-A*02:01/HLA-A*02:02/HLA- | I |
| | | | A*02:03/HLA-A*02:06/HLA-A*68:02 | |
| M | 16973 | FLWLLWPVTL | HLA-A*02:01/HLA-A*02:02/HLA- | I |
| | | | A*02:03/HLA-A*02:06 | |
| M | 16974 | FLWLLWPVTLACFVL | HLA-DRB1*01:01 | II |
| M | 26759 | IKDLPKEITVATSRT | HLA-DRB1*01:01 | II |
| M | 35671 | LEQWNLVIGF | HLA-B*44:02/HLA-B*44:03/HLA- | I |
| | | | B*18:01/HLA-B*40:01/HLA-B*40:02/HLA- | |
| | | | B*45:01 | |
| M | 35797 | LFARTRSMW | HLA-A*23:01/HLA-A*24:02 | I |
| M | 40677 | LWLLWPVTL | HLA-A*23:01/HLA-A*24:02/HLA-A*29:02 | I |
| M | 40685 | LWPVTLACF | HLA-A*23:01/HLA-A*24:02/HLA-A*29:02 | I |
| M | 43024 | MWSFNPETNI | HLA-A*23:01/HLA-A*24:02 | I |
| M | 44913 | NLVIGFLFL | HLA-A*02:01/HLA-A*02:03/HLA- | I |
| | | | A*02:06/HLA-A*02:02/HLA-A*68:02 | |
| M | 48051 | PKEITVATSRTLSYY | HLA-DRB1*01:01 | II |
| M | 5149 | ATSRTLSYY | HLA-A*01:01/HLA-A*03:01/HLA- | I |
| | | | A*11:01/HLA-A*29:02/HLA-A*30:02/HLA- | |
| | | | A*26:01/HLA-A*31:01/HLA-A*68:01 | |
| M | 5150 | ATSRTLSYYK | HLA-A*03:01/HLA-A*11:01/HLA- | I |
| | | | A*31:01/HLA-A*68:01 | |
| M | 52851 | QWNLVIGFLF | HLA-A*23:01/HLA-A*24:02/HLA-A*29:02 | I |
| M | 56634 | RYRIGNYKL | HLA-A*23:01/HLA-A*24:02/HLA-A*30:02 | I |
| M | 57544 | SELVIGAVI | HLA-B*40:01/HLA-B*40:02/HLA- | I |
| | | | B*44:03/HLA-B*45:01/HLA-B*18:01/HLA- | |
| | | | B*44:02 | |
| M | 57856 | SFNPETNIL | HLA-A*23:01/HLA-A*24:02/HLA-A*30:02 | I |
| M | 59778 | SMWSFNPET | HLA-A*02:01/HLA-A*02:02/HLA- | I |
| | | | A*02:03/HLA-A*02:06 | |
| M | 66407 | TSRTLSYYK | HLA-A*03:01/HLA-A*11:01/HLA- | I |
| | | | A*31:01/HLA-A*33:01/HLA-A*68:01 | |
| M | 66952 | TVATSRTLSY | HLA-A*01:01/HLA-A*26:01/HLA- | I |
| | | | A*29:02/HLA-A*30:02 | |
| M | 72759 | WLLWPVTLA | HLA-A*02:01/HLA-A*02:02/HLA- | I |
| | | | A*02:03/HLA-A*02:06/HLA-A*68:02 | |
| M | 72972 | WPVTLACFVL | HLA-B*51:01/HLA-B*35:01/HLA- | I |
| | | | B*53:01/HLA-B*07:02/HLA-B*54:01 | |
| N | 125100 | ILLNKHID | HLA-A*02:01 | I |
| N | 17385 | FPRGQGVPI | HLA-B*07:02/HLA-B*54:01/HLA- | I |
| | | | B*08:01/HLA-B*35:01/HLA-B*51:01/HLA- | |
| | | | A*02:01/HLA-B*53:01 | |
| N | 190494 | MEVTPSGTWL | HLA-B*40:01 | I |
| N | 193464 | GMEVTPSGTWL | HLA-B*40:01 | I |
| N | 193498 | LLLLDRLNQ | HLA-A*02:01 | I |
| N | 21347 | GMSRIGMEV | HLA-A*02:01 | I |
| N | 22856 | GTTLPKGFY | HLA-A*30:02/HLA-A*29:02/HLA-A*26:01 | I |
| N | 2431 | ALALLLLDR | HLA-A*03:01 | I |
| N | 25542 | IDAYKTFPPTEPKKD | HLA-DRB1*01:01 | II |
| N | 27182 | ILLNKHIDA | HLA-A*02:01 | I |
| N | 2802 | ALNTPKDHI | HLA-A*02:01 | I |
| N | 33667 | KTFPPTEPK | HLA-A*11:01/HLA-A*03:01/HLA- | I |
| | | | A*31:01/HLA-A*68:01 | |
| N | 33668 | KTFPPTEPKK | HLA-A*03:01/HLA-A*11:01/HLA- | I |
| | | | A*68:01/HLA-A*31:01 | |
| N | 34851 | LALLLLDRL | HLA-A*02:01 | I |
| N | 37473 | LLLDRLNQL | HLA-A*02:01 | I |
| N | 37515 | LLLLDRLNQL | HLA-A*02:01 | I |
| N | 3810 | APSASAFFGM | HLA-B*07:02/HLA-B*35:01/HLA- | I |
| | | | B*51:01/HLA-B*53:01 | |
| N | 38881 | LQLPQGTTL | HLA-A*02:01 | I |
| N | 3956 | AQFAPSASA | HLA-A*02:02/HLA-A*02:06/HLA- | I |
| | | | A*02:01/HLA-A*02:03 | |
| N | 39576 | LSPRWYFYY | HLA-A*01:01/HLA-A*29:02/HLA- | I |
| | | | A*30:02/HLA-A*11:01/HLA-A*23:01/HLA- | |
| | | | A*24:02/HLA-A*31:01 | |
| N | 42648 | MSRIGMEVTPSGTWL | HLA-DRB1*01:01 | II |
| N | 4307 | ASAFFGMSR | HLA-A*31:01/HLA-A*11:01/HLA- | I |
| | | | A*68:01/HLA-A*03:01 | |
| N | 44501 | NKHIDAYKTFPPTEP | HLA-DRB1*01:01 | II |
| N | 4936 | ATEGALNTPK | HLA-A*11:01/HLA-A*03:01/HLA- | I |
| | | | A*68:01/HLA-A*31:01 | |
| N | 51482 | QLPQGTTLPK | HLA-A*03:01/HLA-A*11:01/HLA-A*68:01 | I |
| N | 52114 | QQQGQTVTK | HLA-A*11:01/HLA-A*31:01 | I |
| N | 52129 | QQQQGQTVTK | HLA-A*11:01 | I |
| N | 56979 | SASAFFGMSR | HLA-A*11:01/HLA-A*31:01/HLA- | I |
| | | | A*68:01/HLA-A*33:01/HLA-A*03:01 | |
| N | 60242 | SPRWYFYYL | HLA-B*07:02/HLA-B*51:01/HLA- | I |
| | | | B*53:01/HLA-B*54:01 | |
| N | 60380 | SQASSRSSSR | HLA-A*31:01/HLA-A*68:01/HLA- | I |
| | | | A*11:01/HLA-A*33:01 | |
| N | 65763 | TPSGTWLTY | HLA-A*29:02/HLA-A*11:01 | I |
| N | 66706 | TTLPKGFYA | HLA-A*02:01 | I |
| N | 69720 | VLQLPQGTTL | HLA-A*02:01 | I |
| N | 69721 | VLQLPQGTTLPKGFY | HLA-DRB1*01:01 | II |
| N | 71461 | VTPSGTWLTY | HLA-A*30:02/HLA-A*01:01/HLA- | I |
| | | | A*26:01/HLA-A*29:02 | |
| N | 956 | AEGSRGGSQA | HLA-B*45:01 | I |
| orf1a | 16537 | FLCLFLLPSL | HLA-A*02:01/HLA-A*02:02/HLA- | I |
| | | | A*02:03/HLA-A*02:06/HLA-A*68:02 | |
| orf1a | 16632 | FLGRYMSAL | HLA-A*02:01/HLA-A*02:02/HLA- | I |
| | | | A*02:03/HLA-A*02:06 | |
| orf1a | 16737 | FLLNKEMYL | HLA-A*02:01/HLA-A*02:02/HLA- | I |
| | | | A*02:03/HLA-A*02:06 | |
| orf1a | 16743 | FLLPSLATV | HLA-A*02:01/HLA-A*02:02/HLA- | I |
| | | | A*02:03/HLA-A*02:06/HLA-A*68:02 | |
| orf1a | 16779 | FLNGSCGSV | HLA-A*02:01/HLA-A*02:02/HLA- | I |
| | | | A*02:03/HLA-A*02:06/HLA-A*68:02 | |
| orf1a | 16786 | FLNRFTTTL | HLA-A*02:01/HLA-A*02:02/HLA- | I |
| | | | A*02:03/HLA-A*02:06/HLA-A*68:02 | |
| orf1a | 16820 | FLPRVFSAV | HLA-A*02:02/HLA-A*02:06/HLA- | I |
| | | | A*02:01/HLA-A*02:03/HLA-A*68:02 | |
| orf1a | 17657 | FRYMNSQGL | HLA-C*06:02 | I |
| orf1a | 18103 | FTYASALWEI | HLA-A*02:01/HLA-A*02:02/HLA- | I |
| | | | A*02:03/HLA-A*02:06/HLA-A*68:02 | |
| orf1a | 1946 | AIILASFSA | HLA-A*02:01/HLA-A*02:02/HLA- | I |
| | | | A*02:03/HLA-A*02:06/HLA-A*68:02 | |
| orf1a | 23215 | GVYDYLVST | HLA-A*02:06/HLA-A*02:01/HLA- | I |
| | | | A*02:02/HLA-A*02:03 | |
| orf1a | 26973 | ILASFSAST | HLA-A*02:01/HLA-A*02:02/HLA- | I |
| | | | A*02:03/HLA-A*02:06/HLA-A*68:02 | |
| orf1a | 27083 | ILGTVSWNL | HLA-A*02:01/HLA-B*58:01 | I |
| orf1a | 28199 | IQPGQTFSV | HLA-A*02:02/HLA-A*02:03/HLA- | I |
| | | | A*02:06/HLA-A*02:01/HLA-A*68:02 | |
| orf1a | 2855 | ALRANSAVK | HLA-A*03:01/HLA-A*31:01/HLA-A*11:01 | I |
| orf1a | 2998 | ALWEIQQVV | HLA-A*02:01/HLA-A*02:02/HLA- | I |
| | | | A*02:03/HLA-A*02:06/HLA-A*68:02/HLA- | |
| | | | A*69:01/HLA-B*15:01 | |
| orf1a | 32240 | KLWAQCVQL | HLA-A*02:01/HLA-A*02:02/HLA- | I |
| | | | A*02:06/HLA-A*02:03/HLA-A*31:01/HLA- | |
| | | | A*03:01 | |
| orf1a | 37766 | LLSAGIFGA | HLA-A*02:01/HLA-A*02:02/HLA- | I |
| | | | A*02:03/HLA-A*02:06/HLA-A*68:02 | |
| orf1a | 42260 | MPASWVMRI | HLA-B*07:02/HLA-B*35:01 | I |
| orf1a | 46414 | NVLAWLYAA | HLA-A*02:01/HLA-A*02:02/HLA- | I |
| | | | A*02:03/HLA-A*02:06/HLA-A*68:02 | |
| orf1a | 51442 | QLMCQPILL | HLA-A*02:01/HLA-A*02:02/HLA- | I |
| | | | A*02:03/HLA-A*02:06/HLA-A*68:02 | |
| orf1a | 51443 | QLMCQPILLL | HLA-A*02:01/HLA-A*02:02/HLA- | I |
| | | | A*02:03/HLA-A*02:06/HLA-A*68:02 | |
| orf1a | 5447 | AVLQSGFRK | HLA-A*03:01/HLA-A*11:01 | I |
| orf1a | 59312 | SLLSVLLSM | HLA-A*02:01/HLA-A*02:02/HLA- | I |
| | | | A*02:06/HLA-A*02:03 | |
| orf1a | 64827 | TLGVYDYLV | HLA-A*02:01/HLA-A*02:02/HLA- | I |
| | | | A*02:06/HLA-A*68:02/HLA-A*02:03 | |
| orf1a | 67078 | TVLSFCAFA | HLA-A*02:01/HLA-A*02:02/HLA- | I |
| | | | A*02:03/HLA-A*02:06/HLA-A*68:02 | |
| orf1a | 69392 | VLAWLYAAV | HLA-A*02:01/HLA-A*02:02/HLA- | I |
| | | | A*02:03/HLA-A*02:06/HLA-A*68:02 | |
| orf1a | 69758 | VLSFCAFAV | HLA-A*02:01/HLA-A*02:02/HLA- | I |
| | | | A*02:03/HLA-A*02:06/HLA-A*68:02 | |
| orf1a | 74209 | YIFFASFYY | HLA-A*03:01/HLA-A*11:01/HLA- | I |
| | | | A*68:01/HLA-A*26:01/HLA-A*31:01/HLA- | |
| | | | A*33:01/HLA-A*69:01/HLA-B*15:01/HLA- | |
| | | | B*58:01 | |
| orf1b | 17354 | FPPTSFGPL | HLA-B*07:02/HLA-A*69:01/HLA- | I |
| | | | B*51:01/HLA-C*14:02/HLA-A*11:01/HLA- | |
| | | | B*15:42/HLA-B*35:01/HLA-B*45:06/HLA- | |
| | | | B*54:01/HLA-C*04:01 | |
| orf1b | 18133 | FVDGVPFVV | HLA-A*02:01/HLA-A*02:02/HLA- | I |
| | | | A*02:06/HLA-A*02:03/HLA-A*68:02 | |
| orf1b | 2027 | AIMTRCLAV | HLA-A*02:01/HLA-A*02:03/HLA- | I |
| | | | A*02:06/HLA-A*02:02/HLA-A*68:02 | |
| orf1b | 22916 | GVAMPNLYK | HLA-A*03:01/HLA-A*11:01/HLA- | I |
| | | | A*68:01/HLA-A*31:01 | |
| orf1b | 2682 | ALLADKFPV | HLA-A*02:01/HLA-A*02:02/HLA- | I |
| | | | A*02:06/HLA-A*02:03 | |
| orf1b | 27078 | ILGLPTQTV | HLA-A*02:01/HLA-B*15:01 | I |
| orf1b | 27089 | ILHCANFNV | HLA-A*02:01/HLA-A*02:02/HLA- | I |
| | | | A*02:03/HLA-A*02:06/HLA-A*68:02/HLA- | |
| | | | A*69:01/HLA-A*31:01 | |
| orf1b | 28050 | IPRRNVATL | HLA-B*07:02/HLA-A*02:01/HLA-B*35:01 | I |
| orf1b | 28464 | ISDYDYYRY | HLA-A*01:01/HLA-A*29:02/HLA-A*30:02 | I |
| orf1b | 29237 | IVDTVSALV | HLA-A*02:02/HLA-A*02:06/HLA- | I |
| | | | A*02:01/HLA-A*68:02/HLA-A*02:03 | |
| orf1b | 31833 | KLFAAETLK | HLA-A*03:01/HLA-A*11:01/HLA- | I |
| | | | A*31:01/HLA-A*68:01 | |
| orf1b | 32060 | KLNVGDYFV | HLA-A*02:01/HLA-A*02:02/HLA- | I |
| | | | A*02:03/HLA-A*02:06/HLA-A*68:02 | |
| orf1b | 32183 | KLSYGIATV | HLA-A*02:01/HLA-A*02:02/HLA- | I |
| | | | A*02:03/HLA-A*02:06/HLA-A*68:02 | |
| orf1b | 32390 | KMQRMLLEK | HLA-A*03:01/HLA-A*11:01/HLA- | I |
| | | | A*31:01/HLA-A*68:01 | |
| orf1b | 32976 | KQFDTYNLW | HLA-A*24:02/HLA-B*15:01/HLA- | I |
| | | | B*58:01/HLA-A*31:01 | |
| orf1b | 37144 | LLDDFVEII | HLA-A*02:01/HLA-A*02:02/HLA- | I |
| | | | A*02:06/HLA-A*02:03 | |
| orf1b | 37468 | LLLDDFVEI | HLA-A*02:01/HLA-A*02:02/HLA- | I |
| | | | A*02:06/HLA-A*02:03 | |
| orf1b | 37583 | LLMPILTLT | HLA-A*02:01/HLA-A*02:02/HLA- | I |
| | | | A*02:03/HLA-A*02:06/HLA-A*68:02 | |
| orf1b | 38043 | LMIERFVSL | HLA-A*02:01/HLA-A*02:02/HLA- | I |
| | | | A*02:03/HLA-A*02:06/HLA-A*68:02 | |
| orf1b | 38874 | LQLGFSTGV | HLA-A*02:01/HLA-A*02:02/HLA- | I |
| | | | A*02:03/HLA-A*02:06/HLA-A*68:02 | |
| orf1b | 40459 | LVLSVNPYV | HLA-A*02:01/HLA-A*02:02/HLA- | I |
| | | | A*02:06/HLA-A*02:03/HLA-A*68:02 | |
| orf1b | 42093 | MLWCKDGHV | HLA-A*02:01/HLA-A*02:02/HLA- | I |
| | | | A*02:03/HLA-A*02:06/HLA-A*68:02 | |
| orf1b | 42128 | MMISAGFSL | HLA-A*02:01/HLA-A*02:02/HLA- | I |
| | | | A*02:03/HLA-A*02:06/HLA-A*68:02 | |
| orf1b | 42972 | MVMCGGSLYV | HLA-A*02:01/HLA-A*02:02/HLA- | I |
| | | | A*02:03/HLA-A*02:06/HLA-A*68:02 | |
| orf1b | 44927 | NLWNTFTRL | HLA-A*02:01/HLA-A*02:02/HLA- | I |
| | | | A*02:03/HLA-A*02:06/HLA-A*68:02 | |
| orf1b | 45000 | NMLRIMASL | HLA-A*02:02/HLA-A*02:03/HLA- | I |
| | | | A*02:06/HLA-A*02:01/HLA-A*68:02 | |
| orf1b | 5209 | ATVVIGTSK | HLA-A*11:01/HLA-A*03:01/HLA- | I |
| | | | A*68:01/HLA-A*31:01 | |
| orf1b | 54183 | RILGAGCFV | HLA-A*02:01/HLA-A*02:02/HLA- | I |
| | | | A*02:06/HLA-A*02:03 | |
| orf1b | 54847 | RLYYDSMSY | HLA-B*15:01/HLA-A*03:01/HLA-A*11:01 | I |
| orf1b | 55444 | RQLLFVVEV | HLA-A*02:01/HLA-A*02:02/HLA- | I |
| | | | A*02:03/HLA-A*02:06/HLA-A*68:02 | |
| orf1b | 61254 | SSNVANYQK | HLA-A*11:01/HLA-A*03:01/HLA- | I |
| | | | A*68:01/HLA-A*31:01 | |
| orf1b | 64850 | TLIGDCATV | HLA-A*02:01/HLA-A*02:02/HLA- | I |
| | | | A*02:03/HLA-A*02:06/HLA-A*68:02 | |
| orf1b | 65132 | TLVPQEHYV | HLA-A*02:01/HLA-A*69:01 | I |
| orf1b | 65176 | TMADLVYAL | HLA-A*02:01/HLA-A*02:02/HLA- | I |
| | | | A*02:03/HLA-A*02:06/HLA-A*68:02 | |
| orf1b | 66710 | TTLPVNVAF | HLA-B*58:01/HLA-A*69:01/HLA- | I |
| | | | A*02:01/HLA-B*15:01 | |
| orf1b | 69716 | VLQAVGACV | HLA-A*02:01/HLA-A*02:02/HLA- | I |
| | | | A*02:03/HLA-A*02:06/HLA-A*68:02 | |
| orf1b | 69850 | VLWAHGFEL | HLA-A*02:01/HLA-A*24:02/HLA- | I |
| | | | A*02:02/HLA-A*02:03/HLA-A*02:06/HLA- | |
| | | | A*31:01/HLA-A*69:01/HLA-A*01:01/HLA- | |
| | | | A*03:01/HLA-A*68:02/HLA-B*08:01/HLA- | |
| | | | B*15:01/HLA-B*58:01 | |
| orf1b | 69929 | VMCGGSLYV | HLA-A*02:01/HLA-A*02:02/HLA- | I |
| | | | A*02:03/HLA-A*02:06/HLA-A*68:02 | |
| orf1b | 71635 | VVDKYFDCY | HLA-A*01:01/HLA-A*29:02/HLA-A*30:02 | I |
| orf1b | 71917 | VVYRGTTTY | HLA-B*15:01/HLA-A*03:01/HLA- | I |
| | | | A*11:01/HLA-B*35:01/HLA-B*58:01 | |
| orf1b | 74593 | YLDAYNMMI | HLA-A*02:01/HLA-A*02:02/HLA- | I |
| | | | A*02:03/HLA-A*02:06 | |
| orf1b | 74850 | YLNTLTLAV | HLA-A*02:01/HLA-A*02:02/HLA- | I |
| | | | A*02:03/HLA-A*02:06/HLA-A*68:02 | |
| orf1b | 75508 | YQKVGMQKY | HLA-B*15:01/HLA-B*27:05 | I |
| orf1b | 76120 | YTMADLVYA | HLA-A*02:01/HLA-A*02:02/HLA- | I |
| | | | A*02:03/HLA-A*02:06/HLA-A*68:02 | |
| orf1b | 76266 | YVFCTVNAL | HLA-A*02:01/HLA-A*02:02/HLA- | I |
| | | | A*02:03/HLA-A*02:06/HLA-A*68:02 | |
| ORF6 | 24313 | HLVDFQVTI | HLA-A*02:01/HLA-A*02:02/HLA- | I |
| | | | A*02:03/HLA-A*02:06/HLA-A*68:02 | |
| ORF7a | 24466 | HPLADNKFAL | HLA-B*07:02/HLA-B*35:01/HLA- | I |
| | | | B*51:01/HLA-B*53:01 | |
| ORF7a | 31846 | KLFIRQEEV | HLA-A*02:01/HLA-A*31:01/HLA-B*08:01 | I |
| ORF7a | 50560 | QECVRGTTVLLKEPC | HLA-DRB1*01:01 | II |
| ORF7a | 6184 | CELYHYQECV | HLA-B*45:01/HLA-B*40:01/HLA-B*44:03 | I |
| ORF7a | 62331 | SVSPKLFIR | HLA-A*31:01/HLA-A*11:01/HLA-A*68:01 | I |
| ORF7a | 73668 | YEGNSPFHPL | HLA-B*40:01/HLA-B*40:02 | I |
| ORF7b | 1349 | AFLLFLVLI | HLA-A*26:01/HLA-A*01:01/HLA- | I |
| | | | A*23:01/HLA-A*29:02 | |
| ORF7b | 1350 | AFLLFLVLIMLIIFW | HLA-DRB1*01:01 | II |
| ORF7b | 16498 | FLAFLLFLV | HLA-A*02:01/HLA-A*02:02/HLA- | I |
| | | | A*02:06/HLA-A*02:03/HLA-A*68:02 | |
| ORF7b | 16499 | FLAFLLFLVL | HLA-A*02:01/HLA-A*02:02/HLA- | I |
| | | | A*02:03/HLA-A*02:06/HLA-A*68:02 | |
| ORF7b | 16500 | FLAFLLFLVLIMLII | HLA-DRB1*01:01 | II |
| ORF7b | 16716 | FLLFLVLIM | HLA-A*02:01/HLA-A*02:02/HLA- | I |
| | | | A*02:06/HLA-A*68:02 | |
| ORF7b | 16717 | FLLFLVLIML | HLA-A*02:01/HLA-A*02:02/HLA- | I |
| | | | A*02:03/HLA-A*02:06 | |
| ORF7b | 16718 | FLLFLVLIMLIIFWF | HLA-DRB1*01:01 | II |
| ORF7b | 16953 | FLVLIMLII | HLA-A*02:01/HLA-A*02:02/HLA- | I |
| | | | A*02:06/HLA-A*68:02 | |
| ORF7b | 16954 | FLVLIMLIIFWFSLE | HLA-DRB1*01:01 | II |
| ORF7b | 18422 | FYLCFLAFL | HLA-A*23:01/HLA-A*24:02 | I |
| ORF7b | 18423 | FYLCFLAFLL | HLA-A*23:01/HLA-A*24:02/HLA-A*29:02 | I |
| ORF7b | 25571 | IDFYLCFLAF | HLA-B*44:02/HLA-B*44:03/HLA- | I |
| | | | B*45:01/HLA-B*18:01/HLA-B*40:01 | |
| ORF7b | 27516 | IMLIIFWFSL | HLA-A*02:01/HLA-A*02:02 | I |
| ORF7b | 34763 | LAFLLFLVLIMLIIF | HLA-DRB1*01:01 | II |
| ORF7b | 35912 | LFLVLIMLIIFWFSL | HLA-DRB1*01:01 | II |
| ORF7b | 36477 | LIDFYLCFL | HLA-A*02:01/HLA-A*02:02/HLA- | I |
| | | | A*02:06/HLA-A*02:03/HLA-A*68:02 | |
| ORF7b | 37279 | LLFLVLIML | HLA-A*02:01/HLA-A*02:02/HLA- | I |
| | | | A*02:03/HLA-A*02:06/HLA-A*68:02 | |
| ORF7b | 37280 | LLFLVLIMLI | HLA-A*02:02/HLA-A*02:01/HLA-A*02:03 | I |
| ORF7b | 37281 | LLFLVLIMLIIFWFS | HLA-DRB1*01:01 | II |
| ORF7b | 41962 | MLIIFWFSL | HLA-A*02:02/HLA-A*02:06/HLA-A*02:01 | I |
| ORF7b | 74577 | YLCFLAFLL | HLA-A*02:01/HLA-A*02:02/HLA- | I |
| | | | A*02:03/HLA-A*02:06/HLA-A*68:02 | |
| ORF7b | 74578 | YLCFLAFLLFLVLIM | HLA-DRB1*01:01 | II |
| S | 10112 | DSFKEELDKY | HLA-A*26:01/HLA-A*29:02/HLA- | I |
| | | | A*30:02/HLA-A*01:01 | |
| S | 1220 | AEVQIDRLI | HLA-B*40:02/HLA-B*44:02/HLA- | I |
| | | | B*44:03/HLA-B*45:01 | |
| S | 1221 | AEVQIDRLIT | HLA-B*40:02/HLA-B*44:03/HLA- | I |
| | | | B*45:01/HLA-B*40:01/HLA-B*44:02 | |
| S | 16156 | FIAGLIAIV | HLA-A*02:01/HLA-A*02:02/HLA- | I |
| | | | A*02:03/HLA-A*02:06/HLA-A*68:02/HLA-A2 | |
| S | 17341 | FPNITNLCPF | HLA-B*35:01/HLA-B*51:01/HLA- | I |
| | | | B*53:01/HLA-B*07:02/HLA-B*54:01 | |
| S | 18514 | GAALQIPFAMQMAYR | HLA-DRB1*01:01 | II |
| S | 20907 | GLIAIVMVTI | HLA-A*02:02/HLA-A*02:03/HLA- | I |
| | | | A*02:01/HLA-A*02:06/HLA-A*68:02 | |
| S | 22144 | GRLQSLQTY | HLA-B*27:05 | I |
| S | 22322 | GSFCTQLNR | HLA-A*11:01/HLA-A*31:01/HLA- | I |
| | | | A*68:01/HLA-A*03:01 | |
| S | 23200 | GVVFLHVTY | HLA-A*11:01 | I |
| S | 23293 | GWTFGAGAALQIPFA | HLA-DRB1*01:01 | II |
| S | 23436 | GYQPYRVVVL | HLA-A*23:01/HLA-A*24:02/HLA-A*29:02 | I |
| S | 25662 | IDRLITGRLQSLQTY | HLA-DRB1*01:01 | II |
| S | 26198 | IGAGICASY | HLA-A*29:02/HLA-A*30:02 | I |
| S | 26710 | IITTDNTFV | HLA-A*02:01 | I |
| S | 2801 | ALNTLVKQL | HLA-A*02:01 | I |
| S | 28511 | ISGINASVVNIQKEI | HLA-DRB1*01:01 | II |
| S | 35205 | LDKYFKNHTSPDVDL | HLA-DRB1*01:01 | II |
| S | 3589 | APHGVVFLHV | HLA-B*07:02/HLA-B*54:01/HLA- | I |
| | | | B*35:01/HLA-B*53:01 | |
| S | 36075 | LGDISGINASVVNIQ | HLA-DRB1*01:01 | II |
| S | 36103 | LGFIAGLIAIVMVTI | HLA-DRB1*01:01 | II |
| S | 36481 | LIDLQELGKY | HLA-A*30:02/HLA-A*01:01/HLA- | I |
| | | | A*26:01/HLA-A*29:02 | |
| S | 36724 | LITGRLQSL | HLA-A2/HLA-A*02:01 | I |
| S | 37544 | LLLQYGSFC | HLA-A*02:01 | I |
| S | 37724 | LLQYGSFCT | HLA-A*02:01 | I |
| S | 38353 | LNTLVKQLSSNFGAI | HLA-DRB1*01:01 | II |
| S | 38831 | LQDVVNQNAQALNTL | HLA-DRB1*01:01 | II |
| S | 38855 | LQIPFAMQM | HLA-B*15:01/HLA-C*15:02/HLA- | I |
| | | | B*40:01/HLA-B*58:01 | |
| S | 38990 | LQSLQTYVTQQLIRA | HLA-DRB1*01:01 | II |
| S | 39003 | LQTYVTQQLIRAAEI | HLA-DRB1*01:01 | II |
| S | 3939 | AQALNTLVK | HLA-A*11:01/HLA-A*03:01/HLA- | I |
| | | | A*31:01/HLA-A*68:01 | |
| S | 3982 | AQKFNGLTVLPPLLT | HLA-DRB1*01:01 | II |
| S | 42873 | MTSCCSCLK | HLA-A*11:01/HLA-A*31:01/HLA- | I |
| | | | A*33:01/HLA-A*68:01/HLA-A*03:01 | |
| S | 4321 | ASANLAATK | HLA-A*11:01/HLA-A*03:01/HLA- | I |
| | | | A*68:01/HLA-A*31:01 | |
| S | 44814 | NLNESLIDL | HLA-A*02:01 | I |
| S | 47041 | PCSFGGVSVITPGTN | HLA-DRB1*01:01 | II |
| S | 50166 | PYRVVVLSF | HLA-A*23:01/HLA-A*24:02/HLA- | I |
| | | | A*01:01/HLA-A*26:01 | |
| S | 50641 | QELGKYEQYI | HLA-B*44:02/HLA-B*44:03/HLA-B*40:02 | I |
| S | 51112 | QIPFAMQMAYRENGI | HLA-DRB1*01:01 | II |
| S | 51999 | QPYRVVVLSF | HLA-B*07:02/HLA-B*53:01 | I |
| S | 52057 | QQLIRAAEIRASANL | HLA-DRB1*01:01 | II |
| S | 52672 | QTYVTQQLIRAAEIR | HLA-DRB1*01:01 | II |
| S | 54507 | RLDKVEAEV | HLA-A*02:01/HLA-A*02:02/HLA- | I |
| | | | A*02:06/HLA-A*02:03/HLA-A*68:02 | |
| S | 54680 | RLNEVAKNL | HLA-A*02:01 | I |
| S | 54725 | RLQSLQTYV | HLA-A*02:01/HLA-A*02:02/HLA- | I |
| | | | A*02:03/HLA-A*02:06/HLA-A*68:02 | |
| S | 56252 | RVDFCGKGY | HLA-A*30:02/HLA-A*01:01/HLA- | I |
| | | | A*03:01/HLA-B*15:01/HLA-B*27:05 | |
| S | 5908 | AYRFNGIGVTQNVLY | HLA-DRB1*01:01 | II |
| S | 59161 | SLIDLQELGK | HLA-A*03:01/HLA-A*11:01/HLA- | I |
| | | | A*31:01/HLA-A*68:01/HLA-A*33:01 | |
| S | 61229 | SSNFGAISSVLNDIL | HLA-DRB1*01:01 | II |
| S | 62221 | SVLNDILSR | HLA-A*11:01/HLA-A*31:01/HLA-A*68:01 | I |
| S | 63951 | TGRLQSLQTYVTQQL | HLA-DRB1*01:01 | II |
| S | 65906 | TQNVLYENQK | HLA-A*11:01/HLA-A*68:01 | I |
| S | 6668 | CMTSCCSCLK | HLA-A*68:01/HLA-A*03:01/HLA- | I |
| | | | A*11:01/HLA-A*31:01/HLA-A*33:01 | |
| S | 69657 | VLNDILSRL | HLA-A*02:01 | I |
| S | 70575 | VQIDRLITGR | HLA-A*31:01/HLA-A*03:01/HLA- | I |
| | | | A*11:01/HLA-A*33:01/HLA-A*68:01 | |
| S | 70718 | VRFPNITNL | HLA-C*14:02/HLA-B*27:05 | I |
| S | 71663 | VVFLHVTYV | HLA-A*02:01/HLA-A*02:02/HLA- | I |
| | | | A*02:03/HLA-A*02:06/HLA-A*68:02 | |
| S | 72717 | WLGFIAGLIAIVMVT | HLA-DRB1*01:01 | II |
| S | 7289 | CVNFNFNGLTGTGVL | HLA-DRB1*01:01 | II |
| S | 73751 | YEQYIKWPWY | HLA-B*44:02/HLA-B*18:01/HLA- | I |
| | | | B*44:03/HLA-B*40:01/HLA-B*45:01 | |
| S | 9006 | DKYFKNHTSPDVDLG | HLA-DRB1*01:01 | II |
| S | 999 | AEIRASANLA | HLA-B*45:01/HLA-B*40:01/HLA- | I |
| | | | B*44:02/HLA-B*44:03/HLA-B*40:02 | |
| S | 100048 | GAALQIPFAMQMAYRF | HLA-DRA*01:01 | II |
| | | | DRB1*07:01 | |
| S | 100300 | MAYRFNGIGVTQNVLY | HLA-DRB1*04:01 | II |
| S | 100428 | QLIRAAEIRASANLAAT | HLA-DRB1*04:01 | II |
| | | K | | |
| S | 50311 | QALNTLVKQLSSNFGAI | HLA-DRB1*04:01 | II |

TABLE 8
Distribution of all SARS-CoV-derived T cell epitopes
obtained using positive MHC binding assays (with associated
MHC allele information available at 4-digit resolution)
and that are identical in SARS-CoV-2.

| Protein | MHC Allele Class I | MHC Allele Class II | Total |
|---|---|---|---|
| S | 40 | 26 | 66 |
| orf1b | 45 | 0 | 45 |
| N | 32 | 4 | 36 |
| orf1a | 29 | 0 | 29 |
| E | 1 | 0 | 1 |
| M | 19 | 3 | 22 |
| ORF7a | 5 | 1 | 6 |
| ORF7b | 15 | 8 | 23 |
| ORF6 | 1 | 0 | 1 |
| Total | 187 | 42 | 229 |

Similar to previous studies on HIV and HCV [35-38], we estimated population coverages for various combinations of MHC alleles associated with these 102 epitopes. Our aim was to determine sets of epitopes associated with MHC alleles with maximum population coverage, potentially aiding the development of vaccines against SARS-CoV-2. For selection, we adopted a greedy computational approach (see Materials and Methods), which identified a set of T cell epitopes estimated to maximize global population coverage. This set comprised of multiple T cell epitopes associated with 20 distinct MHC alleles and was estimated to provide an accumulated population coverage of 96.29% (Table 3). Interestingly, the majority of the T cell epitopes for which a positive immune response has been determined using T cell assays (Table 2) were presented by the globally most-prevalent MHC allele (shown in bold text in Table 3). Moreover, the functionally important epitopes located in the SARS-CoV receptor binding motif were associated with the second and third most-prevalent MHC alleles (underlined in Table 3). Thus, while the ordering of T cell epitopes in Table 3 is based on the estimated global population coverage of the associated MIIHC alleles, it is also a natural order in which these epitopes should be tested experimentally for determining their potential to induce a positive immune response against SARS-CoV-2. We also computed the population coverage of this specific set of epitopes in China, the country most affected by the COVID-19 outbreak, which was estimated to be slightly lower (88.11%), as certain MIIHC alleles (e.g., HLA-A*02:01) associated with some of these epitopes are less frequent in the Chinese population (Table 3). Repeating the same greedy approach but focusing on the Chinese population, instead of a global population, the maximum population coverage was estimated to be 92.76% (Table 9).

TABLE 2
SARS-CoV-derived T cell epitopes obtained using positive T cell assays
that are identical in SARS-CoV-2 (27 epitopes in total).

| | | | | MHC Allele |
|---|---|---|---|---|
| Protein | IEDB ID | Epitope | MHC Allele¹ | Class¹ |
| N | 125100 | ILLNKHID | HLA-A*02:01 | I |
| N | 1295 | AFFGMSRIGMEVTPSGTW | NA | NA |
| N | 190494 | MEVTPSGTWL | HLA-B*40:01 | I |
| N | 21347 | GMSRIGMEV | HLA-A*02:01 | I |
| N | 27182 | ILLNKHIDA | HLA-A*02:01 | I |
| N | 2802 | ALNTPKDHI | HLA-A*02:01 | I |
| N | 28371 | IRQGTDYKHWPQIAQFA | NA | NA |
| N | 31166 | KHWPQIAQFAPSASAFF | NA | NA |
| N | 34851 | LALLLLDRL | HLA-A*02:01 | I |
| N | 37473 | LLLDRLNQL | HLA-A*02:01 | I |
| N | 37611 | LLNKHIDAYKTFPPTEPK | NA | NA |
| N | 38881 | LQLPQGTTL | HLA-A*02:01 | I |
| N | 3957 | AQFAPSASAFFGMSR | NA | II |
| N | 3958 | AQFAPSASAFFGMSRIGM | NA | NA |
| N | 55683 | RRPQGLPNNTASWFT | NA | I |
| N | 74517 | YKTFPPTEPKKDKKKK | NA | NA |
| S | 100048 | GAALQIPFAMQMAYRF | HLA-DRA*01:01, | II |
| | | | HLA-DRB1*07:01 | |
| S | 100300 | MAYRFNGIGVTQNVLY | HLA-DRB1*04:01 | II |
| S | 100428 | QLIRAAEIRASANLAATK | HLA-DRB1*04:01 | II |
| S | 16156 | FIAGLIAIV | HLA-A*02:01 | I |
| S | 2801 | ALNTLVKQL | HLA-A*02:01 | I |
| S | 36724 | LITGRLQSL | HLA-A2 | I |
| S | 44814 | NLNESLIDL | HLA-A*02:01 | I |
| S | 50311 | QALNTLVKQLSSNFGAI | HLA-DRB1*04:01 | II |
| S | 54680 | RLNEVAKNL | HLA-A*02:01 | I |
| S | 69657 | VLNDILSRL | HLA-A*02:01 | I |
| S | 71663 | VVFLHVTYV | HLA-A*02:01 | I |

¹NA: Not available.

TABLE 9
Set of SARS-CoV-derived S and N protein T cell epitopes (obtained using positive MHC
binding assays) that are identical in SARS-CoV-2 and that maximize estimated
population coverage in China (86 distinct epitopes).

| | | | Accumulated |
|---|---|---|---|
| | MHC | | Population |
| | Allele | MHC | Coverage in |
| Epitopes\hat{ }1 | Class | Allele | China\hat{ }2 (%) |
| GSFCTQLNR, GVVFLHVTY, AQALNTLVK, MTSCCSCLK, | I | HLA- | 43.48 |
| ASANLAATK, SLIDLQELGK, SVLNDILSR, TONVLYENQK, | | A*11:01 | |
| CMTSCCSCLK, VQIDRLITGR, KTFPPTEPK, KTFPPTEPKK, | | | |
| LSPRWYFYY, ASAFFGMSR, ATEGALNTPK, QLPQGTTLPK, | | | |
| QQQGQTVTK, QQQQGQTVTK, SASAFFGMSR, | | | |
| SQASSRSSSR, TPSGTWLTY | | | |
| GYQPYRVVVL, PYRVVVLSF, LSPRWYFYY | I | HLA- | 60.68 |
| | | A*24:02 | |
| FIAGLIAIV, GLIAIVMVTI, IITTDNTFV, ALNTLVKQL, LITG | I | HLA- | 69.63 |
| RLQSL, | | A*02:01 | |
| LLLQYGSFC, LLQYGSFCT, NLNESLIDL, RLDKVEAEV, | | | |
| RLNEVAKNL, RLQSLQTYV, VLNDILSRL, VVFLHVTYV, IL | | | |
| LNKHID, | | | |
| FPRGQGVPI, LLLLDRLNQ, GMSRIGMEV, ILLNKHIDA, | | | |
| ALNTPKDHI, LALLLLDRL, LLLDRLNQL, LLLLDRLNQL, | | | |
| LQLPQGTTL, AQFAPSASA, TTLPKGFYA, VLQLPQGTTL | | | |
| FIAGLIAIV, GLIAIVMVTI, IITTDNTFV, ALNTLVKQL, LITG | I | HLA- | 75.22 |
| RLQSL, | | A*02:03 | |
| LLLQYGSFC, LLQYGSFCT, NLNESLIDL, RLDKVEAEV, | | | |
| RLNEVAKNL, RLQSLQTYV, VLNDILSRL, VVFLHVTYV, IL | | | |
| LNKHID, | | | |
| FPRGQGVPI, LLLLDRLNQ, GMSRIGMEV, ILLNKHIDA, AL | | | |
| NTPKDHI, | | | |
| LALLLLDRL, LLLDRLNQL, LLLLDRLNQL, LQLPQGTTL, | | | |
| AQFAPSASA, TTLPKGFYA, VLQLPQGTTL | | | |
| LQIPFAMQM, RVDFCGKGY | I | HLA- | 78.7 |
| | | B*15:01 | |
| AEVQIDRLIT, LQIPFAMQM, YEQYIKWPWY, AEIRASANLA, | I | HLA- | 81.43 |
| MEVTPSGTWL, GMEVTPSGTWL | | B*40:01 | |
| FIAGLIAIV, GLIAIVMVTI, IITTDNTFV, ALNTLVKQL, LITG | I | HLA- | 83.66 |
| RLQSL, | | A*02:06 | |
| LLLQYGSFC, LLQYGSFCT, NLNESLIDL, RLDKVEAEV, | | | |
| RLNEVAKNL, RLQSLQTYV, VLNDILSRL, VVFLHVTYV, IL | | | |
| LNKHID, | | | |
| FPRGQGVPI, LLLLDRLNQ, GMSRIGMEV, ILLNKHIDA, AL | | | |
| NTPKDHI, | | | |
| LALLLLDRL, LLLDRLNQL, LLLLDRLNQL, LQLPQGTTL, | | | |
| AQFAPSASA, TTLPKGFYA, VLQLPQGTTL | | | |
| GSFCTQLNR, GVVFLHVTY, AQALNTLVK, MTSCCSCLK, | I | HLA- | 85.09 |
| ASANLAATK, SLIDLQELGK, SVLNDILSR, TQNVLYENQK, | | A*31:01 | |
| CMTSCCSCLK, VQIDRLITGR, KTFPPTEPK, KTFPPTEPKK, | | | |
| LSPRWYFYY, ASAFFGMSR, ATEGALNTPK, QLPQGTTLPK, | | | |
| QQQGQTVTK, QQQGQTVTKK, QQQQGQTVTK, | | | |
| SASAFFGMSR, SQASSRSSSR, TPSGTWLTY | | | |
| MTSCCSCLK, SLIDLQELGK, CMTSCCSCLK, VQIDRLITGR, | I | HLA- | 86.38 |
| SASAFFGMSR, SQASSRSSSR | | A*33:01 | |
| FPNITNLCPF, APHGVVFLHV, FPRGQGVPI, APSASAFFGM | I | HLA- | 87.54 |
| | | B*51:01 | |
| LQIPFAMQM, RVDFCGKGY | I | HLA- | 88.55 |
| | | B*58:01 | |
| GSFCTQLNR, GVVFLHVTY, AQALNTLVK, MTSCCSCLK, | I | HLA- | 89.43 |
| ASANLAATK, SLIDLQELGK, SVLNDILSR, TONVLYENQK, | | A*03:01 | |
| CMTSCCSCLK, VQIDRLITGR, KTFPPTEPK, KTFPPTEPKK, | | | |
| LSPRWYFYY, ASAFFGMSR, ATEGALNTPK, QLPQGTTLPK, | | | |
| QQQGQTVTK, QQQGQTVTKK, QQQQGQTVTK, | | | |
| SASAFFGMSR, SQASSRSSSR, TPSGTWLTY | | | |
| VRFPNITNL | I | HLA- | 90.16 |
| | | C*14:02 | |
| LQIPFAMQM, RVDFCGKGY | I | HLA- | 90.72 |
| | | C*15:02 | |
| FPNITNLCPF, APHGVVFLHV, FPRGQGVPI, APSASAFFGM | I | HLA- | 91.16 |
| | | B*35:01 | |
| FPNITNLCPF, APHGVVFLHV, FPRGQGVPI, APSASAFFGM | I | HLA- | 91.51 |
| | | B*54:01 | |
| GSFCTQLNR, GVVFLHVTY, AQALNTLVK, MTSCCSCLK, | I | HLA- | 91.84 |
| ASANLAATK, SLIDLQELGK, SVLNDILSR, TQNVLYENQK, | | A*68:01 | |
| CMTSCCSCLK, VQIDRLITGR, KTFPPTEPK, KTFPPTEPKK, | | | |
| LSPRWYFYY, ASAFFGMSR, ATEGALNTPK, QLPQGTTLPK, | | | |
| QQQGQTVTK, QQQGQTVTKK, QQQQGQTVTK, SASAFFGMSR, | | | |
| SQASSRSSSR, TPSGTWLTY | | | |
| FPNITNLCPF, APHGVVFLHV, FPRGQGVPI, APSASAFFGM | I | HLA- | 92.15 |
| | | B*07:02 | |
| GYQPYRVVVL, PYRVVVLSF, LSPRWYFYY | I | HLA- | 92.39 |
| | | A*23:01 | |
| GAALQIPFAMQMAYR, GWTFGAGAALQIPFA, IDRLITGRLQS | II | HLA- | 92.6 |
| LQTY, | | DRB1*01: | |
| ISGINASVVNIQKEI, LDK YFKNHTSPDVDL, LGDISGINASVV | | 01 | |
| NIQ, | | | |
| LGFIAGLIAIVMVTI, LNTLVKQLSSNFGAI, LQDVVNQNAQA | | | |
| LNTL, | | | |
| LQSLQTYVTQQLIRA, LQTYVTQQLIRAAEI, AQKFNGLTVLP | | | |
| PLLT, | | | |
| PCSFGGVSVITPGTN, QIPFAMQMAYRENGI, QQLIRAAEIRAS | | | |
| ANL, | | | |
| QTYVTQQLIRAAEIR, AYRENGIGVTQNVLY, SSNFGAISSVL | | | |
| NDIL, | | | |
| TGRLQSLQTYVTQQL, WLGFIAGLIAIVMVT, CVNFNFNGLT | | | |
| GTGVL, | | | |
| DKYFKNHTSPDVDLG, GQTVTKKSAAEASKK, IDAYKTFPPT | | | |
| EPKKD, | | | |
| MSRIGMEVTPSGTWL, NKHIDA YKTFPPTEP, VLQLPQGTTLP | | | |
| KGFY | | | |
| YEQYIKWPWY | I | HLA- | 92.68 |
| | | B*18:01 | |
| FPRGQGVPI | I | HLA- | 92.76 |
| | | B*08:01 | |

\hat{ }1Multiple SARS-CoV-derived epitopes that were determined using MHC binding assays are shown for each allele. Epitopes that were also tested for positive T cell response (listed also in Table 2) are shown in bold font. Epitopes that lie within the SARS-CoV receptor-binding motif are underlined.
\hat{ }2Epitopes are ordered according to the estimated accumulated population coverage.

Due to the promiscuous nature of binding between peptides and MHC alleles, multiple S and N peptides were reported to bind to individual MHC alleles. Thus, while we list all the S and N epitopes that bind to each MHC allele (Table 3), the estimated maximum population coverage may be achieved by selecting at least one epitope for each listed MHC allele. Likewise, many individual S and N epitopes were found to be presented by multiple alleles and thereby estimated to have varying global population coverage (listed in Table 10).

TABLE 3
Set of the SARS-CoV-derived spike (S) and nucleocapsid (N) protein T cell epitopes
(obtained from positive MHC binding assays) that are identical in SARS-CoV-2 and that
maximize estimated population coverage globally (87 distinct epitopes).

| | | | Global | Accumulated |
|---|---|---|---|---|
| | MHC | | Accumulated | Population |
| | Allele | MHC | Population | Coverage in |
| Epitopes¹ | Class | Allele | Coverage² (%) | China (%) |
| FIAGLIAIV, GLIAIVMVTI, | I | HLA-A*02:01 | 39.08 | 14.62 |
| IITTDNTFV, ALNTLVKQL, | | | | |
| LITGRLQSL, LLLQYGSFC, | | | | |
| LQYGSFCT, NLNESLIDL, | | | | |
| RLDKVEAEV, RLNEVAKNL, | | | | |
| RLQSLQTYV, VLNDILSRL, | | | | |
| VVFLHVTYV, ILLNKHID, | | | | |
| FPRGQGVPI, LLLLDRLNQ, | | | | |
| GMSRIGMEV, ILLNKHIDA, | | | | |
| ALNTPKDHI, LALLLLDRL, | | | | |
| LLLDRLNQL, LLLLDRLNQL, | | | | |
| LQLPQGTTL, AQFAPSASA, | | | | |
| TTLPKGFYA, VLQLPQGTTL | | | | |
| GYQPYRVVVL, PYRVVVLSF, | I | HLA-A*24:02 | 55.48 | 36.11 |
| LSPRWYFYY I | | | | |
| DSFKEELDKY, LIDLQELGKY, | I | HLA-A*01:01 | 66.78 | 39.09 |
| PYRVVVLSF, GTTLPKGFY, | | | | |
| VTPSGTWLTY | | | | |
| GSFCTQLNR, GVVFLHVTY, | I | HLA-A*03:01 | 76.14 | 41.68 |
| AQALNTLVK, MTSCCSCLK, | | | | |
| ASANLAATK, SLIDLQELGK, | | | | |
| SVLNDILSR, TQNVLYENQK, | | | | |
| CMTSCCSCLK, VQIDRLITGR, | | | | |
| KTFPPTEPK, KTFPPTEPKK, | | | | |
| LSPRWYFYY, ASAFFGMSR, | | | | |
| ATEGALNTPK, QLPQGTTLPK, | | | | |
| QQQGQTVTK, QQQQGQTVTK, | | | | |
| SASAFFGMSR, SQASSRSSSR, | | | | |
| TPSGTWLTY | | | | |
| GSFCTQLNR, GVVFLHVTY, | I | HLA-A*11:01 | 83.39 | 73.43 |
| AQALNTLVK, MTSCCSCLK, | | | | |
| ASANLAATK, SLIDLQELGK, | | | | |
| SVLNDILSR, TQNVLYENQK, | | | | |
| CMTSCCSCLK, VQIDRLITGR, | | | | |
| KTFPPTEPK, KTFPPTEPKK, | | | | |
| LSPRWYFYY, ASAFFGMSR, | | | | |
| ATEGALNTPK, QLPQGTTLPK, | | | | |
| QQQGQTVTK, QQQQGQTVTK, | | | | |
| SASAFFGMSR, SQASSRSSSR, | | | | |
| TPSGTWLTY | | | | |
| GSFCTQLNR, GVVFLHVTY, | I | HLA-A*68:01 | 85.71 | 74.25 |
| AQALNTLVK, MTSCCSCLK, | | | | |
| ASANLAATK, SLIDLQELGK, | | | | |
| SVLNDILSR, TQNVLYENQK, | | | | |
| CMTSCCSCLK, VQIDRLITGR, | | | | |
| KTFPPTEPK, KTFPPTEPKK, | | | | |
| LSPRWYFYY, ASAFFGMSR, | | | | |
| ATEGALNTPK, QLPQGTTLPK, | | | | |
| QQQGQTVTK, QQQQGQTVTK, | | | | |
| SASAFFGMSR, SQASSRSSSR, | | | | |
| TPSGTWLTY | | | | |
| GYQPYRVVVL, PYRVVVLSF, | I | HLA-A*23:01 | 87.72 | 74.87 |
| LSPRWYFYY | | | | |
| GSFCTQLNR, GVVFLHVTY, | I | HLA-A*31:01 | 89.55 | 76.93 |
| AQALNTLVK, MTSCCSCLK, | | | | |
| ASANLAATK, SLIDLQELGK, | | | | |
| SVLNDILSR, TQNVLYENQK, | | | | |
| CMTSCCSCLK, VQIDRLITGR, | | | | |
| KTFPPTEPK, KTFPPTEPKK, | | | | |
| LSPRWYFYY, ASAFFGMSR, | | | | |
| ATEGALNTPK, QLPQGTTLPK, | | | | |
| QQQGQTVTK, QQQQGQTVTK, | | | | |
| SASAFFGMSR, SQASSRSSSR, | | | | |
| TPSGTWLTY | | | | |
| FPNITNLCPF, APHGVVFLHV, | I | HLA-B*07:02 | 90.89 | 77.61 |
| FPRGQGVPI, APSASAFFGM | | | | |
| GAALQIPFAMQMAYR, | II | HLA- | 91.94 | 78.23 |
| GWTFGAGAALQIPFA, | | DRB1*01:01 | | |
| IDRLITGRLQSLQTY, | | | | |
| ISGINASVVNIQKEI, | | | | |
| LDKYFKNHTSPDVDL, | | | | |
| LGDISGINASVVNIQ, | | | | |
| LGFIAGLIAIVMVTI, | | | | |
| LNTLVKQLSSNFGAI, | | | | |
| LQDVVNQNAQALNTL, | | | | |
| LQSLQTYVTQQLIRA, | | | | |
| LQTYVTQQLIRAAEI, | | | | |
| AQKFNGLTVLPPLLT, | | | | |
| PCSFGGVSVITPGTN, | | | | |
| QIPFAMQMAYRENGI, | | | | |
| QQLIRAAEIRASANL, | | | | |
| QTYVTQQLIRAAEIR, | | | | |
| AYRFNGIGVTQNVLY, | | | | |
| SSNFGAISSVLNDIL, | | | | |
| TGRLQSLQTYVTQQL, | | | | |
| WLGFIAGLIAIVMVT, | | | | |
| CVNFNFNGLTGTGVL, | | | | |
| DKYFKNHTSPDVDLG, | | | | |
| IDAYKTFPPTEPKKD, | | | | |
| MSRIGMEVTPSGTWL, | | | | |
| NKHIDAYKTFPPTEP, | | | | |
| VLQLPQGTTLPKGFY | | | | |
| FPRGQGVPI | I | HLA-B*08:01 | 92.85 | 78.41 |
| FPNITNLCPF, APHGVVFLHV, | I | HLA-B*35:01 | 93.53 | 79.23 |
| FPRGQGVPI, APSASAFFGM | | | | |
| LQIPFAMQM, RVDFCGKGY | I | HLA-B*15:01 | 94.18 | 82.26 |
| FPNITNLCPF, APHGVVFLHV, | I | HLA-B*51:01 | 94.72 | 83.73 |
| FPRGQGVPI, APSASAFFGM | | | | |
| YEQYIKWPWY | I | HLA-B*18:01 | 95.23 | 83.88 |
| GRLQSLQTY, RVDFCGKGY, | I | HLA-B*27:05 | 95.55 | 84 |
| VRFPNITNL | | | | |
| MTSCCSCLK, SLIDLQELGK, | I | HLA-A*33:01 | 95.79 | 85.28 |
| CMTSCCSCLK, VQIDRLITGR, | | | | |
| SASAFFGMSR, | | | | |
| SQASSRSSSR | | | | |
| LQIPFAMQM, RVDFCGKGY | I | HLA-B*58:01 | 95.99 | 86.45 |
| LQIPFAMQM, RVDFCGKGY | I | HLA-C*15:02 | 96.17 | 87.22 |
| VRFPNITNL | I | HLA-C*14:02 | 96.29 | 88.11 |

¹Multiple SARS-CoV-derived epitopes that were determined using MHC binding assays are shown for each allele. Epitopes that were also tested for positive T cell response (listed also in Table 2) are shown in bold text. Epitopes that lie within the SARS-CoV receptor-binding motif are underlined.
²Epitopes are ordered according to the estimated global accumulated population coverage.

TABLE 10
Estimated global and Chinese population coverages for the individual SARS-CoV-
derived S or N protein T cell epitopes (obtained using positive MHC binding assays)
that are identical in SARS-CoV-2.

| | | | | Global | Population |
|---|---|---|---|---|---|
| | | | | Population | Coverage |
| | | | | Coverage | in China |
| Protein | IEDB | Epitope | MHC Allele\hat{ }1 | \hat{ }2 (%) | (%) |
| N | 17385 | FPRGQGVPI | HLA-B*07:02, HLA-B*54:01, HLA- | 62.89 | 29.97 |
| | | | B*08:01, HLA-B*35:01, HLA- | | |
| | | | B*51:01, HLA-A*02:01, HLA-B*53:01 | | |
| N | 39576 | LSPRWYFYY | HLA-A*01:01, HLA-A*29:02, HLA- | 60.51 | 66.71 |
| | | | A*30:02, HLA-A*11:01, HLA- | | |
| | | | A*23:01, HLA-A*24:02, HLA-A*31:01 | | |
| S | 50166 | PYRVVVLSF | HLA-A*23:01, HLA-A*24:02, HLA- | 45.41 | 31.33 |
| | | | A*01:01, HLA-A*26:01 | | |
| S | 16156 | FIAGLIAIV | HLA-A*02:01, HLA-A*02:02, HLA- | 43.68 | 30.09 |
| | | | A*02:03, HLA-A*02:06, HLA- | | |
| | | | A*68:02, HLA-A2 | | |
| S | 20907 | GLIAIVMVTI | HLA-A*02:02, HLA-A*02:03, HLA- | 43.68 | 30.09 |
| | | | A*02:01, HLA-A*02:06, HLA-A*68:02 | | |
| S | 54507 | RLDKVEAEV | HLA-A*02:01, HLA-A*02:02, HLA- | 43.68 | 30.09 |
| | | | A*02:06, HLA-A*02:03, HLA-A*68:02 | | |
| S | 54725 | RLQSLQTYV | HLA-A*02:01, HLA-A*02:02, HLA- | 43.68 | 30.09 |
| | | | A*02:03, HLA-A*02:06, HLA-A*68:02 | | |
| S | 71663 | VVFLHVTYV | HLA-A*02:01, HLA-A*02:02, HLA- | 43.68 | 30.09 |
| | | | A*02:03, HLA-A*02:06, HLA-A*68:02 | | |
| S | 56252 | RVDFCGKGY | HLA-A*30:02, HLA-A*01:01, HLA- | 43.07 | 20.97 |
| | | | A*03:01, HLA-B*15:01, HLA-B*27:05 | | |
| N | 3956 | AQFAPSASA | HLA-A*02:02, HLA-A*02:06, HLA- | 41.78 | 29.89 |
| | | | A*02:01, HLA-A*02:03 | | |
| S | 42873 | MTSCCSCLK | HLA-A*11:01, HLA-A*31:01, HLA- | 41.4 | 52.89 |
| | | | A*33:01, HLA-A*68:01, HLA-A*03:01 | | |
| S | 59161 | SLIDLQELGK | HLA-A*03:01, HLA-A*11:01, HLA- | 41.4 | 52.89 |
| | | | A*31:01, HLA-A*68:01, HLA-A*33:01 | | |
| S | 6668 | CMTSCCSCLK | HLA-A*68:01, HLA-A*03:01, HLA- | 41.4 | 52.89 |
| | | | A*11:01, HLA-A*31:01, HLA-A*33:01 | | |
| S | 70575 | VQIDRLITGR | HLA-A*31:01, HLA-A*03:01, HLA- | 41.4 | 52.89 |
| | | | A*11:01, HLA-A*33:01, HLA-A*68:01 | | |
| N | 56979 | SASAFFGMSR | HLA-A*11:01, HLA-A*31:01, HLA- | 41.4 | 52.89 |
| | | | A*68:01, HLA-A*33:01, HLA-A*03:01 | | |
| S | 22322 | GSFCTQLNR | HLA-A*11:01, HLA-A*31:01, HLA- | 40.03 | 50.15 |
| | | | A*68:01, HLA-A*03:01 | | |
| S | 3939 | AQALNTLVK | HLA-A*11:01, HLA-A*03:01, HLA- | 40.03 | 50.15 |
| | | | A*31:01, HLA-A*68:01 | | |
| S | 4321 | ASANLAATK | HLA-A*11:01, HLA-A*03:01, HLA- | 40.03 | 50.15 |
| | | | A*68:01, HLA-A*31:01 | | |
| N | 33667 | KTFPPTEPK | HLA-A*11:01, HLA-A*03:01, HLA- | 40.03 | 50.15 |
| | | | A*31:01, HLA-A*68:01 | | |
| N | 33668 | KTFPPTEPKK | HLA-A*03:01, HLA-A*11:01, HLA- | 40.03 | 50.15 |
| | | | A*68:01, HLA-A*31:01 | | |
| N | 4307 | ASAFFGMSR | HLA-A*31:01, HLA-A*11:01, HLA- | 40.03 | 50.15 |
| | | | A*68:01, HLA-A*03:01 | | |
| N | 4936 | ATEGALNTPK | HLA-A*11:01, HLA-A*03:01, HLA- | 40.03 | 50.15 |
| | | | A*68:01, HLA-A*31:01 | | |
| S | 26710 | IITTDNTFV | HLA-A*02:01 | 39.08 | 14.62 |
| S | 2801 | ALNTLVKQL | HLA-A*02:01 | 39.08 | 14.62 |
| S | 36724 | LITGRLQSL | HLA-A2, HLA-A*02:01 | 39.08 | 14.62 |
| S | 37544 | LLLQYGSFC | HLA-A*02:01 | 39.08 | 14.62 |
| S | 37724 | LLQYGSFCT | HLA-A*02:01 | 39.08 | 14.62 |
| S | 44814 | NLNESLIDL | HLA-A*02:01 | 39.08 | 14.62 |
| S | 54680 | RLNEVAKNL | HLA-A*02:01 | 39.08 | 14.62 |
| S | 69657 | VLNDILSRL | HLA-A*02:01 | 39.08 | 14.62 |
| N | 125100 | ILLNKHID | HLA-A*02:01 | 39.08 | 14.62 |
| N | 193498 | LLLLDRLNQ | HLA-A*02:01 | 39.08 | 14.62 |
| N | 21347 | GMSRIGMEV | HLA-A*02:01 | 39.08 | 14.62 |
| N | 27182 | ILLNKHIDA | HLA-A*02:01 | 39.08 | 14.62 |
| N | 2802 | ALNTPKDHI | HLA-A*02:01 | 39.08 | 14.62 |
| N | 34851 | LALLLLDRL | HLA-A*02:01 | 39.08 | 14.62 |
| N | 37473 | LLLDRLNQL | HLA-A*02:01 | 39.08 | 14.62 |
| N | 37515 | LLLLDRLNQL | HLA-A*02:01 | 39.08 | 14.62 |
| N | 38881 | LQLPQGTTL | HLA-A*02:01 | 39.08 | 14.62 |
| N | 66706 | TTLPKGFYA | HLA-A*02:01 | 39.08 | 14.62 |
| N | 69720 | VLQLPQGTTL | HLA-A*02:01 | 39.08 | 14.62 |
| N | 51482 | QLPQGTTLPK | HLA-A*03:01, HLA-A*11:01, HLA- | 35.75 | 47.14 |
| | | | A*68:01 | | |
| S | 17341 | FPNITNLCPF | HLA-B*35:01, HLA-B*51:01, HLA- | 30.32 | 17.23 |
| | | | B*53:01, HLA-B*07:02, HLA-B*54:01 | | |
| S | 23436 | GYQPYRVVVL | HLA-A*23:01, HLA-A*24:02, HLA- | 29.52 | 24.77 |
| | | | A*29:02 | | |
| N | 3810 | APSASAFFGM | HLA-B*07:02, HLA-B*35:01, HLA- | 29.39 | 14.4 |
| | | | B*51:01, HLA-B*53:01 | | |
| S | 73751 | YEQYIKWPWY | HLA-B*44:02, HLA-B*18:01, HLA- | 28.45 | 16.31 |
| | | | B*44:03, HLA-B*40:01, HLA-B*45:01 | | |
| S | 10112 | DSFKEELDKY | HLA-A*26:01, HLA-A*29:02, HLA- | 28.12 | 8.7 |
| | | | A*30:02, HLA-A*01:01 | | |
| S | 36481 | LIDLQELGKY | HLA-A*30:02, HLA-A*01:01, HLA- | 28.12 | 8.7 |
| | | | A*26:01, HLA-A*29:02 | | |
| N | 71461 | VTPSGTWLTY | HLA-A*30:02, HLA-A*01:01, HLA- | 28.12 | 8.7 |
| | | | A*26:01, HLA-A*29:02 | | |
| N | 60380 | SQASSRSSSR | HLA-A*31:01, HLA-A*68:01, HLA- | 27.17 | 50.57 |
| | | | A*11:01, HLA-A*33:01 | | |
| S | 62221 | SVLNDILSR | HLA-A*11:01, HLA-A*31:01, HLA- | 25.64 | 47.76 |
| | | | A*68:01 | | |
| S | 1221 | AEVQIDRLIT | HLA-B*40:02, HLA-B*44:03, HLA- | 25.05 | 18.12 |
| | | | B*45:01, HLA-B*40:01, HLA-B*44:02 | | |
| S | 999 | AEIRASANLA | HLA-B*45:01, HLA-B*40:01, HLA- | 25.05 | 18.12 |
| | | | B*44:02, HLA-B*44:03, HLA-B*40:02 | | |
| S | 3589 | APHGVVFLHV | HLA-B*07:02, HLA-B*54:01, HLA- | 23.86 | 10.24 |
| | | | B*35:01, HLA-B*53:01 | | |
| N | 60242 | SPRWYFYYL | HLA-B*07:02, HLA-B*51:01, HLA- | 22.96 | 13.78 |
| | | | B*53:01, HLA-B*54:01 | | |
| S | 38855 | LQIPFAMQM | HLA-B*15:01, HLA-C*15:02, HLA- | 22.62 | 34.66 |
| | | | B*40:01, HLA-B*58:01 | | |
| S | 65906 | TQNVLYENQK | HLA-A*11:01, HLA-A*68:01 | 20.88 | 44.68 |
| N | 52114 | QQQGQTVTK | HLA-A*11:01, HLA-A*31:01 | 20.45 | 46.6 |
| N | 65763 | TPSGTWLTY | HLA-A*29:02, HLA-A*11:01 | 19.09 | 43.75 |
| S | 100048 | GAALQIPFAMQMAY | HLA-DRA*01:01, DRB1*07:01 | 18.23 | 8.26 |
| | | RF | | | |
| S | 1220 | AEVQIDRLI | HLA-B*40:02, HLA-B*44:02, HLA- | 17.99 | 6.65 |
| | | | B*44:03, HLA-B*45:01 | | |
| S | 50641 | QELGKYEQYI | HLA-B*44:02, HLA-B*44:03, HLA- | 16.84 | 6.32 |
| | | | B*40:02 | | |
| N | 2431 | ALALLLLDR | HLA-A*03:01 | 16.81 | 3.32 |
| S | 23200 | GVVFLHVTY | HLA-A*11:01 | 15.53 | 43.48 |
| N | 52129 | QQQQGQTVTK | HLA-A*11:01 | 15.53 | 43.48 |
| S | 51999 | QPYRVVVLSF | HLA-B*07:02, HLA-B*53:01 | 15.14 | 3.62 |
| N | 22856 | GTTLPKGFY | HLA-A*30:02, HLA-A*29:02, HLA- | 11.9 | 5.06 |
| | | | A*26:01 | | |
| S | 18514 | GAALQIPFAMQMAYR | HLA-DRB1*01:01 | 11.53 | 2.77 |
| S | 23293 | GWTFGAGAALQIPFA | HLA-DRB1*01:01 | 11.53 | 2.77 |
| S | 25662 | IDRLITGRLQSLQTY | HLA-DRB1*01:01 | 11.53 | 2.77 |
| S | 28511 | ISGINASVVNIQKEI | HLA-DRB1*01:01 | 11.53 | 2.77 |
| S | 35205 | LDKYFKNHTSPDVDL | HLA-DRB1*01:01 | 11.53 | 2.77 |
| S | 36075 | LGDISGINASVVNIQ | HLA-DRB1*01:01 | 11.53 | 2.77 |
| S | 36103 | LGFIAGLIAIVMVTI | HLA-DRB1*01:01 | 11.53 | 2.77 |
| S | 38353 | LNTLVKQLSSNFGAI | HLA-DRB1*01:01 | 11.53 | 2.77 |
| S | 38831 | LQDVVNQNAQALNTL | HLA-DRB1*01:01 | 11.53 | 2.77 |
| S | 38990 | LQSLQTYVTQQLIRA | HLA-DRB1*01:01 | 11.53 | 2.77 |
| S | 39003 | LQTYVTQQLIRAAEI | HLA-DRB1*01:01 | 11.53 | 2.77 |
| S | 3982 | AQKFNGLTVLPPLLT | HLA-DRB1*01:01 | 11.53 | 2.77 |
| S | 47041 | PCSFGGVSVITPGTN | HLA-DRB1*01:01 | 11.53 | 2.77 |
| S | 51112 | QIPFAMQMAYRENGI | HLA-DRB1*01:01 | 11.53 | 2.77 |
| S | 52057 | QQLIRAAEIRASANL | HLA-DRB1*01:01 | 11.53 | 2.77 |
| S | 52672 | QTYVTQQLIRAAEIR | HLA-DRB1*01:01 | 11.53 | 2.77 |
| S | 5908 | AYRFNGIGVTQNVLY | HLA-DRB1*01:01 | 11.53 | 2.77 |
| S | 61229 | SSNFGAISSVLNDIL | HLA-DRB1*01:01 | 11.53 | 2.77 |
| S | 63951 | TGRLQSLQTYVTQQL | HLA-DRB1*01:01 | 11.53 | 2.77 |
| S | 72717 | WLGFIAGLIAIVMVT | HLA-DRB1*01:01 | 11.53 | 2.77 |
| S | 7289 | CVNFNFNGLTGTGVL | HLA-DRB1*01:01 | 11.53 | 2.77 |
| S | 9006 | DKYFKNHTSPDVDLG | HLA-DRB1*01:01 | 11.53 | 2.77 |
| N | 25542 | IDAYKTFPPTEPKKD | HLA-DRB1*01:01 | 11.53 | 2.77 |
| N | 42648 | MSRIGMEVTPSGTWL | HLA-DRB1*01:01 | 11.53 | 2.77 |
| N | 44501 | NKHIDAYKTFPPTEP | HLA-DRB1*01:01 | 11.53 | 2.77 |
| N | 69721 | VLQLPQGTTLPKGFY | HLA-DRB1*01:01 | 11.53 | 2.77 |
| S | 100300 | MAYRFNGIGVTQNV | HLA-DRB1*04:01 | 11.21 | 1.92 |
| | | LY | | | |
| S | 100428 | QLIRAAEIRASANLA | HLA-DRB1*04:01 | 11.21 | 1.92 |
| | | ATK | | | |
| S | 50311 | QALNTLVKQLSSNF | HLA-DRB1*04:01 | 11.21 | 1.92 |
| | | GAI | | | |
| N | 190494 | MEVTPSGTWL | HLA-B*40:01 | 7.81 | 11.88 |
| N | 193464 | GMEVTPSGTWL | HLA-B*40:01 | 7.81 | 11.88 |
| S | 70718 | VRFPNITNL | HLA-C*14:02, HLA-B*27:05 | 7.68 | 7.49 |
| S | 26198 | IGAGICASY | HLA-A*29:02, HLA-A*30:02 | 6.27 | 0.81 |
| S | 22144 | GRLQSLQTY | HLA-B*27:05 | 4.78 | 0.63 |
| N | 956 | AEGSRGGSQA | HLA-B*45:01 | 1.26 | 0.35 |

Mapping the SARS-CoV-Derived B cell Epitopes that Are Identical in SARS-CoV-2. Similar to T cell epitopes, we used in our study the SARS-CoV-derived B cell epitopes that have been experimentally-determined from positive B cell assays [29]. These epitopes were classified as: (i) Linear B cell epitopes (antigenic peptides), and (ii) discontinuous B cell epitopes (conformational epitopes with resolved structural determinants).

We aligned the 298 linear B cell epitopes (Table 1) across the SARS-CoV-2 proteins and found that 49 epitope-sequences, all derived from structural proteins, have an identical match and comprised no mutation in the available SARS-CoV-2 protein sequences (as of 21 Feb. 2020). Interestingly, a large number (45) of these were derived from either the S (23) or N (22) protein (Table 4), while the remaining (4) were from the M protein (Table 11).

TABLE 4
SARS-COV-derived linear B cell epitopes from S (23; 20 of which
are located in subunit S2) and N (22) proteins
that are identical in SARS-CoV-2 (45 epitopes in total).

| Pro- | Sub- | IEDB | | Pro- | IEDB | Epitope |
|---|---|---|---|---|---|---|
| tein | unit | ID | Epitope | tein | ID | |
| S | S2 | 10778 | DVVNQNAQALNTLVKQL | N | 15814 | FFGMSRIGMEVTPSGTW |
| S | S2 | 11038 | EAEVQIDRLITGRLQSL | N | 21065 | GLPNNTASWFTALTQHGK |
| S | S2 | 12426 | EIDRLNEVAKNLNESLI | N | 22855 | GTTLPK |
| | | | DLQELGKYEQY | | | |
| S | S2 | 14626 | EVAKNLNESLIDLQELG | N | 28371 | IRQGTDYKHWPQIAQFA |
| S | S2 | 18515 | GAALQIPFAMQMAYRFN | N | 31116 | KHIDAYKTFPPTEPKKDKKK |
| S | S1 | 18594 | GAGICASY | N | 31166 | KHWPQIAQFAPSASAFF |
| S | S2 | 2092 | AISSVLNDILSRLDKVE | N | 75235 | YNVTQAFGRRGPEQTQGNF |
| S | S2 | 22321 | GSFCTQLN | N | 33669 | KTFPPTEPKKDKKKK |
| S | S2 | 27357 | ILSRLDKVEAEVQIDRL | N | 37640 | LLPAAD |
| S | S1 | 30987 | KGIYQTSN | N | 38249 | LNKHIDAYKTFPPTEPK |
| S | S2 | 3176 | AMQMAYRF | N | 38648 | LPQGTTLPKG |
| S | S2 | 32508 | KNHTSPDVDLGDISGIN | N | 38657 | LPQRQKKQ |
| S | S2 | 41177 | MAYRFNGIGVTQNVLYE | N | 48067 | PKGFYAEGSRGGSQASSR |
| S | S2 | 462 | AATKMSECVLGQSKRVD | N | 50741 | QFAPSASAFFGMSRIGM |
| S | S2 | 47479 | PFAMQMAYRENGIGVTQ | N | 50965 | QGTDYKHW |
| S | S2 | 50311 | QALNTLVKQLSSNFGAI | N | 51483 | QLPQGTTLPKGFYAE |
| S | S2 | 51379 | QLIRAAEIRASANLAAT | N | 51484 | QLPQGTTLPKGFYAEGSR |
| S | S1 | 52020 | QQFGRD | N | 51485 | QLPQGTTLPKGFYAEGSRGG |
| | | | | | | SQ |
| S | S2 | 53202 | RASANLAATKMSECVLG | N | 63729 | TFPPTEPK |
| S | S2 | 54599 | RLITGRLQSLQTYVTQQ | N | 55683 | RRPQGLPNNTASWFT |
| S | S2 | 558417 | EIDRLNEVAKNLNESLI | N | 60379 | SQASSRSS |
| | | | DLQELGKYEQY | | | |
| S | S2 | 59425 | SLQTYVTQQLIRAAEIR | N | 60669 | SRGGSQASSRSSSRSR |
| S | S2 | 9094 | DLGDISGINASVVNIQK | | | |

TABLE 11
SARS-CoV-derived linear B cell
epitopes, excluding those in
S and N proteins, that are
identical in SARS-CoV-2.

| Pro- | IEDB | |
|---|---|---|
| tein | ID | Epitope |
| M | 21996 | GRCDIKDLPKEITVATSR |
| M | 29127 | ITVATSRT |
| M | 48052 | PKEITVATSRTLSYYKL |
| M | 66409 | TSRTLSYYKLGASQRV |

On the other hand, all 6 SARS-CoV-derived discontinuous B cell epitopes obtained from the ViPR database (Table 5) were derived from the S protein. Based on the pairwise alignment between the SARS-CoV and SARS-CoV-2 reference sequences (FIG. 5), we found that none of these mapped identically to the SARS-CoV-2 S protein, in contrast to the linear epitopes. For 3 of these discontinuous B cell epitopes (corresponding to antibodies 5230, m396, and 80R [39-41]), there was a partial mapping, with at least one site having an identical residue at the corresponding site in the SARS-CoV-2 S protein (Table 5).

TABLE 5
SARS-CoV-derived discontinuous B cell epitopes (and associated known antibodies [39-41])
that have at least one site with an identical amino acid to the corresponding site in SARS-CoV-2.

| | Associated | |
|---|---|---|
| IEDB ID | known antibody | SARS-CoV S protein residues^{1,2} |
| 910052 | S230 | G446, P462, D463, Y475 |
| 77444 | m396 | T359, T363, K365, K390, G391, D392, R395, R426, Y436, G482, Y484, T485, |
| | | T486, T487, G488, I489, G490, Y491, Q492, Y494 |
| 77442 | 80R | R426, S432, T433, Y436, N437, K439, Y440, Y442, P469, P470, A471, L472, |
| | | N473, C474, Y475, W476, L478, N479, D480, Y481, G482, Y484, T485, |
| | | T486, T487, G488, I489, Y491, Q492 |

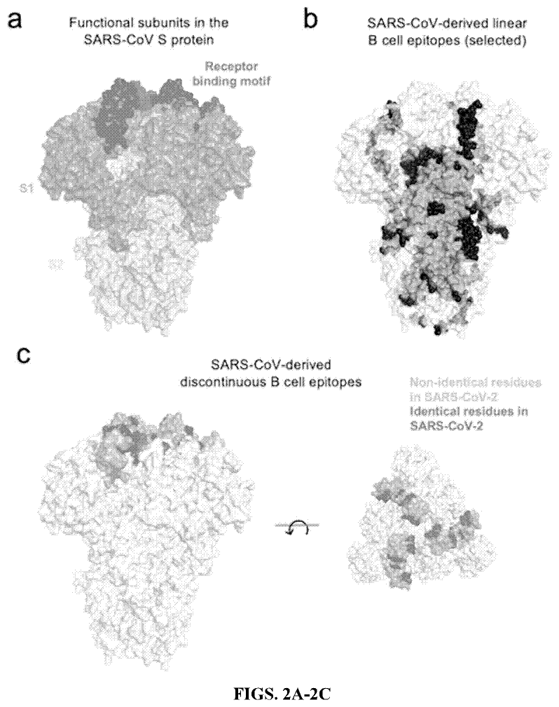

Mapping the residues of the linear and discontinuous B cell epitopes onto the available structure of the SARS-CoV S protein revealed their distinct association with the two functional subunits of the S protein [42]: S1, important for interaction with the host cell receptor, and S2, involved in fusion of the cellular and virus membranes (FIG. 2A). Specifically, 20 of the 23 linear epitopes (Table 4) mapped to S2 (FIG. 2B). Thus, the antibodies targeting the identified linear epitopes in the S2 subunit might cross-react and neutralize both SARS-CoV and SARS-CoV-2, as suggested in a very recent study [43]. While S2 is comparatively less exposed than S1, it may be accessible to antibodies during the complex conformational changes involved in viral entry of coronaviruses [44-46]; though this remains to be more clearly understood. In contrast, the 3 discontinuous B cell epitopes (Table 5) mapped onto the more exposed S1 subunit (FIG. 2C, left panel), which contains the receptor-binding motif of the SARS-CoV S protein [34]. We observed that very few residues of the 3 discontinuous epitopes were identical within SARS-CoV and SARS-CoV-2 (FIG. 2c, right panel). These differences suggest that the SARS-CoV-specific antibodies S230, m396, and 80R known to bind to these epitopes in SARS-CoV might not be able to bind to the same regions in SARS-CoV-2 S protein. Interestingly, while this paper was under review, this has been confirmed experimentally [47]. Further studies are currently under way to identify other SARS-CoV antibodies that may bind to discontinuous epitopes of the SARS-CoV-2 S protein [48].

### Discussion

The quest for a vaccine against the novel SARS-CoV-2 is recognized as an urgent problem. Effective vaccination could indeed play a significant role in curbing the spread of the virus, and help to eliminate it from the human population. However, scientific efforts to address this challenge are only just beginning. Much remains to be learnt about the virus, its biological properties, epidemiology, etc. At this early stage, there is also a lack of information about specific immune responses against SARS-CoV-2, which presents a challenge for vaccine development.

This study has sought to assist with the initial phase of vaccine development by providing recommendations of epitopes that may potentially be considered for incorporation in vaccine designs. Despite having limited understanding of how the human immune system responds naturally to SARS-CoV-2, these epitopes are motivated by responses they have recorded in SARS-CoV (or, for the case of T cell epitopes, to at least confer MHC binding), and the fact that they map identically to SARS-CoV-2, based on the available sequence data (as of 21 Feb. 2020). This important observation should not be taken for granted. Despite the apparent similarity between SARS-CoV and SARS-CoV-2, there is still considerable genetic variation between the two, and it is not obvious a-prior if epitopes that elicit an immune response against SARS-CoV are likely to be effective against SARS-CoV-2. We found that only 23% and 16% of known SARS-CoV T cell and B cell epitopes map identically to SARS-CoV-2, respectively, and with no mutation having been observed in these epitopes among the available SARS-CoV-2 sequences (as of 21 Feb. 2020). This provides a strong indication of their potential for eliciting a robust T cell or antibody response in SARS-CoV-2.

### Example 2—Optimized Peptide Pools for Assessing Region-Specific SARS-COV2 CD8+ T Cell Responses and Uses Thereof

### Introduction

Global efforts to combat COVID-19 have led to the rapid development of multiple vaccines. These vaccines have been shown to induce a robust neutralizing antibody response and provide protection against severe disease and hospitalization (Hall et al. 2021; Mor et al. 2021). As the virus continues to circulate worldwide, virus variants have emerged in several regions, concerns about their potential to escape vaccine-induced antibody responses. Preliminary results suggest that most current vaccines remain effective against emerging virus variants (Abdool Karim and de Oliveira 2021; Abu-Raddad et al. 2021; Collier et al. 2021; Emary et al. 2021; Liu et al. 2021; Planas et al. 2021). However, specific variants such as Beta (B.1.351), Gamma (P.1) and Delta (B1.617.2), that first emerged in South Africa, Brazil, and India respectively, are currently under investigation due to the observed reduction in neutralizing antibody titres against them in sera of vaccinated individuals (Abdool Karim and de Oliveira 2021; Liu et al. 2021; Madhi et al. 2021; Planas et al. 2021; Wall et al. 2021; Zhou et al. 2021).

In addition to eliciting neutralizing antibodies, SARS-CoV-2 vaccines in use or development also stimulate T cell responses. There is increasing evidence of the role of T cells in protection from severe disease in SARS-CoV-2 infected patients (Altmann and Boyton 2020; Chen and John Wherry 2020; Liao et al. 2020; Mazzoni et al. 2020; Reynolds et al. 2020; Rydyznski Moderbacher et al. 2020; Wyllie et al. 2020; Bergamaschi et al. 2021; Bertoletti et al. 2021; Cohen et al. 2021), and of their robustness to mutations associated with SARS-CoV-2 variants (Quadeer et al. 2021; Tarke et al. 2021; Woldemeskel et al. 2021). However, in contrast to neutralizing antibody responses (Piccoli et al. 2020; Pinto et al. 2020; Fedry et al. 2021), specific T cell responses have not been characterized in detail for any COVID-19 vaccine thus far.

For most currently administered SARS-CoV-2 vaccines, T cell responses have been coarsely measured using immune assays that stimulate blood samples of vaccinated individuals using overlapping peptide pools (Anderson et al. 2020; Folegatti et al. 2020; Keech et al. 2020; Logunov et al. 2020; Ramasamy et al. 2020; Sahin et al. 2020; Zhu et al. 2020; Ella et al. 2021; Klasse et al. 2021; Sadoff et al. 2021). Use of these pools, however, may underestimate T cell responses due to peptide competition, where immunogenic peptides compete with a large number of irrelevant peptides in the pool that are not recognized by T cells (Pala et al. 1988; Sahin et al. 2021). In contrast, assays based on optimized peptide pools would be more efficient at estimating T cell responses as these comprise of a selected set of most relevant peptides against which a T cell response is expected to be stimulated. Such pools also enable identifying precise T cell epitopes in the context of the associated human leukocyte antigen (HLA) alleles presenting them.

Designing optimized peptides pools for assessing T cell responses is challenging due to the diversity of these responses. This is because T cells recognize peptides restricted by an individual's HLA alleles which are highly diverse across the global population (albeit with some commonalities in a given region). Consequently, the peptides restricted by these HLA alleles are also different, and hence T cell responses are expected to differ between geographical regions, even for the same vaccine. Moreover, current vaccines employ different SARS-CoV-2 antigens (e.g., based on the spike (S) protein only or employing the whole inactivated virion), and these are expected to elicit distinct T cell responses. To our knowledge, no tool or platform is currently available that provides peptide pools for measuring SARS-CoV-2-specific T cell responses in a particular region where population is immunized by a specific COVID-19 vaccine. Here, we fill this important gap by developing a software platform, SARS2TPools, that provides optimized peptide pools for assessing region-specific vaccine-induced SARS-CoV-2 T cell responses. These pools are designed by exploiting information of prevalent HLA alleles in a population, the experimentally-determined and in-silico-predicted SARS-CoV-2 T cell epitopes associated with these alleles, and the antigen employed in the vaccine. The optimized pools provided by SARS2TPools, in addition to characterizing the vaccine-induced T cell responses in detail, can be useful for designing T cell based diagnostics, monitoring durability of T cell response, and any change in T cell responses due to emerging SARS-CoV-2 variants.

### Methods and Materials

Data Collection. We downloaded experimentally-determined HLA class I and class II restricted SARS-CoV-2 T cell epitope data (CD8⁺ and CD4⁺, respectively) from the immune epitope database (IEDB) (Vita et al. 2019) on Mar. 10, 2021. We included all epitopes that were reported in positive T cell assays with associated HLA information available. The data consisted of 768 and 445 unique class I and class II epitope-HLA pairs, respectively. Majority of the HLA class I restricted epitopes (474/768) were nine residues long, which is the canonical length of epitopes restricted by HLA class I alleles. The epitope data was found to be biased towards a handful of HLA alleles, with only 10 HLA class I alleles (HLA-A*02:01, HLA-A*03:01, HLA-A*11:01, HLA-A*24:02, HLA-A*29:02, HLA-A*68:01, HLA-B*07:02, HLA-B*35:01, HLA-B*51:01, HLA-B*57:01) having 20 or more nine-residue-long epitopes. Collectively, the epitopes restricted by these 10 HLA alleles corresponded to ˜62% (295/474) of nine-residue-long epitopes in the data. In the case of HLA class II restricted epitopes, all the available epitopes were 15 resides long, and only 3 HLA alleles had more than 20 epitopes in the data.

In Silico Prediction Methods. Performance of several in silico epitope prediction methods were benchmarked against the set of experimentally-determined SARS-CoV-2 epitopes associated with the 10 HLA class I alleles having the most data. The considered methods included the current state-of-the-art methods such as MHCflurry (O'Donnell et al. 2020), NetMHCpan4.1 (Reynisson et al. 2020), HLAthena (Sarkizova et al. 2020), NetMHCpan4.0 (Jurtz et al. 2017), NetMHC4.0 (Andreatta and Nielsen 2016), along with other common prediction methods that have been employed for predicting SARS-CoV-2 epitopes (Sohail et al. 2021) such as NetMHCpan3.0 (Nielsen and Andreatta 2016), SMM (Peters and Sette 2005), SMMPMBEC (Kim et al. 2009), and IEDB consensus (Moutaftsi et al. 2006). We considered the eluted ligand and binding affinity predictions (denoted by suffix BA and EL respectively) of NetMHCpan4.1 and NetMHCpan4.0 as separate methods, as was done for the latter method in (Sarkizova et al. 2020) and (Paul et al. 2020). Similarly, we considered the binding affinity and the presentation score predictions of MHCflurry as two separate methods, referred to as MHCflurry2.0BA and MHCflurry2.0P. In cases where a method required an input other than the protein sequence, HLA allele, and length of the predicted peptides, we used the default parameter settings for that method.

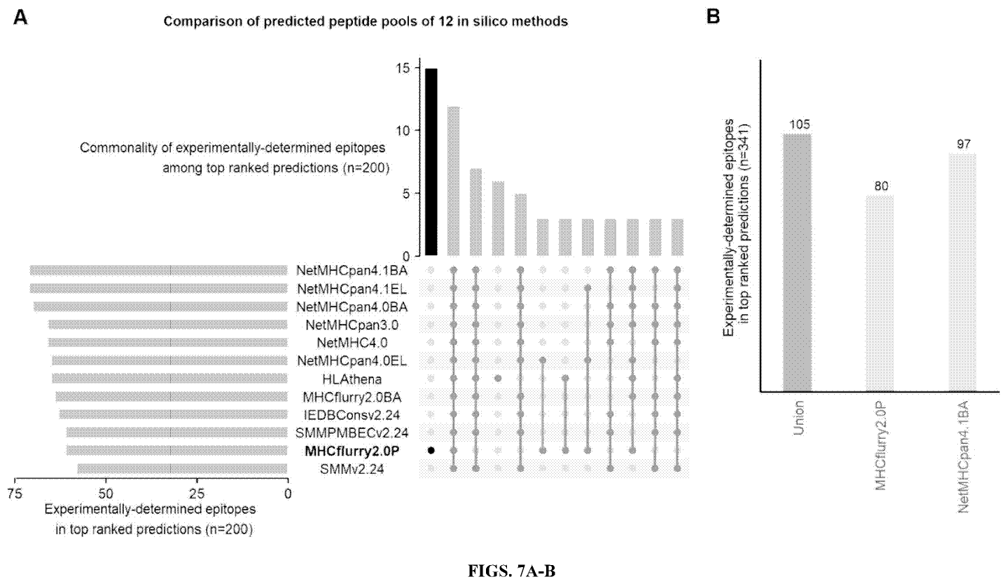

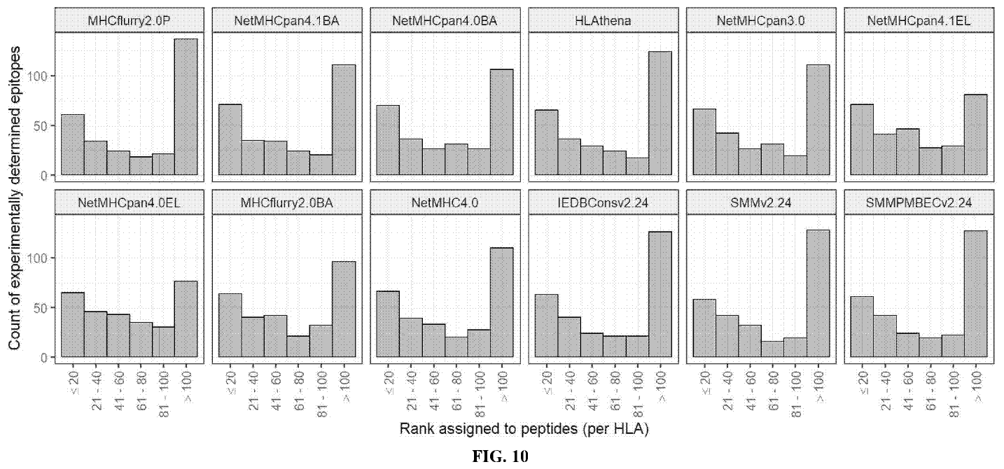

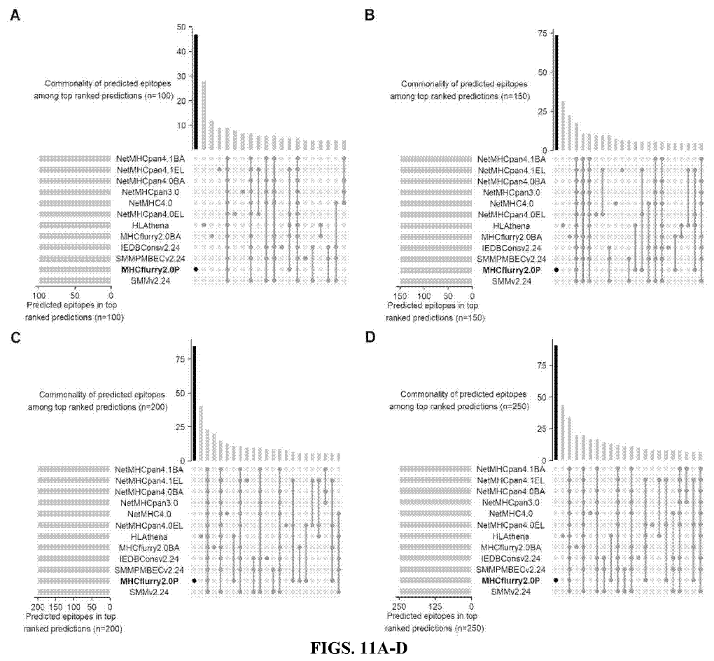

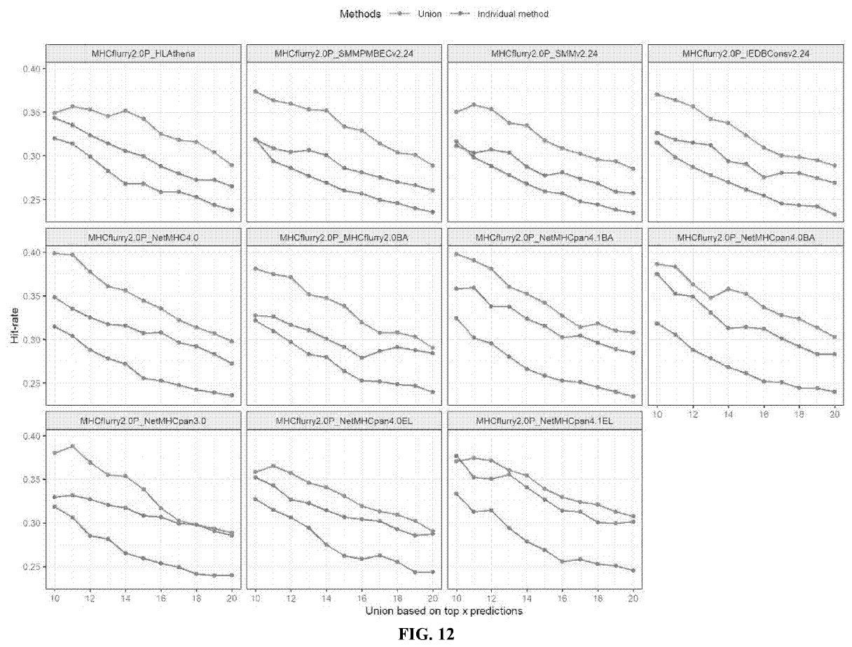

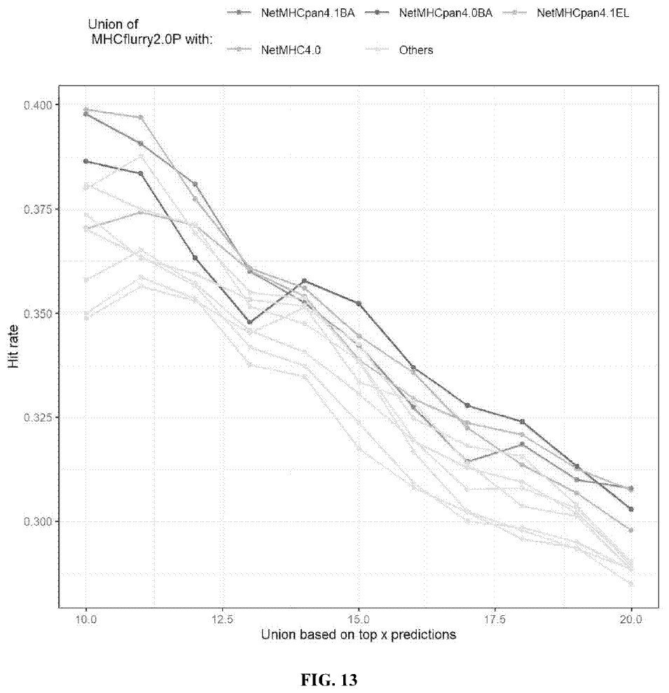

Union Approach. In this work, we have proposed a union approach based on combining the top-ranked predictions of MHCflurry2.0P and NetMHCpan4.1BA to obtain a set of peptides restricted by a given HLA. This approach was motivated by performance comparison analysis of the 12 in silico epitope prediction methods listed above. Briefly, we ranked peptides in ascending order of their predicted score using each method and compared the histograms of ranks of experimentally-determined SARS-CoV-2 CD8+ T cell epitopes associated with the 10 HLA class I alleles with the most data. We found that these histograms were bi-modal for all 12 methods. That is, while the top predictions of each method contained a large number of experimentally-determined epitopes, a good number of epitopes were also ranked quite low by each method (FIG. 10). Exploring the relationships among the set of top 20 ranked peptides per HLA allele predicted by these methods revealed that the predictions of MHCflurry2.0P were most distinct from those of other methods (FIGS. 11A-D). Consistent results were obtained when this set was constructed by pooling the top 10 to top 25 ranked peptides restricted by each HLA allele (FIGS. 11A-D). Predictions of MHCflurry2.0P also contained a large number of experimentally-determined SARS-CoV-2 epitopes that were not present in the set of top-ranked peptides predicted by any other method (FIG. 7A). Given the uniqueness of the predictions of MHCflurry2.0P, we asked if a strategy that combines the predictions of MHCflurry2.0P with any of the other 11 methods would work better than any individual method. The union strategy combines the top x predictions of any two methods and provides a set of peptides whose size can vary between x and 2x depending on the number of common peptides predicted by each method. We fixed one of the methods as MHCflurry2.0P and predicted 11 peptide pools by combining predictions of MHCflurry2.0P with those of the other 11 methods. Our analysis showed that the pool predicted by the union approach always had a higher hit-rate (the fraction of experimentally-determined SARS-CoV-2 epitopes present in the set of top-ranked peptides) than those predicted by the individual methods (FIG. 12). While comparison among the various union approaches did not readily reveal a clear winner, the unions of MHCflurry2.0P with the in silico methods NetMHC4.0, NetMHCpan4.0BA, NetMHCpan4.1BA, and NetMHCpan4.1EL ranked among the top (FIG. 13, Table 13).

The union method implemented in SARS2TPools combines the predictions of MHCflurry2.0P with NetMHCpan4.1BA. SARS2TPools provides optimized peptide pools by supplementing experimentally-determined epitopes with small or large sized group of in silico predicted epitopes corresponding respectively to top 10 and top 20 predictions of each method being combined. We used the default thresholds of MHCflurry2.0P and NetMHCpan4.1BA to assess whether or not a peptide is predicted to be an epitope. However, the platform also provides a relaxed threshold which can be particularly useful for specific proteins with very limited number of predicted epitopes.

Statistical Analysis. Statistical analyses were performed using the R language (version 3.6) on the RStudio server (version 1.3). The software platform was developed using the open source R Shiny (version 1.5) development framework.

### Results

SARS2TPools provides optimized peptide pools for assessing T cell responses leveraging experimentally-determined SARS-CoV-2 epitope data. The designed CD8+ T cell peptide pools also include in silico predictions obtained using a computational approach optimized for predicting SARS-CoV-2 CD8⁺ T cell epitopes. This approach is based on benchmarking predictions of state-of-the-art in silico methods against the ample experimentally-determined SARS-CoV-2 CD8+ epitope data that is now available (Methods).

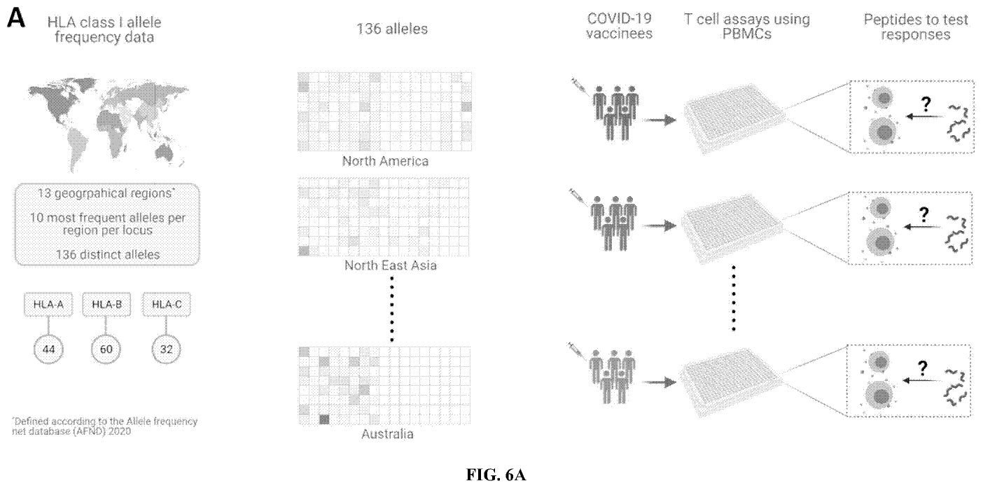

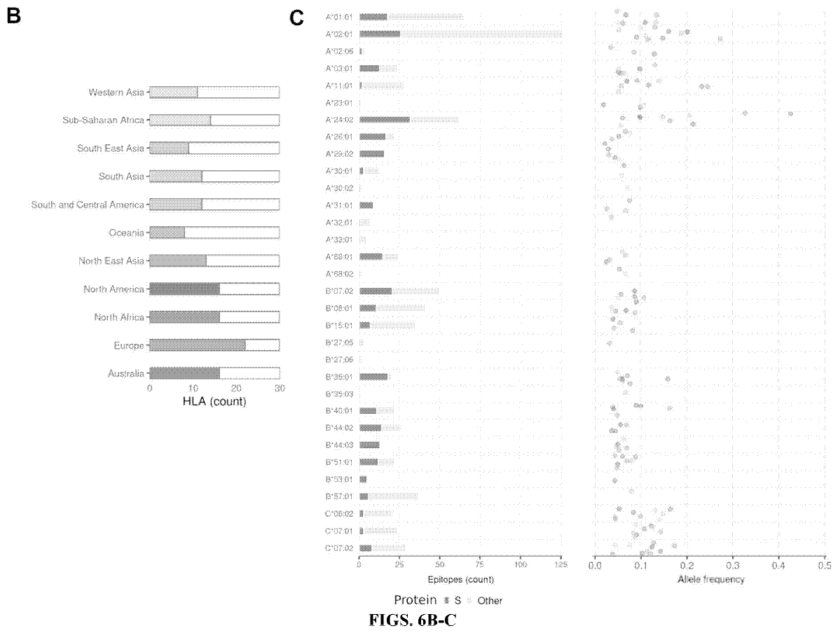

Global HLA class I diversity and summary of experimentally-determined SARS-CoV-2 CD8+ T cell epitope data. Each individual possesses three major HLA class I alleles, HLA-A, HLA-B, and HLA-C, which are among the most polymorphic loci of the human genome (Jin et al. 2018). In fact, more than 6,500 alleles for each of these three loci have been identified so far (Robinson et al. 2015). In order to design a peptide pool for assessing T cell response in a specific geographical region, information of the set of most common HLA alleles in that population is required. Based on extensive population studies, the allele frequency net database (AFND) has curated a list of ten most common HLA alleles per locus for 11 distinct geographical regions encompassing the global population (Gonzalez-Galarza et al. 2019). These sets of HLA alleles have a population coverage of 96% or more in the respective regions. By combining alleles prevalent in all regions, we obtained a total of 136 distinct HLA class I alleles (FIG. 6A, Table 12). Ideally, a peptide pool for assessing T cell responses in a specific region should include a set of experimentally-determined epitopes against all HLA alleles prevalent in that region. However, the available experimental data of SARS-CoV-2 CD8+ T cell epitopes with associated HLA class I information is limited (see Methods). Comparing HLA class I alleles for which experimental data is available with the compiled list of 30 most prevalent HLA alleles per region suggested that a peptide pool comprising of only experimentally-determined epitopes does not cover more than half of the alleles prevalent in majority of the regions (FIG. 6B). This is because epitopes associated with several of the prevalent HLA alleles in different regions have not been experimentally determined yet. Further analysis showed that even at the level of individual HLA alleles, there is large disparity in the number of experimentally reported epitopes, particularly for proteins other than S (FIG. 6C). These data limitations are addressed in the developed platform SARS2TPools by supplementing experimentally-determined SARS-CoV-2 epitope data with in silico predictions to design peptide pools optimized for assessing vaccine-induced T cell responses in a specific region.

TABLE 12
List of 136 HLA alleles that are ranked in the top
10 most-prevalent HLA alleles in at least one of
the 13 geographical regions defined by AFND.

| Sr. | HLA |
|---|---|
| no. | Allele |
| 1 | A*01:01 |
| 2 | A*02:01 |
| 3 | A*02:02 |
| 4 | A*02:03 |
| 5 | A*02:04 |
| 6 | A*02:05 |
| 7 | A*02:06 |
| 8 | A*02:07 |
| 9 | A*02:11 |
| 10 | A*02:12 |
| 11 | A*02:19 |
| 12 | A*02:24 |
| 13 | A*02:264 |
| 14 | A*02:52 |
| 15 | A*03:01 |
| 16 | A*03:02 |
| 17 | A*03:27 |
| 18 | A*11:01 |
| 19 | A*11:02 |
| 20 | A*11:06 |
| 21 | A*23:01 |
| 22 | A*24:02 |
| 23 | A*24:06 |
| 24 | A*24:07 |
| 25 | A*24:41 |
| 26 | A*25:01 |
| 27 | A*26:01 |
| 28 | A*26:03 |
| 29 | A*29:02 |
| 30 | A*29:10 |
| 31 | A*29:25 |
| 32 | A*29:50 |
| 33 | A*30:01 |
| 34 | A*30:02 |
| 35 | A*31:01 |
| 36 | A*31:03 |
| 37 | A*31:08 |
| 38 | A*31:29 |
| 39 | A*32:01 |
| 40 | A*33:03 |
| 41 | A*34:01 |
| 42 | A*68:01 |
| 43 | A*68:02 |
| 44 | A*68:03 |
| 45 | A*74:01 |
| 46 | B*07:02 |
| 47 | B*07:05 |
| 48 | B*07:06 |
| 49 | B*07:07 |
| 50 | B*07:08 |
| 51 | B*07:12 |
| 52 | B*07:17 |
| 53 | B*08:01 |
| 54 | B*08:02 |
| 55 | B*08:03 |
| 56 | B*13:01 |
| 57 | B*13:02 |
| 58 | B*13:07 |
| 59 | B*14:02 |
| 60 | B*14:05 |
| 61 | B*15:01 |
| 62 | B*15:02 |
| 63 | B*15:06 |
| 64 | B*15:13 |
| 65 | B*15:21 |
| 66 | B*15:25 |
| 67 | B*18:01 |
| 68 | B*18:07 |
| 69 | B*27:05 |
| 70 | B*35:01 |
| 71 | B*35:03 |
| 72 | B*35:05 |
| 73 | B*35:08 |
| 74 | B*35:19 |
| 75 | B*35:27 |
| 76 | B*35:43 |
| 77 | B*38:01 |
| 78 | B*38:02 |
| 79 | B*39:03 |
| 80 | B*40:01 |
| 81 | B*40:02 |
| 82 | B*40:06 |
| 83 | B*40:10 |
| 84 | B*41:01 |
| 85 | B*42:01 |
| 86 | B*44:02 |
| 87 | B*44:03 |
| 88 | B*44:04 |
| 89 | B*44:06 |
| 90 | B*45:01 |
| 91 | B*46:01 |
| 92 | B*48:03 |
| 93 | B*50:01 |
| 94 | B*51:01 |
| 95 | B*51:10 |
| 96 | B*52:01 |
| 97 | B*53:01 |
| 98 | B*54:01 |
| 99 | B*54:18 |
| 100 | B*55:02 |
| 101 | B*56:01 |
| 102 | B*56:02 |
| 103 | B*56:43 |
| 104 | B*57:01 |
| 105 | B*58:01 |
| 106 | C*01:02 |
| 107 | C*01:57 |
| 108 | C*02:02 |
| 109 | C*02:03 |
| 110 | C*02:09 |
| 111 | C*02:10 |
| 112 | C*03:02 |
| 113 | C*03:03 |
| 114 | C*03:04 |
| 115 | C*03:05 |
| 116 | C*03:135 |
| 117 | C*04:01 |
| 118 | C*04:03 |
| 119 | C*04:43 |
| 120 | C*05:01 |
| 121 | C*05:09 |
| 122 | C*06:02 |
| 123 | C*06:03 |
| 124 | C*07:01 |
| 125 | C*07:02 |
| 126 | C*07:03 |
| 127 | C*07:17 |
| 128 | C*08:01 |
| 129 | C*08:02 |
| 130 | C*12:02 |
| 131 | C*12:03 |
| 132 | C*14:02 |
| 133 | C*14:03 |
| 134 | C*15:02 |
| 135 | C*15:03 |
| 136 | C*16:01 |

In silico prediction of CD8+ T cell epitopes leveraging SARS-CoV-2 experimental epitope data. We have developed an in silico strategy optimized to predict CD8⁺ T cell epitopes by leveraging experimentally-determined SARS-CoV-2 immunological data. Briefly, we used the available information of experimentally-determined SARS-CoV-2 immunological data. Briefly, we used the available information of experimentally-determined SARS-CoV-2 CD8⁺ T cell epitopes as ground-truth data for comparing the sets of top-ranked peptides predicted by 12 in silico HLA class I epitope prediction methods (Methods). Using the number of experimentally-determined epitopes present in the set of top ranked peptides as a metric to quantify the ability of a method to predict SARS-CoV-2 epitopes, we found that the NetMHCpan family of methods outperformed others (FIG. 7A). Importantly, this comparison revealed the uniqueness of MHCflurry2.0P (O'Donnell et al. 2020) predictions; i.e., it predicted the most number of SARS-CoV-2 epitopes that were not predicted by any other method (FIG. 7A). Our analysis showed that the optimum SARS-CoV-2 epitope prediction strategy was one that combined the predictions of MHCflurry2.0P and NetMHCpan4.1BA (Reynisson et al. 2020) (see Methods for details). This union strategy performed better than either of the individual methods (FIG. 7B) and was thus used to obtain a set of in silico predicted epitopes to supplement the experimentally-determined epitopes in the optimized peptide pools provided by SARS2TPools. We note that a similar approach to predict CD4⁺ T cell epitopes could not be pursued at present due to the scarcity of experimentally-determined SARS-CoV-2 CD4⁺ epitopes having the information of cognate HLA allele (Methods).

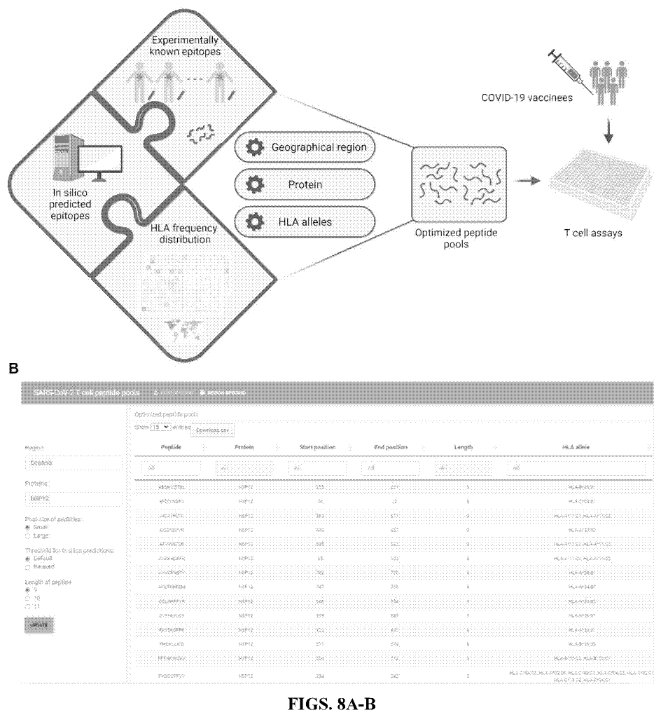

Optimized peptide pools from SARSTPools—Software platform. The platform SARS2TPools integrates experimentally-determined SARS-CoV-2 epitope data, in silico predictions, and information of prevalent HLA alleles across regions to provide optimized peptide pools for assessing vaccine-induced SARS-CoV-2 T cell responses (FIG. 8A). It enables users to obtain region-specific, host-specific, and protein-specific optimized peptide pools through a simple and user-friendly interface (FIG. 8B). Peptide pools optimized for each of the 11 regions, by taking into account the information of HLA alleles prevalent in these regions (FIG. 6A), can be obtained by selecting the ‘Region-specific’ tab on SARS2TPools interface (FIG. 8B). This optimization is important due to the heterogeneity of prevalent HLA alleles among regions that may result in presentation of different epitopes and consequently different T cell responses (FIG. 6A). Region-specific pools can be useful to contrast T cell responses induced by the same vaccine in different geographical regions, and to understand the role of population heterogeneity in mediating different disease outcomes.

Host-specific (or cohort-specific) peptide pools, for a range of peptide lengths (8-11 residues), optimized for HLA haplotypes of the host (or cohort) can be obtained by selecting the ‘Host-specific’ tab on SARS2TPools. This optimization can be useful for cases when host (or cohort) HLA typing has been performed.

SARS2TPools also provides the flexibility to select protein-specific pools derived from the entire SARS-CoV-2 proteome or any number of specific proteins for both region-specific and host-specific options. Moreover, the user can also select peptides belonging to a specific domain of a protein (e.g., receptor binding domain of the spike protein) by specifying a range of amino-acid positions. Protein-specific peptide pools are important for assessing T cell responses induced by vaccine comprising of different antigens, e.g., S only, S with other proteins, or whole-virion.

The CD8⁺ T cell peptide pools provided by SARS2TPools comprise of both experimentally-determined and in silico predicted epitopes, while those for measuring CD4⁺ T cell responses comprise only of experimentally-determined epitopes. In the former case, the platform indicates whether a peptide is an experimentally-determined epitope, predicted epitope, or both. It also provides users the flexibility to select peptide pools consisting exclusively of experimentally-determined CD8⁺ T cell epitopes. SARS2TPools ranks peptides within an optimized pool based on HLA promiscuity, which can be used as an additional prioritization criterion among peptides. In summary, SARS2TPools allows users to obtain pools for assessing T cell responses by flexibly selecting various optimization criteria.

Region-specific CD8+ T cell pools from the whole SARS-CoV-2 proteome. As an illustrative example, we used SARS2TPools to obtain region-specific optimized pools, comprising of peptides derived from the whole SARS-CoV-2 proteome, for measuring CD8⁺ T cell responses for each of the 11 regions defined by AFND. These pools were designed to include around 20 peptides corresponding to each HLA allele prevalent in a region as follows: (i) If an HLA allele prevalent in a region had 20 or more associated experimentally-determined SARS-CoV-2 epitopes, we selected 20 of these based on response frequency (proportion of responding donors (Quadeer et al. 2020)); (ii) if an HLA allele had less than 20 associated experimentally-determined epitopes, we complemented them with in silico predictions based on the union approach; and (iii) if an HLA allele had no associated experimentally-determined epitopes, all peptides associated with it were predicted based on the union approach.

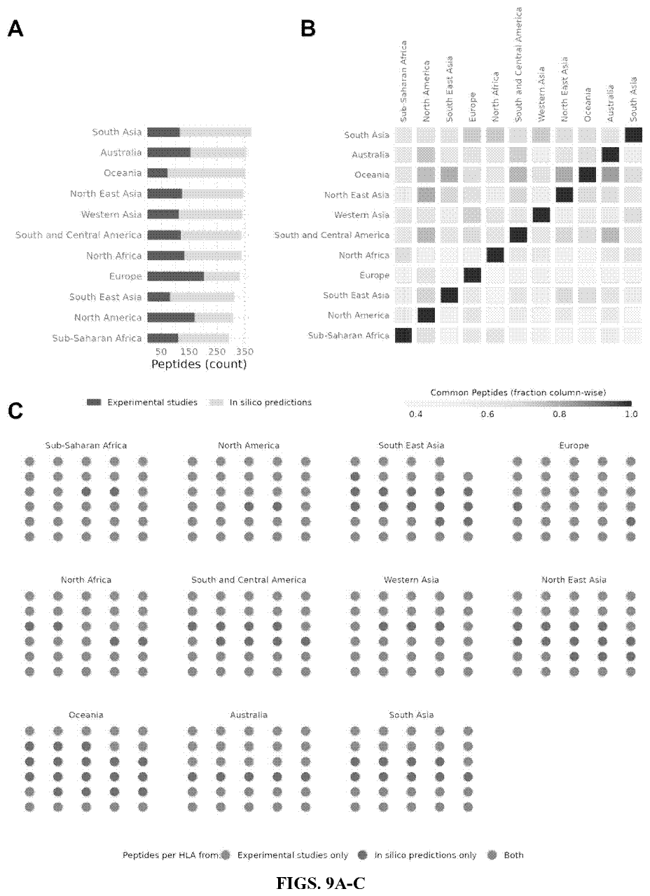

We found that the number of peptides in each of the region-specific optimized pools was roughly similar, with the fraction of experimentally-determined epitopes in these pools varying between ˜35% for Oceania to ˜78% for Europe (FIG. 9A). The HLA alleles covered by each optimized pool can be grouped into 3 classes based on whether their associated peptides in the pool are all (i) experimentally-determined, (ii) in silico predicted, or (iii) a mix of both (FIG. 9B). Importantly, in silico predictions help to fill the gap for alleles for which limited or no experimentally-determined epitopes are available at present. Comparing the 11 region-specific optimized pools, we found that these pools are largely distinct from each other (FIG. 9C). These differences in region-specific pools underscore further the importance of designing optimized peptide pools for assessing T cell responses in different regions.

Listing of all region-specific optimized pools. In addition to providing pools for the whole SARS-CoV-2 proteome, SARS2TPools provides optimized peptide pools for assessing CD8⁺ T cell responses against any of the 30 individual proteins of SARS-CoV-2 for each of the 11 regions. Thus, a total of 31×11=341 region-specific optimized peptide pools are provided by the platform. We list all the 3,860 peptides belonging to any of the 341 pools in Table 15, while the peptides belonging to each specific pools are indicated Table 16 (whole proteome) and Tables 17-47 (individual proteins).

TABLE 15
List of all peptide sequences
(with their IDs) for CD8 T cell pools.

| Seq. ID | Peptide |
|---|---|
| 1 | STECSNLLLQY |
| 2 | FADDLNQLTGY |
| 3 | VTVKNGSIHLY |
| 4 | SSANNCTFEY |
| 5 | FSAVGNICY |
| 6 | VVDYGARFY |
| 7 | YKIEELFYSY |
| 8 | SSEAFLIGCNY |
| 9 | LADAGFIKQY |
| 10 | TDEMIAQY |
| 11 | SSPDDQIGYYR |
| 12 | AGDSGFAAY |
| 13 | TSEDMLNPNY |
| 14 | YTELEPPCRF |
| 15 | AAISDYDYY |
| 16 | FTCASEYTGNY |
| 17 | LGDVRETMSY |
| 18 | TIEVNSFSGY |
| 19 | TITQMNLKY |
| 20 | TSSGDATTAY |
| 21 | WLDMVDTSL |
| 22 | FLYENAFLP |
| 23 | FLPGVYSV |
| 24 | YLITPVHV |
| 25 | YLTNDVSFLA |
| 26 | AQFAPSASA |
| 27 | KLDDKDPNF |
| 28 | KLNDLCFTNV |
| 29 | FLAFVVFL |
| 30 | YLGTGPEAGL |
| 31 | NTASWFTAL |
| 32 | VLQLPQGTTL |
| 33 | ALWEIQQV |
| 34 | SLIYSTAAL |
| 35 | TLMNVLTLV |
| 36 | KLKDCVMYA |
| 37 | WLLWPVTLA |
| 38 | TVYSHLLLV |
| 39 | VLSEARQHL |
| 40 | GLEAPFLYL |
| 41 | FVVPGLPGT |
| 42 | FVENPDILRV |
| 43 | YQDVNCTEV |
| 44 | FLAHIQWMV |
| 45 | VVFLHVTYV |
| 46 | ASFDNFKFV |
| 47 | VMYMGTLSY |
| 48 | QVVNVVTTK |
| 49 | VVTTKIALK |
| 50 | KSAGFPFNK |
| 51 | AIVSTIQRK |
| 52 | YMSALNHTK |
| 53 | RIAGHHLGR |
| 54 | QLRARSVSPK |
| 55 | SVYAWNRKR |
| 56 | TLADAGFIK |
| 57 | GVYYHKNNK |
| 58 | AIVSTIQRKYK |
| 59 | ALDPLSETK |
| 60 | RASANLAATK |
| 61 | IQITISSFK |
| 62 | RMYIFFASFY |
| 63 | TSFGPLVRK |
| 64 | KTFPPTEPK |
| 65 | VTNNTFTLK |
| 66 | KLFDRYFKY |
| 67 | ATVVIGTSK |
| 68 | FAVSKGFFK |
| 69 | AISDYDYYR |
| 70 | VVSTGYHFR |
| 71 | YIATNGPLK |
| 72 | KVAGFAKFLK |
| 73 | QTVKPGNFNK |
| 74 | KSAAEASKK |
| 75 | AGFSLWVYK |
| 76 | VVNARLRAK |
| 77 | ASMPTTIAK |
| 78 | STFNVPMEK |
| 79 | GTHWFVTQR |
| 80 | SASKIITLK |
| 81 | KTIQPRVEK |
| 82 | SAFAMMFVK |
| 83 | GVYFASTEK |
| 84 | TISLAGSYK |
| 85 | QYIKWPWYIW |
| 86 | LYDKLVSSF |
| 87 | SYATHSDKF |
| 88 | VYDPLQPELDSF |
| 89 | YYVGYLQPRTF |
| 90 | IYLYLTFYL |
| 91 | YYKKDNSYF |
| 92 | LYLYALVYF |
| 93 | IYQTSNFRV |
| 94 | MFTPLVPFW |
| 95 | SFLPGVYSV |
| 96 | AYILFTRF |
| 97 | AYVDNSSLTI |
| 98 | AYVNTFSSTF |
| 99 | KMFDAYVNTF |
| 100 | GNYNYLYRLF |
| 101 | VYSSANNCTF |
| 102 | VYRGTTTYKL |
| 103 | AYILFTRFF |
| 104 | EYHDVRVVL |
| 105 | IFFITGNTL |
| 106 | DVFYKENSY |
| 107 | NTVKSVGKF |
| 108 | DTFCAGSTF |
| 109 | FTISVTTEI |
| 110 | YVNTFSSTF |
| 111 | STAALGVLM |
| 112 | DSAEVAVKM |
| 113 | DTIANYAKPF |
| 114 | DVVAIDYKHY |
| 115 | ETIQITISSF |
| 116 | EVARDLSLQF |
| 117 | EVAVKMFDAY |
| 118 | EVGHTDLMAAY |
| 119 | NSTNVTIATY |
| 120 | SVPWDTIANY |
| 121 | TVKNGSIHLY |
| 122 | ELIRQGTDY |
| 123 | EVTPSGTWLTY |
| 124 | ETKCTLKSF |
| 125 | WTFGAGAAL |
| 126 | TLKEILVTY |
| 127 | YIFFASFYY |
| 128 | HSYFTSDYY |
| 129 | LEAPFLYLY |
| 130 | SFYYVWKSY |
| 131 | AGLEAPFLY |
| 132 | VGGNYNYLY |
| 133 | KFCLEASFNY |
| 134 | WFVTQRNFY |
| 135 | VLKGVKLHY |
| 136 | GAAAYYVGY |
| 137 | KVGGNYNYLY |
| 138 | SFKEELDKY |
| 139 | SWMESEFRVY |
| 140 | LVAEWFLAY |
| 141 | VFAQVKQIY |
| 142 | CVADYSVLY |
| 143 | WTAGAAAYY |
| 144 | FVFKNIDGY |
| 145 | VASQSIIAY |
| 146 | RIKASMPTT |
| 147 | RLFARTRSM |
| 148 | KASMPTTIA |
| 149 | MSALNHTKK |
| 150 | TTIAKNTVK |
| 151 | YSRYRIGNYK |
| 152 | RNRFLYIIK |
| 153 | GTRNPANNA |
| 154 | AGLPYGANK |
| 155 | RVYSTGSNV |
| 156 | VTYVPAQEK |
| 157 | RYRIGNYKL |
| 158 | ASRELKVTF |
| 159 | KVFRSSVLH |
| 160 | ATSRTLSYYK |
| 161 | QLTPTWRVY |
| 162 | VTPSGTWLTY |
| 163 | LAYYFMRFR |
| 164 | GAMDTTSYR |
| 165 | WVLNNDYYR |
| 166 | WFFSNYLKR |
| 167 | GSVAYESLR |
| 168 | KSNLKPFER |
| 169 | YNYLYRLFR |
| 170 | QTNSPRRAR |
| 171 | KFLPFQQFGR |
| 172 | RFASVYAWNR |
| 173 | LSYFIASFR |
| 174 | RLFRKSNLK |
| 175 | STGSNVFQTR |
| 176 | KLMGHFAWW |
| 177 | YVMHANYIF |
| 178 | KLINIIIWF |
| 179 | KVAGFAKFL |
| 180 | AMYTPHTVL |
| 181 | SLDNVLSTF |
| 182 | GVVFLHVTY |
| 183 | STNVTIATY |
| 184 | HVTFFIYNK |
| 185 | MASLVLARK |
| 186 | FTIGTVTLK |
| 187 | AVILRGHLR |
| 188 | HVSGTNGTK |
| 189 | AAISDYDYYR |
| 190 | FVVSTGYHFR |
| 191 | YAISAKNRAR |
| 192 | QIAPGQTGK |
| 193 | NSASFSTFK |
| 194 | LVIGAVILR |
| 195 | FASVYAWNR |
| 196 | SVLNDILSR |
| 197 | NASVVNIQK |
| 198 | GTITVEELK |
| 199 | FVIRGDEVR |
| 200 | DSGFAAYSR |
| 201 | LTAVVIPTK |
| 202 | ESKPSVEQR |
| 203 | DTVIEVQGYK |
| 204 | SSSDNIALL |
| 205 | FVLAAVYRI |
| 206 | VPHISRQRL |
| 207 | RARSVSPKL |
| 208 | FPLKLRGTA |
| 209 | LPKEITVAT |
| 210 | LPFAMGIIAM |
| 211 | NPIQLSSYSL |
| 212 | RPLLESEL |
| 213 | KPFERDISTEI |
| 214 | LPNNTASWF |
| 215 | KPVETSNSFDVL |
| 216 | RPQGLPNNTA |
| 217 | VPGLPGTIL |
| 218 | QPYRVVVLSF |
| 219 | VPLHGTIL |
| 220 | RIRGGDGKM |
| 221 | APHGVVFLHV |
| 222 | QPGQTFSVL |
| 223 | LEIPRRNVATL |
| 224 | APHGVVFL |
| 225 | SPIFLIVAA |
| 226 | SAMVRMYIF |
| 227 | TPKYKFVRI |
| 228 | TLKKRWQLA |
| 229 | HLRIAGHHL |
| 230 | SLYVNKHAF |
| 231 | HLKDGTCGL |
| 232 | TFKVSIWNL |
| 233 | LTIKKPNEL |
| 234 | NLKTLLSL |
| 235 | MLRIMASL |
| 236 | FRLFARTRSM |
| 237 | HPLADNKFAL |
| 238 | INITRFQTL |
| 239 | KIYSKHTPI |
| 240 | ANRNRFLYI |
| 241 | NITRFQTL |
| 242 | MFDAYVNTF |
| 243 | LLADKFPVL |
| 244 | LIIMRTFKV |
| 245 | VPQEHYVRI |
| 246 | SSAKSASVY |
| 247 | VVAIDYKHY |
| 248 | RLYYDSMSY |
| 249 | YLFDESGEF |
| 250 | EIKESVQTF |
| 251 | VLAAECTIF |
| 252 | KLVSSFLEM |
| 253 | VVYRAFDIY |
| 254 | ITILDGISQY |
| 255 | LVKQGDDYVY |
| 256 | QLYLGGMSYY |
| 257 | VLTESNKKF |
| 258 | SQRVAGDSGF |
| 259 | LVQMAPISAM |
| 260 | FVVEVVDKY |
| 261 | YLKLTDNVY |
| 262 | KQFDTYNLW |
| 263 | LLNKHIDAY |
| 264 | KIEELFYSY |
| 265 | VVQQLPETY |
| 266 | QRNAPRITF |
| 267 | LVSDIDITF |
| 268 | VVVNAANVY |
| 269 | VPFWITIAY |
| 270 | TPSKLIEY |
| 271 | QIPFAMQMAY |
| 272 | TPSGTWLTY |
| 273 | SPDDQIGYY |
| 274 | DVLLPLTQY |
| 275 | VAAGLEAPF |
| 276 | LPAADLDDF |
| 277 | LPLTQYNRY |
| 278 | SIIQFPNTY |
| 279 | DASGKPVPY |
| 280 | FAPSASAFF |
| 281 | TNVLEGSVAY |
| 282 | FAMQMAYRF |
| 283 | LGAENSVAY |
| 284 | NATRFASVY |
| 285 | LPPLLTDEM |
| 286 | IPFAMQMAY |
| 287 | SEIIGYKAI |
| 288 | FGEYSHVVAF |
| 289 | YENFNQHEV |
| 290 | LEMELTPVV |
| 291 | VEVQPQLEM |
| 292 | SELLTPLGI |
| 293 | FELDERIDKVL |
| 294 | LEFGATSAAL |
| 295 | MELTPVVQTI |
| 296 | SEDAQGMDNL |
| 297 | FDEDDSEPVL |
| 298 | FEYVSQPFLM |
| 299 | SEPVLKGVKL |
| 300 | WEPEFYEAM |
| 301 | LEYHDVRVVL |
| 302 | GETLPTEVL |
| 303 | TEVVGDIIL |
| 304 | FERDISTEI |
| 305 | AEVQIDRL |
| 306 | NESLIDLQEL |
| 307 | FEPSTQYEY |
| 308 | IEVNSFSGY |
| 309 | VELGTEVNEF |
| 310 | QELIRQGTDY |
| 311 | AELAKNVSL |
| 312 | SELVIGAVI |
| 313 | YERHSLSHF |
| 314 | AEVAVKMF |
| 315 | AEWFLAYILF |
| 316 | EESSAKSASVY |
| 317 | SEYKGPITDVFY |
| 318 | SEYTGNYQCGHY |
| 319 | TETDLTKGPHEF |
| 320 | VENPDILRVY |
| 321 | VENPHLMGWD |
| 322 | YSDVENPHLMGW |
| 323 | AAGLEAPFLYLY |
| 324 | ADAGFIKQY |
| 325 | YENQKLIANQF |
| 326 | YEQYIKWPW |
| 327 | VENPDILRV |
| 328 | EEVVENPTI |
| 329 | EEVGHTDLMAAY |
| 330 | VENMTPRDL |
| 331 | REGVFVSNGTHW |
| 332 | EEIAIILASF |
| 333 | EEAIRHVRAW |
| 334 | QEILGTVSW |
| 335 | SEFSSLPSY |
| 336 | MEVTPSGTW |
| 337 | AEAELAKNV |
| 338 | AEVQIDRLI |
| 339 | AEIRASANL |
| 340 | KEIDRLNEV |
| 341 | GEVFNATRF |
| 342 | QELGKYEQY |
| 343 | KQEILGTVSW |
| 344 | QEYADVFHLY |
| 345 | AEHVNNSY |
| 346 | IAAVITREV |
| 347 | DAVNLLTNM |
| 348 | LPGVYSVI |
| 349 | LPRVFSAV |
| 350 | DAMRNAGIV |
| 351 | YLPYPDPSRI |
| 352 | IPYNSVTSSIVI |
| 353 | LPFGWLIV |
| 354 | APYIVGDVV |
| 355 | VPMEKLKTL |
| 356 | EGYLNSTNV |
| 357 | EAKKVKPTV |
| 358 | MPTTIAKNTV |
| 359 | MPYFFTLL |
| 360 | YPQVNGLTSI |
| 361 | LPYGANKDGI |
| 362 | LPYPDPSRI |
| 363 | LPLVSSQCV |
| 364 | FAYTKRNVI |
| 365 | EVFAQVKQI |
| 366 | NPLLYDANY |
| 367 | FLPFFSNVTW |
| 368 | IHADQLTPTW |
| 369 | LPPAYTNSF |
| 370 | IAIPTNFTI |
| 371 | FPQSAPHGV |
| 372 | AVHFISNSW |
| 373 | NSIAIPTNF |
| 374 | KMKDLSPRW |
| 375 | ATIPIQASL |
| 376 | VAMPNLYKM |
| 377 | RSVASOSII |
| 378 | VARDLSLQF |
| 379 | STVFPPTSF |
| 380 | AKSHNIALIW |
| 381 | ITFDNLKTL |
| 382 | LTAFGLVAEW |
| 383 | NKATYKPNTW |
| 384 | SAKSASVYY |
| 385 | ISTKHFYW |
| 386 | RTTNGDFLHF |
| 387 | KSAGFPFNKW |
| 388 | LTNDNTSRYW |
| 389 | CATVHTANKW |
| 390 | GVAPGTAVLRQW |
| 391 | GVFVSNGTHW |
| 392 | VRSIFSRTL |
| 393 | YRGTTTYKL |
| 394 | YASAVVLLI |
| 395 | GFMGRIRSV |
| 396 | YVYSRVKNL |
| 397 | AHAEETRKL |
| 398 | LRKHFSMMI |
| 399 | CRSKNPLLY |
| 400 | NSFSGYLKL |
| 401 | YAKPFLNKV |
| 402 | VRQALLKTV |
| 403 | ERHSLSHFV |
| 404 | SAKNRARTV |
| 405 | MYKGLPWNV |
| 406 | YQPYRVVVL |
| 407 | YRYNLPTMC |
| 408 | LHKPIVWHV |
| 409 | VKNGSIHLY |
| 410 | LRPDTRYV |
| 411 | VGYQPYRVV |
| 412 | VRIQPGQTF |
| 413 | WRNTNPIQL |
| 414 | VRNLOHRLY |
| 415 | SRVLGLKTL |
| 416 | LRVEAFEYY |
| 417 | VRETMSYLF |
| 418 | FKNLREFVF |
| 419 | NVIPTITQM |
| 420 | ARAGEAANF |
| 421 | WKYPQVNGL |
| 422 | FWRNTNPIQL |
| 423 | FRSSVLHST |
| 424 | LRGTAVMSL |
| 425 | GRVDGQVDL |
| 426 | TANPKTPKY |
| 427 | KKQQTVTLL |
| 428 | MKDLSPRWY |
| 429 | YRSLPGVF |
| 430 | SRYWEPEF |
| 431 | RNRFLYIIKL |
| 432 | MYASAVVLL |
| 433 | TRTQLPPAY |
| 434 | YADVFHLYL |
| 435 | FLYLYALVY |
| 436 | KFADDLNQL |
| 437 | YFDKAGQKTY |
| 438 | YYKKDNSY |
| 439 | YDYLVSTQEF |
| 440 | QSAPHGVVF |
| 441 | LRIMASLVL |
| 442 | YFTSDYYQL |
| 443 | VRIIMRLWL |
| 444 | LVKPSFYVY |
| 445 | FYYVWKSY |
| 446 | ARLYYDSMSY |
| 447 | IYKTPPIKDF |
| 448 | YFIKGLNNL |
| 449 | TRFASVYAW |
| 450 | FYLITPVHV |
| 451 | IYDEPTTTT |
| 452 | FLLPSLATV |
| 453 | FLLNKEMYL |
| 454 | TMADLVYAL |
| 455 | YLQPRTFLL |
| 456 | YLNSTNVTI |
| 457 | HLVDFQVTI |
| 458 | VLNDILSRL |
| 459 | ALWEIQQVV |
| 460 | SVVSKVVKV |
| 461 | ALSKGVHFV |
| 462 | NLIDSYFVV |
| 463 | KIADYNYKL |
| 464 | LLYDANYFL |
| 465 | FVNEFYAYL |
| 466 | YLYALVYFL |
| 467 | ILFTRFFYV |
| 468 | FLNRFTTTL |
| 469 | YLNTLTLAV |
| 470 | YLTNDVSFL |
| 471 | FLPRVFSAV |
| 472 | KLNIKLLGV |
| 473 | FIAGLIAIV |
| 474 | FLNGSCGSV |
| 475 | KLSYGIATV |
| 476 | VLAWLYAAV |
| 477 | RTIKVFTTV |
| 478 | VLWAHGFEL |
| 479 | LMIERFVSL |
| 480 | SMWALIISV |
| 481 | YTMADLVYA |
| 482 | FVAAIFYLI |
| 483 | SLPGVFCGV |
| 484 | RIMTWLDMV |
| 485 | LQLGFSTGV |
| 486 | AVIKTLQPV |
| 487 | KVDGVVQQL |
| 488 | FVDGVPFVV |
| 489 | YMPYFFTLL |
| 490 | YLDAYNMMI |
| 491 | MLDMYSVML |
| 492 | GLMWLSYFI |
| 493 | NLSDRVVFV |
| 494 | FLARGIVFM |
| 495 | AMDEFIERY |
| 496 | TLKSFTVEK |
| 497 | HLMGWDYPK |
| 498 | LLFFRALPK |
| 499 | KLFAAETLK |
| 500 | RLISMMGFK |
| 501 | KMQRMLLEK |
| 502 | AVAKHDFFK |
| 503 | HVVGPNVNK |
| 504 | TMLFTMLRK |
| 505 | MTSCCSCLK |
| 506 | AQCFKMFYK |
| 507 | HLYLQYIRK |
| 508 | RQFHQKLLK |
| 509 | TTIKPVTYK |
| 510 | GVAMPNLYK |
| 511 | SSTCMMCYK |
| 512 | GTLSYEQFK |
| 513 | QTMLFTMLR |
| 514 | QTFFKLVNK |
| 515 | NYMPYFFTL |
| 516 | IYNDKVAGF |
| 517 | VYMPASWVM |
| 518 | YFVVKRHTF |
| 519 | VYIGDPAQL |
| 520 | VYDPLQPEL |
| 521 | QYIKWPWYI |
| 522 | YFIASFRLF |
| 523 | YYTSNPTTF |
| 524 | YYQLYSTQL |
| 525 | TYKPNTWCI |
| 526 | VYFLQSINF |
| 527 | WSMATYYLF |
| 528 | SYYSLLMPI |
| 529 | TYACWHHSI |
| 530 | SYFIASFRL |
| 531 | DYQGKPLEF |
| 532 | NYNYLYRLF |
| 533 | FFASFYYVW |
| 534 | TYASALWEI |
| 535 | LYSPIFLIV |
| 536 | RYLALYNKY |
| 537 | EYADVFHLY |
| 538 | MYIFFASFY |
| 539 | QYNRYLALY |
| 540 | YYPSARIVY |
| 541 | ETISLAGSY |
| 542 | DTYNLWNTF |
| 543 | DVTDVTQLY |
| 544 | ETKAIVSTI |
| 545 | EIVDTVSAL |
| 546 | EAIRHVRAW |
| 547 | EIAIILASF |
| 548 | VVIPDYNTY |
| 549 | DAQSFLNGF |
| 550 | DVRETMSYL |
| 551 | MTYRRLISM |
| 552 | FTSDYYQLY |
| 553 | MVMCGGSLY |
| 554 | EVNSFSGYL |
| 555 | EVVGDIILK |
| 556 | SFYEDFLEY |
| 557 | VVYRGTTTY |
| 558 | YILFTRFFY |
| 559 | FAIGLALYY |
| 560 | SMMGFKMNY |
| 561 | GVYSVIYLY |
| 562 | ATSRTLSYY |
| 563 | ASHMYCSFY |
| 564 | KMNYQVNGY |
| 565 | AVKTQFNYY |
| 566 | ALCEKALKY |
| 567 | MMSAPPAQY |
| 568 | RISNCVADY |
| 569 | GTFTCASEY |
| 570 | VYYPDKVFR |
| 571 | CSLSHRFYR |
| 572 | HFYSKWYIR |
| 573 | AYYFMRFRR |
| 574 | AVHECFVKR |
| 575 | KAIDGGVTR |
| 576 | RVVRSIFSR |
| 577 | NSLLTPFAR |
| 578 | RVKNLNSSR |
| 579 | NFYGPFVDR |
| 580 | VTHSKGLYR |
| 581 | ALHFLLFFR |
| 582 | CMMCYKRNR |
| 583 | MSKFPLKLR |
| 584 | RTIKGTHHW |
| 585 | KAYNVTQAF |
| 586 | KLLHKPIVW |
| 587 | RMYIFFASF |
| 588 | KFYGGWHNM |
| 589 | KTPKYKFVR |
| 590 | LFALLQRYR |
| 591 | RAMPNMLRI |
| 592 | KLAKKFDTF |
| 593 | KSYELQTPF |
| 594 | RTNVYLAVF |
| 595 | NVFAFPFTI |
| 596 | KVYPIILRL |
| 597 | VMFTPLVPF |
| 598 | DYGDAVVYR |
| 599 | DFDTWFSQR |
| 600 | DFYDFAVSK |
| 601 | VYADSFVIR |
| 602 | MTQMYKQAR |
| 603 | NYAKPFLNK |
| 604 | IASFRLFAR |
| 605 | NTVIWDYKR |
| 606 | YAFASEAAR |
| 607 | TVIEVQGYK |
| 608 | TTDPSFLGR |
| 609 | FSSEIIGYK |
| 610 | MSAFAMMFV |
| 611 | NATNVVIKV |
| 612 | STSAFVETV |
| 613 | NTFSSTFNV |
| 614 | SVAALTNNV |
| 615 | QSFLNGFAV |
| 616 | ETFKLSYGI |
| 617 | YTACSHAAV |
| 618 | FSASTSAFV |
| 619 | HTIDGSSGV |
| 620 | HVISTSHKL |
| 621 | FSYFAVHFI |
| 622 | DAQSFLNRV |
| 623 | NTQEVFAQV |
| 624 | TTFDSEYCR |
| 625 | LSTFISAAR |
| 626 | FLAYILFTR |
| 627 | RVYANLGER |
| 628 | FPRGQGVPI |
| 629 | IPRRNVATL |
| 630 | SPRRARSVA |
| 631 | KPNELSRVL |
| 632 | SPRWYFYYL |
| 633 | SPYNSQNAV |
| 634 | IPVAYRKVL |
| 635 | RPDTRYVLM |
| 636 | KPCIKVATV |
| 637 | MARKTLNSL |
| 638 | HPTQAPTHL |
| 639 | IPIGAGICA |
| 640 | LPQNAVVKI |
| 641 | MPNMLRIMA |
| 642 | IPIQASLPF |
| 643 | KPVETSNSF |
| 644 | IPTNFTISV |
| 645 | TPAFDKSAF |
| 646 | FPPTSFGPL |
| 647 | MIAQYTSAL |
| 648 | NPAWRKAVF |
| 649 | TPKGPKVKY |
| 650 | QPRTFLLKY |
| 651 | KPREQIDGY |
| 652 | KPRQKRTAT |
| 653 | LPSLATVAY |
| 654 | YLRKHFSMM |
| 655 | YLKLRSDVL |
| 656 | FVKHKHAFL |
| 657 | CLLNRYFRL |
| 658 | DLFMRIFTI |
| 659 | YFMRFRRAF |
| 660 | FLKTNCCRF |
| 661 | DAPAHISTI |
| 662 | TQMNLKYAI |
| 663 | SQLGGLHLL |
| 664 | GEYSHVVAF |
| 665 | MMISAGFSL |
| 666 | TQWSLFFFL |
| 667 | REHEHEIAW |
| 668 | RLVDPQIQL |
| 669 | NQMCLSTLM |
| 670 | RQWLPTGTL |
| 671 | MQTMLFTML |
| 672 | FQFCNDPFL |
| 673 | RELHLSWEV |
| 674 | MQVESDDYI |
| 675 | YELQTPFEI |
| 676 | RQLLFVVEV |
| 677 | RSLKVPATV |
| 678 | KQIYKTPPI |
| 679 | KQLIKVTLV |
| 680 | MQLFFSYFA |
| 681 | SQNAVASKI |
| 682 | TQYNRYLAL |
| 683 | NRFLYIIKL |
| 684 | DQFKHLIPL |
| 685 | EHYVRITGL |
| 686 | TRFQTLLAL |
| 687 | DKVFRSSVL |
| 688 | YRLFRKSNL |
| 689 | SHVVAFNTL |
| 690 | EHFIETISL |
| 691 | VRFPNITNL |
| 692 | FRNARNGVL |
| 693 | TRFFYVLGL |
| 694 | HHMELPTGV |
| 695 | NNLNRGMVL |
| 696 | SHFVNLDNL |
| 697 | QHEETIYNL |
| 698 | QHMVVKAAL |
| 699 | FRYMNSQGL |
| 700 | SHFAIGLAL |
| 701 | MKIILFLAL |
| 702 | FAVDAAKAY |
| 703 | FLHFLPRVF |
| 704 | QLYLGGMSY |
| 705 | LMNVLTLVY |
| 706 | YLVQQESPF |
| 707 | VQMAPISAM |
| 708 | ILMTARTVY |
| 709 | MISAGFSLW |
| 710 | NMVYMPASW |
| 711 | LPFFSNVTW |
| 712 | MSMTYGQQF |
| 713 | FISNSWLMW |
| 714 | NMMVTNNTF |
| 715 | HADQLTPTW |
| 716 | NVLEGSVAY |
| 717 | HMLDMYSVM |
| 718 | FMGRIRSVY |
| 719 | VPWDTIANY |
| 720 | DEWSMATYY |
| 721 | VEHVTFFIY |
| 722 | DEISMATNY |
| 723 | FELEDFIPM |
| 724 | HEFCSQHTM |
| 725 | LEWLAMAVM |
| 726 | NETLVTMPL |
| 727 | LEIKDTEKY |
| 728 | SEVGPEHSL |
| 729 | TEETFKLSY |
| 730 | EEFEPSTQY |
| 731 | SEFDRDAAM |
| 732 | TELEPPCRF |
| 733 | MPYFFTLLL |
| 734 | LPFNDGVYF |
| 735 | NPHLMGWDY |
| 736 | YPNASFDNF |
| 737 | LPGVYSVIY |
| 738 | CPDGVKHVY |
| 739 | VPFVVSTGY |
| 740 | KRWQLALSK |
| 741 | RRLISMMGF |
| 742 | GRWVLNNDY |
| 743 | KRVDWTIEY |
| 744 | ARFYFYTSK |
| 745 | ARYMRSLKV |
| 746 | NRFNVAITR |
| 747 | MRIMTWLDM |
| 748 | GRIRSVYPV |
| 749 | SRYRIGNYK |
| 750 | ARTRSMWSF |
| 751 | SRLSFKELL |
| 752 | RRVWTLMNV |
| 753 | LPVNVAFEL |
| 754 | FPFTIYSLL |
| 755 | SAPPAQYEL |
| 756 | MPASWVMRI |
| 757 | FPDLNGDVV |
| 758 | YPSLETIQI |
| 759 | FGADPIHSL |
| 760 | VADAVIKTL |
| 761 | EAVGTNLPL |
| 762 | YPLECIKDL |
| 763 | FSSTFNVPM |
| 764 | QPTESIVRF |
| 765 | FVSLAIDAY |
| 766 | YPGQGLNGY |
| 767 | YANRNRFLY |
| 768 | FAYANRNRF |
| 769 | LAKDTTEAF |
| 770 | HHSIGFDYV |
| 771 | MHAASGNLL |
| 772 | YHTTDPSFL |
| 773 | LHSTQDLFL |
| 774 | THHWLLLTI |
| 775 | VRDPQTLEI |
| 776 | KHITSKETL |
| 777 | THTGTGQAI |
| 778 | THLSVDTKF |
| 779 | VHFVCNLLL |
| 780 | IHFYSKWYI |
| 781 | YKVYYGNAL |
| 782 | LRSDVLLPL |
| 783 | NRALTGIAV |
| 784 | AEWFLAYIL |
| 785 | HEGKTFYVL |
| 786 | GEAANFCAL |
| 787 | AEAAVKPLL |
| 788 | LENVAFNVV |
| 789 | TEVPVAIHA |
| 790 | NESGLKTIL |
| 791 | HEVLLAPLL |
| 792 | AECTIFKDA |
| 793 | REFLTRNPA |
| 794 | SEFRVYSSA |
| 795 | YENAFLPFA |
| 796 | IELKFNPPA |
| 797 | MEIDFLELA |
| 798 | GECPNFVFP |
| 799 | VELKHFFFA |
| 800 | LEFGATSAA |
| 801 | GEQKSILSP |
| 802 | TERLKLFAA |
| 803 | REAACCHLA |
| 804 | LQAAVGELL |
| 805 | FEYVSQPFL |
| 806 | FQVTIAEIL |
| 807 | AEIVDTVSA |
| 808 | LEPEYFNSV |
| 809 | MEKLKTLVA |
| 810 | APFLYLYAL |
| 811 | KPTVVVNAA |
| 812 | VENPHLMGW |
| 813 | SEKQVEQKI |
| 814 | SEMHPALRL |
| 815 | SEDMLNPNY |
| 816 | CPIHFYSKW |
| 817 | TAFGLVAEW |
| 818 | TASDTYACW |
| 819 | TPGDSSSGW |
| 820 | GETLGVLVP |
| 821 | REAVGTNLP |
| 822 | QEAYEQAVA |
| 823 | SEYDYVIFT |
| 824 | EELFYSYAT |
| 825 | NEYRLYLDA |
| 826 | SEAGVCVST |
| 827 | YLITPVHVM |
| 828 | YSSANNCTF |
| 829 | FASEAARVV |
| 830 | MELPTGVHA |
| 831 | FENKTTLPV |
| 832 | MPLSAPTLV |
| 833 | CPAEIVDTV |
| 834 | SPFELEDFI |
| 835 | MPTIFFAGI |
| 836 | FPLCANGQV |
| 837 | SAFYILPSI |
| 838 | LAWLYAAVI |
| 839 | MAYITGGVV |
| 840 | VEYCPIFFI |
| 841 | CQYLNTLTL |
| 842 | TQFNYYKKV |
| 843 | IQLSSYSLF |
| 844 | LAAVNSVPW |
| 845 | MPILTLTRA |
| 846 | LPFAMGIIA |
| 847 | MPVCVETKA |
| 848 | FPFNKWGKA |
| 849 | FPREGVFVS |
| 850 | CPFGEVFNA |
| 851 | DAYNMMISA |
| 852 | LPFKLTCAT |
| 853 | SPSGVYQCA |
| 854 | KSHNIALIW |
| 855 | LSDLQDLKW |
| 856 | RSFIEDLLF |
| 857 | VSFLAHIQW |
| 858 | MACLVGLMW |
| 859 | KAYKIEELF |
| 860 | LAAVYRINW |
| 861 | LAGTITSGW |
| 862 | VMPLSAPTL |
| 863 | SAPHGVVFL |
| 864 | GVAPGTAVL |
| 865 | HANEYRLYL |
| 866 | FLPGVYSVI |
| 867 | VSPTKLNDL |
| 868 | NVPLHGTIL |
| 869 | QLPAPRTLL |
| 870 | TAPHGHVMV |
| 871 | IGPERTCCL |
| 872 | SANNCTFEY |
| 873 | FVLTSHTVM |
| 874 | IAMSAFAMM |
| 875 | VATSRTLSY |
| 876 | FAQDGNAAI |
| 877 | FSNSGSDVL |
| 878 | YSTAALGVL |
| 879 | FVSDADSTL |
| 880 | ISTSHKLVL |
| 881 | FCYMHHMEL |
| 882 | VAKSHNIAL |
| 883 | RTAPHGHVM |
| 884 | TFDNLKTLL |
| 885 | RFDNPVLPF |
| 886 | ILDITPCSF |
| 887 | KYDFTEERL |
| 888 | LFDMSKFPL |
| 889 | YGDFSHSQL |
| 890 | YFTEQPIDL |
| 891 | VYDDGARRV |
| 892 | VTDVTQLYL |
| 893 | YVDNSSLTI |
| 894 | YSDVENPHL |
| 895 | VVDSYYSLL |
| 896 | YIDIGNYTV |
| 897 | MADQAMTQM |
| 898 | ANDPVGFTL |
| 899 | VSDIDITFL |
| 900 | ISDEVARDL |
| 901 | ISDEFSSNV |
| 902 | VAFNTLLFL |
| 903 | YYRYNLPTM |
| 904 | YYHTTDPSF |
| 905 | FYLTNDVSF |
| 906 | LYYQNNVFM |
| 907 | AADPAMHAA |
| 908 | LSDGLLLAL |
| 909 | ITDAVDCAL |
| 910 | VSDIDYVPL |
| 911 | VAYFNMVYM |
| 912 | SFSASTSAF |
| 913 | ISAMVRMYI |
| 914 | KTLLSLREV |
| 915 | ISAGFSLWV |
| 916 | YTNSFTRGV |
| 917 | HAASGNLLL |
| 918 | AVASKILGL |
| 919 | ALNNIINNA |
| 920 | KLWAQCVQL |
| 921 | SLSHRFYRL |
| 922 | TLIGDCATV |
| 923 | QMAPISAMV |
| 924 | VLSDRELHL |
| 925 | YLATALLTL |
| 926 | MLAKALRKV |
| 927 | HSIGFDYVY |
| 928 | NYSGVVTTV |
| 929 | YQKVGMQKY |
| 930 | TVAYFNMVY |
| 931 | ALNTLVKQL |
| 932 | YFKYWDQTY |
| 933 | VFLGIITTV |
| 934 | VTWFHAIHV |
| 935 | KVQIGEYTF |
| 936 | FLTENLLLY |
| 937 | SSDNIALLV |
| 938 | RSVSPKLFI |
| 939 | IVDTVSALV |
| 940 | KYTQLCQYL |
| 941 | TSDLATNNL |
| 942 | TVASLINTL |
| 943 | VSDADSTLI |
| 944 | VLYENQKLI |
| 945 | NYLKRRVVF |
| 946 | EAMYTPHTV |
| 947 | NOKLIANQF |
| 948 | FLAFVVFLL |
| 949 | LLLDDFVEI |
| 950 | ALLADKFPV |
| 951 | GQVDLFRNA |
| 952 | AMRPNFTIK |
| 953 | EILPVSMTK |
| 954 | TVYDDGARR |
| 955 | TIDYTEISF |
| 956 | YTRYVDNNF |
| 957 | YYRSLPGVF |
| 958 | TVNVLAWLY |
| 959 | KLIEYTDFA |
| 960 | VLLSVLQQL |
| 961 | PYNMRVIHF |
| 962 | TTIQTIVEV |
| 963 | LATNNLVVM |
| 964 | VLVPHVGEI |
| 965 | SQSIIAYTM |
| 966 | NTVCTVCGM |
| 967 | EAFEKMVSL |
| 968 | YFYTSKTTV |
| 969 | LLDDFVEII |
| 970 | HLDGEVITF |
| 971 | HFYWFFSNY |
| 972 | FTVLCLTPV |
| 973 | KSVNITFEL |
| 974 | TTAAKLMVV |
| 975 | LSDDAVVCF |
| 976 | LPNDDTLRV |
| 977 | YTVELGTEV |
| 978 | LEYHDVRVV |
| 979 | IADKYVRNL |
| 980 | THVQLSLPV |
| 981 | LLLALHFLL |
| 982 | KLLEQWNLV |
| 983 | YITGGVVQL |
| 984 | NVLTLVYKV |
| 985 | ALCTFLLNK |
| 986 | RVDGQVDLF |
| 987 | YGIATVREV |
| 988 | LLFNKVTLA |
| 989 | ERSEKSYEL |
| 990 | HHWLLLTIL |
| 991 | YFNSVCRLM |
| 992 | VSNGTHWFV |
| 993 | FVSNGTHWF |
| 994 | ISNSWLMWL |
| 995 | DTVIEVQGY |
| 996 | FLAFLLFLV |
| 997 | KLNVGDYFV |
| 998 | AYANSVFNI |
| 999 | YYVGYLQPR |
| 1000 | DALFAYTKR |
| 1001 | HSSGVTREL |
| 1002 | VTRELMREL |
| 1003 | QSASKIITL |
| 1004 | VGYLQPRTF |
| 1005 | LSDRVVFVL |
| 1006 | EAANFCALI |
| 1007 | MGYINVFAF |
| 1008 | KVDGVDVEL |
| 1009 | FGDDTVIEV |
| 1010 | LLLDRLNQL |
| 1011 | LLLEWLAMA |
| 1012 | KIFVDGVPF |
| 1013 | MLVYCFLGY |
| 1014 | MPLKAPKEI |
| 1015 | DGYFKIYSK |
| 1016 | DTLKNLSDR |
| 1017 | VMAYITGGV |
| 1018 | EAVMYMGTL |
| 1019 | FGDSVEEVL |
| 1020 | VYSTGSNVF |
| 1021 | VVNVVTTKI |
| 1022 | ELTPVVQTI |
| 1023 | ELPDEFVVV |
| 1024 | YFPLQSYGF |
| 1025 | AIKITEHSW |
| 1026 | ALDQAISMW |
| 1027 | SLIDFYLCF |
| 1028 | RVESSSKLW |
| 1029 | YKKPASREL |
| 1030 | LMDGSIIQF |
| 1031 | LAVPYNMRV |
| 1032 | SGFAAYSRY |
| 1033 | QEYADVFHL |
| 1034 | FAFACPDGV |
| 1035 | QVVDMSMTY |
| 1036 | FTNVYADSF |
| 1037 | KTIGPDMFL |
| 1038 | NRGMVLGSL |
| 1039 | YVFCTVNAL |
| 1040 | TLKNTVCTV |
| 1041 | NLWNTFTRL |
| 1042 | SALWEIQQV |
| 1043 | KNFKSVLYY |
| 1044 | EAAVKPLLV |
| 1045 | ILFALLQRY |
| 1046 | YLCFLAFLL |
| 1047 | LANECAQVL |
| 1048 | LPQLEQPYV |
| 1049 | SWVMRIMTW |
| 1050 | RTATKAYNV |
| 1051 | KVFTTVDNI |
| 1052 | KKFLPFQQF |
| 1053 | FHQKLLKSI |
| 1054 | NHTSPDVDL |
| 1055 | AHIQWMVMF |
| 1056 | TTTIKPVTY |
| 1057 | KYKYFSGAM |
| 1058 | QFAPSASAF |
| 1059 | YTNDKACPL |
| 1060 | VPHHVVATV |
| 1061 | KWDLTAFGL |
| 1062 | FVTVYSHLL |
| 1063 | LRLGSPLSL |
| 1064 | VINGDRWFL |
| 1065 | DILSRLDKV |
| 1066 | YECDIPIGA |
| 1067 | KSHKPPISF |
| 1068 | TVEEAKTVL |
| 1069 | AVKPLLVPH |
| 1070 | ATLPKGIMM |
| 1071 | HTDLMAAYV |
| 1072 | VPYCYDTNV |
| 1073 | NRNRFLYII |
| 1074 | VLYYQNNVF |
| 1075 | KTQFNYYKK |
| 1076 | REETGLLMP |
| 1077 | QVDVVNFNL |
| 1078 | VMYASAVVL |
| 1079 | AAYVDNSSL |
| 1080 | HFAIGLALY |
| 1081 | SLVKPSFYV |
| 1082 | LOLPQGTTL |
| 1083 | KLVNKFLAL |
| 1084 | NVAFNVVNK |
| 1085 | SRYWEPEFY |
| 1086 | TSNPTTFHL |
| 1087 | FYAYLRKHF |
| 1088 | QLFFSYFAV |
| 1089 | AMSAFAMMF |
| 1090 | WLPTGTLLV |
| 1091 | ETAQNSVRV |
| 1092 | TYFTQSRNL |
| 1093 | FVVSTGYHF |
| 1094 | SQLMCQPIL |
| 1095 | GSIHLYFDK |
| 1096 | LQTPFEIKL |
| 1097 | TPCNGVEGF |
| 1098 | AHGFELTSM |
| 1099 | GTSKFYGGW |
| 1100 | WMESEFRVY |
| 1101 | MSDVKCTSV |
| 1102 | DEFIERYKL |
| 1103 | ISMDNSPNL |
| 1104 | NPNYEDLLI |
| 1105 | SPNLAWPLI |
| 1106 | LIISVTSNY |
| 1107 | MSYEDQDAL |
| 1108 | SSLPSYAAF |
| 1109 | ASANLAATK |
| 1110 | VQQESPFVM |
| 1111 | IVSTIQRKY |
| 1112 | ETMSYLFQH |
| 1113 | FEEAALCTF |
| 1114 | YLASGGQPI |
| 1115 | MSNLGMPSY |
| 1116 | MVDTSLSGF |
| 1117 | RAFGEYSHV |
| 1118 | TQAPTHLSV |
| 1119 | AQLPAPRTL |
| 1120 | SAFFGMSRI |
| 1121 | FLGRYMSAL |
| 1122 | CAMRPNFTI |
| 1123 | AIMTRCLAV |
| 1124 | YYKLGASQR |
| 1125 | TVVIGTSKF |
| 1126 | LVAVPTGYV |
| 1127 | YSGVVTTVM |
| 1128 | ILSPLYAFA |
| 1129 | MSLSEQLRK |
| 1130 | FEIKLAKKF |
| 1131 | ILHCANFNV |
| 1132 | IGAGICASY |
| 1133 | LRPDTRYVL |
| 1134 | FSKQLQQSM |
| 1135 | AANTVIWDY |
| 1136 | TPLIQPIGA |
| 1137 | NYDLSVVNA |
| 1138 | YKTPPIKDF |
| 1139 | AWPLIVTAL |
| 1140 | QSINFVRII |
| 1141 | KHAFHTPAF |
| 1142 | LLMPLKAPK |
| 1143 | TVKPGNFNK |
| 1144 | YKGPITDVF |
| 1145 | MFLARGIVF |
| 1146 | ALAYYNTTK |
| 1147 | GTYEGNSPF |
| 1148 | LIDFYLCFL |
| 1149 | TLDSKTQSL |
| 1150 | KWDLIISDM |
| 1151 | FEYYHTTDP |
| 1152 | YVWKSYVHV |
| 1153 | LASHMYCSF |
| 1154 | SVNPYVCNA |
| 1155 | DFNLVAMKY |
| 1156 | RLRAKHYVY |
| 1157 | STTTNIVTR |
| 1158 | DIQLLKSAY |
| 1159 | KENSYTTTI |
| 1160 | VLSFCAFAV |
| 1161 | FLALCADSI |
| 1162 | ETICAPLTV |
| 1163 | FLFVAAIFY |
| 1164 | RYFRLTLGV |
| 1165 | AIDAYPLTK |
| 1166 | SAGFPFNKW |
| 1167 | IAIAMACLV |
| 1168 | ETTADIVVF |
| 1169 | HFISNSWLM |
| 1170 | HSWNADLYK |
| 1171 | AIMQLFFSY |
| 1172 | QHEVLLAPL |
| 1173 | MYDPKTKNV |
| 1174 | LSPRWYFYY |
| 1175 | KVSIWNLDY |
| 1176 | CSFGGVSVI |
| 1177 | FSTFEEAAL |
| 1178 | EETGLLMPL |
| 1179 | MTNRQFHQK |
| 1180 | SLRPDTRYV |
| 1181 | SYLFQHANL |
| 1182 | DYKHYTPSF |
| 1183 | ESGLKTILR |
| 1184 | LSFKELLVY |
| 1185 | NAANVYLKH |
| 1186 | AFPFTIYSL |
| 1187 | KATEETFKL |
| 1188 | TSMKYFVKI |
| 1189 | IINNTVYTK |
| 1190 | SFKWDLTAF |
| 1191 | MFVKHKHAF |
| 1192 | KLIFLWLLW |
| 1193 | WVYKQFDTY |
| 1194 | LALGGSVAI |
| 1195 | FAAYSRYRI |
| 1196 | FVSEETGTL |
| 1197 | STKHFYWFF |
| 1198 | INFVRIIMR |
| 1199 | TTIVYLTIV |
| 1200 | DTYPSLETI |
| 1201 | KDLPKEITV |
| 1202 | TTKGGRFVL |
| 1203 | LVLSVNPYV |
| 1204 | NVLAWLYAA |
| 1205 | WEIQQVVDA |
| 1206 | ASAFFGMSR |
| 1207 | IANQFNSAI |
| 1208 | WLMWLIINL |
| 1209 | LEIPRRNVA |
| 1210 | ILTSLLVLV |
| 1211 | TIVEVQPQL |
| 1212 | ELYHYQECV |
| 1213 | IIIGGAKLK |
| 1214 | VFAFPFTIY |
| 1215 | VTANVNALL |
| 1216 | ALLTKSSEY |
| 1217 | LTDEMIAQY |
| 1218 | LLALHRSYL |
| 1219 | LLNKEMYLK |
| 1220 | SILSPLYAF |
| 1221 | VVNAANVYL |
| 1222 | GFDYVYNPF |
| 1223 | IFTIGTVTL |
| 1224 | RVVFNGVSF |
| 1225 | QQWGFTGNL |
| 1226 | REVGFVVPG |
| 1227 | SAQTGIAVL |
| 1228 | HSLSHFVNL |
| 1229 | RVFSAVGNI |
| 1230 | LYLDAYNMM |
| 1231 | SASVYYSQL |
| 1232 | NLHSSRLSF |
| 1233 | SYEDQDALF |
| 1234 | FELTSMKYF |
| 1235 | VLGSLAATV |
| 1236 | NVLSTFISA |
| 1237 | STEKSNIIR |
| 1238 | GIITTVAAF |
| 1239 | IELSLIDFY |
| 1240 | FTDGVCLFW |
| 1241 | KATYKPNTW |
| 1242 | FRKSNLKPF |
| 1243 | VLSGHNLAK |
| 1244 | QLIKVTLVF |
| 1245 | RVCGVSAAR |
| 1246 | STVLSFCAF |
| 1247 | MATNYDLSV |
| 1248 | KLVLSVNPY |
| 1249 | TLVTMPLGY |
| 1250 | VQLHNDILL |
| 1251 | GLPWNVVRI |
| 1252 | NLLKDCPAV |
| 1253 | DYLVSTQEF |
| 1254 | DSKEGFFTY |
| 1255 | KVNSTLEQY |
| 1256 | NELSPVALR |
| 1257 | APISAMVRM |
| 1258 | FACPDGVKH |
| 1259 | FYWFFSNYL |
| 1260 | IAATRGATV |
| 1261 | IAQYTSALL |
| 1262 | NSFTRGVYY |
| 1263 | FNATRFASV |
| 1264 | IMASLVLAR |
| 1265 | WQLALSKGV |
| 1266 | VLSTFISAA |
| 1267 | KAIVSTIQR |
| 1268 | LYQPPQTSI |
| 1269 | STDTCFANK |
| 1270 | RYKLEGYAF |
| 1271 | TTITVNVLA |
| 1272 | MLQSCYNFL |
| 1273 | FLKKDAPYI |
| 1274 | DIAANTVIW |
| 1275 | RVWTLMNVL |
| 1276 | MVYMPASWV |
| 1277 | LTNMFTPLI |
| 1278 | QAWQPGVAM |
| 1279 | ASLPFGWLI |
| 1280 | MSYYCKSHK |
| 1281 | QTFSVLACY |
| 1282 | VSFCYMHHM |
| 1283 | AFDIYNDKV |
| 1284 | FPLNSIIKT |
| 1285 | VHFISNSWL |
| 1286 | ALLQRYRYK |
| 1287 | LESELVIGA |
| 1288 | VSIINNTVY |
| 1289 | RELKVTFFP |
| 1290 | DGARRVWTL |
| 1291 | LTNDNTSRY |
| 1292 | LYENAFLPF |
| 1293 | YPANSIVCR |
| 1294 | EVNEFACVV |
| 1295 | EVFNATRFA |
| 1296 | TLKATEETF |
| 1297 | VLNEKCSAY |
| 1298 | RDLPQGFSA |
| 1299 | LLFLMSFTV |
| 1300 | LVYAADPAM |
| 1301 | LLIIMRTFK |
| 1302 | GQQFGPTYL |
| 1303 | FVMMSAPPA |
| 1304 | QWSLFFFLY |
| 1305 | FVNLKQLPF |
| 1306 | SLPSYAAFA |
| 1307 | FTINCQEPK |
| 1308 | DVFHLYLQY |
| 1309 | YFVKIGPER |
| 1310 | HVGEIPVAY |
| 1311 | VAELEGIQY |
| 1312 | TEQPIDLVP |
| 1313 | LPKGIMMNV |
| 1314 | YTPSKLIEY |
| 1315 | LLLTILTSL |
| 1316 | NRQFHQKLL |
| 1317 | RLQSLQTYV |
| 1318 | KHAFLCLFL |
| 1319 | AAAYYVGYL |
| 1320 | MGIIAMSAF |
| 1321 | AIASEFSSL |
| 1322 | FKHLIPLMY |
| 1323 | EAFLIGCNY |
| 1324 | AHSCNVNRF |
| 1325 | KHADFDTWF |
| 1326 | EPKLGSLVV |
| 1327 | LPFGWLIVG |
| 1328 | IAQVDVVNF |
| 1329 | RFRRAFGEY |
| 1330 | VYANGGKGF |
| 1331 | EVQELYSPI |
| 1332 | VMVELVAEL |
| 1333 | YPIILRLGS |
| 1334 | KATNNAMQV |
| 1335 | DISGINASV |
| 1336 | RTIAFGGCV |
| 1337 | EVITFDNLK |
| 1338 | HSMQNCVLK |
| 1339 | HFDGQQGEV |
| 1340 | VTVYSHLLL |
| 1341 | TLGVLVPHV |
| 1342 | DTANPKTPK |
| 1343 | VEVEKGVLP |
| 1344 | AARVVRSIF |
| 1345 | SSRVPDLLV |
| 1346 | REQIDGYVM |
| 1347 | TSVDCTMYI |
| 1348 | RHHANEYRL |
| 1349 | YIVDSVTVK |
| 1350 | TILDGISQY |
| 1351 | TLAVPYNMR |
| 1352 | RQEEVQELY |
| 1353 | HEETIYNLL |
| 1354 | GYLPQNAVV |
| 1355 | VGTNLPLQL |
| 1356 | LYLGGMSYY |
| 1357 | TLNDLNETL |
| 1358 | EAARYMRSL |
| 1359 | STYASQGLV |
| 1360 | FEHIVYGDF |
| 1361 | TAQNSVRVL |
| 1362 | FVFPLNSII |
| 1363 | MKFLVFLGI |
| 1364 | KAPKEIIFL |
| 1365 | SYSGQSTQL |
| 1366 | ATVHTANKW |
| 1367 | MFYKGVITH |
| 1368 | IIFWFSLEL |
| 1369 | AQVLSEMVM |
| 1370 | FYGGWHNML |
| 1371 | CVPLNIIPL |
| 1372 | NDLCFTNVY |
| 1373 | YTDFATSAC |
| 1374 | DEFTPFDVV |
| 1375 | FFGMSRIGM |
| 1376 | ETLGVLVPH |
| 1377 | YWFFSNYLK |
| 1378 | ITVATSRTL |
| 1379 | WPVTLACFV |
| 1380 | LPSYAAFAT |
| 1381 | MVTNNTFTL |
| 1382 | ELLHAPATV |
| 1383 | KHDFFKFRI |
| 1384 | VYNPFMIDV |
| 1385 | KSFDLGDEL |
| 1386 | ILIVTTIVY |
| 1387 | RLWLCWKCR |
| 1388 | CVMYASAVV |
| 1389 | KLOFTSLEI |
| 1390 | HTDFSSEII |
| 1391 | KQGDDYVYL |
| 1392 | LTYTGAIKL |
| 1393 | ETSWQTGDF |
| 1394 | DAVTAYNGY |
| 1395 | QEIQLQAAV |
| 1396 | NALDQAISM |
| 1397 | GHMLDMYSV |
| 1398 | RYMNSQGLL |
| 1399 | LLDKRTTCF |
| 1400 | LAHIQWMVM |
| 1401 | RNYVFTGYR |
| 1402 | AVDAAKAYK |
| 1403 | ITHDVSSAI |
| 1404 | NTWCIRCLW |
| 1405 | FQTRAGCLI |
| 1406 | AIFYLITPV |
| 1407 | YLEGSVRVV |
| 1408 | SIIAYTMSL |
| 1409 | FQPTNGVGY |
| 1410 | TDYKHWPQI |
| 1411 | HDIGNPKAI |
| 1412 | LLLQILFAL |
| 1413 | YFVLTSHTV |
| 1414 | SVIYLYLTF |
| 1415 | VMHANYIFW |
| 1416 | MESEFRVYS |
| 1417 | YTEISFMLW |
| 1418 | RLSFKELLV |
| 1419 | SVFNICQAV |
| 1420 | VFVSNGTHW |
| 1421 | DSIIIGGAK |
| 1422 | GEVITFDNL |
| 1423 | DAMMFTSDL |
| 1424 | SAFVNLKQL |
| 1425 | DQAISMWAL |
| 1426 | VAVKMFDAY |
| 1427 | RVCTNYMPY |
| 1428 | KTSVDCTMY |
| 1429 | EKALKYLPI |
| 1430 | GHFAWWTAF |
| 1431 | SIIKTIQPR |
| 1432 | YYFMRFRRA |
| 1433 | TSHKLVLSV |
| 1434 | FIASFRLFA |
| 1435 | AYLRKHFSM |
| 1436 | LYYPSARIV |
| 1437 | DLQDLKWAR |
| 1438 | CTDDNALAY |
| 1439 | TPRDLGACI |
| 1440 | NEEIAIILA |
| 1441 | TLAILTALR |
| 1442 | RTILGSALL |
| 1443 | VPHVGEIPV |
| 1444 | NYTVSCLPF |
| 1445 | FLCWHTNCY |
| 1446 | EVPANSTVL |
| 1447 | QVVSDIDYV |
| 1448 | SVSPKLFIR |
| 1449 | VETVKGLDY |
| 1450 | TESNKKFLP |
| 1451 | IEDLLFNKV |
| 1452 | IAKNTVKSV |
| 1453 | CPACHNSEV |
| 1454 | YSLFDMSKF |
| 1455 | GLNDNLLEI |
| 1456 | DKAYKIEEL |
| 1457 | WPWYIWLGF |
| 1458 | SLEDKAFQL |
| 1459 | KISEMHPAL |
| 1460 | DEFVVVTVK |
| 1461 | ASAVVLLIL |
| 1462 | LLTNMFTPL |
| 1463 | KWYIRVGAR |
| 1464 | TSNSFDVLK |
| 1465 | LQIPFAMQM |
| 1466 | VYSVIYLYL |
| 1467 | SLLSVLLSM |
| 1468 | GVRRSFYVY |
| 1469 | TTIVNGVRR |
| 1470 | RQVVNVVTT |
| 1471 | KIQEGVVDY |
| 1472 | MYTPHTVLQ |
| 1473 | RLYLDAYNM |
| 1474 | LKKRWQLAL |
| 1475 | ATVAYFNMV |
| 1476 | ALVYFLQSI |
| 1477 | DEMIAQYTS |
| 1478 | ATAEAELAK |
| 1479 | SEYTGNYQC |
| 1480 | TVYTKVDGV |
| 1481 | VVDKYFDCY |
| 1482 | DVRVVLDFI |
| 1483 | TTLPVNVAF |
| 1484 | LPLQLGFST |
| 1485 | KNFTTAPAI |
| 1486 | NAAISDYDY |
| 1487 | DLPQGFSAL |
| 1488 | NSQGLLPPK |
| 1489 | LTNIFGTVY |
| 1490 | GPFVDRQTA |
| 1491 | SALNHTKKW |
| 1492 | VGDSAEVAV |
| 1493 | TTFTYASAL |
| 1494 | QSSYIVDSV |
| 1495 | AVDCALDPL |
| 1496 | LVASIKNFK |
| 1497 | TTFHLDGEV |
| 1498 | DHVDILGPL |
| 1499 | QLCQYLNTL |
| 1500 | KLFIRQEEV |
| 1501 | GVYDYLVST |
| 1502 | GERVRQALL |
| 1503 | IDYVPLKSA |
| 1504 | NFCALILAY |
| 1505 | YHFRELGVV |
| 1506 | HKPPISFPL |
| 1507 | SFRLFARTR |
| 1508 | ILGTVSWNL |
| 1509 | SSNVANYQK |
| 1510 | TPVCINGLM |
| 1511 | FYYVWKSYV |
| 1512 | IVAGGIVAI |
| 1513 | KLMPVCVET |
| 1514 | AFATAQEAY |
| 1515 | WFSQRGGSY |
| 1516 | TFEYVSQPF |
| 1517 | MLRIMASLV |
| 1518 | YRARAGEAA |
| 1519 | YPSARIVYT |
| 1520 | LVYDNKLKA |
| 1521 | AAVDALCEK |
| 1522 | TLRVEAFEY |
| 1523 | FRVQPTESI |
| 1524 | YVLPNDDTL |
| 1525 | LHDELTGHM |
| 1526 | WVPRASANI |
| 1527 | IVVTCLAYY |
| 1528 | TLLALHRSY |
| 1529 | TTPGSGVPV |
| 1530 | VMCGGSLYV |
| 1531 | QMCLSTLMK |
| 1532 | NLKTLLSLR |
| 1533 | STQDLFLPF |
| 1534 | TLSEQLDFI |
| 1535 | FLEYHDVRV |
| 1536 | KRFDNPVLP |
| 1537 | REVLSDREL |
| 1538 | QEKNFTTAP |
| 1539 | IVDEPEEHV |
| 1540 | AQYTSALLA |
| 1541 | ETVKGLDYK |
| 1542 | AHVASCDAI |
| 1543 | MGFKMNYQV |
| 1544 | QALLKTVQF |
| 1545 | VPYNMRVIH |
| 1546 | SHTVMPLSA |
| 1547 | EEAIRHVRA |
| 1548 | VPNQPYPNA |
| 1549 | STDTGVEHV |
| 1550 | QAISMWALI |
| 1551 | TLGVYDYLV |
| 1552 | TTVDNINLH |
| 1553 | STDVVYRAF |
| 1554 | LTILTSLLV |
| 1555 | QLMCQPILL |
| 1556 | ARNGVLITE |
| 1557 | KHTDFSSEI |
| 1558 | AEYHNESGL |
| 1559 | LEQYVFCTV |
| 1560 | KPVPEVKIL |
| 1561 | AKPPPGDQF |
| 1562 | MMFVKHKHA |
| 1563 | AMPNMLRIM |
| 1564 | IQYIDIGNY |
| 1565 | STCMMCYKR |
| 1566 | MVSLLSVLL |
| 1567 | SFLAHIQWM |
| 1568 | GTTTLNGLW |
| 1569 | ALLAVFQSA |
| 1570 | TEVPANSTV |
| 1571 | DESGEFKLA |
| 1572 | GVFCGVDAV |
| 1573 | FMRFRRAFG |
| 1574 | YKQFDTYNL |
| 1575 | IATNGPLKV |
| 1576 | HVVAFNTLL |
| 1577 | RLANECAQV |
| 1578 | YVVDDPCPI |
| 1579 | EEIAIILAS |
| 1580 | LAMAVMLLL |
| 1581 | RVVVLSFEL |
| 1582 | EFTPFDVVR |
| 1583 | NQHEVLLAP |
| 1584 | CEFCGTENL |
| 1585 | FLQSINFVR |
| 1586 | LQSINFVRI |
| 1587 | SVLYNSASF |
| 1588 | IANYAKPFL |
| 1589 | LPGCDGGSL |
| 1590 | MRIFTIGTV |
| 1591 | VLLAPLLSA |
| 1592 | LQGPPGTGK |
| 1593 | LYLQYIRKL |
| 1594 | DMFLGTCRR |
| 1595 | MPSYCTGYR |
| 1596 | FKMFYKGVI |
| 1597 | YYSLLMPIL |
| 1598 | NLKQLPFFY |
| 1599 | GFAAYSRYR |
| 1600 | DYGARFYFY |
| 1601 | SFNPETNIL |
| 1602 | FLRDGWEIV |
| 1603 | YMPASWVMR |
| 1604 | KVKYLYFIK |
| 1605 | LTFYLTNDV |
| 1606 | FALLQRYRY |
| 1607 | WLTNIFGTV |
| 1608 | LAAECTIFK |
| 1609 | WHNMLKTVY |
| 1610 | VSQPFLMDL |
| 1611 | GLFKDCSKV |
| 1612 | NELSRVLGL |
| 1613 | GADLKSFDL |
| 1614 | AMQTMLFTM |
| 1615 | TPVVQTIEV |
| 1616 | NYYKKDNSY |
| 1617 | LVSTQEFRY |
| 1618 | CASEYTGNY |
| 1619 | GHSMQNCVL |
| 1620 | LSVCLGSLI |
| 1621 | TIAFGGCVF |
| 1622 | KVVKVTIDY |
| 1623 | EQPYVFIKR |
| 1624 | LEILQKEKV |
| 1625 | EPVLKGVKL |
| 1626 | SIWNLDYII |
| 1627 | SRTLSYYKL |
| 1628 | LQSCYNFLK |
| 1629 | SVLLFLAFV |
| 1630 | KMVSLLSVL |
| 1631 | LNDFNLVAM |
| 1632 | NSPNLAWPL |
| 1633 | VVNQNAQAL |
| 1634 | VTIAEILLI |
| 1635 | SEYCRHGTC |
| 1636 | KYVRNLQHR |
| 1637 | KMAFPSGKV |
| 1638 | VVIGTSKFY |
| 1639 | QTYVTQQLI |
| 1640 | DKFKVNSTL |
| 1641 | KLMVVIPDY |
| 1642 | VYQLRARSV |
| 1643 | SGDGTTSPI |
| 1644 | FELLHAPAT |
| 1645 | QELYSPIFL |
| 1646 | TFTYASALW |
| 1647 | TENLTKEGA |
| 1648 | TLVPQEHYV |
| 1649 | HLMSFPQSA |
| 1650 | AKYTQLCQY |
| 1651 | GELGDVRET |
| 1652 | VEAPLVGTP |
| 1653 | SVAIKITEH |
| 1654 | RNAGIVGVL |
| 1655 | GYYRRATRR |
| 1656 | LWLLWPVTL |
| 1657 | GSFCTQLNR |
| 1658 | GIATVREVL |
| 1659 | QECVRGTTV |
| 1660 | KKNNLPFKL |
| 1661 | RLDKVEAEV |
| 1662 | TTAYANSVF |
| 1663 | TGDSCNNYM |
| 1664 | QESPFVMMS |
| 1665 | GYKSVNITF |
| 1666 | EYVSQPFLM |
| 1667 | KGLPWNVVR |
| 1668 | KEKVNINIV |
| 1669 | AAFHQECSL |
| 1670 | LAYILFTRF |
| 1671 | KFLKTNCCR |
| 1672 | GVKHVYQLR |
| 1673 | EAALCTFLL |
| 1674 | AQKFNGLTV |
| 1675 | ITLATCELY |
| 1676 | VPLKSATCI |
| 1677 | IPTITQMNL |
| 1678 | AALQIPFAM |
| 1679 | KLNEEIAII |
| 1680 | ILGLPTQTV |
| 1681 | TTLKGVEAV |
| 1682 | EEVQELYSP |
| 1683 | KPLLVPHHV |
| 1684 | FYVLGLAAI |
| 1685 | MDNSPNLAW |
| 1686 | EVGKPRPPL |
| 1687 | AAVINGDRW |
| 1688 | IAKKPTETI |
| 1689 | KHWPQIAQF |
| 1690 | LAILTALRL |
| 1691 | AFGGCVFSY |
| 1692 | ASFSTFKCY |
| 1693 | LHCANFNVL |
| 1694 | TRVLSNLNL |
| 1695 | ISAARQGFV |
| 1696 | CEESSAKSA |
| 1697 | CGPKKSTNL |
| 1698 | SSTASALGK |
| 1699 | DHSSSSDNI |
| 1700 | AYANRNRFL |
| 1701 | LFLMSFTVL |
| 1702 | NYMLTYNKV |
| 1703 | ADSIIIGGA |
| 1704 | TQLGIEFLK |
| 1705 | VGMQKYSTL |
| 1706 | LAPLLSAGI |
| 1707 | AAKKNNLPF |
| 1708 | ISTIGVCSM |
| 1709 | TFFKLVNKF |
| 1710 | GLLLALHFL |
| 1711 | AQVAKSHNI |
| 1712 | KVTLVFLFV |
| 1713 | VRIKIVQML |
| 1714 | ARVECFDKF |
| 1715 | SYIVDSVTV |
| 1716 | YQVNGYPNM |
| 1717 | TVLCLTPVY |
| 1718 | KWGKARLYY |
| 1719 | LEGNFYGPF |
| 1720 | CPAVAKHDF |
| 1721 | RYRYKPHSL |
| 1722 | TLSYEQFKK |
| 1723 | AFEKMVSLL |
| 1724 | SLVPGFNEK |
| 1725 | LVLVQSTQW |
| 1726 | DTTEAFEKM |
| 1727 | HEIAWYTER |
| 1728 | DEVARDLSL |
| 1729 | GEFKLASHM |
| 1730 | TPSDFVRAT |
| 1731 | GSLAATVRL |
| 1732 | WMVMFTPLV |
| 1733 | SNYLKRRVV |
| 1734 | AYSNNSIAI |
| 1735 | ANGQVFGLY |
| 1736 | VYSFLPGVY |
| 1737 | LNRYFRLTL |
| 1738 | LSPVALRQM |
| 1739 | WEIVKFIST |
| 1740 | LIVNSVLLF |
| 1741 | VFLFVAAIF |
| 1742 | IEELFYSYA |
| 1743 | LQTYVTQQL |
| 1744 | IVNSVLLFL |
| 1745 | LYIDINGNL |
| 1746 | DLSPRWYFY |
| 1747 | NPKTPKYKF |
| 1748 | KPYIKWDLL |
| 1749 | DADSKIVQL |
| 1750 | RFKESPFEL |
| 1751 | NIFGTVYEK |
| 1752 | ALPETTADI |
| 1753 | FFLYENAFL |
| 1754 | KTLQPVSEL |
| 1755 | SAQCFKMFY |
| 1756 | IMRTFKVSI |
| 1757 | HVVATVQEI |
| 1758 | KLTDNVYIK |
| 1759 | DTVRTNVYL |
| 1760 | AEETRKLMP |
| 1761 | SIIIGGAKL |
| 1762 | FFFLYENAF |
| 1763 | MADLVYALR |
| 1764 | YINVFAFPF |
| 1765 | SEAKCWTET |
| 1766 | CTSCCFSER |
| 1767 | YVPLKSATC |
| 1768 | QQGEVPVSI |
| 1769 | VHTANKWDL |
| 1770 | KDTEKYCAL |
| 1771 | VSTSGRWVL |
| 1772 | FTALTQHGK |
| 1773 | QASLNGVTL |
| 1774 | YFDKAGQKT |
| 1775 | LLSAGIFGA |
| 1776 | YSLRLIDAM |
| 1777 | NKHAFHTPA |
| 1778 | MFTPLIQPI |
| 1779 | AVPYNMRVI |
| 1780 | DFVNEFYAY |
| 1781 | SANLAATKM |
| 1782 | FAFPFTIYS |
| 1783 | TQSRNLQEF |
| 1784 | GTITSGWTF |
| 1785 | AAVGELLLL |
| 1786 | LTSHTVMPL |
| 1787 | LAAIMQLFF |
| 1788 | LSVLQQLRV |
| 1789 | IFWRNTNPI |
| 1790 | DLYKLMGHF |
| 1791 | YSQLMCQPI |
| 1792 | TIDGSSGVV |
| 1793 | LWPVTLACF |
| 1794 | WLAMAVMLL |
| 1795 | FLNKVVSTT |
| 1796 | VFVLWAHGF |
| 1797 | FTPLVPFWI |
| 1798 | HFVNLDNLR |
| 1799 | ASKIITLKK |
| 1800 | IIAMSAFAM |
| 1801 | EAVKTQFNY |
| 1802 | STASALGKL |
| 1803 | RITGLYPTL |
| 1804 | DPFLGVYYH |
| 1805 | QKFNGLTVL |
| 1806 | MNVAKYTQL |
| 1807 | YYVWKSYVH |
| 1808 | NLYDKLVSS |
| 1809 | ALDISASIV |
| 1810 | KWADNNCYL |
| 1811 | EQKSILSPL |
| 1812 | RKSAPLIEL |
| 1813 | IAIVMVTIM |
| 1814 | AANFCALIL |
| 1815 | MVPHISRQR |
| 1816 | SEAVEAPLV |
| 1817 | HWFVTQRNF |
| 1818 | IMQLFFSYF |
| 1819 | IQPGQTFSV |
| 1820 | IIMRLWLCW |
| 1821 | SLPINVIVF |
| 1822 | KIITLKKRW |
| 1823 | SKVGGNYNY |
| 1824 | IQASLPFGW |
| 1825 | ITLKKRWQL |
| 1826 | LMSFTVLCL |
| 1827 | LAWPLIVTA |
| 1828 | ETIQITISS |
| 1829 | AVVCFNSTY |
| 1830 | IERFVSLAI |
| 1831 | VTDTPKGPK |
| 1832 | LPIGINITR |
| 1833 | QAITVTPEA |
| 1834 | ILKPANNSL |
| 1835 | TVQFCDAMR |
| 1836 | QPILLLDQA |
| 1837 | YSKHTPINL |
| 1838 | HMVVKAALL |
| 1839 | KRAKVTSAM |
| 1840 | IPYNSVTSS |
| 1841 | ASCDAIMTR |
| 1842 | TGSNVFQTR |
| 1843 | VEQKIAEIP |
| 1844 | NIDYDCVSF |
| 1845 | KSPNFSKLI |
| 1846 | YYHKNNKSW |
| 1847 | TTCCSLSHR |
| 1848 | VSEETGTLI |
| 1849 | STECSNLLL |
| 1850 | TVYEKLKPV |
| 1851 | SLAIDAYPL |
| 1852 | VVENPTIQK |
| 1853 | GYAFEHIVY |
| 1854 | QQLIRAAEI |
| 1855 | VANYQKVGM |
| 1856 | KYTMADLVY |
| 1857 | YAADPAMHA |
| 1858 | YLAVFDKNL |
| 1859 | SPFVMMSAP |
| 1860 | ETKDVVECL |
| 1861 | SSPDAVTAY |
| 1862 | IGYYRRATR |
| 1863 | KSWMESEFR |
| 1864 | IFFASFYYV |
| 1865 | KVQHMVVKA |
| 1866 | TTLNDFNLV |
| 1867 | IPKEEVKPF |
| 1868 | NYQHEETIY |
| 1869 | YIKWPWYIW |
| 1870 | RAAEIRASA |
| 1871 | YVLGLAAIM |
| 1872 | GMSRIGMEV |
| 1873 | ASIKNFKSV |
| 1874 | AFLPFAMGI |
| 1875 | GVDIAANTV |
| 1876 | KFKEGVEFL |
| 1877 | NSYFTEQPI |
| 1878 | FNPETNILL |
| 1879 | TSLSGFKLK |
| 1880 | FSTFKCYGV |
| 1881 | INDMILSLL |
| 1882 | TEKYCALAP |
| 1883 | QINDMILSL |
| 1884 | YTVEEAKTV |
| 1885 | KPFLNKVVS |
| 1886 | KQGNFKNLR |
| 1887 | KPHSLSDGL |
| 1888 | RLIDAMMFT |
| 1889 | NTLTLAVPY |
| 1890 | NENGTITDA |
| 1891 | LTYNKVENM |
| 1892 | VAYRKVLLR |
| 1893 | IEYTDFATS |
| 1894 | CYFGLFCLL |
| 1895 | NPPALQDAY |
| 1896 | SDRVVFVLW |
| 1897 | LTKHPNQEY |
| 1898 | NCYDYCIPY |
| 1899 | TVREVLSDR |
| 1900 | QPITNCVKM |
| 1901 | IAMACLVGL |
| 1902 | LAFVVFLLV |
| 1903 | TSRTLSYYK |
| 1904 | TLNDFNLVA |
| 1905 | YLKSPNFSK |
| 1906 | ELKINAACR |
| 1907 | QTVTLLPAA |
| 1908 | NEKCSAYTV |
| 1909 | VAFELWAKR |
| 1910 | SLSDGLLLA |
| 1911 | LEQPTSEAV |
| 1912 | KTILRKGGR |
| 1913 | VAAIVFITL |
| 1914 | QFTSLEIPR |
| 1915 | GPKVYPIIL |
| 1916 | HYVRITGLY |
| 1917 | LPIDKCSRI |
| 1918 | QIYKTPPIK |
| 1919 | EAARVVRSI |
| 1920 | VLCNSQTSL |
| 1921 | LTSMKYFVK |
| 1922 | MLSDTLKNL |
| 1923 | LTNDVSFLA |
| 1924 | VIYLYLTFY |
| 1925 | CEIVGGQIV |
| 1926 | SLLMPILTL |
| 1927 | LLKSIAATR |
| 1928 | AYWVPRASA |
| 1929 | RNFYEPQII |
| 1930 | HEHEIAWYT |
| 1931 | LVDSDLNDF |
| 1932 | LLMPILTLT |
| 1933 | YGQQFGPTY |
| 1934 | HFLLFFRAL |
| 1935 | ALYNKYKYF |
| 1936 | GDMVPHISR |
| 1937 | QSTQWSLFF |
| 1938 | LOKAAITIL |
| 1939 | VLDMCASLK |
| 1940 | KEGATTCGY |
| 1941 | HSLSDGLLL |
| 1942 | WLSYFIASF |
| 1943 | FLPFAMGII |
| 1944 | SMWSFNPET |
| 1945 | GSVRVVTTF |
| 1946 | MLFTMLRKL |
| 1947 | YEAMYTPHT |
| 1948 | ILLLDQALV |
| 1949 | RIMASLVLA |
| 1950 | QEGVVDYGA |
| 1951 | EKMVSLLSV |
| 1952 | ISTKHFYWF |
| 1953 | SNVTWFHAI |
| 1954 | TSNQVAVLY |
| 1955 | RNIKPVPEV |
| 1956 | APLLSAGIF |
| 1957 | LVSSFLEMK |
| 1958 | FSNYLKRRV |
| 1959 | LTTAAKLMV |
| 1960 | ISDYDYYRY |
| 1961 | RLNEVAKNL |
| 1962 | GVYYPDKVF |
| 1963 | NVAFELWAK |
| 1964 | LSYGIATVR |
| 1965 | VRATATIPI |
| 1966 | YLVSTQEFR |
| 1967 | NMLRIMASL |
| 1968 | MMILSDDAV |
| 1969 | FLLVTLAIL |
| 1970 | GQGLNGYTV |
| 1971 | GPKVKYLYF |
| 1972 | VLKLKVDTA |
| 1973 | EVGFVVPGL |
| 1974 | FIDTKRGVY |
| 1975 | AFQLTPIAV |
| 1976 | GTTTYKLNV |
| 1977 | EEMLDNRAT |
| 1978 | LAFLLFLVL |
| 1979 | YLALYNKYK |
| 1980 | DLVYALRHF |
| 1981 | TVSCLPFTI |
| 1982 | TPGSGVPVV |
| 1983 | DKAFQLTPI |
| 1984 | REVRTIKVF |
| 1985 | LOHRLYECL |
| 1986 | EEHVQIHTI |
| 1987 | ADQAMTQMY |
| 1988 | AGIVGVLTL |
| 1989 | SEISMDNSP |
| 1990 | NLDYIINLI |
| 1991 | ELFENKTTL |
| 1992 | VVPGLPGTI |
| 1993 | SLENVAFNV |
| 1994 | KFTDGVCLF |
| 1995 | AFLIGCNYL |
| 1996 | LPETTADIV |
| 1997 | KYLYFIKGL |
| 1998 | TVMPLSAPT |
| 1999 | ITLCFTLKR |
| 2000 | ISQYSLRLI |
| 2001 | NIDGYFKIY |
| 2002 | DLKWARFPK |
| 2003 | YFRLTLGVY |
| 2004 | AALTNNVAF |
| 2005 | ESSAKSASV |
| 2006 | LLQLCTFTR |
| 2007 | LLTKSSEYK |
| 2008 | EEAALCTFL |
| 2009 | CVDIPGIPK |
| 2010 | EYFNSVCRL |
| 2011 | FFSYFAVHF |
| 2012 | LPDEFVVVT |
| 2013 | NYIAQVDVV |
| 2014 | EANMDQESF |
| 2015 | RPLLESELV |
| 2016 | NTYLEGSVR |
| 2017 | DLKGKYVQI |
| 2018 | KLPDDFTGC |
| 2019 | QMAYRFNGI |
| 2020 | ASQGLVASI |
| 2021 | WHHSIGFDY |
| 2022 | SEETGTLIV |
| 2023 | KYAISAKNR |
| 2024 | LAVFDKNLY |
| 2025 | YDANYFLCW |
| 2026 | FTTVDNINL |
| 2027 | AQALNTLVK |
| 2028 | VEKGVLPQL |
| 2029 | IAEIPKEEV |
| 2030 | KALNLGETF |
| 2031 | LPRVFSAVG |
| 2032 | STQWSLFFF |
| 2033 | DNLKTLLSL |
| 2034 | KWDLLKYDF |
| 2035 | LIDSYFVVK |
| 2036 | GDCATVHTA |
| 2037 | KSAQCFKMF |
| 2038 | RKGGRTIAF |
| 2039 | SHEGKTFYV |
| 2040 | LLTILTSLL |
| 2041 | QWMVMFTPL |
| 2042 | LRAKHYVYI |
| 2043 | NFGAISSVL |
| 2044 | FAMMFVKHK |
| 2045 | MRNAGIVGV |
| 2046 | SALEPLVDL |
| 2047 | TVMFLARGI |
| 2048 | KPANNSLKI |
| 2049 | ESNKKFLPF |
| 2050 | TALLTLQQI |
| 2051 | YIKWDLLKY |
| 2052 | GTSTDVVYR |
| 2053 | LYIIKLIFL |
| 2054 | EYPIIGDEL |
| 2055 | SVCLGSLIY |
| 2056 | NQTTTIQTI |
| 2057 | RIIPARARV |
| 2058 | VVISSDVLV |
| 2059 | AYKIEELFY |
| 2060 | QIDRLITGR |
| 2061 | ESVQTFFKL |
| 2062 | MQMAYRFNG |
| 2063 | ASVYAWNRK |
| 2064 | LTALRLCAY |
| 2065 | MSFPQSAPH |
| 2066 | TIAEILLII |
| 2067 | VVFDEISMA |
| 2068 | VTFFIYNKI |
| 2069 | LATCELYHY |
| 2070 | RLKLFDRYF |
| 2071 | TSTDVVYRA |
| 2072 | QHGKEDLKF |
| 2073 | HDELTGHML |
| 2074 | LPTGVHAGT |
| 2075 | TYRRLISMM |
| 2076 | CALAPNMMV |
| 2077 | FLHVTYVPA |
| 2078 | YSRYRIGNY |
| 2079 | IAPGQTGKI |
| 2080 | CYKRNRATR |
| 2081 | FSSLPSYAA |
| 2082 | KEILVTYNC |
| 2083 | LALSKGVHF |
| 2084 | YEYGTEDDY |
| 2085 | KLKPVLDWL |
| 2086 | KIILFLALI |
| 2087 | AESHVDTDL |
| 2088 | FYEPQIITT |
| 2089 | GYPNMFITR |
| 2090 | HSDKFTDGV |
| 2091 | IRKSNHNFL |
| 2092 | LTLVYKVYY |
| 2093 | SVKGLQPSV |
| 2094 | DYIINLIIK |
| 2095 | VDILGPLSA |
| 2096 | GPKKSTNLV |
| 2097 | VLDWLEEKF |
| 2098 | VFITLCFTL |
| 2099 | VVYCPRHVI |
| 2100 | LVKNKCVNF |
| 2101 | YREAACCHL |
| 2102 | KPASRELKV |
| 2103 | GTDLEGNFY |
| 2104 | NLAKHCLHV |
| 2105 | GELLLLEWL |
| 2106 | DEFSSNVAN |
| 2107 | TAAKLMVVI |
| 2108 | KARLYYDSM |
| 2109 | VFDEISMAT |
| 2110 | TCDGTTFTY |
| 2111 | GHNLAKHCL |
| 2112 | ITPVHVMSK |
| 2113 | PQNAVVKIY |
| 2114 | RKAVFISPY |
| 2115 | SRIIPARAR |
| 2116 | PYPDPSRIL |
| 2117 | TIYSLLLCR |
| 2118 | VPATVSVSS |
| 2119 | CSMTDIAKK |
| 2120 | VLHDIGNPK |
| 2121 | RVVISSDVL |
| 2122 | FLYIIKLIF |
| 2123 | VESSSKLWA |
| 2124 | NLVAVPTGY |
| 2125 | LSAQTGIAV |
| 2126 | MLKTVYSDV |
| 2127 | IPLTTAAKL |
| 2128 | EQWNLVIGF |
| 2129 | MCDIRQLLF |
| 2130 | VQAGNVQLR |
| 2131 | GLQPSVGPK |
| 2132 | QIGEYTFEK |
| 2133 | SSVELKHFF |
| 2134 | YNSASFSTF |
| 2135 | NVVTTKIAL |
| 2136 | QIGGYTEKW |
| 2137 | TSQWLTNIF |
| 2138 | SLPFGWLIV |
| 2139 | KPSFYVYSR |
| 2140 | TTNGDFLHF |
| 2141 | VVAFNTLLF |
| 2142 | ASLNGVTLI |
| 2143 | WICLLQFAY |
| 2144 | GPEHSLAEY |
| 2145 | IFVDGVPFV |
| 2146 | VTSNYSGVV |
| 2147 | CVEEVTTTL |
| 2148 | IRQEEVQEL |
| 2149 | NFNKDFYDF |
| 2150 | YPVASPNEC |
| 2151 | KVVSTTTNI |
| 2152 | MLLLQILFA |
| 2153 | NLKYAISAK |
| 2154 | LDYKAFKQI |
| 2155 | LPAPRTLLT |
| 2156 | FVKRVDWTI |
| 2157 | FPNTYLEGS |
| 2158 | FVLALLSDL |
| 2159 | NRNYVFTGY |
| 2160 | YHPNCVNCL |
| 2161 | YIRKLHDEL |
| 2162 | LALYYPSAR |
| 2163 | RLIIRENNR |
| 2164 | YQIGGYTEK |
| 2165 | NNAAIVLQL |
| 2166 | LAVVVCNSL |
| 2167 | VRQCSGVTF |
| 2168 | SQLDEEQPM |
| 2169 | GQQQQGQTV |
| 2170 | CTNYMPYFF |
| 2171 | GLFCLLNRY |
| 2172 | NLDSCKRVL |
| 2173 | ATTAYANSV |
| 2174 | LGDIAARDL |
| 2175 | VEIIKSQDL |
| 2176 | TEVNEFACV |
| 2177 | VYRGTTTYK |
| 2178 | NPTIQKDVL |
| 2179 | RHVRAWIGF |
| 2180 | RINWITGGI |
| 2181 | TLQCIMLVY |
| 2182 | DIASTDTCF |
| 2183 | NVVIKVCEF |
| 2184 | RDLICAQKF |
| 2185 | LIPLMYKGL |
| 2186 | CFANKHADF |
| 2187 | NSWLMWLII |
| 2188 | WTNAGDYIL |
| 2189 | ALRANSAVK |
| 2190 | LFVTVYSHL |
| 2191 | ALLSDLQDL |
| 2192 | NYQKVGMQK |
| 2193 | DTKFKTEGL |
| 2194 | TEKSNIIRG |
| 2195 | GDELKINAA |
| 2196 | YNLWNTFTR |
| 2197 | TNNLVVMAY |
| 2198 | LLSDLQDLK |
| 2199 | WSFNPETNI |
| 2200 | PYNSVTSSI |
| 2201 | NIIPLTTAA |
| 2202 | EQFKKGVQI |
| 2203 | AVITREVGF |
| 2204 | FWITIAYII |
| 2205 | YAFEHIVYG |
| 2206 | DFQENWNTK |
| 2207 | ARTVAGVSI |
| 2208 | TPINLVRDL |
| 2209 | IMRLWLCWK |
| 2210 | EISFMLWCK |
| 2211 | YAAVINGDR |
| 2212 | GVVREFLTR |
| 2213 | KRGDKSVYY |
| 2214 | KHIDAYKTF |
| 2215 | TAHSCNVNR |
| 2216 | FACVVADAV |
| 2217 | SSRSRNSSR |
| 2218 | LATHGLAAV |
| 2219 | RWVLNNDYY |
| 2220 | LEKMADQAM |
| 2221 | LIVTTIVYL |
| 2222 | SIDAFKLNI |
| 2223 | DIADTTDAV |
| 2224 | SNFGAISSV |
| 2225 | YNMMISAGF |
| 2226 | ALNLGETFV |
| 2227 | NLGERVRQA |
| 2228 | QLSLPVLQV |
| 2229 | ILFLALITL |
| 2230 | QPRVEKKKL |
| 2231 | VPVSIINNT |
| 2232 | DFVKATCEF |
| 2233 | LEKCDLQNY |
| 2234 | CLTPVYSFL |
| 2235 | SLNVAKSEF |
| 2236 | ADIVVFDEI |
| 2237 | SNYQHEETI |
| 2238 | FSYVGCHNK |
| 2239 | EPEFYEAMY |
| 2240 | FVSGNCDVV |
| 2241 | TPVYSFLPG |
| 2242 | WLIVGVALL |
| 2243 | NVTQAFGRR |
| 2244 | KKLDGFMGR |
| 2245 | FEKMVSLLS |
| 2246 | GMPSYCTGY |
| 2247 | LEILDITPC |
| 2248 | VLKKCKSAF |
| 2249 | QEGVLTAVV |
| 2250 | LTLAVPYNM |
| 2251 | SEQLDFIDT |
| 2252 | ELSLIDFYL |
| 2253 | LSKGRLIIR |
| 2254 | GVKDCVVLH |
| 2255 | AGILIVTTI |
| 2256 | SEMVMCGGS |
| 2257 | WEVGKPRPP |
| 2258 | QITISSFKW |
| 2259 | YTSALLAGT |
| 2260 | FADDLNQLT |
| 2261 | TFVTHSKGL |
| 2262 | RAFDIYNDK |
| 2263 | EFLTRNPAW |
| 2264 | HLAKALNDF |
| 2265 | VVDADSKIV |
| 2266 | WKCRSKNPL |
| 2267 | SYGADLKSF |
| 2268 | MADSNGTIT |
| 2269 | AENVTGLFK |
| 2270 | EEVGHTDLM |
| 2271 | MLIIFWFSL |
| 2272 | CRMNSRNYI |
| 2273 | KQLSSNFGA |
| 2274 | LVGLMWLSY |
| 2275 | NEFACVVAD |
| 2276 | TLACFVLAA |
| 2277 | SVQTFFKLV |
| 2278 | DEPTTTTSV |
| 2279 | TSCCFSERF |
| 2280 | VSTQEFRYM |
| 2281 | VIPDYNTYK |
| 2282 | TMCDIRQLL |
| 2283 | RGWIFGTTL |
| 2284 | CQVHGNAHV |
| 2285 | KFVCDNIKF |
| 2286 | QEFKPRSQM |
| 2287 | QPSVGPKQA |
| 2288 | IISDMYDPK |
| 2289 | LALCADSII |
| 2290 | YPDKVFRSS |
| 2291 | NPDILRVYA |
| 2292 | FKEGSSVEL |
| 2293 | VIGFLFLTW |
| 2294 | VTLAILTAL |
| 2295 | MNVLTLVYK |
| 2296 | TSAMQTMLF |
| 2297 | GIYQTSNFR |
| 2298 | LLALHFLLF |
| 2299 | FSHSQLGGL |
| 2300 | GAWNIGEQK |
| 2301 | DATPSDFVR |
| 2302 | NRYLALYNK |
| 2303 | ASPNECNQM |
| 2304 | GRTILGSAL |
| 2305 | VLQKAAITI |
| 2306 | WTLMNVLTL |
| 2307 | GTTLPKGFY |
| 2308 | CTTIVNGVR |
| 2309 | LHSYFTSDY |
| 2310 | SLSSTASAL |
| 2311 | ELEGIQYGR |
| 2312 | LTSQWLTNI |
| 2313 | NVYLAVFDK |
| 2314 | YSHLLLVAA |
| 2315 | VVQEGVLTA |
| 2316 | PYRVVVLSF |
| 2317 | YELKHGTFT |
| 2318 | FVFLVLLPL |
| 2319 | TVQEIQLQA |
| 2320 | LYRKCVKSR |
| 2321 | SAGFSLWVY |
| 2322 | IIQFPNTYL |
| 2323 | EFSSNVANY |
| 2324 | SKSLTENKY |
| 2325 | AHFPREGVF |
| 2326 | FLPFQQFGR |
| 2327 | NSPRRARSV |
| 2328 | KGFCKLHNW |
| 2329 | SMQNCVLKL |
| 2330 | ELKKLLEQW |
| 2331 | VVTTFDSEY |
| 2332 | RKVQHMVVK |
| 2333 | RPNFTIKGS |
| 2334 | NASFDNFKF |
| 2335 | VTTIVYLTI |
| 2336 | NSRNYIAQV |
| 2337 | RRIRGGDGK |
| 2338 | IEVQGYKSV |
| 2339 | AVLDMCASL |
| 2340 | ADAQSFLNR |
| 2341 | FKLKDCVMY |
| 2342 | YYLGTGPEA |
| 2343 | CAFAVDAAK |
| 2344 | FTEQPIDLV |
| 2345 | CRFVTDTPK |
| 2346 | ITPCSFGGV |
| 2347 | VSLVKPSFY |
| 2348 | ALGGSVAIK |
| 2349 | CPRHVICTS |
| 2350 | STGYHFREL |
| 2351 | LYKMQRMLL |
| 2352 | ALYYPSARI |
| 2353 | TSEAVEAPL |
| 2354 | CIMSDRDLY |
| 2355 | AIVVTCLAY |
| 2356 | VQPQLEMEL |
| 2357 | RYVLMDGSI |
| 2358 | EWFLAYILF |
| 2359 | VLITEGSVK |
| 2360 | TTCFSVAAL |
| 2361 | GANKDGIIW |
| 2362 | SSQGSEYDY |
| 2363 | MLWCKDGHV |
| 2364 | EVVLKTGDL |
| 2365 | SLRVCVDTV |
| 2366 | CFVLAAVYR |
| 2367 | VRGTTVLLK |
| 2368 | VQHMVVKAA |
| 2369 | IPARARVEC |
| 2370 | VLLRKNGNK |
| 2371 | FASFYYVWK |
| 2372 | GDFLHFLPR |
| 2373 | SICSTMTNR |
| 2374 | TGVEHVTFF |
| 2375 | YIIKLIFLW |
| 2376 | FGAGAALQI |
| 2377 | EAACCHLAK |
| 2378 | LPVLQVRDV |
| 2379 | LSLQFKRPI |
| 2380 | NPTDQSSYI |
| 2381 | KGDYGDAVV |
| 2382 | CSQHTMLVK |
| 2383 | WADNNCYLA |
| 2384 | QFAYANRNR |
| 2385 | LRANSAVKL |
| 2386 | ATNNLVVMA |
| 2387 | LFLPFFSNV |
| 2388 | ETKFLTENL |
| 2389 | TYVTQQLIR |
| 2390 | RLNQLESKM |
| 2391 | ASSSEAFLI |
| 2392 | VLSFELLHA |
| 2393 | SVAYSNNSI |
| 2394 | SLREVRTIK |
| 2395 | ALILAYCNK |
| 2396 | HVMSKHTDF |
| 2397 | GGDAALALL |
| 2398 | YQTQTNSPR |
| 2399 | VIAWNSNNL |
| 2400 | YYRRATRRI |
| 2401 | VLKKLKKSL |
| 2402 | IDHPNPKGF |
| 2403 | LSLREVRTI |
| 2404 | SVTSNYSGV |
| 2405 | SSSSDNIAL |
| 2406 | FCSQHTMLV |
| 2407 | FLGIITTVA |
| 2408 | VVNPVMEPI |
| 2409 | GACIRRPFL |
| 2410 | FLGYFCTCY |
| 2411 | TITVNVLAW |
| 2412 | MRSLKVPAT |
| 2413 | LKLFAAETL |
| 2414 | GVVTTVMFL |
| 2415 | TANKWDLII |
| 2416 | LPDDFTGCV |
| 2417 | KENDSKEGF |
| 2418 | LPFFYYSDS |
| 2419 | KLDGFMGRI |
| 2420 | KACPLIAAV |
| 2421 | LRKVPTDNY |
| 2422 | TRPLLESEL |
| 2423 | ISMATNYDL |
| 2424 | TAGAAAYYV |
| 2425 | LALYNKYKY |
| 2426 | LLQFAYANR |
| 2427 | RILGAGCFV |
| 2428 | KQLPFFYYS |
| 2429 | ELTSMKYFV |
| 2430 | SSRLSFKEL |
| 2431 | LDYIINLII |
| 2432 | TGPEAGLPY |
| 2433 | GDSEVVLKK |
| 2434 | YVRNLQHRL |
| 2435 | SREETGLLM |
| 2436 | MLAHAEETR |
| 2437 | KEGFFTYIC |
| 2438 | QQESPFVMM |
| 2439 | KITEHSWNA |
| 2440 | TPFEIKLAK |
| 2441 | DPSFLGRYM |
| 2442 | LLFLAFVVF |
| 2443 | ITREVGFVV |
| 2444 | GLCVDIPGI |
| 2445 | TQDLFLPFF |
| 2446 | FVTHSKGLY |
| 2447 | WVMRIMTWL |
| 2448 | RFLYIIKLI |
| 2449 | FNVLFSTVF |
| 2450 | SEAARVVRS |
| 2451 | ARGIVFMCV |
| 2452 | LNVPLHGTI |
| 2453 | FYLCFLAFL |
| 2454 | NLVAMKYNY |
| 2455 | VGFTLKNTV |
| 2456 | CTSVVLLSV |
| 2457 | TSAFVETVK |
| 2458 | VVHNQDVNL |
| 2459 | LPVSMTKTS |
| 2460 | TEHSWNADL |
| 2461 | CISTKHFYW |
| 2462 | LLAKDTTEA |
| 2463 | SLCLQLAVV |
| 2464 | IVNNWLKQL |
| 2465 | HVASCDAIM |
| 2466 | VTSAMQTML |
| 2467 | TVLSFCAFA |
| 2468 | NSVLLFLAF |
| 2469 | IYSLLLCRM |
| 2470 | NVNRFNVAI |
| 2471 | ALAPNMMVT |
| 2472 | FSLWVYKQF |
| 2473 | NARDGCVPL |
| 2474 | FLELAMDEF |
| 2475 | ITSGWTFGA |
| 2476 | ITVNVLAWL |
| 2477 | VLLSMQGAV |
| 2478 | LTLGVYDYL |
| 2479 | RTCCLCDRR |
| 2480 | YPQVNGLTS |
| 2481 | RVDFCGKGY |
| 2482 | TTLPKGFYA |
| 2483 | FVENPDILR |
| 2484 | TETAHSCNV |
| 2485 | LVTMPLGYV |
| 2486 | FSTGVNLVA |
| 2487 | GCDGGSLYV |
| 2488 | RENNRVVIS |
| 2489 | QPYRVVVLS |
| 2490 | TVVVNAANV |
| 2491 | VLLFLAFVV |
| 2492 | VYEKLKPVL |
| 2493 | FPNITNLCP |
| 2494 | DGYVMHANY |
| 2495 | QILFALLQR |
| 2496 | KSDGTGTIY |
| 2497 | SSRGTSPAR |
| 2498 | NVAKSEFDR |
| 2499 | FTSLEIPRR |
| 2500 | CLEASFNYL |
| 2501 | HGFELTSMK |
| 2502 | MIDVQQWGF |
| 2503 | CRKVQHMVV |
| 2504 | YFLQSINFV |
| 2505 | MMPTIFFAG |
| 2506 | NPYVCNAPG |
| 2507 | MHANYIFWR |
| 2508 | ESPFVMMSA |
| 2509 | LMWLIINLV |
| 2510 | GLWLDDVVY |
| 2511 | CYGVSPTKL |
| 2512 | YQCAMRPNF |
| 2513 | HFVCNLLLL |
| 2514 | VQMLSDTLK |
| 2515 | FFSNVTWFH |
| 2516 | TFLLNKEMY |
| 2517 | ARHINAQVA |
| 2518 | RIDKVLNEK |
| 2519 | SIVCRFDTR |
| 2520 | SLLSKGRLI |
| 2521 | FMCVEYCPI |
| 2522 | EYRLYLDAY |
| 2523 | SVTTEILPV |
| 2524 | KTLNSLEDK |
| 2525 | VFKNIDGYF |
| 2526 | TGNYQCGHY |
| 2527 | GTILTRPLL |
| 2528 | FAMGIIAMS |
| 2529 | VESCGNFKV |
| 2530 | VRTNVYLAV |
| 2531 | YEDQDALFA |
| 2532 | TNDKACPLI |
| 2533 | NFKDQVILL |
| 2534 | HPLADNKFA |
| 2535 | DYVYNPFMI |
| 2536 | FLMSFTVLC |
| 2537 | ELTGHMLDM |
| 2538 | KHYTPSFKK |
| 2539 | FHPLADNKF |
| 2540 | DYDYYRYNL |
| 2541 | ELYSPIFLI |
| 2542 | RNLQEFKPR |
| 2543 | DIGNPKAIK |
| 2544 | VAKHDFFKF |
| 2545 | LVDPQIQLA |
| 2546 | CWHTNCYDY |
| 2547 | DRYPANSIV |
| 2548 | RQRLTKYTM |
| 2549 | LFTRFFYVL |
| 2550 | NFTIKGSFL |
| 2551 | NRVCGVSAA |
| 2552 | VFQTRAGCL |
| 2553 | NVAITRAKV |
| 2554 | IDFYLCFLA |
| 2555 | AYIICISTK |
| 2556 | YVQIPTTCA |
| 2557 | GFIQQKLAL |
| 2558 | KSILSPLYA |
| 2559 | FCLLNRYFR |
| 2560 | LALHFLLFF |
| 2561 | CLAVHECFV |
| 2562 | AMAVMLLLL |
| 2563 | ALKYLPIDK |
| 2564 | DELKINAAC |
| 2565 | GVVQLTSQW |
| 2566 | DDTLRVEAF |
| 2567 | RFTTTLNDF |
| 2568 | RNPANNAAI |
| 2569 | SWNADLYKL |
| 2570 | TASWFTALT |
| 2571 | LSWEVGKPR |
| 2572 | SSGDATTAY |
| 2573 | LFWNCNVDR |
| 2574 | RKMAFPSGK |
| 2575 | RLTLGVYDY |
| 2576 | LIVAAIVFI |
| 2577 | DVEGCHATR |
| 2578 | VPLNIIPLT |
| 2579 | HFLPRVFSA |
| 2580 | WPQIAQFAP |
| 2581 | MGHFAWWTA |
| 2582 | VQSTQWSLF |
| 2583 | RLCAYCCNI |
| 2584 | DVNCTEVPV |
| 2585 | LTVFFDGRV |
| 2586 | KLHDELTGH |
| 2587 | NTLQCIMLV |
| 2588 | VDDPCPIHF |
| 2589 | IIWFLLLSV |
| 2590 | WESGVKDCV |
| 2591 | AELEGIQYG |
| 2592 | TPKDHIGTR |
| 2593 | LATVAYFNM |
| 2594 | MRFRRAFGE |
| 2595 | LTRNPAWRK |
| 2596 | KEIKESVQT |
| 2597 | LFARTRSMW |
| 2598 | LRKQIRSAA |
| 2599 | MPLGYVTHG |
| 2600 | MELTPVVQT |
| 2601 | VAGFAKFLK |
| 2602 | TYLEGSVRV |
| 2603 | ASEAARVVR |
| 2604 | ITGGIAIAM |
| 2605 | DLDDFSKQL |
| 2606 | PFFSNVTWF |
| 2607 | NVYADSFVI |
| 2608 | NQPYPNASF |
| 2609 | HISRQRLTK |
| 2610 | RRATCFSTA |
| 2611 | FVVFLLVTL |
| 2612 | LAMDEFIER |
| 2613 | KPFERDIST |
| 2614 | TPLVPFWIT |
| 2615 | ILAYCNKTV |
| 2616 | YQCGHYKHI |
| 2617 | FAWWTAFVT |
| 2618 | IWLGFIAGL |
| 2619 | GGNYNYLYR |
| 2620 | FISTCACEI |
| 2621 | FFSNYLKRR |
| 2622 | EVVLKKLKK |
| 2623 | QLGGLHLLI |
| 2624 | LKVGGSCVL |
| 2625 | RANNTKGSL |
| 2626 | KYCALAPNM |
| 2627 | SLVVRCSFY |
| 2628 | VRMYIFFAS |
| 2629 | HVICTSEDM |
| 2630 | RKHFSMMIL |
| 2631 | KESVQTFFK |
| 2632 | SPLSLNMAR |
| 2633 | PQADVEWKF |
| 2634 | VQQLPETYF |
| 2635 | TEILPVSMT |
| 2636 | NFVFPLNSI |
| 2637 | YSYATHSDK |
| 2638 | MLNPNYEDL |
| 2639 | AFGEYSHVV |
| 2640 | FATSACVLA |
| 2641 | MAYCWRCTS |
| 2642 | GFELTSMKY |
| 2643 | QKKQQTVTL |
| 2644 | NKYKYFSGA |
| 2645 | FAQVKQIYK |
| 2646 | HFAWWTAFV |
| 2647 | NRKRISNCV |
| 2648 | SSQAWQPGV |
| 2649 | SQCVNLTTR |
| 2650 | RFQNHNPQK |
| 2651 | AENSVAYSN |
| 2652 | FLPFFSNVT |
| 2653 | HPALRLVDP |
| 2654 | FNEKTHVQL |
| 2655 | LVKQLSSNF |
| 2656 | FLKRGDKSV |
| 2657 | NLNESLIDL |
| 2658 | QLHNDILLA |
| 2659 | GYLQPRTFL |
| 2660 | NASSSEAFL |
| 2661 | FITESKPSV |
| 2662 | RELGVVHNQ |
| 2663 | TFTRSTNSR |
| 2664 | MFITREEAI |
| 2665 | DAVRDPQTL |
| 2666 | ATATIPIQA |
| 2667 | SDRELHLSW |
| 2668 | EWSMATYYL |
| 2669 | VGPKVYPII |
| 2670 | ETGLLMPLK |
| 2671 | IINLIIKNL |
| 2672 | SINFVRIIM |
| 2673 | RPPLNRNYV |
| 2674 | DGISQYSLR |
| 2675 | TFISDEVAR |
| 2676 | RRAFGEYSH |
| 2677 | TTEILPVSM |
| 2678 | RQGFVDSDV |
| 2679 | MLVKQGDDY |
| 2680 | MPNLYKMQR |
| 2681 | HHANEYRLY |
| 2682 | VAPGTAVLR |
| 2683 | FELWAKRNI |
| 2684 | SRIKASMPT |
| 2685 | GEIKDATPS |
| 2686 | DKRAKVTSA |
| 2687 | TNSFTRGVY |
| 2688 | KWPWYIWLG |
| 2689 | TCFSTQFAF |
| 2690 | YLGKPREQI |
| 2691 | SEDNQTTTI |
| 2692 | LRARSVSPK |
| 2693 | CEFQFCNDP |
| 2694 | IGFLFLTWI |
| 2695 | KSPIQYIDI |
| 2696 | YGDSATLPK |
| 2697 | AFVETVKGL |
| 2698 | TPTWRVYST |
| 2699 | CGYLPQNAV |
| 2700 | EAPLVGTPV |
| 2701 | KAALLADKF |
| 2702 | LRKLDNDAL |
| 2703 | VSALVYDNK |
| 2704 | ATNYDLSVV |
| 2705 | RTLLTKGTL |
| 2706 | YPKCDRAMP |
| 2707 | NYVFTGYRV |
| 2708 | VDTVRTNVY |
| 2709 | NGDSEVVLK |
| 2710 | YLYLTFYLT |
| 2711 | IVNNATNVV |
| 2712 | SAPLIELCV |
| 2713 | NVGPKVYPI |
| 2714 | TGYKKPASR |
| 2715 | SVGPKQASL |
| 2716 | SQDLSVVSK |
| 2717 | SLINTLNDL |
| 2718 | NLREMLAHA |
| 2719 | QYGSFCTQL |
| 2720 | EMLDNRATL |
| 2721 | CPLIAAVIT |
| 2722 | FMSLSEQLR |
| 2723 | TIFKDASGK |
| 2724 | DALCEKALK |
| 2725 | APHGHVMVE |
| 2726 | LPKGFYAEG |
| 2727 | LKQLIKVTL |
| 2728 | TQALPQRQK |
| 2729 | DFLEYHDVR |
| 2730 | ISEMHPALR |
| 2731 | LRVESSSKL |
| 2732 | DHIGTRNPA |
| 2733 | VTTVMFLAR |
| 2734 | TWLTYTGAI |
| 2735 | VMFLARGIV |
| 2736 | TPFDVVRQC |
| 2737 | ILASFSAST |
| 2738 | LGFSTGVNL |
| 2739 | FVCNLLLLF |
| 2740 | TNILLNVPL |
| 2741 | RDAAMQRKL |
| 2742 | NRARTVAGV |
| 2743 | QESFGGASC |
| 2744 | YDKLQFTSL |
| 2745 | YYKKVDGVV |
| 2746 | LTQDHVDIL |
| 2747 | YICGFIQQK |
| 2748 | AQTGSSKCV |
| 2749 | MAPISAMVR |
| 2750 | NPANNAAIV |
| 2751 | ALQDAYYRA |
| 2752 | FAAETLKAT |
| 2753 | TLQPVSELL |
| 2754 | LACFVLAAV |
| 2755 | WNVKDFMSL |
| 2756 | LLEWLAMAV |
| 2757 | IFGTVYEKL |
| 2758 | YDDGARRVW |
| 2759 | QPTSEAVEA |
| 2760 | WAKRNIKPV |
| 2761 | IRASANLAA |
| 2762 | SERFQNHNP |
| 2763 | VSKVVKVTI |
| 2764 | TVCTVCGMW |
| 2765 | SSKCVCSVI |
| 2766 | FEKGDYGDA |
| 2767 | MVRMYIFFA |
| 2768 | VYANLGERV |
| 2769 | FCDLKGKYV |
| 2770 | VSIWNLDYI |
| 2771 | FLIVAAIVF |
| 2772 | NYLYRLFRK |
| 2773 | FVDRQTAQA |
| 2774 | TAFVTNVNA |
| 2775 | KLKTLVATA |
| 2776 | NNCYLATAL |
| 2777 | QQTVTLLPA |
| 2778 | TNNVAFQTV |
| 2779 | IAYTMSLGA |
| 2780 | KEMYLKLRS |
| 2781 | SWMESEFRV |
| 2782 | AKHDFFKFR |
| 2783 | CKDGHVETF |
| 2784 | KEGQINDMI |
| 2785 | MVLGSLAAT |
| 2786 | LLSVCLGSL |
| 2787 | SCDQLREPM |
| 2788 | VAGGIVAIV |
| 2789 | QWLPTGTLL |
| 2790 | GRLQSLQTY |
| 2791 | SAVVLLILM |
| 2792 | SEKSYELQT |
| 2793 | AMKYNYEPL |
| 2794 | TLKNLSDRV |
| 2795 | DFSSEIIGY |
| 2796 | DPAQLPAPR |
| 2797 | NKDFYDFAV |
| 2798 | YWEPEFYEA |
| 2799 | QLNRALTGI |
| 2800 | LLKDCPAVA |
| 2801 | GSSGVVNPV |
| 2802 | KRSFIEDLL |
| 2803 | AYRKVLLRK |
| 2804 | CEKALKYLP |
| 2805 | VAKNLNESL |
| 2806 | FLALITLAT |
| 2807 | AVFQSASKI |
| 2808 | KVEGCMVQV |
| 2809 | CVDTVRTNV |
| 2810 | HAFLCLFLL |
| 2811 | EDQDALFAY |
| 2812 | QIVESCGNF |
| 2813 | HSQLGGLHL |
| 2814 | GALDISASI |
| 2815 | LYDANYFLC |
| 2816 | AAMQRKLEK |
| 2817 | VFIKRSDAR |
| 2818 | GLALYYPSA |
| 2819 | LQPRTFLLK |
| 2820 | NGMNGRTIL |
| 2821 | GRFVLALLS |
| 2822 | KMFDAYVNT |
| 2823 | ATRGATVVI |
| 2824 | FGGCVFSYV |
| 2825 | VRDVLVRGF |
| 2826 | AGTDTTITV |
| 2827 | KGTHHWLLL |
| 2828 | VDWTIEYPI |
| 2829 | AMACLVGLM |
| 2830 | GVTLIGEAV |
| 2831 | AQEAYEQAV |
| 2832 | GDCLGDIAA |
| 2833 | GSKSPIQYI |
| 2834 | SGLKTILRK |
| 2835 | QLTPIAVQM |
| 2836 | DMSKFPLKL |
| 2837 | VYDNKLKAH |
| 2838 | VRVLQKAAI |
| 2839 | PFAMQMAYR |
| 2840 | SAYENFNQH |
| 2841 | NLQSNHDLY |
| 2842 | FYSKWYIRV |
| 2843 | GLVEVEKGV |
| 2844 | NIVTRCLNR |
| 2845 | CEEMLDNRA |
| 2846 | AAITILDGI |
| 2847 | LLSVLQQLR |
| 2848 | GVLTESNKK |
| 2849 | DTTITVNVL |
| 2850 | FLFLTWICL |
| 2851 | MPTTIAKNT |
| 2852 | NIVNVSLVK |
| 2853 | ISPYNSQNA |
| 2854 | GYQPYRVVV |
| 2855 | YMRSLKVPA |
| 2856 | VATVQEIQL |
| 2857 | ICAPLTVFF |
| 2858 | NQFNSAIGK |
| 2859 | NLLLLFVTV |
| 2860 | NFVRIIMRL |
| 2861 | KPLEFGATS |
| 2862 | MQNCVLKLK |
| 2863 | FFKLVNKFL |
| 2864 | LVQAGNVQL |
| 2865 | FTEERLKLF |
| 2866 | VFTGYRVTK |
| 2867 | CFSTASDTY |
| 2868 | VLPQLEQPY |
| 2869 | MAFPSGKVE |
| 2870 | HTANKWDLI |
| 2871 | LANTCTERL |
| 2872 | DVNLHSSRL |
| 2873 | KKLLEQWNL |
| 2874 | LELAMDEFI |
| 2875 | TDTPKGPKV |
| 2876 | SGAMDTTSY |
| 2877 | FWNCNVDRY |
| 2878 | AVANGDSEV |
| 2879 | DVVYCPRHV |
| 2880 | SDVENPHLM |
| 2881 | SEEVVENPT |
| 2882 | AQEKNFTTA |
| 2883 | YKLNVGDYF |
| 2884 | YNGSPSGVY |
| 2885 | AMMFVKHKH |
| 2886 | NRPQIGVVR |
| 2887 | CDQLREPML |
| 2888 | SEHDYQIGG |
| 2889 | ITVEELKKL |
| 2890 | FKELLVYAA |
| 2891 | IPKDMTYRR |
| 2892 | DIVKTDGTL |
| 2893 | TLLSLREVR |
| 2894 | NWYDFGDFI |
| 2895 | LVIGFLFLT |
| 2896 | RDAPAHIST |
| 2897 | LGYFCTCYF |
| 2898 | LLLCRMNSR |
| 2899 | FNYLKSPNF |
| 2900 | TREAVGTNL |
| 2901 | TICAPLTVF |
| 2902 | SATLPKGIM |
| 2903 | VVTCLAYYF |
| 2904 | VFHLYLQYI |
| 2905 | DEDDSEPVL |
| 2906 | KKADETQAL |
| 2907 | APGQTGKIA |
| 2908 | HRLYECLYR |
| 2909 | FPKSDGTGT |
| 2910 | TETDLTKGP |
| 2911 | PPISFPLCA |
| 2912 | LTENKYSQL |
| 2913 | RSGETLGVL |
| 2914 | QPYVVDDPC |
| 2915 | EKTHVQLSL |
| 2916 | QKLLKSIAA |
| 2917 | SSRSSSRSR |
| 2918 | QAAVGELLL |
| 2919 | VTDFNAIAT |
| 2920 | KSREETGLL |
| 2921 | RDLSLQFKR |
| 2922 | EHSWNADLY |
| 2923 | EAPFLYLYA |
| 2924 | DAFKLNIKL |
| 2925 | TYKLNVGDY |
| 2926 | VGARKSAPL |
| 2927 | KRRVVFNGV |
| 2928 | TSVVLLSVL |
| 2929 | VFQSASKII |
| 2930 | CANGQVFGL |
| 2931 | KAGQKTYER |
| 2932 | HLLIGLAKR |
| 2933 | EIDFLELAM |
| 2934 | FYEAMYTPH |
| 2935 | ITIAYIICI |
| 2936 | STGVNLVAV |
| 2937 | RALTAESHV |
| 2938 | AVVKIYCPA |
| 2939 | YYNTTKGGR |
| 2940 | TACTDDNAL |
| 2941 | YVLMDGSII |
| 2942 | QVDLFRNAR |
| 2943 | VECFDKFKV |
| 2944 | LPWNVVRIK |
| 2945 | ECAQVLSEM |
| 2946 | HPNQEYADV |
| 2947 | DFYLCFLAF |
| 2948 | TIFFAGILI |
| 2949 | VTLIGEAVK |
| 2950 | ALTCFSTQF |
| 2951 | CIRCLWSTK |
| 2952 | NTLLFLMSF |
| 2953 | LIRKSNHNF |
| 2954 | IVNGVRRSF |
| 2955 | NVSLVKPSF |
| 2956 | LLEDEFTPF |
| 2957 | VFMSEAKCW |
| 2958 | IVESCGNFK |
| 2959 | KPTETICAP |
| 2960 | FYEDFLEYH |
| 2961 | TTEELPDEF |
| 2962 | KQIRSAAKK |
| 2963 | DMILSLLSK |
| 2964 | MLLLLCCCL |
| 2965 | NLYKMQRML |
| 2966 | GPLVRKIFV |
| 2967 | SFGGASCCL |
| 2968 | CHNKCAYWV |
| 2969 | NSVFNICQA |
| 2970 | VANGDSEVV |
| 2971 | QLLFVVEVV |
| 2972 | ALRLVDPQI |
| 2973 | ITEGSVKGL |
| 2974 | GFFTYICGF |
| 2975 | CSHAAVDAL |
| 2976 | DKRTTCFSV |
| 2977 | SGKPVPYCY |
| 2978 | FCGPDGYPL |
| 2979 | SVEEVLSEA |
| 2980 | GIMMNVAKY |
| 2981 | EELPDEFVV |
| 2982 | GYVMHANYI |
| 2983 | HLSVDTKFK |
| 2984 | STIGVCSMT |
| 2985 | VLTLVYKVY |
| 2986 | LLVPHHVVA |
| 2987 | DYDCVSFCY |
| 2988 | SALVYDNKL |
| 2989 | HVETFYPKL |
| 2990 | NRYFRLTLG |
| 2991 | YHNESGLKT |
| 2992 | YRINWITGG |
| 2993 | FMRIFTIGT |
| 2994 | NLVIGFLFL |
| 2995 | NIKPVPEVK |
| 2996 | AMMFTSDLA |
| 2997 | VAIVVTCLA |
| 2998 | KVITGLHPT |
| 2999 | YTVSCLPFT |
| 3000 | SKQRRPQGL |
| 3001 | EVTPSGTWL |
| 3002 | FFYYSDSPC |
| 3003 | VYAWNRKRI |
| 3004 | LEPLVDLPI |
| 3005 | GTPVCINGL |
| 3006 | LLKSAYENF |
| 3007 | ROMSCAAGT |
| 3008 | ENAFLPFAM |
| 3009 | SLRCGACIR |
| 3010 | DYTEISFML |
| 3011 | KVTSAMQTM |
| 3012 | RKHTTCCSL |
| 3013 | EVAKNLNES |
| 3014 | VQELYSPIF |
| 3015 | LLFLVLIML |
| 3016 | LPQGTTLPK |
| 3017 | TEVLTEEVV |
| 3018 | IWVATEGAL |
| 3019 | LLQILFALL |
| 3020 | LLVAAGLEA |
| 3021 | YNVTQAFGR |
| 3022 | ILANTCTER |
| 3023 | LECIKDLLA |
| 3024 | LVRGFGDSV |
| 3025 | VLTEEVVLK |
| 3026 | LKFPRGQGV |
| 3027 | DGVKHVYQL |
| 3028 | LPGTILRTT |
| 3029 | TIQPRVEKK |
| 3030 | FSNVTWFHA |
| 3031 | QASSRSSSR |
| 3032 | RRSFYVYAN |
| 3033 | QLRVIGHSM |
| 3034 | EMHPALRLV |
| 3035 | LSSYSLFDM |
| 3036 | DKCSRIIPA |
| 3037 | APRITFGGP |
| 3038 | TSTLQGCSL |
| 3039 | NLLLQYGSF |
| 3040 | TRMENAVGR |
| 3041 | PPQTSITSA |
| 3042 | YRFNGIGVT |
| 3043 | LLLLFVTVY |
| 3044 | APLVGTPVC |
| 3045 | FGLFCLLNR |
| 3046 | DAAKAYKDY |
| 3047 | AAKAYKDYL |
| 3048 | NPIQLSSYS |
| 3049 | CDHCGETSW |
| 3050 | NGVGYQPYR |
| 3051 | NWITGGIAI |
| 3052 | RVVFVLWAH |
| 3053 | TALRANSAV |
| 3054 | HLLLVAAGL |
| 3055 | YLGGMSYYC |
| 3056 | FPSGKVEGC |
| 3057 | IPCTCGKQA |
| 3058 | ARLRAKHYV |
| 3059 | TKLATTEEL |
| 3060 | CVCSVIDLL |
| 3061 | LRTTNGDFL |
| 3062 | NINIVGDFK |
| 3063 | LKLRGTAVM |
| 3064 | MMFTSDLAT |
| 3065 | AAVYRINWI |
| 3066 | YAISAKNRA |
| 3067 | FGGASCCLY |
| 3068 | FITLCFTLK |
| 3069 | LEGYAFEHI |
| 3070 | IIFLEGETL |
| 3071 | MSFTVLCLT |
| 3072 | RAKVGILCI |
| 3073 | GSVGFNIDY |
| 3074 | ILLIIMRTF |
| 3075 | FDTRVLSNL |
| 3076 | LKLRSDVLL |
| 3077 | IILRLGSPL |
| 3078 | EGYAFEHIV |
| 3079 | SDRDLYDKL |
| 3080 | QYGRSGETL |
| 3081 | LEGSVRVVT |
| 3082 | MDTTSYREA |
| 3083 | YRVTKNSKV |
| 3084 | GASCCLYCR |
| 3085 | LDISASIVA |
| 3086 | CESHGKQVV |
| 3087 | LNDNLLEIL |
| 3088 | FGPLVRKIF |
| 3089 | IQWMVMFTP |
| 3090 | KEPCSSGTY |
| 3091 | ARSVSPKLF |
| 3092 | VIDLLLDDF |
| 3093 | PHGHVMVEL |
| 3094 | YPDPSRILG |
| 3095 | TQLSTDTGV |
| 3096 | LIRQGTDYK |
| 3097 | NSYECDIPI |
| 3098 | VGLMWLSYF |
| 3099 | CQEPKLGSL |
| 3100 | NSKVQIGEY |
| 3101 | LSMQGAVDI |
| 3102 | HQKLLKSIA |
| 3103 | LTWICLLQF |
| 3104 | PHSLSDGLL |
| 3105 | YGNALDQAI |
| 3106 | ASKKPRQKR |
| 3107 | LYECLYRNR |
| 3108 | EGFNCYFPL |
| 3109 | ERLKLFDRY |
| 3110 | SSGVVNPVM |
| 3111 | LQDLKWARF |
| 3112 | RNSTPGSSR |
| 3113 | SHMYCSFYP |
| 3114 | CSLCLQLAV |
| 3115 | SEDKRAKVT |
| 3116 | PPTSFGPLV |
| 3117 | KGLDYKAFK |
| 3118 | TQKGAEAAV |
| 3119 | IERYKLEGY |
| 3120 | ILRTTNGDF |
| 3121 | AVTANVNAL |
| 3122 | GFCDLKGKY |
| 3123 | GDYGDAVVY |
| 3124 | DAIMTRCLA |
| 3125 | LSLPVLQVR |
| 3126 | GDAALALLL |
| 3127 | TRNPANNAA |
| 3128 | GVGGKPCIK |
| 3129 | LPPKNSIDA |
| 3130 | LVQSTQWSL |
| 3131 | YEGNSPFHP |
| 3132 | WDYKRDAP |
| 3133 | IVVFDEISM |
| 3134 | SPVALRQMS |
| 3135 | QQQGQTVT |
| 3136 | LNNIINNAR |
| 3137 | QEPKLGSLV |
| 3138 | VPLHGTILT |
| 3139 | FLTRNPAWR |
| 3140 | KGVAPGTAV |
| 3141 | QLRVESSSK |
| 3142 | QEHYVRITG |
| 3143 | YMHHMELPT |
| 3144 | SEGLNDNLL |
| 3145 | AQSFLNGFA |
| 3146 | VCVSTSGRW |
| 3147 | RTVYDDGAR |
| 3148 | SSKLWAQCV |
| 3149 | TFNGECPNF |
| 3150 | VEGCMVQVT |
| 3151 | KVTKGKAK |
| 3152 | YKLMGHFAW |
| 3153 | DAALALLLL |
| 3154 | NSVTSSIVI |
| 3155 | LVFLFVAAI |
| 3156 | VPTDNYITT |
| 3157 | DTGVEHVTF |
| 3158 | AAFATAQEA |
| 3159 | TPSKLIEYT |
| 3160 | NVTDFNAIA |
| 3161 | LAVFQSASK |
| 3162 | CERSEAGVC |
| 3163 | IAEILLIIM |
| 3164 | LSVVNARLR |
| 3165 | LEDKAFQLT |
| 3166 | LLLLEWLAM |
| 3167 | KYVQIPTTC |
| 3168 | LITLATCEL |
| 3169 | KNIDGYFKI |
| 3170 | RKSNHNFLV |
| 3171 | FGWLIVGVA |
| 3172 | SKIVQLSEI |
| 3173 | SQGSEYDYV |
| 3174 | KRVLNVVCK |
| 3175 | TEAFEKMVS |
| 3176 | LGSPLSLNM |
| 3177 | KQVVSDIDY |
| 3178 | ITVTPEANM |
| 3179 | DKFTDGVCL |
| 3180 | GIVFMCVEY |
| 3181 | IIKNLSKSL |
| 3182 | HVQLSLPVL |
| 3183 | TEELPDEFV |
| 3184 | YVFTGYRVT |
| 3185 | AIVFITLCF |
| 3186 | GRSGETLGV |
| 3187 | LTGHMLDMY |
| 3188 | TLIVNSVLL |
| 3189 | VILRGHLRI |
| 3190 | SSAINRPQI |
| 3191 | RGTAVMSLK |
| 3192 | VLQVRDVLV |
| 3193 | KSTNLVKNK |
| 3194 | SKEGFFTYI |
| 3195 | VGEIPVAYR |
| 3196 | AQPCSDKAY |
| 3197 | GTGTIYTEL |
| 3198 | ELVAELEGI |
| 3199 | TLCFTLKRK |
| 3200 | PLMYKGLPW |
| 3201 | PVNVAFELW |
| 3202 | KLKKSLNVA |
| 3203 | LELQDHNET |
| 3204 | KHKHAFLCL |
| 3205 | PFTIYSLLL |
| 3206 | QTSITSAVL |
| 3207 | KFDTFNGEC |
| 3208 | CWRCTSCCF |
| 3209 | SVLYYQNNV |
| 3210 | LMKTIGPDM |
| 3211 | LPTQTVDSS |
| 3212 | DTSLSGFKL |
| 3213 | FDKSAFVNL |
| 3214 | KGPKVKYLY |
| 3215 | YKFVRIQPG |
| 3216 | TETICAPLT |
| 3217 | LVPHHVVAT |
| 3218 | YFNMVYMPA |
| 3219 | IGDPAQLPA |
| 3220 | YTEKWESGV |
| 3221 | GMVLGSLAA |
| 3222 | RSEDKRAKV |
| 3223 | NAQALNTLV |
| 3224 | YNKYKYFSG |
| 3225 | TPCGTGTST |
| 3226 | CAAGTTQTA |
| 3227 | VYTACSHAA |
| 3228 | IFFAGILIV |
| 3229 | VPANSTVLS |
| 3230 | LRLIDAMMF |
| 3231 | HTNCYDYCI |
| 3232 | QPGVAMPNL |
| 3233 | VAYSNNSIA |
| 3234 | RSGARSKQR |
| 3235 | ILRKGGRTI |
| 3236 | CELYHYQEC |
| 3237 | IQPIGALDI |
| 3238 | RVECFDKFK |
| 3239 | LADNKFALT |
| 3240 | SNIIRGWIF |
| 3241 | APRTLLTKG |
| 3242 | ASIVAGGIV |
| 3243 | VVTTVMFLA |
| 3244 | FLKEQHCQK |
| 3245 | GVCVSTSGR |
| 3246 | ESELVIGAV |
| 3247 | NPKAIKCVP |
| 3248 | ELKFNPPAL |
| 3249 | GHHLGRCDI |
| 3250 | YFCTCYFGL |
| 3251 | GSLVVRCSF |
| 3252 | QAGNVQLRV |
| 3253 | GKSHFAIGL |
| 3254 | ACPDGVKHV |
| 3255 | NKDGIIWVA |
| 3256 | CAQVLSEMV |
| 3257 | ALLSTDGNK |
| 3258 | VLLILMTAR |
| 3259 | DEAGSKSPI |
| 3260 | GPLSAQTGI |
| 3261 | FLLFLVLIM |
| 3262 | KCSRIIPAR |
| 3263 | WYIRVGARK |
| 3264 | FKLNEEIAI |
| 3265 | VFLLVTLAI |
| 3266 | GLHLLIGLA |
| 3267 | LYHYQECVR |
| 3268 | DAYKTFPPT |
| 3269 | FVRIIMRLW |
| 3270 | MVVKAALLA |
| 3271 | FLWLLWPVT |
| 3272 | ILRGHLRIA |
| 3273 | YEDLLIRKS |
| 3274 | VVRIKIVQM |
| 3275 | ELFYSYATH |
| 3276 | DVVRQCSGV |
| 3277 | DRRATCFST |
| 3278 | NSQNAVASK |
| 3279 | DTNVLEGSV |
| 3280 | DVKCTSVVL |
| 3281 | IVQMLSDTL |
| 3282 | PFMIDVQQW |
| 3283 | NGVRRSFYV |
| 3284 | MQGAVDINK |
| 3285 | GPEQTQGNF |
| 3286 | NLIIKNLSK |
| 3287 | SFYVYSRVK |
| 3288 | LLIGLAKRF |
| 3289 | SHAAVDALC |
| 3290 | IKDFGGFNF |
| 3291 | SLNMARKTL |
| 3292 | IKGTHHWLL |
| 3293 | VRITGLYPT |
| 3294 | NKSWMESEF |
| 3295 | RAGKASCTL |
| 3296 | LGIITTVAA |
| 3297 | YANSVFNIC |
| 3298 | AYCCNIVNV |
| 3299 | VPINTNSSP |
| 3300 | HKDKSAQCF |
| 3301 | FYYLGTGPE |
| 3302 | KCDRAMPNM |
| 3303 | LLEQWNLVI |
| 3304 | SFGPLVRKI |
| 3305 | TGLYPTLNI |
| 3306 | TVCGMWKGY |
| 3307 | LLQNGMNGR |
| 3308 | TEIYQAGST |
| 3309 | FQQFGRDIA |
| 3310 | SQAWQPGVA |
| 3311 | LITGRLQSL |
| 3312 | LMPILTLTR |
| 3313 | SIKNFKSVL |
| 3314 | VELFENKTT |
| 3315 | DPNFKDQVI |
| 3316 | RPQGLPNNT |
| 3317 | VTLKQGEIK |
| 3318 | LTPFARCCW |
| 3319 | RIRSVYPVA |
| 3320 | MDSTVKNYF |
| 3321 | QEFRYMNSQ |
| 3322 | IRENNRVVI |
| 3323 | VGVALLAVF |
| 3324 | CLAYYFMRF |
| 3325 | YLKRRVVFN |
| 3326 | YCALAPNMM |
| 3327 | IFLWLLWPV |
| 3328 | CLFLLPSLA |
| 3329 | TESIVRFPN |
| 3330 | CTQHQPYVV |
| 3331 | DGWEIVKFI |
| 3332 | IVGVALLAV |
| 3333 | VEWKFYDAQ |
| 3334 | LIGCNYLGK |
| 3335 | TLQGCSLCL |
| 3336 | APAHISTIG |
| 3337 | TEISFMLWC |
| 3338 | REFVFKNID |
| 3339 | SGVYQCAMR |
| 3340 | KQQTVTLLP |
| 3341 | LFKDCSKVI |
| 3342 | RQKRTATKA |
| 3343 | CSTMTNRQF |
| 3344 | LEGSVAYES |
| 3345 | TILTSLLVL |
| 3346 | HGTILTRPL |
| 3347 | TCLAYYFMR |
| 3348 | VAALTNNVA |
| 3349 | LMWLSYFIA |
| 3350 | ALALLLLDR |
| 3351 | QRVAGDSGF |
| 3352 | YSPIFLIVA |
| 3353 | VPRASANIG |
| 3354 | ISSVLNDIL |
| 3355 | GLTVLPPLL |
| 3356 | IVQLSEISM |
| 3357 | TLETAQNSV |
| 3358 | LFLTWICLL |
| 3359 | FLIGCNYLG |
| 3360 | SQWLTNIFG |
| 3361 | LCDRRATCF |
| 3362 | LLWPVTLAC |
| 3363 | ICISTKHFY |
| 3364 | DFATSACVL |
| 3365 | QAAGTDTTI |
| 3366 | QSRNLQEFK |
| 3367 | CNDPFLGVY |
| 3368 | GSSKCVCSV |
| 3369 | DDYFNKKDW |
| 3370 | FITREEAIR |
| 3371 | MCVEYCPIF |
| 3372 | LPTEVLTEE |
| 3373 | QRQKKQQTV |
| 3374 | DLTKPYIKW |
| 3375 | AISMWALII |
| 3376 | KSAPLIELC |
| 3377 | SETKCTLKS |
| 3378 | TGTLIVNSV |
| 3379 | MFHLVDFQV |
| 3380 | SSGVTRELM |
| 3381 | VEGFNCYFP |
| 3382 | EQYIKWPWY |
| 3383 | LNEEIAIIL |
| 3384 | NRVVISSDV |
| 3385 | KLALGGSVA |
| 3386 | MRTFKVSIW |
| 3387 | KGFYAEGSR |
| 3388 | IIRENNRVV |
| 3389 | LGTVSWNLR |
| 3390 | NHNFLVQAG |
| 3391 | QSCTQHQPY |
| 3392 | SDFVRATAT |
| 3393 | LLPLTQYNR |
| 3394 | FIKQYGDCL |
| 3395 | CVRGTTVLL |
| 3396 | NYYKKVDGV |
| 3397 | TTTLNGLWL |
| 3398 | QKEMATSTL |
| 3399 | VGELLLLEW |
| 3400 | SMDNSPNLA |
| 3401 | CAKEIKESV |
| 3402 | IIPLTTAAK |
| 3403 | LLSKGRLII |
| 3404 | ECPNFVFPL |
| 3405 | GDYILANTC |
| 3406 | SGWTAGAAA |
| 3407 | LVYCFLGYF |
| 3408 | QRNFYEPQI |
| 3409 | VRAWIGFDV |
| 3410 | FPVLHDIGN |
| 3411 | CTLSEQLDF |
| 3412 | QENWNTKHS |
| 3413 | ICYTPSKLI |
| 3414 | TEGLCVDIP |
| 3415 | TDFVNEFYA |
| 3416 | QMEIDFLEL |
| 3417 | NSTLEQYVF |
| 3418 | RPFLCCKCC |
| 3419 | QECSLQSCT |
| 3420 | DQAMTQMYK |
| 3421 | AISAKNRAR |
| 3422 | PANSTVLSF |
| 3423 | KEQHCQKAS |
| 3424 | QVNGYPNMF |
| 3425 | NKHADFDTW |
| 3426 | CYNGSPSGV |
| 3427 | CNLGGAVCR |
| 3428 | ITRAKVGIL |
| 3429 | YDFAVSKGF |
| 3430 | CAYWVPRAS |
| 3431 | EGNSPFHPL |
| 3432 | DEVRQIAPG |
| 3433 | KAFQLTPIA |
| 3434 | NEFYAYLRK |
| 3435 | GPLKVGGSC |
| 3436 | LOFTSLEIP |
| 3437 | AGNGGDAAL |
| 3438 | FKLSYGIAT |
| 3439 | NGQVFGLYK |
| 3440 | NTDFSRVSA |
| 3441 | VPVVDSYYS |
| 3442 | RMNSRNYIA |
| 3443 | WPLIVTALR |
| 3444 | ERSEAGVCV |
| 3445 | RTRSMWSFN |
| 3446 | MRVIHFGAG |
| 3447 | ACFVLAAVY |
| 3448 | KDLSPRWYF |
| 3449 | QLQQSMSSA |
| 3450 | LAVHECFVK |
| 3451 | SIINNTVYT |
| 3452 | ESIVRFPNI |
| 3453 | ACPLIAAVI |
| 3454 | RRATRRIRG |
| 3455 | QLQAAVGEL |
| 3456 | KAKKGAWNI |
| 3457 | VDRQTAQAA |
| 3458 | SPLYAFASE |
| 3459 | TKRNVIPTI |
| 3460 | SNSGSDVLY |
| 3461 | SQTSLRCGA |
| 3462 | YNLPTMCDI |
| 3463 | EVLTEEVVL |
| 3464 | HGHVMVELV |
| 3465 | GAEAAVKPL |
| 3466 | LKTGDLQPL |
| 3467 | FSTASDTYA |
| 3468 | RFQTLLALH |
| 3469 | VRCSFYEDF |
| 3470 | GARRVWTLM |
| 3471 | FMSEAKCWT |
| 3472 | LIGLAKRFK |
| 3473 | GAKLKALNL |
| 3474 | NEKTHVQLS |
| 3475 | GRYMSALNH |
| 3476 | WDLIISDMY |
| 3477 | SVCRLMKTI |
| 3478 | TRCNLGGAV |
| 3479 | RNYIAQVDV |
| 3480 | KRTTCFSVA |
| 3481 | LSKSLTENK |
| 3482 | KGIMMNVAK |
| 3483 | YDYVIFTQT |
| 3484 | MAGNGGDAA |
| 3485 | SYYKLGASQ |
| 3486 | EKFKEGVEF |
| 3487 | VTLLPAADL |
| 3488 | LPETYFTQS |
| 3489 | SAMQTMLFT |
| 3490 | NTCVGSDNV |
| 3491 | SLRLIDAMM |
| 3492 | KCYGVSPTK |
| 3493 | LHFLLFFRA |
| 3494 | QASLPFGWL |
| 3495 | LMGHFAWW |
| 3496 | YNSVTSSIV |
| 3497 | NNLVVMAYI |
| 3498 | LVPQEHYVR |
| 3499 | SVRVLQKAA |
| 3500 | QVTCGTTTL |
| 3501 | ERVRQALLK |
| 3502 | QVVDADSKI |
| 3503 | EFLRDGWEI |
| 3504 | CVLAAECTI |
| 3505 | TPFARCCWP |
| 3506 | AACRKVQHM |
| 3507 | KSHFAIGLA |
| 3508 | NRDVDTDFV |
| 3509 | FKDASGKPV |
| 3510 | ELWAKRNIK |
| 3511 | ALRQMSCAA |
| 3512 | ILSLLSKGR |
| 3513 | LVDFQVTIA |
| 3514 | NGECPNFVF |
| 3515 | KPGNFNKDF |
| 3516 | IRRPFLCCK |
| 3517 | CGACIRRPF |
| 3518 | PQLEQPYVF |
| 3519 | NTTKGGRFV |
| 3520 | MVMFTPLVP |
| 3521 | ECIKDLLAR |
| 3522 | LSEARQHLK |
| 3523 | FGEYSHVVA |
| 3524 | LDKSAGFPF |
| 3525 | TSWQTGDFV |
| 3526 | DELTGHMLD |
| 3527 | RMLLEKCDL |
| 3528 | YTERSEKSY |
| 3529 | VDIAANTVI |
| 3530 | LNNDYYRSL |
| 3531 | PAFDKSAFV |
| 3532 | QGSEYDYVI |
| 3533 | CVPQADVEW |
| 3534 | LEQWNLVIG |
| 3535 | FDYVYNPFM |
| 3536 | NKGAGGHSY |
| 3537 | MKTIGPDMF |
| 3538 | FFTYICGFI |
| 3539 | TDFATSACV |
| 3540 | CRFDTRVLS |
| 3541 | SEAFLIGCN |
| 3542 | GNICYTPSK |
| 3543 | HKHAFLCLF |
| 3544 | GDFKLNEEI |
| 3545 | TDDNALAYY |
| 3546 | EIPVAYRKV |
| 3547 | TKYTMADLV |
| 3548 | LYAFASEAA |
| 3549 | PWNVVRIKI |
| 3550 | VPTGYVDTP |
| 3551 | GDDYVYLPY |
| 3552 | FMIDVQQWG |
| 3553 | QPYVFIKRS |
| 3554 | SRILGAGCF |
| 3555 | KDCVVLHSY |
| 3556 | EPEEHVQIH |
| 3557 | KSFTVEKGI |
| 3558 | YPNMFITRE |
| 3559 | ILNNLGVDI |
| 3560 | KELLVYAAD |
| 3561 | QWNLVIGFL |
| 3562 | KPRPPLNRN |
| 3563 | SYSLFDMSK |
| 3564 | LNKEMYLKL |
| 3565 | DILGPLSAQ |
| 3566 | GTLIVNSVL |
| 3567 | RSLPGVFCG |
| 3568 | NMFITREEA |
| 3569 | VTLACFVLA |
| 3570 | YRYKPHSLS |
| 3571 | LVEVEKGVL |
| 3572 | AARYMRSLK |
| 3573 | TFFIYNKIV |
| 3574 | LRVCVDTVR |
| 3575 | IPLMYKGLP |
| 3576 | SYAAFATAQ |
| 3577 | NGLWLDDVV |
| 3578 | TSDYYQLYS |
| 3579 | VSFSTFEEA |
| 3580 | TITVEELKK |
| 3581 | MIELSLIDF |
| 3582 | LLPSLATVA |
| 3583 | KFLVFLGII |
| 3584 | CNVNRFNVA |
| 3585 | RRARSVASQ |
| 3586 | LFLVLIMLI |
| 3587 | AGSKSPIQY |
| 3588 | RWFLNRFTT |
| 3589 | FNVAITRAK |
| 3590 | VEKGIYQTS |
| 3591 | PPLNRNYVF |
| 3592 | YDYCIPYNS |
| 3593 | ALCADSIII |
| 3594 | QNYGDSATL |
| 3595 | LPFTINCQE |
| 3596 | SHLLLVAAG |
| 3597 | CRHHANEYR |
| 3598 | MDQESFGGA |
| 3599 | TPGSSRGTS |
| 3600 | RYPANSIVC |
| 3601 | PYEDFQENW |
| 3602 | GARKSAPLI |
| 3603 | INNTVYTKV |
| 3604 | FKPRSQMEI |
| 3605 | NSSRVPDLL |
| 3606 | ALVYDNKLK |
| 3607 | SIFSRTLET |
| 3608 | NNFCGPDGY |
| 3609 | FNAIATCDW |
| 3610 | QLESKMSGK |
| 3611 | QPIGALDIS |
| 3612 | DPCPIHFYS |
| 3613 | CPNFVFPLN |
| 3614 | GSEYDYVIF |
| 3615 | AMORKLEKM |
| 3616 | DGLLLALHF |
| 3617 | ASFSASTSA |
| 3618 | APATVCGPK |
| 3619 | IGNYTVSCL |
| 3620 | GEVPVSIIN |
| 3621 | NNELSPVAL |
| 3622 | VKCTSVVLL |
| 3623 | FCAFAVDAA |
| 3624 | NHNPQKEMA |
| 3625 | VLIMLIIFW |
| 3626 | LKQLPFFYY |
| 3627 | AVINGDRWF |
| 3628 | YQLRARSVS |
| 3629 | RTTCFSVAA |
| 3630 | FHLVDFQVT |
| 3631 | RSDARTAPH |
| 3632 | YVFIKRSDA |
| 3633 | RELMRELNG |
| 3634 | FVTNVNASS |
| 3635 | DCVVLHSYF |
| 3636 | PSLATVAYF |
| 3637 | VCNSLLTPF |
| 3638 | PFLYLYALV |
| 3639 | VDTVSALVY |
| 3640 | WALIISVTS |
| 3641 | VYSHLLLVA |
| 3642 | YCKSHKPPI |
| 3643 | SPNECNQMC |
| 3644 | EETGTLIVN |
| 3645 | GLPNNTASW |
| 3646 | VQMTKLATT |
| 3647 | KEMATSTLQ |
| 3648 | CATVHTANK |
| 3649 | LEQPYVFIK |
| 3650 | CSKVITGLH |
| 3651 | YSDSPCESH |
| 3652 | LTCFSTQFA |
| 3653 | SARIVYTAC |
| 3654 | NYFLCWHTN |
| 3655 | MSDRDLYDK |
| 3656 | ISRQRLTKY |
| 3657 | HMELPTGVH |
| 3658 | EWLAMAVML |
| 3659 | QKSILSPLY |
| 3660 | WNLDYIINL |
| 3661 | IELCVDEAG |
| 3662 | LFLAFVVFL |
| 3663 | DQIGYYRRA |
| 3664 | KDMTYRRLI |
| 3665 | KDGHVETFY |
| 3666 | KDCVMYASA |
| 3667 | YKRDAPAHI |
| 3668 | VAGDSGFAA |
| 3669 | IAVQMTKLA |
| 3670 | LAVTRMENA |
| 3671 | VGCHNKCAY |
| 3672 | RSQMEIDFL |
| 3673 | YAAFATAQE |
| 3674 | TAQEAYEQA |
| 3675 | PEAGLPYGA |
| 3676 | IMMNVAKYT |
| 3677 | GSEGLNDNL |
| 3678 | CNIVNVSLV |
| 3679 | ILDGISQYS |
| 3680 | WFLNRFTTT |
| 3681 | EQIDGYVMH |
| 3682 | MHHMELPTG |
| 3683 | SDYYQLYST |
| 3684 | ECVRGTTVL |
| 3685 | MNLKYAISA |
| 3686 | KAIKCVPQA |
| 3687 | YDFGDFIQT |
| 3688 | RNARNGVLI |
| 3689 | IVAAIVFIT |
| 3690 | SGETLGVLV |
| 3691 | YDYYRYNLP |
| 3692 | YAYLRKHFS |
| 3693 | RVAGDSGFA |
| 3694 | GIPKDMTYR |
| 3695 | PMDSTVKNY |
| 3696 | VCRHHANEY |
| 3697 | YRRLISMMG |
| 3698 | PLADNKFAL |
| 3699 | DFVRATATI |
| 3700 | ASANIGCNH |
| 3701 | AVNLLTNMF |
| 3702 | YSKWYIRVG |
| 3703 | LSVVSKVVK |
| 3704 | LIMLIIFWF |
| 3705 | KINAACRKV |
| 3706 | GILCIMSDR |
| 3707 | IKVTLVFLF |
| 3708 | LRCGACIRR |
| 3709 | VVQLTSQWL |
| 3710 | SQMEIDFLE |
| 3711 | YALVYFLOS |
| 3712 | YKLGASQRV |
| 3713 | VQEIQLQAA |
| 3714 | RGGSQASSR |
| 3715 | DEPEEHVQI |
| 3716 | YTKVDGVDV |
| 3717 | AKVGILCIM |
| 3718 | GIAIAMACL |
| 3719 | YRRATRRIR |
| 3720 | NDVSFLAHI |
| 3721 | LNKHIDAYK |
| 3722 | WLCWKCRSK |
| 3723 | DNKFALTCF |
| 3724 | NMRVIHFGA |
| 3725 | FSTQFAFAC |
| 3726 | FNICQAVTA |
| 3727 | RCLAVHECF |
| 3728 | TPIAVQMTK |
| 3729 | MESLVPGFN |
| 3730 | NLAWPLIVT |
| 3731 | MRLWLCWKC |
| 3732 | VTCLAYYFM |
| 3733 | WNLVIGFLF |
| 3734 | KQLQQSMSS |
| 3735 | ISDMYDPKT |
| 3736 | YQLYSTQLS |
| 3737 | LPTGTLLVD |
| 3738 | KQASLNGVT |
| 3739 | EETIYNLLK |
| 3740 | WLTYTGAIK |
| 3741 | PEVKILNNL |
| 3742 | SVLLSMQGA |
| 3743 | FALTCFSTQ |
| 3744 | KPVPYCYDT |
| 3745 | TENKYSQLD |
| 3746 | RDGCVPLNI |
| 3747 | PFLCCKCCY |
| 3748 | CPIFFITGN |
| 3749 | TIKGTHHWL |
| 3750 | APLTVFFDG |
| 3751 | MVVIPDYNT |
| 3752 | LFRNARNGV |
| 3753 | LLEILQKEK |
| 3754 | EELKKLLEQ |
| 3755 | TTTLNDFNL |
| 3756 | EHDYQIGGY |
| 3757 | GTAVLRQWL |
| 3758 | NPQKEMATS |
| 3759 | FARTRSMWS |
| 3760 | EGNFYGPFV |
| 3761 | GYRVTKNSK |
| 3762 | EPIYDEPTT |
| 3763 | VDRYPANSI |
| 3764 | NGRTILGSA |
| 3765 | YHYQECVRG |
| 3766 | AIRHVRAWI |
| 3767 | SLLTPFARC |
| 3768 | LVTLAILTA |
| 3769 | GSSVELKHF |
| 3770 | QLEQPYVFI |
| 3771 | SRGTSPARM |
| 3772 | SDEFSSNVA |
| 3773 | DDNLIDSYF |
| 3774 | LALLLLDRL |
| 3775 | SQASSRSSS |
| 3776 | LSVLLSMQG |
| 3777 | NKCAYWVPR |
| 3778 | IPMDSTVKN |
| 3779 | FKLVNKFLA |
| 3780 | DTYACWHHS |
| 3781 | TPVHVMSKH |
| 3782 | FDMSKFPLK |
| 3783 | TDFNAIATC |
| 3784 | RSMWSFNPE |
| 3785 | HCANFNVLF |
| 3786 | RGMVLGSLA |
| 3787 | FRKMAFPSG |
| 3788 | ISASIVAGG |
| 3789 | FAGILIVTT |
| 3790 | YRSLPGVFC |
| 3791 | TLATCELYH |
| 3792 | NQDLNGNWY |
| 3793 | WWTAFVTNV |
| 3794 | LLFVTVYSH |
| 3795 | MDLFMRIFT |
| 3796 | VDTDLTKPY |
| 3797 | DQVILLNKH |
| 3798 | APSASAFFG |
| 3799 | IKDATPSDF |
| 3800 | RRVVFNGVS |
| 3801 | KVTIDYTEI |
| 3802 | NSTVLSFCA |
| 3803 | YDCVSFCYM |
| 3804 | GTTFTYASA |
| 3805 | YSFVSEETG |
| 3806 | ESPFELEDF |
| 3807 | KTVQFCDAM |
| 3808 | KTEGLCVDI |
| 3809 | VDSSQGSEY |
| 3810 | WITGGIAIA |
| 3811 | REGVFVSNG |
| 3812 | VYPIILRLG |
| 3813 | IVTTIVYLT |
| 3814 | WAHGFELTS |
| 3815 | LDQAISMWA |
| 3816 | FNVVNKGHF |
| 3817 | LLLFVTVYS |
| 3818 | AWYTERSEK |
| 3819 | VNGYPNMFI |
| 3820 | TLVFLFVAA |
| 3821 | MQRKLEKMA |
| 3822 | LCNSQTSLR |
| 3823 | ATPSDFVRA |
| 3824 | TQFAFACPD |
| 3825 | GAVILRGHL |
| 3826 | ELLLLEWLA |
| 3827 | KHLIPLMYK |
| 3828 | LGGSVAIKI |
| 3829 | FHQECSLOS |
| 3830 | YCRHGTCER |
| 3831 | ELQTPFEIK |
| 3832 | DDPCPIHFY |
| 3833 | CDWTNAGDY |
| 3834 | GSALLEDEF |
| 3835 | SKWYIRVGA |
| 3836 | MAVMLLLLC |
| 3837 | KDFYDFAVS |
| 3838 | SGVKDCVVL |
| 3839 | AQSFLNRVC |
| 3840 | SSSEAFLIG |
| 3841 | GVEHVTFFI |
| 3842 | MLRKLDNDA |
| 3843 | CVEYCPIFF |
| 3844 | SPIQYIDIG |
| 3845 | LDMYSVMLT |
| 3846 | LNVGDYFVL |
| 3847 | TRAGCLIGA |
| 3848 | MCASLKELL |
| 3849 | GQTFSVLAC |
| 3850 | VMSLKEGQI |
| 3851 | LRIAGHHLG |
| 3852 | HIVYGDFSH |
| 3853 | QLDFIDTKR |
| 3854 | IPDYNTYKN |
| 3855 | SNHNFLVQA |
| 3856 | NKTTLPVNV |
| 3857 | WHTNCYDYC |
| 3858 | QIAQFAPSA |
| 3859 | FRLFARTRS |
| 3860 | LEDFIPMDS |
| 3851 | LRIAGHHLG |
| 3852 | HIVYGDFSH |
| 3853 | QLDFIDTKR |
| 3854 | IPDYNTYKN |
| 3855 | SNHNFLVQA |
| 3856 | NKTTLPVNV |
| 3857 | WHTNCYDYC |
| 3858 | QIAQFAPSA |
| 3859 | FRLFARTRS |
| 3860 | LEDFIPMDS |
| 3851 | LRIAGHHLG |
| 3852 | HIVYGDFSH |
| 3853 | QLDFIDTKR |
| 3854 | IPDYNTYKN |
| 3855 | SNHNFLVQA |
| 3856 | NKTTLPVNV |
| 3857 | WHTNCYDYC |
| 3858 | QIAQFAPSA |
| 3859 | FRLFARTRS |
| 3860 | LEDFIPMDS |

TABLE 16
Region-specific peptide pools derived from whole proteome for CD8 T cell assays.

| Region | Seq. IDs of peptides |
|---|---|
| Australia | 1-11, 13-14, 16-20, 23-25, 28-35, 38, 47-49, 54-55, 58-60, 62, 64-67, 71-73, 77- |
| | 82, 85-86, 88-90, 92, 96-103, 106, 109, 113-121, 123, 125-127, 133, 137-139, |
| | 143-144, 184-186, 189-193, 203, 206, 208, 210-213, 215-216, 218-219, 221-225, |
| | 233-237, 241-243, 248-250, 262, 280, 288, 290, 293-299, 301-302, 305-306, 309- |
| | 311, 314-323, 325-327, 335, 337, 339-340, 346, 354-355, 362, 364, 370, 392, 394, |
| | 400-401, 406, 408-413, 419-423, 429-431, 434, 436-440, 444-447, 454, 456-460, |
| | 463, 465, 471, 477, 487-491, 495, 503, 515-530, 541, 543, 548, 551-553, 555-557, |
| | 567, 575, 584-585, 587, 591-592, 597, 607-611, 618, 621, 628-629, 632-633, 639, |
| | 641-642, 644, 661-675, 677, 702-705, 707-708, 714, 716-719, 723-724, 728, 733, |
| | 736, 753-756, 759-760, 763, 768, 784-791, 810-811, 829, 832, 835, 845-849, 852, |
| | 862-871, 873-874, 876-879, 883-901, 911, 913-916 |
| Europe | 1-11, 13-14, 16-20, 23-25, 28-30, 32-35, 38, 47-49, 54-55, 58-60, 62, 64-67, 71- |
| | 73, 77-82, 86, 88-89, 96-102, 106, 109-110, 113-121, 123, 125-127, 129, 133, |
| | 137-139, 163, 171-173, 175-176, 181-182, 184-185, 189-193, 203, 206, 210-213, |
| | 215-216, 218-219, 221-224, 233-237, 241-243, 247-248, 254-259, 262, 268-272, |
| | 281-282, 286, 288, 293-299, 301-302, 305-306, 309-310, 314-323, 325-326, 329- |
| | 338, 343-346, 348-349, 351-355, 357-362, 364, 370, 372, 392, 394, 400-401, 408- |
| | 413, 419-423, 429-431, 434, 436-440, 445-447, 449, 465, 477, 487-488, 490-491, |
| | 513, 516, 519-520, 524, 548, 552, 557, 559, 570-587, 591-597, 611, 621, 664, |
| | 667, 683, 686, 691, 693, 699, 702, 720-732, 740-752, 755, 759-760, 763, 767-768, |
| | 829, 864-865, 873-874, 876-880, 884-901, 911 |
| North Africa | 1-11, 13-14, 16-21, 23-25, 28-35, 38, 44, 47-49, 54, 58-60, 62, 64-66, 77, 85-86, |
| | 88-90, 92, 96-103, 109-110, 126-127, 133, 137-139, 145-146, 148-152, 158-160, |
| | 206, 214, 222, 233-237, 241-243, 268-272, 281-282, 286, 289-290, 309-310, 314- |
| | 323, 325-327, 337, 340, 346, 348-349, 351-355, 357-362, 364, 367-368, 370, 376, |
| | 392, 394, 400-401, 408-413, 420-423, 429-431, 434, 436-440, 445-447, 452-468, |
| | 471, 473, 477-483, 487-488, 490-491, 515-530, 544, 546, 548, 552, 557, 559, 585, |
| | 591, 595, 610-623, 628-634, 637, 639-642, 644, 648, 653, 664, 673, 676-677, 702, |
| | 711-712, 715, 719, 728, 733-734, 736, 738-739, 753-756, 759-760, 763-764, 767- |
| | 768, 787-789, 792-798, 800-801, 803, 807-811, 816-818, 820-826, 829-831, 837- |
| | 844, 864-865, 875, 883-894, 896-900, 902, 907-911, 913-917 |
| North America | 1-11, 13-14, 16-20, 23-25, 28-30, 32-35, 38, 47-49, 53-55, 58-60, 62, 64-67, 70- |
| | 73, 77-82, 86, 88-90, 96-102, 125-127, 173-174, 206, 208-213, 215-216, 218-219, |
| | 221-225, 233-237, 241-243, 247-248, 254-260, 268-272, 280-281, 286, 288, 293- |
| | 299, 301-302, 305-306, 309-310, 314-323, 325-326, 329-338, 343-345, 348-349, |
| | 351-355, 357-362, 370, 392, 400, 406, 408-413, 419-423, 429-431, 434, 436-440, |
| | 444-447, 471, 487-491, 495-496, 498-501, 503-504, 507-508, 513, 515-524, 527, |
| | 536-540, 548, 552-553, 556-561, 570-576, 578-581, 585, 589-590, 628-629, 633, |
| | 639, 641, 644, 661, 733, 755-756, 759-760, 763, 768, 811, 829, 832, 845-853, |
| | 862-871, 873-874, 876-880, 884-901 |
| North East Asia | 23-25, 28-30, 32-35, 38, 42, 44, 47, 55, 64-67, 70-73, 77-82, 86, 88-89, 96-102, |
| | 106-108, 110, 113-121, 123, 125-127, 143-144, 163, 165, 169, 171-173, 175, 195, |
| | 206, 208, 210-213, 215-216, 218-219, 221-225, 242, 247-250, 254-260, 268-272, |
| | 274, 280-282, 286, 288, 290, 293-299, 301-302, 305-306, 311, 326-327, 329-340, |
| | 343-345, 348-349, 351-355, 357-362, 364, 370, 394, 406, 419, 432, 434, 436-440, |
| | 444-448, 452-457, 459-467, 471, 477, 481, 483-491, 495, 513, 515-520, 522-524, |
| | 536-538, 540-549, 552-553, 556-561, 570-583, 585, 591, 595, 598-606, 611, 633, |
| | 637, 639, 641, 644, 659, 661, 664, 667, 673, 675-677, 702-703, 723-724, 728, |
| | 733, 755-756, 759-760, 763, 767-769, 784-791, 808, 811, 827-829, 832, 837-843, |
| | 845-852, 862-871, 873-874, 876-894, 897, 903-904, 906-908, 911-912 |
| Oceania | 23-25, 28-35, 38, 42, 44, 47-50, 55, 64-67, 70-73, 77-82, 86, 88-90, 96-102, 106, |
| | 110, 113-121, 123, 125-127, 143-144, 163, 165, 169, 173, 177, 186, 195, 208, |
| | 225, 242, 248-250, 260-262, 268-272, 280-282, 286, 288, 290, 293-299, 301-302, |
| | 305-306, 311, 327, 335, 337, 339-340, 354-355, 362, 370, 394, 406, 419, 432, |
| | 434, 436-441, 444-448, 452-457, 459-463, 465, 467, 471, 477, 481, 484-491, 495- |
| | 496, 502-503, 509-513, 515-530, 534-535, 540-541, 543, 545, 548, 550-557, 559, |
| | 567, 570-572, 574-575, 577, 579, 584-585, 587, 591, 597-611, 618, 633, 639, |
| | 641-642, 644, 649-650, 653, 659, 661-662, 664-665, 667, 673, 675, 677, 682-684, |
| | 686, 689-691, 693-694, 696-700, 702-715, 719, 723-724, 728, 733-734, 736-739, |
| | 753, 755-756, 759-760, 763-764, 767-768, 770, 776, 781-791, 811, 829, 845-849, |
| | 852-853, 862-871, 873-874, 876-880, 883-900, 903-904, 906-908, 911-916 |
| South and Central America | 1-11, 13-14, 16-20, 23-25, 28-30, 32-35, 38, 44, 47-49, 54-55, 58-60, 62, 64-66, |
| | 77-81, 86, 88-90, 96-102, 110, 125-126, 163, 165, 171-173, 175, 184-185, 189- |
| | 193, 195, 203, 225, 242-243, 260, 268-272, 280-282, 286, 290, 302, 311, 326-327, |
| | 329-340, 343-345, 348-349, 351-355, 357-362, 364, 370, 392, 394, 400, 406, 408- |
| | 413, 419-423, 429-432, 434, 436-440, 445-447, 452-464, 466-467, 469, 471, 477, |
| | 487-491, 493, 503, 513, 515-527, 529, 531-534, 548, 553, 555, 557, 559, 570-583, |
| | 585, 591, 605-606, 608-611, 618, 621, 624-626, 628-629, 632-633, 637, 639, 641- |
| | 642, 644, 649-651, 653, 661-665, 667, 670-673, 675-677, 682, 702, 707, 719, |
| | 723-724, 728, 731, 733-734, 736-739, 753-756, 759-760, 763-764, 768-769, 784- |
| | 791, 804-806, 810-811, 827, 829, 832, 835, 846, 862-871, 873-874, 876-901, 913- |
| | 916 |
| South Asia | 1-11, 13-14, 16-20, 23-25, 28-30, 32-35, 38, 44, 47-49, 54-55, 58-60, 62, 64-67, |
| | 70-73, 77-82, 86, 88-90, 96-102, 109-110, 126, 129, 163, 165, 169, 173, 176, 184- |
| | 185, 189-193, 195, 203, 214, 242, 262, 269, 272-273, 282, 286, 290, 326-327, |
| | 334-337, 340, 346, 348-349, 351-355, 357-362, 364, 370, 372, 380-392, 394, 400- |
| | 401, 408-413, 420-423, 429-432, 434, 436-441, 444-448, 452-467, 469, 472, 477, |
| | 479-480, 487-488, 490-492, 513, 515-524, 527-528, 530, 536-537, 540, 546, 548, |
| | 552, 556-557, 559, 570-572, 574-575, 577, 579, 584-588, 591, 595, 598-606, 610- |
| | 611, 618, 621, 629, 633, 639-640, 642, 644, 646, 649, 653, 659, 661, 664, 667, |
| | 673, 676-677, 682-686, 688-694, 696-703, 709, 711-712, 715, 719-720, 722-723, |
| | 727-730, 733-739, 753-763, 767-768, 787-789, 792-803, 808, 812-819, 828-829, |
| | 832-843, 865, 873-875, 883-904, 906, 911-916 |
| South East Asia | 23-25, 28-30, 32-35, 38, 42, 44, 47-50, 55, 64-65, 67, 70-73, 77-82, 86, 88-89, 96- |
| | 102, 110, 125-126, 163, 165, 169, 173, 193, 195, 208, 225, 242-243, 247-249, |
| | 254-262, 280, 282, 288, 293-299, 301-302, 305-306, 335, 348-349, 351-355, 357- |
| | 362, 370, 372, 392, 400, 406, 408-411, 419, 434, 436-440, 444-447, 452-469, |
| | 471-477, 481, 483-491, 496, 502-503, 509-520, 522-524, 527, 540, 548, 552-553, |
| | 556-557, 559, 567, 570-572, 574-575, 577, 579, 584-586, 591-592, 598-606, 611, |
| | 633, 639, 641, 644, 659, 661-675, 689-690, 694, 696-700, 702-706, 709, 712, 715, |
| | 733, 743, 755-756, 759-760, 763, 767-772, 774-778, 811, 817, 827-829, 832, 844- |
| | 852, 854-871, 873-880, 884-894, 897, 903-908 |
| Sub-Saharan Africa | 1-11, 13-14, 16-21, 23-25, 28-35, 38, 47-50, 53-55, 58-60, 62, 64-67, 71-73, 77- |
| | 82, 85-86, 90, 92, 103, 109-110, 125-128, 133, 137-139, 145-152, 158-160, 162, |
| | 173-174, 206, 210-213, 215-216, 218-219, 221-224, 226, 233-237, 241-243, 248, |
| | 282, 286, 355, 362, 364, 370, 376, 392, 394, 400, 408-413, 419-423, 429-431, |
| | 434, 436-440, 444-447, 455, 458, 460, 465, 467-468, 477, 479, 482, 487-488, |
| | 490-491, 495-497, 513, 515-530, 536-537, 544, 548, 551-553, 556-557, 559-560, |
| | 562-569, 571, 574-576, 581, 585, 595, 610-623, 627-661, 702, 712, 755, 759-760, |
| | 763, 767-768, 829, 864-865, 873-880, 883-894, 896-900, 902, 907-910, 917 |
| Western Asia | 1-11, 13-14, 16-21, 23-25, 28-30, 32-35, 38, 44, 48, 50, 53, 55, 64-67, 69-73, 77- |
| | 82, 86, 88-90, 96-102, 106, 109-110, 113-121, 123, 126, 129, 173-174, 176, 233- |
| | 237, 241-243, 262, 268-273, 276, 281-282, 286, 326-327, 329-338, 343-346, 355, |
| | 364, 370, 376, 392, 394, 400-401, 408-413, 420-423, 429-431, 434, 436-440, 444- |
| | 447, 452-471, 477, 479, 485, 487-488, 490-491, 494-506, 513, 515-521, 524, 527- |
| | 528, 530, 536-537, 548, 551-552, 556-557, 559, 570-576, 581, 584-588, 591, 595, |
| | 610-611, 618, 621, 642, 649-651, 653, 662, 664, 666-668, 670, 673, 676-698, 700, |
| | 702, 716, 719-734, 736-739, 755, 759-760, 763-768, 770-780, 808, 829, 837-843, |
| | 865, 872, 883-901, 907-911, 913-916 |

TABLE 17
Region-specific peptide pools derived from S protein for CD8 T cell assays.

| Region | Seq. IDs of peptides |
|---|---|
| Australia | 1, 4, 9-10, 28, 43, 45, 55-57, 59-60, 79, 83, 85, 88-89, 93, 100-101, 109, 124-125, |
| | 132, 134-139, 141-145, 155-156, 161, 188, 192-193, 195-197, 199, 213, 218, 221, |
| | 224, 238-239, 241, 282-286, 297-299, 304-306, 324-326, 338-341, 363, 365, 369- |
| | 371, 373, 377, 406, 411, 418, 423, 433, 440, 447, 449, 455, 458, 463, 473, 496, |
| | 505, 520-521, 532, 611, 623, 630, 639, 644, 647, 650, 672, 678, 691, 711, 715, |
| | 734, 764, 775, 789, 794, 805, 828, 849-850, 856, 863, 867, 872, 885-886, 909, |
| | 916, 931, 934, 944, 947, 953, 965, 992-993, 1004, 1015, 1020, 1024, 1036, 1052, |
| | 1066, 1097, 1100, 1149, 1176, 1207, 1217-1218, 1237, 1261-1263, 1295, 1298, |
| | 1317, 1319, 1347, 1405, 1408-1409, 1416, 1420, 1451, 1457, 1465, 1487, 1495, |
| | 1516, 1528, 1533, 1538, 1581, 1587, 1610, 1625, 1633, 1639, 1644, 1657, 1661, |
| | 1666, 1678, 1692, 1697, 1743, 1781, 1784, 1813, 1817, 1823, 1832, 1837, 1842, |
| | 1846, 1849, 1854, 1870, 1929, 1961-1962, 2001, 2046, 2060, 2062, 2065, 2077, |
| | 2079, 2088, 2096, 2134, 2174, 2184, 2208, 2247, 2290, 2310, 2316, 2318, 2327, |
| | 2346, 2376, 2387, 2399, 2416, 2445, 2459, 2489, 2493, 2525, 2606-2607, 2613, |
| | 2618, 2635, 2655, 2659, 2677, 2688, 2698, 2719, 2779, 3004, 3406 |
| Europe | 1, 4, 9-10, 28, 43, 45, 55-57, 59-60, 83, 88-89, 93, 100-101, 109, 124-125, 132, |
| | 134-139, 141-145, 155, 168-172, 174-175, 182, 188, 192, 195-197, 199, 213, 218, |
| | 221, 224, 238-239, 241, 257, 271, 282-286, 297-299, 304-306, 324-326, 331, 339- |
| | 342, 345, 363, 365, 370, 373, 406, 411, 418, 423, 433, 440, 447, 449, 455, 458, |
| | 463, 473, 496, 505, 520-521, 570, 601, 611, 623, 647, 672, 678, 686, 688, 691, |
| | 715, 775, 783, 789, 794, 805, 828, 856, 863, 867, 872, 885-886, 909, 916, 931, |
| | 934, 944, 947, 965, 992-993, 999, 1004, 1020, 1024, 1036, 1052, 1066, 1149, |
| | 1176, 1207, 1217, 1237, 1242, 1261-1262, 1317, 1319, 1372, 1408, 1416, 1465, |
| | 1477, 1495, 1516, 1523, 1533, 1536, 1581, 1587, 1633, 1639, 1644, 1657, 1661, |
| | 1678, 1781, 1784, 1813, 1837, 1842, 1849, 1863, 1869, 1886, 1954, 1961, 2001, |
| | 2046, 2088, 2174, 2283, 2297, 2318, 2398, 2445, 2619, 2649, 2677, 2772, 2790, |
| | 2802, 3042, 3329, 3382, 3408, 3432, 3585, 3847 |
| North Africa | 1, 4, 9-10, 28, 43, 45, 55-57, 59-60, 85, 88-89, 93, 100-101, 109, 125, 132, 134- |
| | 139, 141-145, 155-156, 159, 238-239, 241, 271, 282-286, 304, 324-325, 338-341, |
| | 363, 365, 367-371, 373, 377, 406, 411, 418, 423, 433, 440, 447, 449, 455, 458, |
| | 463, 473, 520-521, 532, 611, 623, 630, 639, 644, 647, 650, 672, 678, 691, 711, |
| | 715, 734, 764, 775, 789, 794, 805, 819, 828, 849, 856, 861, 863, 867, 872, 885- |
| | 886, 909, 916, 931, 934, 944, 947, 965, 988, 992-993, 1004, 1020, 1024, 1036, |
| | 1065-1066, 1097, 1149, 1176, 1207, 1217-1218, 1261-1263, 1295, 1298, 1317, |
| | 1319, 1335, 1347, 1382, 1405, 1408, 1416, 1420, 1450-1451, 1457, 1465, 1477, |
| | 1485, 1495, 1516, 1533, 1538, 1540, 1581, 1610, 1625, 1639, 1644, 1649, 1661, |
| | 1666, 1678, 1743, 1781, 1802, 1813, 1817, 1837, 1846, 1849, 1854, 1870, 1880, |
| | 1890, 1929, 1953-1954, 2046, 2077, 2088, 2096, 2174, 2194, 2208, 2223-2224, |
| | 2240, 2247, 2259, 2273, 2283, 2316, 2318, 2376, 2387, 2393, 2424, 2445, 2475, |
| | 2523, 2525, 2584, 2606-2607, 2613, 2618, 2635, 2651, 2657, 2659, 2677, 2688, |
| | 2693, 2698, 2719, 2832, 2882, 2905, 3308-3309, 3355, 3377, 3381 |
| North America | 1, 4, 9-10, 28, 43, 55-57, 59-60, 79, 83, 88-89, 93, 100-101, 109, 125, 132, 134- |
| | 136, 141-145, 156, 159, 168-170, 174, 182, 192-193, 195, 213, 218, 221, 224, |
| | 238-239, 241, 257, 271, 282-286, 297-299, 304-306, 324-325, 331, 339-342, 345, |
| | 363, 365, 369-371, 406, 411, 418, 423, 433, 440, 447, 449, 455, 458, 463, 496, |
| | 505, 520-521, 532, 570, 601, 611, 623, 630, 639, 644, 647, 650, 672, 691, 715, |
| | 734, 764, 775, 828, 849-850, 856, 863, 867, 872, 885-886, 909, 931, 953, 992- |
| | 993, 999, 1004, 1020, 1024, 1036, 1100, 1109, 1132, 1149, 1176, 1207, 1217- |
| | 1218, 1261-1262, 1317, 1319, 1408-1409, 1420, 1487, 1495, 1516, 1528, 1533, |
| | 1625, 1633, 1649, 1657, 1661, 1666, 1678, 1692, 1697-1698, 1781, 1813, 1817, |
| | 1823, 1832, 1837, 1846, 1849, 1863, 1869-1870, 1886, 1918, 1954, 2001, 2027, |
| | 2046, 2063, 2077, 2079, 2088, 2174, 2290, 2297, 2310, 2318, 2327, 2346, 2399, |
| | 2445, 2459, 2489, 2493, 2515, 2525, 2613, 2619, 2677, 2688, 2698, 2772, 2779, |
| | 2781, 2819, 2839, 3030, 3193, 3233, 3468, 3492 |
| North East Asia | 28, 43, 45, 55-56, 79, 83, 88-89, 93, 100-101, 109, 124-125, 132, 134-136, 141- |
| | 145, 168-172, 174-175, 182, 193, 195-196, 213, 218, 221, 224, 257, 271, 282-286, |
| | 297-299, 304-306, 324, 326, 331, 338-342, 345, 363, 365, 369-371, 373, 406, 411, |
| | 433, 440, 447, 449, 455, 458, 463, 473, 496, 505, 520-521, 532, 570, 601, 611, |
| | 623, 630, 639, 644, 647, 650, 672, 678, 691, 715, 734, 775, 789, 794, 805, 828, |
| | 849-850, 856, 863, 867, 872, 885-886, 909, 916, 931, 934, 944, 947, 953, 965, |
| | 988, 993, 999, 1004, 1015, 1020, 1024, 1036, 1065-1066, 1100, 1132, 1149, 1176, |
| | 1207, 1217-1218, 1237, 1261-1262, 1298, 1317, 1319, 1335, 1347, 1382, 1405, |
| | 1408-1409, 1416, 1420, 1451, 1465, 1485, 1487, 1516, 1528, 1533, 1538, 1581, |
| | 1587, 1633, 1639, 1644, 1649, 1657, 1661, 1666, 1678, 1692, 1697, 1734, 1743, |
| | 1781, 1802, 1813, 1817, 1823, 1832, 1837, 1842, 1846, 1849, 1854, 1863, 1870, |
| | 1886, 1929, 1953-1954, 2001, 2018, 2043, 2046, 2049, 2060, 2065, 2077, 2079, |
| | 2088, 2174, 2183-2184, 2223-2224, 2240, 2247, 2283, 2290, 2297, 2310, 2318, |
| | 2326-2327, 2346, 2389, 2398-2399, 2459, 2489, 2493, 2511, 2515, 2523, 2525, |
| | 2552, 2613, 2619, 2635, 2649, 2652, 2655, 2659, 2698, 2719, 2772, 2779, 2805, |
| | 2839, 2854, 3004, 3030, 3050, 3233 |
| Oceania | 28, 43, 45, 55-57, 79, 83, 88-89, 93, 100-101, 109, 124-125, 134-135, 141-145, |
| | 155-156, 161, 168-170, 174, 188, 193, 195-196, 199, 271, 282-286, 297-299, 304- |
| | 306, 324, 326, 338-341, 363, 365, 369-371, 373, 377, 406, 423, 433, 440, 447, |
| | 449, 455, 458, 463, 473, 496, 505, 520-521, 532, 570, 601, 611, 623, 630, 639, |
| | 644, 647, 650, 672, 678, 686-688, 691, 711, 715, 734, 764, 775, 783, 789, 794, |
| | 805, 828, 849-850, 856, 861, 863, 867, 872, 885-886, 909, 916, 931, 934, 944, |
| | 947, 953, 965, 988, 992-993, 999, 1004, 1015, 1020, 1024, 1036, 1052, 1054, |
| | 1066, 1097, 1100, 1109, 1149, 1176, 1207, 1217-1218, 1237, 1261-1263, 1295, |
| | 1298, 1317, 1319, 1347, 1382, 1408-1409, 1416, 1420, 1451, 1457, 1465, 1487, |
| | 1495, 1516, 1523, 1528, 1533, 1538, 1581, 1587, 1610, 1625, 1633, 1639, 1644, |
| | 1649, 1657, 1661, 1666, 1678, 1692, 1697-1698, 1734, 1781, 1784, 1802, 1805, |
| | 1813, 1817, 1832, 1837, 1842, 1846, 1849, 1854, 1870, 1918, 1929, 1953-1954, |
| | 1962, 2001, 2027, 2043, 2046, 2049, 2060, 2063, 2065, 2077, 2079, 2088, 2096, |
| | 2134, 2174, 2184, 2208, 2240, 2247, 2290, 2297, 2310, 2316, 2318, 2325-2327, |
| | 2346, 2376, 2387, 2389, 2398-2399, 2445, 2459, 2489, 2493, 2511, 2515, 2523, |
| | 2525, 2552, 2606, 2613, 2635, 2645, 2649, 2655, 2659, 2677, 2688, 2698, 2719, |
| | 2761, 2772, 2779, 2781, 2839, 2848, 2854, 2907, 3003-3004, 3013, 3042, 3050, |
| | 3406 |
| South and Central America | 1, 4, 9-10, 28, 43, 45, 55-57, 59-60, 79, 88-89, 93, 100-101, 109, 125, 142-145, |
| | 155-156, 168-172, 174-175, 188, 192-193, 195-197, 199, 271, 282-286, 304, 326, |
| | 331, 338-342, 345, 363, 365, 369-371, 373, 377, 406, 411, 418, 423, 433, 440, |
| | 447, 449, 455, 458, 463, 473, 496, 505, 520-521, 532, 570, 601, 611, 623, 639, |
| | 644, 647, 650, 672, 678, 686, 691, 711, 715, 734, 764, 775, 789, 794, 805, 828, |
| | 849-850, 863, 867, 872, 885-886, 909, 916, 931, 934, 944, 953, 965, 988, 992- |
| | 993, 999, 1004, 1015, 1020, 1024, 1036, 1066, 1097, 1149, 1176, 1207, 1217- |
| | 1218, 1237, 1261-1262, 1298, 1317, 1319, 1347, 1382, 1405, 1408, 1416, 1420, |
| | 1451, 1457, 1465, 1487, 1495, 1516, 1538, 1581, 1587, 1610, 1625, 1633, 1639, |
| | 1644, 1649, 1657, 1661, 1666, 1674, 1678, 1697, 1743, 1781, 1805, 1813, 1817, |
| | 1832, 1837, 1842, 1846, 1849, 1863, 1869, 1886, 1929, 1961, 2001, 2046, 2060, |
| | 2065, 2077, 2079, 2088, 2134, 2174, 2184, 2208, 2224, 2247, 2290, 2297, 2310, |
| | 2316, 2318, 2326-2327, 2346, 2376, 2392, 2398-2399, 2416, 2445, 2523, 2525, |
| | 2606, 2619, 2635, 2649, 2657, 2677, 2688, 2698, 2711, 2719, 2772, 2805, 3003- |
| | 3004, 3050, 3355 |
| South Asia | 1, 4, 9-10, 28, 43, 45, 55-57, 59-60, 79, 83, 88-89, 93, 100-101, 109, 125, 134, |
| | 141-145, 155, 168-170, 188, 192-193, 195-197, 199, 238, 282-286, 304, 324, 326, |
| | 338-342, 363, 365, 369-371, 373, 377, 391, 406, 411, 418, 423, 433, 440, 447, |
| | 449, 455, 458, 463, 473, 496, 505, 520-521, 532, 570, 601, 611, 623, 639, 644, |
| | 647, 650, 672, 678, 686-688, 691, 711, 715, 734, 764, 775, 783, 789, 794, 805, |
| | 819, 828, 849-850, 856, 861, 863, 867, 872, 885-886, 909, 916, 931, 934, 944, |
| | 947, 953, 965, 988, 992-993, 999, 1004, 1015, 1020, 1024, 1036, 1052, 1054, |
| | 1065-1066, 1097, 1100, 1149, 1176, 1207, 1217, 1237, 1242, 1261-1263, 1298, |
| | 1317, 1319, 1347, 1372, 1382, 1405, 1408-1409, 1416, 1420, 1450-1451, 1457, |
| | 1465, 1485, 1495, 1516, 1523, 1533, 1536, 1538, 1540, 1587, 1610, 1625, 1639, |
| | 1644, 1649, 1657, 1661, 1666, 1678, 1734, 1743, 1784, 1804-1805, 1813, 1817, |
| | 1832, 1837, 1842, 1846, 1849, 1854, 1869, 1890, 1929, 1953-1954, 1961, 2001, |
| | 2043, 2060, 2077, 2088, 2174, 2184, 2194, 2208, 2224, 2247, 2283, 2297, 2318, |
| | 2325-2326, 2376, 2387, 2389, 2392, 2398, 2416, 2424, 2445, 2511, 2515, 2525, |
| | 2552, 2607, 2618, 2635, 2647, 2649, 2651, 2657, 2659, 2665, 2677, 2693, 2719, |
| | 2772, 2839, 2854, 3042, 3050, 3097, 3223, 3329, 3338, 3355, 3377, 3590, 3811 |
| South East Asia | 28, 43, 45, 55-57, 79, 83, 88-89, 93, 100-101, 109, 125, 141-145, 156, 161, 168- |
| | 170, 174, 193, 195-196, 239, 257, 282-284, 286, 297-299, 304-306, 324, 326, 340, |
| | 363, 365, 369-371, 373, 377, 406, 411, 418, 433, 440, 447, 449, 455, 458, 463, |
| | 473, 496, 505, 520, 532, 570, 601, 611, 623, 630, 639, 644, 647, 650, 672, 678, |
| | 686, 688, 691, 711, 715, 734, 764, 773, 775, 783, 805, 828, 849-850, 856, 861, |
| | 863, 867, 872, 885-886, 909, 916, 931, 934, 944, 947, 953, 965, 988, 992-993, |
| | 999, 1004, 1015, 1020, 1024, 1036, 1052, 1054, 1100, 1109, 1132, 1138, 1149, |
| | 1176, 1207, 1217-1218, 1237, 1242, 1261-1262, 1317, 1319, 1347, 1382, 1405, |
| | 1408-1409, 1420, 1428, 1465, 1487, 1516, 1523, 1528, 1533, 1581, 1587, 1633, |
| | 1639, 1649, 1657, 1661, 1678, 1697-1698, 1743, 1781, 1784, 1805, 1813, 1832, |
| | 1837, 1842, 1849, 1854, 1869-1870, 1918, 1954, 1961, 2001, 2018-2019, 2027, |
| | 2046, 2060, 2062-2063, 2065, 2077, 2079, 2088, 2134, 2174, 2240, 2290, 2297, |
| | 2310, 2318, 2325-2327, 2346, 2389, 2392, 2398-2399, 2445, 2459, 2489, 2493, |
| | 2515, 2523, 2607, 2613, 2645, 2649, 2652, 2698, 2772, 2779, 2799, 2802, 2819, |
| | 2839, 2848, 2858, 3030, 3050, 3233, 3294, 3354, 3408, 3557 |
| Sub-Saharan Africa | 1, 4, 9-10, 28, 43, 45, 55-57, 59-60, 79, 83, 85, 93, 109, 125, 132, 134-139, 141- |
| | 145, 155-156, 159, 161, 168-169, 174, 193, 195-196, 213, 218, 221, 224, 238-239, |
| | 241, 282, 284-286, 324, 326, 363, 365, 369-371, 373, 406, 411, 418, 423, 433, |
| | 440, 447, 449, 455, 458, 463, 473, 496, 505, 520-521, 532, 568, 570, 601, 611, |
| | 623, 630, 639, 644, 647, 650, 672, 688, 711, 715, 734, 764, 775, 828, 849, 856, |
| | 863, 867, 872, 885-886, 909, 916, 931, 934, 944, 947, 953, 992-993, 999, 1004, |
| | 1020, 1024, 1036, 1065, 1097, 1109, 1132, 1149, 1176, 1207, 1217-1218, 1237, |
| | 1242, 1261-1263, 1295, 1317, 1319, 1335, 1347, 1382, 1408, 1420, 1428, 1457, |
| | 1465, 1495, 1516, 1528, 1533, 1587, 1625, 1633, 1639, 1657, 1661, 1666, 1678, |
| | 1692, 1781, 1802, 1813, 1817, 1837, 1846, 1849, 1854, 1863, 1870, 1880, 1886, |
| | 1954, 2019, 2046, 2049, 2060, 2062, 2065, 2088, 2096, 2100, 2174, 2208, 2223, |
| | 2259, 2290, 2297, 2310, 2316, 2318, 2376, 2387, 2393, 2398, 2424, 2445, 2475, |
| | 2481, 2525, 2584, 2606-2607, 2613, 2618, 2649, 2655, 2659, 2677, 2687-2688, |
| | 2698, 2719, 2907, 3039, 3108, 3240, 3311, 3367, 3394, 3452, 3618 |
| Western Asia | 1, 4, 9-10, 28, 43, 45, 55-57, 59, 79, 83, 88-89, 93, 100-101, 109, 124-125, 141- |
| | 145, 155-156, 168-170, 174, 192-193, 195-196, 238-239, 241, 271, 282-286, 304, |
| | 324, 326, 331, 339-342, 345, 365, 369-371, 373, 377, 406, 411, 418, 423, 433, |
| | 440, 447, 449, 455, 458, 463, 473, 496, 505, 520-521, 532, 570, 601, 611, 623, |
| | 639, 644, 647, 650, 672, 678, 686-688, 691, 711, 715, 734, 764, 773, 775, 783, |
| | 789, 794, 805, 828, 856, 863, 867, 872, 885-886, 909, 916, 931, 934, 944, 947, |
| | 953, 965, 988, 992-993, 999, 1004, 1015, 1020, 1024, 1036, 1052, 1054, 1066, |
| | 1097, 1109, 1149, 1176, 1207, 1217-1218, 1237, 1242, 1261-1263, 1317, 1319, |
| | 1347, 1372, 1382, 1405, 1408-1409, 1416, 1420, 1457, 1465, 1477, 1485, 1495, |
| | 1516, 1523, 1533, 1540, 1587, 1610, 1625, 1639, 1644, 1649, 1657, 1661, 1674, |
| | 1678, 1698, 1743, 1784, 1805, 1813, 1817, 1837, 1842, 1846, 1849, 1854, 1863, |
| | 1869, 1886, 1918, 1929, 1953-1954, 1961, 2001, 2027, 2062-2063, 2077, 2088, |
| | 2174, 2224, 2240, 2273, 2283, 2297, 2310, 2318, 2325, 2376, 2387, 2392, 2445, |
| | 2525, 2607, 2618-2619, 2645, 2647, 2649, 2657, 2677, 2772, 2802, 3108, 3169, |
| | 3290, 3294, 3329, 3355, 3382, 3432 |

TABLE 18
Region-specific peptide pools derived from NSP3 protein for CD8 T cell assays.

| Region | Seq. IDs of peptides |
|---|---|
| Australia | 2-3, 16-18, 34, 46-49, 51-52, 58, 61-62, 71, 77-78, 84, 86, 91, 96-99, 103, 107- |
| | 108, 110-121, 127, 130, 133, 140, 181, 183, 201-203, 215, 226, 233-234, 242, |
| | 249, 252, 261, 268, 289-296, 303, 307-309, 311, 313-318, 334, 337, 354-355, 372, |
| | 378, 381, 384, 397, 400-401, 403, 408-409, 415-417, 420-421, 436-438, 445, 456, |
| | 477, 489, 503, 509, 515, 523, 525, 527, 531, 533, 541, 544, 550, 554-555, 567, |
| | 586-587, 594, 607-608, 613, 621, 625-626, 631, 643, 674, 677, 680, 706-707, 712, |
| | 719, 726, 730, 733, 736, 755, 757-760, 763, 784-786, 791, 796, 800, 811, 813, |
| | 837, 847, 852, 878, 882, 884, 890, 893, 899-900, 904, 913-914, 925, 936, 942, |
| | 962, 970, 973, 976-977, 994, 1009, 1021-1022, 1035, 1051, 1056, 1061, 1068, |
| | 1071, 1079, 1086, 1094, 1110-1112, 1115, 1121, 1153, 1157, 1159, 1169, 1171, |
| | 1181-1182, 1185, 1211, 1216, 1221, 1228, 1231, 1236, 1257, 1267, 1297, 1302- |
| | 1303, 1312, 1330, 1334, 1337, 1349, 1365, 1422, 1426-1427, 1439, 1452, 1469, |
| | 1471, 1479, 1492, 1524, 1548, 1555, 1575, 1588, 1595, 1612, 1615, 1651, 1663, |
| | 1665, 1695, 1702, 1706, 1711, 1715, 1728-1729, 1745, 1754, 1774, 1810, 1818, |
| | 1821, 1834, 1836, 1845, 1859, 1861, 1864, 1867, 1871, 1884-1885, 1908, 1956, |
| | 1984, 2011, 2029, 2056, 2118, 2147, 2172, 2218, 2260, 2270, 2285, 2315, 2338, |
| | 2344, 2356, 2358, 2383, 2480, 2500, 2549, 2599, 2608, 2767, 2809, 2851, 2861, |
| | 3156 |
| Europe | 2-3, 16-18, 34, 46-49, 51-52, 58, 61-62, 71, 77-78, 84, 86, 91, 96-99, 103, 107- |
| | 108, 110-121, 130, 133, 140, 178, 181, 183, 201-203, 215, 226, 233-234, 242, |
| | 246-247, 249, 252, 259, 261, 267-268, 289-296, 303, 307-309, 311, 313-318, 328- |
| | 330, 334, 343, 355-360, 372, 378, 381, 384, 397, 400-401, 403, 408-409, 415-417, |
| | 420-421, 436-438, 445, 456, 467, 477, 489, 503, 509, 511, 515, 523, 527, 531, |
| | 555, 582, 586-587, 594, 603, 608, 621, 625-626, 677, 693, 707, 712, 720, 726, |
| | 730, 744-745, 755, 757, 759-760, 763, 784-785, 791, 800, 813, 837, 854, 878, |
| | 882, 884, 890, 893, 899-900, 904, 913-914, 925, 936, 942, 970, 973, 977, 1009, |
| | 1051, 1056, 1061, 1068, 1071, 1079, 1086, 1111, 1115, 1153, 1157, 1171, 1221, |
| | 1231, 1241, 1267, 1365, 1404, 1452, 1469, 1491-1492, 1524, 1532, 1565, 1588, |
| | 1612, 1618, 1660, 1663, 1704, 1728-1729, 1754, 1774, 1798, 1810, 1814, 1871, |
| | 1884, 1905, 1908, 1984, 2006, 2029, 2080, 2084, 2147, 2151, 2170, 2172, 2213, |
| | 2218, 2260, 2270, 2275, 2344, 2383, 2394, 2421, 2500, 2530, 2566, 2628, 2708, |
| | 2714, 2809, 2893, 2921, 2931, 2951, 3032, 3174, 3389, 3475, 3572, 3574 |
| North Africa | 2-3, 16-18, 34, 46, 51-52, 58, 61-62, 86, 91, 96-99, 103, 110, 130, 133, 140, 148- |
| | 150, 158, 181, 183, 226, 233-234, 242, 246, 249, 252, 267-268, 289-292, 307-309, |
| | 311, 313-318, 328, 337, 354-360, 372, 378, 381, 384, 397, 400-401, 403, 408-409, |
| | 415-417, 420-421, 436-438, 445, 456, 467, 477, 486, 489, 503, 515, 523, 525, |
| | 527, 531, 533, 544, 554-555, 586-587, 613, 621, 631, 643, 674, 677, 680, 712- |
| | 713, 719, 730, 733, 736, 755, 757-760, 763, 766, 784-786, 791, 796, 800, 809, |
| | 811, 813, 817, 837, 844, 852, 854, 878, 882, 884, 890, 893, 899-900, 904, 913- |
| | 914, 923, 925-926, 936, 942, 962, 970, 973, 976-977, 994, 1006, 1009, 1021- |
| | 1022, 1051, 1056, 1061, 1068, 1071, 1086, 1088, 1111, 1115, 1121, 1151-1153, |
| | 1159, 1169, 1181-1182, 1200, 1208, 1211, 1221, 1228, 1231, 1236, 1257, 1273, |
| | 1289, 1294, 1303, 1312, 1330, 1334, 1357, 1365, 1404, 1422, 1439, 1452, 1479, |
| | 1491-1492, 1494, 1497, 1508, 1548, 1555, 1571, 1575, 1583, 1588, 1612, 1615, |
| | 1618, 1620, 1651, 1663-1665, 1670, 1681, 1695-1696, 1702, 1707, 1711, 1715, |
| | 1728-1729, 1745, 1754, 1759-1760, 1774, 1787, 1791, 1810, 1818, 1834, 1843, |
| | 1860, 1864, 1867, 1877, 1884, 1908, 1948, 1950, 1956, 2005, 2011, 2026, 2029, |
| | 2048, 2050, 2056, 2102, 2135, 2147, 2172, 2176, 2178, 2187, 2202, 2218, 2249, |
| | 2258, 2260, 2285, 2317, 2334, 2338, 2358, 2379, 2383, 2388, 2485, 2490, 2500, |
| | 2508-2509, 2549, 2589, 2600, 2625, 2881 |
| North America | 2-3, 16-18, 34, 47-49, 51-52, 58, 61-62, 71, 77-78, 84, 86, 91, 96-99, 103, 110, |
| | 127, 130, 140, 181, 183, 201, 215, 226, 233-234, 242, 246-247, 249, 252, 259, |
| | 261, 267-268, 289-296, 303, 307-309, 311, 313-318, 328-330, 343, 354-360, 381, |
| | 384, 397, 400-401, 403, 408-409, 415-417, 420-421, 436-438, 445, 456, 467, 477, |
| | 489, 503, 509, 511-512, 515, 523, 525, 527, 531, 533, 538, 555, 558, 563, 567, |
| | 569, 582, 586, 603, 607-608, 621, 625-626, 631, 643, 707, 712, 719, 733, 736, |
| | 755, 757-760, 763, 811, 837, 847, 852, 878, 882, 884, 890, 893, 899-900, 904, |
| | 925, 936, 942, 970, 976-977, 1009, 1035, 1056, 1061, 1068, 1071, 1079, 1086, |
| | 1111, 1115, 1121, 1129, 1157, 1169, 1171, 1181-1182, 1216, 1221, 1228, 1231, |
| | 1236, 1243, 1249, 1257, 1267, 1297, 1303, 1330, 1349, 1365, 1426-1427, 1439, |
| | 1471, 1492, 1504, 1522, 1524, 1532, 1548, 1565, 1588, 1600, 1615, 1663, 1665, |
| | 1702, 1706, 1745, 1758, 1774, 1798, 1810, 1814, 1821, 1834, 1836, 1845, 1852, |
| | 1859, 1864, 1871, 1885, 1905, 2006, 2029, 2055, 2080, 2118, 2147, 2172, 2216, |
| | 2260, 2315, 2344, 2356, 2358, 2383, 2394-2395, 2480, 2500, 2518, 2599, 2608, |
| | 2663, 2668, 2767, 2809, 2851, 2855, 2893, 2931, 2951, 2962, 3389 |
| North East Asia | 34, 46-49, 71, 77-78, 84, 86, 91, 96-99, 103, 106-108, 110-121, 127, 130, 140, |
| | 181, 183, 201-202, 215, 226, 242, 246-247, 249, 252, 259, 261, 267-268, 289-296, |
| | 303, 311, 328-330, 334, 337, 343, 354-360, 372, 378, 381, 384, 400-401, 436-438, |
| | 445, 456, 467, 477, 486, 489, 503, 509, 511, 515, 523, 525, 527, 531, 538, 541, |
| | 544, 550, 554-555, 558, 563, 567, 569, 582, 587, 603, 607-608, 613, 621, 625- |
| | 626, 674, 677, 680, 706-707, 712, 719, 726, 730, 733, 755, 757-760, 763, 784- |
| | 786, 791, 796, 800, 811, 813, 817, 837, 847, 852, 878, 882, 884, 890, 893, 899- |
| | 900, 904, 913-914, 923, 925-926, 936, 942, 962, 968, 970, 973, 976-977, 995, |
| | 1009, 1018, 1021-1022, 1035, 1051, 1056, 1061, 1068, 1071, 1079, 1086, 1088, |
| | 1111-1112, 1115, 1121, 1152-1153, 1157-1159, 1169, 1171, 1181-1182, 1185, |
| | 1190, 1200, 1208, 1211, 1216, 1228, 1231, 1236, 1249, 1267, 1297, 1303, 1312, |
| | 1330, 1358, 1365, 1394, 1422, 1426-1427, 1468-1469, 1471, 1479, 1504, 1508, |
| | 1511, 1522, 1524, 1532, 1548, 1565, 1583, 1588, 1595, 1612, 1615-1616, 1620, |
| | 1651, 1663, 1665, 1670, 1684, 1704, 1706-1707, 1711, 1715, 1728-1729, 1745, |
| | 1759, 1774-1775, 1791, 1798, 1810, 1814, 1821, 1834, 1836, 1845, 1859-1861, |
| | 1864, 1871, 1877, 1905, 1908, 1948, 1984, 2006, 2050, 2055-2056, 2080, 2118, |
| | 2187, 2202, 2216, 2260, 2270, 2308, 2315, 2338, 2356, 2379, 2388, 2394, 2436, |
| | 2480, 2500, 2509, 2589, 2599, 2608, 2663, 2675, 2714, 2722, 2767, 2840, 2844, |
| | 2851, 2893, 2921, 2931, 2951, 3389, 3572 |
| Oceania | 34, 46-49, 51, 61, 71, 77-78, 84, 86, 91, 96-99, 103, 106-108, 110-121, 127, 130, |
| | 140, 181, 183, 201-202, 226, 242, 246, 249, 252, 261, 267-268, 289-296, 303, |
| | 311, 334, 337, 354-355, 372, 378, 381, 384, 397, 400-401, 408, 415, 421, 436- |
| | 438, 445, 456, 467, 477, 486, 489, 503, 509, 511-512, 515, 523, 525, 527, 531, |
| | 533, 538, 541, 544, 550, 554-555, 567, 582, 586-587, 594, 603, 607-608, 613, |
| | 621, 625-626, 631, 643, 674, 677, 680, 690, 693, 696, 706-707, 712-713, 719, |
| | 726, 730, 733, 736, 745, 755, 757-760, 763, 766, 772, 776, 784-786, 791, 796, |
| | 800, 811, 813, 817, 837, 844, 847, 852, 854, 878, 882, 884, 890, 893, 899-900, |
| | 904, 913-914, 923, 925-926, 936, 942, 962, 968, 970, 973, 976-977, 994-995, |
| | 1009, 1018, 1021, 1029, 1035, 1051, 1056, 1061, 1068, 1071, 1079, 1086, 1088, |
| | 1095, 1110-1112, 1115, 1121, 1129, 1152-1153, 1157-1159, 1169, 1171-1172, |
| | 1181-1182, 1185, 1190, 1208, 1211, 1216, 1221, 1228, 1231, 1236, 1249, 1257, |
| | 1267, 1297, 1303, 1312, 1330, 1334, 1337, 1349, 1365, 1394, 1404, 1422, 1426- |
| | 1427, 1464, 1469, 1471, 1478-1479, 1492, 1511, 1518, 1522, 1524, 1532, 1548, |
| | 1552, 1565, 1575, 1588, 1595, 1612, 1615-1616, 1651, 1663, 1665, 1684, 1695, |
| | 1702, 1704, 1706, 1715, 1728-1729, 1745, 1754, 1758, 1774-1775, 1798, 1810, |
| | 1814, 1818, 1821, 1828, 1834, 1836, 1845, 1852, 1859, 1864, 1867, 1871, 1885, |
| | 1908, 1957, 1984, 2006, 2011, 2029, 2080, 2111, 2118, 2147, 2172, 2202, 2216, |
| | 2260, 2270, 2285, 2308, 2315, 2338, 2344, 2356, 2412, 2436, 2480, 2500, 2517, |
| | 2530, 2549, 2599, 2608, 2624, 2663, 2675, 2684, 2714, 2722, 2755, 2767, 2844, |
| | 2851, 2855, 2861, 2893, 3057, 3156 |
| South and Central America | 2-3, 16-18, 34, 46-49, 51-52, 58, 61-62, 77-78, 86, 91, 96-99, 103, 106, 110, 127, |
| | 140, 181, 183, 201-203, 226, 242, 249, 267-268, 289-292, 303, 311, 328-330, 334, |
| | 337, 343, 354-360, 378, 381, 384, 397, 400-401, 403, 408-409, 415-417, 420-421, |
| | 436-438, 445, 456, 467, 477, 486, 489, 503, 509, 511, 515, 523, 525, 527, 531, |
| | 533, 544, 555, 567, 582, 587, 603, 607-608, 613, 621, 625-626, 631, 643, 674, |
| | 677, 706-707, 712, 719, 726, 730, 733, 736, 755, 757-760, 763, 766, 784-786, |
| | 791, 796, 800, 811, 813, 837, 847, 852, 878, 882, 884, 890, 893, 899-900, 904, |
| | 913-914, 923, 925-926, 936, 942, 962, 970, 973, 976-977, 994, 1009, 1021, 1035, |
| | 1061, 1068, 1071, 1079, 1086, 1088, 1094, 1110, 1112, 1115, 1121, 1152-1153, |
| | 1157-1159, 1169, 1181-1182, 1185, 1208, 1221, 1228, 1231, 1257, 1266-1267, |
| | 1273, 1302-1303, 1312, 1330, 1334, 1337, 1349, 1357, 1365, 1422, 1426, 1439, |
| | 1469, 1479, 1492, 1508, 1513, 1524, 1532, 1548, 1555, 1565, 1575, 1588, 1595, |
| | 1612, 1615, 1618, 1651, 1663, 1665, 1684, 1695, 1702, 1704, 1706, 1728-1729, |
| | 1745, 1774-1775, 1791, 1798, 1810, 1814, 1818, 1821, 1834, 1845, 1861, 1864, |
| | 1867, 1871, 1895, 1905, 1908, 1948, 1956, 1984, 2006, 2011, 2029, 2080, 2104, |
| | 2118, 2147, 2172, 2260, 2270, 2285, 2308, 2338, 2344, 2356, 2358, 2371, 2383, |
| | 2394, 2436, 2438, 2480, 2500, 2509, 2549, 2608, 2624, 2661, 2691, 2714, 2722, |
| | 2749, 2809, 2893, 2921, 2931, 2951, 3389, 3572 |
| South Asia | 2-3, 16-18, 34, 46-49, 51-52, 58, 61-62, 71, 77-78, 84, 86, 91, 96-99, 103, 106, |
| | 110, 127, 130, 140, 181, 183, 201-203, 226, 242, 246, 249, 252, 267-268, 289- |
| | 292, 307-308, 311, 313, 328, 334, 337, 354-360, 372, 378, 380-384, 397, 400-401, |
| | 403, 408-409, 415-417, 420-421, 436-438, 445, 456, 467, 477, 486, 489, 503, 509, |
| | 515, 523, 525, 527, 531, 533, 538, 544, 555, 582, 586-587, 594, 603, 607-608, |
| | 613, 621, 625-626, 631, 643, 677, 680, 690, 693, 696, 707, 712-713, 719-720, |
| | 726, 730, 733, 736, 745, 755, 757-760, 763, 766, 772, 776, 784-786, 791, 796, |
| | 800, 809, 813, 817, 837, 844, 847, 852, 854, 878, 882, 884, 890, 893, 899-900, |
| | 904, 913-914, 923, 925-926, 936, 942, 962, 968, 970, 973, 976-977, 994, 1006, |
| | 1009, 1021-1022, 1029, 1035, 1051, 1056, 1061, 1068, 1071, 1086, 1088, 1111- |
| | 1112, 1115, 1121, 1151-1153, 1157, 1159, 1169, 1181-1182, 1185, 1190, 1200, |
| | 1208, 1211, 1221, 1228, 1231, 1257, 1266-1267, 1273, 1289, 1303, 1312, 1330, |
| | 1334, 1357, 1365, 1394, 1404, 1422, 1426, 1452, 1469, 1471, 1479, 1491-1492, |
| | 1508, 1511, 1513, 1518, 1524, 1532, 1555, 1565, 1571, 1575, 1583, 1591, 1595, |
| | 1612, 1615-1616, 1620, 1651, 1663, 1665, 1670, 1684, 1695, 1711, 1715, 1728- |
| | 1729, 1745, 1754, 1760, 1774-1775, 1791, 1798, 1810, 1843, 1861, 1864, 1867, |
| | 1871, 1877, 1884, 1891, 1908, 1933, 1948, 1984, 2006, 2029, 2033, 2048, 2050, |
| | 2056, 2080, 2147, 2172, 2178, 2187, 2202, 2216, 2249, 2258, 2260, 2270, 2308, |
| | 2334, 2338, 2344, 2379-2380, 2383, 2412, 2436, 2441, 2500, 2509, 2517, 2530, |
| | 2598, 2600, 2628, 2663, 2675, 2691, 2714, 2722, 2751, 2755, 2809, 2822, 2844, |
| | 2893 |
| South East Asia | 34, 46-49, 51, 61, 71, 77-78, 84, 86, 91, 96-99, 103, 110, 127, 140, 181, 183, 201- |
| | 202, 226, 242, 246-247, 249, 252, 259, 261, 268, 289-296, 303, 311, 354-360, |
| | 372, 378, 381, 384, 397, 400-401, 403, 408-409, 415-417, 421, 436-438, 445, 456, |
| | 467, 477, 486, 489, 503, 509, 511-512, 515, 523, 525, 527, 531, 538, 541, 555, |
| | 567, 582, 586-587, 594, 603, 607-608, 613, 621, 625-626, 643, 674, 677, 680, |
| | 690, 693, 696, 706-707, 712-713, 719, 726, 730, 733, 745, 755, 757-760, 763, |
| | 772, 776, 784-785, 791, 811, 813, 817, 837, 844, 847, 852, 854, 878, 882, 884, |
| | 890, 893, 899-900, 904, 913-914, 923, 925-926, 936, 942, 962, 968, 970, 973, |
| | 976-977, 994, 1009, 1022, 1029, 1035, 1051, 1056, 1061, 1068, 1071, 1079, 1086, |
| | 1088, 1094-1095, 1110-1112, 1115, 1121, 1129, 1144, 1152-1153, 1157-1159, |
| | 1169, 1171-1172, 1182, 1208, 1211, 1231, 1236, 1241, 1249, 1266-1267, 1273, |
| | 1285, 1297, 1302-1303, 1330, 1349, 1365, 1404, 1426, 1464, 1469, 1471, 1478, |
| | 1491, 1508, 1511, 1513, 1522, 1524, 1532, 1548, 1555, 1565, 1588, 1591, 1595, |
| | 1615-1616, 1660, 1663, 1704, 1706-1707, 1711, 1758, 1774-1775, 1787, 1795, |
| | 1798, 1807-1808, 1810, 1814, 1821, 1834, 1836, 1845, 1852, 1859, 1861, 1864, |
| | 1871, 1933, 1948, 1957, 2006, 2039, 2056, 2080, 2104, 2111, 2118, 2170, 2202, |
| | 2216, 2258, 2260, 2308, 2313, 2315, 2328, 2356, 2365, 2403, 2436, 2480, 2500, |
| | 2509, 2589, 2599, 2608, 2615, 2656, 2663, 2675, 2714, 2718, 2722, 2767, 2775, |
| | 2840, 2844, 2851, 2893 |
| Sub-Saharan Africa | 2-3, 16-18, 34, 47-49, 51-52, 58, 61-62, 71, 77-78, 84, 86, 91, 103, 110, 127, 130, |
| | 133, 140, 148-150, 158, 181, 183, 201, 215, 226, 233-234, 242, 246, 249, 268, |
| | 354-355, 378, 381, 384, 397, 400-401, 403, 408-409, 415-417, 420-421, 436-438, |
| | 445, 456, 467, 477, 489, 503, 509, 511-512, 515, 523, 525, 527, 531, 533, 538, |
| | 544, 554-555, 558, 563, 567, 569, 582, 586-587, 603, 607-608, 613, 621, 625-626, |
| | 631, 643, 677, 707, 712, 719, 733, 736, 755, 757-760, 763, 766, 811, 837, 852, |
| | 878, 882, 884, 890, 893, 899-900, 904, 923, 925, 936, 942, 962, 970, 973, 976- |
| | 977, 994, 1006, 1009, 1021-1022, 1035, 1056, 1061, 1068, 1071, 1079, 1086, |
| | 1088, 1111, 1115, 1121, 1153, 1157, 1169, 1171, 1181-1182, 1200, 1216, 1221, |
| | 1228, 1231, 1236, 1257, 1267, 1273, 1294, 1297, 1330, 1349, 1358, 1365, 1427, |
| | 1439, 1468, 1471, 1492, 1494, 1497, 1522, 1524, 1548, 1555, 1565, 1588, 1615, |
| | 1618, 1663, 1665, 1681, 1702, 1704, 1707, 1715, 1745, 1758-1759, 1774, 1791, |
| | 1810, 1814, 1818, 1834, 1860, 1864, 1867, 1871, 1884, 1905, 1956, 2005-2006, |
| | 2011, 2026, 2029, 2048, 2102, 2104, 2147, 2172, 2178, 2246, 2248, 2260, 2285, |
| | 2358, 2383, 2388, 2394, 2485, 2490, 2500, 2508, 2526, 2549, 2625, 2656, 2855, |
| | 2899, 2951, 2954, 3006, 3248, 3283, 3435 |
| Western Asia | 2-3, 16-18, 34, 46-49, 51-52, 61, 71, 77-78, 84, 86, 91, 96-99, 103, 106-108, 110- |
| | 121, 127, 130, 140, 181, 183, 201, 226, 233-234, 242, 246, 249, 252, 267-268, |
| | 289-291, 307-308, 311, 313, 328-330, 334, 337, 343, 354-355, 372, 378, 381, 384, |
| | 397, 400-401, 403, 408-409, 415-417, 420-421, 436-438, 445, 456, 467, 477, 486, |
| | 489, 503, 509, 511-512, 515, 523, 525, 527, 531, 533, 538, 555, 567, 582, 586- |
| | 587, 594, 603, 607-608, 613, 621, 625-626, 631, 643, 674, 677, 680, 690, 693, |
| | 696, 707, 712, 719-720, 726, 730, 733, 736, 745, 755, 757-760, 763, 766, 772, |
| | 776, 784-785, 791, 800, 813, 837, 844, 854, 878, 882, 884, 890, 893, 899-900, |
| | 904, 913-914, 923, 925-926, 936, 942, 962, 970, 973, 976-977, 994, 1009, 1021- |
| | 1022, 1029, 1035, 1051, 1056, 1061, 1068, 1071, 1086, 1088, 1094-1095, 1110- |
| | 1111, 1115, 1121,1144, 1152-1153, 1157, 1159, 1169, 1172, 1181-1182, 1185, |
| | 1200, 1208, 1211, 1221, 1228, 1243, 1257, 1266-1267, 1273, 1285, 1303, 1334, |
| | 1349, 1357-1358, 1365, 1426, 1452, 1464, 1469-1471, 1492, 1508, 1513, 1532, |
| | 1555, 1565, 1575, 1583, 1612, 1618, 1620, 1663, 1665, 1670, 1695, 1704, 1711, |
| | 1722, 1728-1729, 1754, 1758, 1774-1775, 1787, 1791, 1798, 1810, 1852, 1858, |
| | 1861, 1864, 1867, 1877, 1884, 1895, 1905, 1908, 1957, 1970, 1984, 2006-2007, |
| | 2024, 2029, 2033, 2039, 2050, 2056, 2084, 2111, 2147, 2172, 2187, 2202, 2218, |
| | 2260, 2270, 2275, 2344, 2379, 2383, 2394-2395, 2412, 2500, 2518, 2538, 2566, |
| | 2615-2616, 2678, 2708, 2717, 2722, 2753, 2776, 2786, 2809, 2893, 2931, 3113, |
| | 3389 |

TABLE 19
Region-specific peptide pools derived from NSP4 protein for CD8 T cell assays.

| Region | Seq. IDs of peptides |
|---|---|
| Australia | 5, 23-25, 44, 90, 94-95, 163-165, 217, 251, 269, 274, 278, 287-288, 346, 410, 429, |
| | 450, 453, 470-471, 482-483, 536, 575, 597, 599, 609, 624, 635, 655, 659, 664, |
| | 679, 682, 689, 703, 716, 737, 792, 803, 826-827, 857, 866, 877, 902, 905, 945, |
| | 957, 971-972, 1030, 1055, 1059, 1072, 1113, 1117, 1136, 1177, 1197, 1224, 1226, |
| | 1229, 1244, 1259, 1268-1269, 1277, 1299, 1314, 1373, 1390, 1400, 1406-1407, |
| | 1414, 1462, 1466, 1512, 1515, 1527, 1576, 1608, 1635, 1673, 1712, 1717, 1732, |
| | 1741, 1771, 1778, 1797, 1809, 1826, 1893, 1935, 1945, 1952, 1958, 1992, 2008, |
| | 2016, 2031, 2041, 2101, 2140-2141, 2157, 2203-2204, 2234, 2241, 2264, 2322, |
| | 2331, 2355, 2377, 2396, 2420, 2443, 2464, 2532, 2602, 2614, 2640, 2721, 2723, |
| | 2780, 2788, 2903, 2935, 2941, 2997, 3028, 3081, 3120, 3159, 3206, 3237, 3242, |
| | 3453, 3520, 3579, 3611, 3720, 3744 |
| Europe | 5, 23-25, 44, 90, 94-95, 164-167, 217, 251, 269-270, 274, 277-279, 281, 287-288, |
| | 346-349, 410, 429, 450, 453, 470-471, 482, 536, 561, 573, 575, 597, 599, 609, |
| | 624, 635, 659, 664, 682, 703, 716, 737, 742, 782, 826-827, 857, 866, 877, 902, |
| | 905, 971-972, 985, 1030, 1059, 1113, 1117, 1133, 1177, 1197, 1224, 1226, 1229, |
| | 1244, 1259, 1268-1269, 1277, 1299, 1314, 1373, 1377, 1390, 1400, 1406-1407, |
| | 1414, 1462, 1512, 1576, 1635, 1717, 1771, 1809, 1893, 1935, 1945, 1958, 2008, |
| | 2041, 2101, 2140-2141, 2302, 2322, 2331, 2372, 2420, 2443, 2461, 2464, 2532, |
| | 2555, 2594, 2621, 2676, 2788, 2903, 2927, 2941, 2952, 3155, 3206, 3245, 3344, |
| | 3347, 3393, 3707, 3800 |
| North Africa | 5, 23-25, 41, 44, 90, 94-95, 217, 269-270, 274, 277-279, 281, 287, 346-349, 410, |
| | 429, 450, 453, 470-471, 482-483, 536, 575, 597, 635, 655, 659, 664, 679, 682, |
| | 689, 703, 716, 737, 792, 803, 826-827, 857, 866, 877, 902, 905, 945, 957, 959, |
| | 972, 1030, 1055, 1059, 1072, 1113, 1117, 1136, 1177, 1180, 1197, 1224, 1226, |
| | 1229, 1259, 1268, 1277, 1299, 1314, 1373, 1390, 1400, 1406-1407, 1414, 1462, |
| | 1466, 1512, 1572, 1576, 1605, 1635, 1673, 1712, 1717, 1732, 1741, 1771, 1778, |
| | 1797, 1809, 1826, 1893, 1923, 1945, 1952, 1958, 1973, 2008, 2031, 2041, 2101, |
| | 2140-2141, 2203-2204, 2234, 2241, 2322, 2331, 2396, 2420, 2425, 2443, 2461, |
| | 2464, 2532, 2536, 2602, 2640, 2710, 2780, 2788, 2814, 2903, 2935, 3028, 3071, |
| | 3081-3082, 3085, 3089, 3155, 3162, 3206, 3237, 3242, 3279, 3344, 3413, 3504, |
| | 3509, 3523, 3539 |
| North America | 5, 23-25, 44, 90, 94-95, 163-167, 217, 251, 269-270, 274, 277-279, 281, 287-288, |
| | 346-349, 410, 429, 450, 453, 470-471, 482-483, 536, 539, 561, 573, 575, 597, |
| | 599, 609, 624, 635, 655, 659, 664, 682, 703, 716, 827, 866, 877, 902, 905, 945, |
| | 957, 971-972, 985, 1030, 1059, 1072, 1117, 1136, 1163, 1177, 1197, 1219, 1224, |
| | 1259, 1268-1269, 1277, 1314, 1373, 1377, 1390, 1400, 1407, 1462, 1466, 1512, |
| | 1515, 1527, 1576, 1608, 1717, 1736, 1741, 1771, 1797, 1809, 1923-1924, 1979, |
| | 1992, 2031, 2041, 2101, 2112, 2141, 2157, 2186, 2204, 2219, 2241, 2322, 2331, |
| | 2355, 2420, 2425, 2464, 2516, 2532, 2555, 2614, 2621, 2640, 2721, 2723, 2795, |
| | 2903, 2941, 2997, 3028, 3071, 3159, 3206, 3237, 3245, 3347, 3393, 3453, 3520, |
| | 3523, 3579, 3611 |
| North East Asia | 5, 23-25, 41, 44, 90, 94-95, 163-167, 217, 251, 269-270, 274, 277-279, 281, 287- |
| | 288, 346-349, 450, 453, 470-471, 482-483, 536, 539, 561, 573, 575, 597, 599, |
| | 609, 624, 635, 655, 659, 664, 679, 682, 689, 703, 716, 792, 803, 826-827, 866, |
| | 877, 902, 905, 945, 957, 959, 971-972, 985, 1030, 1057, 1059, 1072, 1113, 1117, |
| | 1136, 1163, 1177, 1197, 1224, 1226, 1229, 1244, 1259, 1268-1269, 1277, 1299, |
| | 1314, 1329, 1373, 1377, 1390, 1400, 1406-1407, 1414, 1462, 1512, 1515, 1527, |
| | 1567, 1572, 1576, 1635, 1701, 1712, 1717, 1732, 1736, 1771, 1778, 1797, 1809, |
| | 1826, 1893, 1923-1924, 1945, 1973, 1992, 2008, 2016, 2031, 2041, 2101, 2140- |
| | 2141, 2157, 2182, 2186, 2203, 2234, 2241, 2322, 2331, 2355, 2372, 2377, 2396, |
| | 2420, 2425, 2464, 2516, 2536, 2555, 2579, 2614, 2621, 2639-2640, 2710, 2721, |
| | 2780, 2795, 2814, 2903, 2935, 2941, 2997, 3028, 3081, 3089, 3155, 3159, 3206, |
| | 3237, 3245, 3347, 3393, 3413, 3453, 3504, 3520, 3523, 3579, 3611, 3720, 3830 |
| Oceania | 5, 23-25, 41, 44, 90, 94-95, 163-167, 251, 269-270, 274, 277-279, 281, 287-288, |
| | 346-347, 450, 453, 470-471, 482-483, 536, 539, 561, 573, 575, 597, 599, 609, |
| | 624, 635, 655, 659, 664, 679, 682, 689, 703, 716, 737, 782, 792, 803, 826-827, |
| | 857, 866, 877, 902, 905, 945, 957, 959, 971-972, 985, 1030, 1055, 1057, 1059, |
| | 1072, 1113, 1117, 1133, 1136, 1163, 1177, 1197, 1219, 1224, 1226, 1229, 1244, |
| | 1259, 1268-1269, 1277, 1299, 1314, 1329, 1373, 1377, 1390, 1400, 1406-1407, |
| | 1414, 1462, 1466, 1512, 1515, 1527, 1557, 1567, 1572, 1576, 1608, 1635, 1673, |
| | 1701, 1712, 1717, 1732, 1736, 1741, 1771, 1778, 1797, 1809, 1826, 1893, 1923- |
| | 1924, 1935, 1945, 1952, 1958, 1973, 1979, 1992, 2008, 2016, 2031, 2041, 2101, |
| | 2112, 2140-2141, 2157, 2182, 2186, 2203-2204, 2234, 2241, 2264, 2322, 2331, |
| | 2355, 2357, 2372, 2377, 2396, 2420, 2425, 2443, 2461, 2464, 2516, 2532, 2579, |
| | 2602, 2614, 2621, 2639-2640, 2644, 2721, 2723, 2727, 2780, 2876, 2903, 2935, |
| | 2941, 2997, 3028, 3041, 3061, 3076, 3081, 3120, 3159, 3206, 3237, 3242, 3245, |
| | 3347, 3393, 3444, 3453, 3509, 3520, 3542, 3579, 3611, 3720, 3744, 3790, 3820, |
| | 3830 |
| South and Central America | 5, 23-25, 44, 90, 94-95, 163-167, 217, 269-270, 274, 277-279, 281, 287, 346-349, |
| | 410, 429, 450, 453, 470-471, 482-483, 536, 573, 575, 597, 599, 609, 624, 635, |
| | 655, 659, 664, 679, 682, 689, 703, 716, 737, 782, 792, 803, 826-827, 866, 877, |
| | 902, 905, 945, 957, 959, 972, 985, 1030, 1055, 1059, 1072, 1113, 1117, 1136, |
| | 1163, 1177, 1197, 1224, 1226, 1229, 1259, 1268-1269, 1277, 1299, 1314, 1373, |
| | 1377, 1390, 1400, 1406-1407, 1414, 1462, 1466, 1512, 1527, 1572, 1576, 1605, |
| | 1608, 1635, 1673, 1712, 1717, 1732, 1741, 1771, 1778, 1797, 1809, 1826, 1893, |
| | 1952, 1958, 1979, 1992, 2008, 2016, 2031, 2041, 2101, 2141, 2157, 2182, 2186, |
| | 2204, 2234, 2241, 2322, 2331, 2357, 2372, 2377, 2396, 2420, 2425, 2443, 2464, |
| | 2532, 2536, 2555, 2614, 2621, 2710, 2721, 2723, 2727, 2780, 2788, 2876, 2903, |
| | 2935, 2941, 3076, 3081, 3159, 3206, 3237, 3245, 3347, 3393, 3453, 3720 |
| South Asia | 5, 23-25, 41, 44, 90, 94-95, 163-167, 217, 269, 274, 277-279, 287, 346-349, 385- |
| | 386, 410, 429, 450, 453, 470-471, 482-483, 536, 539, 561, 573, 575, 597, 599, |
| | 609, 624, 635, 655, 659, 664, 679, 682, 689, 703, 716, 737, 782, 792, 803, 826- |
| | 827, 857, 866, 877, 902, 905, 945, 957, 959, 971-972, 1030, 1055, 1057, 1059, |
| | 1072, 1113, 1117, 1133, 1136, 1177, 1180, 1197, 1224, 1226, 1229, 1259, 1268, |
| | 1277, 1299, 1314, 1329, 1373, 1377, 1390, 1400, 1406-1407, 1414, 1432, 1462, |
| | 1466, 1512, 1515, 1527, 1567, 1572, 1576, 1605, 1635, 1673, 1701, 1712, 1717, |
| | 1732-1733, 1736, 1741, 1771, 1778, 1809, 1826, 1893, 1935, 1945, 1958, 2008, |
| | 2016, 2041, 2101, 2140-2141, 2186, 2203-2204, 2234, 2241, 2322, 2331, 2357, |
| | 2372, 2377, 2396, 2420, 2425, 2443, 2461, 2464, 2516, 2532, 2536, 2579, 2614, |
| | 2621, 2639-2640, 2644, 2710, 2721, 2727, 2780, 2788, 2814, 2903, 2935, 2941, |
| | 3081, 3085, 3089, 3155, 3162, 3206, 3237, 3242, 3245, 3344, 3347, 3393, 3413, |
| | 3444, 3504, 3539, 3701, 3720, 3830 |
| South East Asia | 5, 23-25, 41, 44, 90, 94-95, 163-167, 251, 269, 274, 278, 287-288, 346-349, 410, |
| | 450, 453, 470-471, 482-483, 536, 561, 573, 575, 597, 599, 609, 624, 635, 655, |
| | 659, 664, 679, 682, 689, 703, 716, 737, 742, 782, 827, 857, 866, 877, 902, 905, |
| | 945, 957, 959, 971-972, 985, 1030, 1055, 1057, 1059, 1072, 1113, 1117, 1133, |
| | 1136, 1163, 1177, 1180, 1197, 1219, 1224, 1229, 1244, 1259, 1268-1269, 1277, |
| | 1299, 1314, 1325, 1373, 1377, 1390, 1400, 1406-1407, 1414, 1432, 1462, 1512, |
| | 1515, 1527, 1557, 1567, 1572, 1576, 1608, 1712, 1717, 1732, 1736, 1771, 1797, |
| | 1809, 1826, 1923-1924, 1945, 1952, 1979, 1992, 2008, 2016, 2031, 2041, 2101, |
| | 2112, 2140-2141, 2157, 2182, 2203, 2234, 2241, 2264, 2322, 2331, 2355, 2372, |
| | 2377, 2396, 2420, 2425, 2461, 2464, 2536, 2614, 2621, 2640, 2710, 2721, 2723, |
| | 2727, 2903, 2935, 2941, 2997, 3028, 3061, 3071, 3076, 3146, 3159, 3206, 3237, |
| | 3245, 3347, 3393, 3425, 3444, 3453, 3509, 3520, 3523, 3542, 3567, 3579, 3611, |
| | 3707, 3732, 3788, 3830 |
| Sub-Saharan Africa | 5, 23-25, 41, 44, 90, 94-95, 163-167, 217, 269, 277-278, 346, 410, 429, 450, 453, |
| | 470-471, 482-483, 536, 539, 561, 573, 575, 597, 599, 609, 624, 635, 655, 659, |
| | 664, 682, 703, 716, 737, 827, 857, 866, 877, 902, 905, 945, 957, 971-972, 985, |
| | 1030, 1055, 1059, 1072, 1117, 1136, 1177, 1197, 1219, 1224, 1229, 1259, 1268- |
| | 1269, 1277, 1314, 1329, 1373, 1377, 1390, 1400, 1406-1407, 1414, 1432, 1462, |
| | 1466, 1512, 1515, 1527, 1572-1573, 1576, 1605, 1608, 1673, 1712, 1717, 1733, |
| | 1736, 1741, 1771, 1778, 1797, 1809, 1826, 1923-1924, 1935, 1945, 1952, 1973, |
| | 1979, 2031, 2041, 2101, 2112, 2140-2141, 2203-2204, 2219, 2234, 2241, 2322, |
| | 2331, 2396, 2420, 2425, 2461, 2464, 2532, 2602, 2640, 2814, 2876, 2903, 2935, |
| | 2941, 2977, 3071, 3206, 3224, 3245, 3279, 3324-3325, 3363, 3393, 3460, 3509, |
| | 3523, 3564, 3781 |
| Western Asia | 5, 23-25, 41, 44, 90, 94-95, 163-167, 269-270, 274, 277-279, 281, 287, 346, 410, |
| | 429, 450, 453, 470-471, 482-483, 536, 539, 561, 573, 575, 597, 599, 609, 624, |
| | 635, 655, 659, 664, 679, 682, 689, 703, 716, 737, 782, 826-827, 857, 866, 877, |
| | 902, 905, 945, 957, 959, 971-972, 985, 1030, 1055, 1059, 1113, 1117, 1133, 1163, |
| | 1177, 1180, 1197, 1219, 1224, 1226, 1229, 1259, 1268-1269, 1277, 1299, 1314, |
| | 1325, 1373, 1377, 1390, 1400, 1406-1407, 1414, 1432, 1462, 1466, 1512, 1527, |
| | 1557, 1572, 1576, 1608, 1635, 1712, 1717, 1732-1733, 1736, 1741, 1771, 1778, |
| | 1809, 1826, 1893, 1924, 1935, 1945, 1952, 1958, 1979, 2008, 2041, 2101, 2112, |
| | 2140-2141, 2182, 2203-2204, 2234, 2241, 2322, 2331, 2357, 2372, 2420, 2425, |
| | 2443, 2464, 2532, 2555, 2579, 2621, 2644, 2710, 2723, 2727, 2788, 2814, 2876, |
| | 2903, 2935, 3061, 3076, 3089, 3155, 3206, 3237, 3242, 3245, 3344, 3347, 3364, |
| | 3393, 3413, 3425, 3444, 3504, 3509, 3523, 3530, 3707 |

TABLE 20
Region-specific peptide pools derived from NSP14 protein for CD8 T cell assays.

| Region | Seq. IDs of peptides |
|---|---|
| Australia | 7, 75, 87, 230, 243, 262, 264, 405, 478, 485, 490, 493, 529, 542, 546, 551, 560, |
| | 564, 574, 638, 645, 665, 684, 709, 743, 761, 778, 818, 821, 824-825, 851, 859, |
| | 865, 871, 910, 915, 927, 950, 1005, 1016, 1041, 1118, 1123, 1126, 1141, 1188, |
| | 1193, 1222, 1225, 1230, 1234, 1240, 1251, 1293, 1305, 1322, 1355, 1384, 1411, |
| | 1424, 1447, 1473, 1484, 1503, 1543, 1561, 1589, 1676, 1716, 1742, 1767, 1796, |
| | 1838, 1841, 1906, 1922, 1994, 2009, 2021, 2054, 2090, 2120, 2124, 2156, 2179, |
| | 2185, 2193, 2225, 2269, 2284, 2321, 2418, 2465, 2472, 2486-2487, 2501-2502, |
| | 2519, 2522, 2535, 2543, 2577, 2633, 2637, 2681, 2738, 2828, 2841, 2867, 2936, |
| | 2944, 3075, 3086, 3124, 3177, 3196, 3213, 3247, 3270, 3275, 3281-3282, 3361, |
| | 3410, 3440, 3535, 3549, 3558, 3568, 3575, 3600, 3651, 3664, 3686, 3705, 3808, |
| | 3819 |
| Europe | 7, 75, 87, 230, 243, 262, 264, 333, 405, 478, 490, 493, 500, 529, 542, 546, 551, |
| | 560, 564, 574, 665, 684, 709, 741, 743, 761, 818, 824-825, 859, 865, 871, 910, |
| | 915, 927, 1005, 1041, 1118, 1123, 1126, 1188, 1193, 1222, 1225, 1234, 1240, |
| | 1305, 1309, 1322, 1355, 1384, 1424, 1473, 1598, 1667, 1694, 1713, 1716, 1742, |
| | 1841, 1921, 1994, 2009, 2021, 2054, 2089-2090, 2156, 2196, 2225, 2269, 2321, |
| | 2332, 2428, 2465, 2472, 2479, 2486-2487, 2502-2503, 2519, 2522, 2564, 2573, |
| | 2610, 2738, 3052, 3200, 3281, 3333, 3361, 3409, 3440, 3478, 3535, 3540, 3597, |
| | 3626, 3651, 3697, 3808 |
| North Africa | 7, 87, 230, 243, 262, 264, 405, 478, 485, 490, 493, 529, 542, 546, 551, 560, 638, |
| | 645, 665, 684, 709, 743, 761, 818, 821, 824-825, 851, 859, 865, 871, 910, 915, |
| | 927, 950, 1005, 1041, 1118, 1123, 1126, 1188, 1193, 1222, 1225, 1230, 1234, |
| | 1240, 1251, 1305, 1355, 1384, 1411, 1424, 1447, 1473, 1484, 1503, 1543, 1547, |
| | 1589, 1611, 1676, 1716, 1742, 1777, 1796, 1865, 1922, 1994, 2054, 2090, 2156, |
| | 2179, 2195, 2225, 2269, 2284, 2321, 2368, 2428-2429, 2444, 2465, 2472, 2486- |
| | 2487, 2502, 2535, 2561, 2564, 2610, 2701, 2738, 2828, 2936, 2998, 3086, 3270, |
| | 3274, 3281-3282, 3361, 3410, 3414, 3440, 3467, 3506, 3533, 3549, 3552, 3651, |
| | 3705, 3763, 3780, 3819 |
| North America | 7, 75, 87, 230, 243, 262, 264, 333, 405, 478, 490, 493, 500, 529, 551, 560, 564, |
| | 574, 638, 665, 709, 743, 761, 851, 859, 865, 871, 910, 915, 927, 1005, 1041, |
| | 1118, 1123, 1126, 1188, 1193, 1222, 1230, 1240, 1251, 1293, 1305, 1309, 1322, |
| | 1355, 1384, 1424, 1484, 1543, 1589, 1598, 1667, 1716, 1767, 1796, 1838, 1841, |
| | 1921-1922, 1994, 2009, 2021, 2054, 2059, 2075, 2089-2090, 2120, 2124, 2185, |
| | 2196, 2225, 2321, 2418, 2465, 2472, 2479, 2486-2487, 2501-2502, 2514, 2519, |
| | 2522, 2535, 2573, 2637, 2642, 2738, 2841, 2867, 2877, 2944, 2983, 3052, 3124, |
| | 3247, 3270, 3281-3282, 3361, 3410, 3440, 3467, 3550, 3558, 3568, 3575, 3600, |
| | 3651, 3808, 3814 |
| North East Asia | 75, 87, 230, 243, 262, 264, 333, 478, 485, 490, 493, 500, 529, 542, 546, 551, 560, |
| | 564, 574, 638, 665, 684, 709, 743, 761, 818, 821, 824-825, 851, 859, 865, 871, |
| | 910, 915, 927, 950, 1005, 1016, 1041, 1118, 1123, 1126, 1141, 1188, 1193, 1222, |
| | 1225, 1230, 1234, 1240, 1251, 1293, 1305, 1309, 1322, 1355, 1384, 1411, 1424, |
| | 1447, 1484, 1503, 1543, 1561, 1598, 1611, 1667, 1716, 1742, 1767, 1796, 1838, |
| | 1841, 1906, 1921-1922, 1994, 2009, 2021, 2054, 2059, 2075, 2089-2090, 2124, |
| | 2156, 2185, 2193, 2196, 2225, 2269, 2284, 2321, 2368, 2418, 2428, 2465, 2472, |
| | 2479, 2486, 2502, 2519, 2522, 2561, 2573, 2577, 2642, 2664, 2738, 2828, 2841, |
| | 2867, 2877, 2891, 2936, 2944, 2998, 3002, 3052, 3075, 3086, 3124, 3213, 3270, |
| | 3274, 3281, 3341, 3410, 3424, 3427, 3467, 3533, 3535, 3550, 3558, 3568, 3575, |
| | 3600, 3664, 3763, 3814 |
| Oceania | 75, 87, 230, 243, 262, 264, 405, 478, 485, 490, 493, 500, 529, 542, 546, 551, 560, |
| | 564, 574, 638, 645, 665, 684, 698, 709, 743, 761, 770, 778, 818, 821, 824-825, |
| | 851, 859, 865, 871, 910, 915, 927, 950, 1005, 1016, 1041, 1098, 1118, 1123, |
| | 1126, 1141, 1188, 1193, 1222, 1225, 1230, 1234, 1240, 1251, 1293, 1305, 1309, |
| | 1322, 1348, 1355, 1384, 1411, 1424, 1447, 1456, 1473, 1484, 1503, 1543, 1561, |
| | 1574, 1598, 1611, 1667, 1676, 1694, 1713, 1716, 1742, 1767, 1777, 1796, 1838, |
| | 1841, 1896, 1906, 1921-1922, 1994, 2009, 2054, 2059, 2075, 2089-2090, 2120, |
| | 2124, 2156, 2179, 2185, 2193, 2196, 2225, 2269, 2284, 2321, 2418, 2428, 2465, |
| | 2472, 2479, 2486-2487, 2501-2502, 2514, 2519, 2522, 2535, 2543, 2547, 2573, |
| | 2577, 2637, 2664, 2738, 2828, 2841, 2867, 2891, 2900, 2936, 2944, 2983, 2998, |
| | 3002, 3075, 3086, 3124, 3177, 3196, 3200, 3213, 3247, 3270, 3275, 3281, 3341, |
| | 3361, 3409-3410, 3427, 3450, 3467, 3533, 3535, 3549, 3558, 3568, 3575, 3600, |
| | 3664, 3686, 3705, 3819, 3827 |
| South and Central America | 7, 75, 87, 230, 243, 262, 264, 333, 405, 478, 485, 490, 493, 500, 529, 542, 546, |
| | 551, 560, 574, 638, 645, 665, 684, 698, 709, 743, 761, 818, 821, 824-825, 851, |
| | 859, 865, 871, 910, 915, 927, 950, 1005, 1016, 1041, 1118, 1123, 1126, 1141, |
| | 1188, 1193, 1222, 1225, 1230, 1234, 1240, 1251, 1293, 1305, 1309, 1348, 1355, |
| | 1384, 1411, 1424, 1447, 1473, 1484, 1503, 1542-1543, 1574, 1589, 1611, 1667, |
| | 1676, 1716, 1742, 1767, 1796, 1838, 1841, 1906, 1921-1922, 1994, 2054, 2075, |
| | 2089-2090, 2124, 2156, 2179, 2185, 2196, 2225, 2284, 2321, 2418, 2428-2429, |
| | 2444, 2465, 2472, 2479, 2486-2487, 2501-2502, 2519, 2535, 2543, 2561, 2573, |
| | 2577, 2637, 2738, 2828, 2936, 3052, 3075, 3086, 3213, 3281-3282, 3361, 3370, |
| | 3410, 3440, 3467, 3535, 3558, 3600, 3651, 3664, 3694, 3705, 3808, 3819 |
| South Asia | 7, 75, 87, 230, 243, 262, 264, 405, 478, 485, 490, 493, 529, 542, 546, 551, 560, |
| | 574, 638, 645, 665, 684, 698, 709, 743, 761, 770, 778, 818, 821, 824-825, 851, |
| | 859, 865, 871, 910, 915, 927, 950, 1005, 1016, 1041, 1098, 1118, 1123, 1126, |
| | 1141, 1188, 1193, 1222, 1225, 1230, 1234, 1240, 1251, 1293, 1305, 1309, 1322, |
| | 1355, 1384, 1411, 1424, 1447, 1456, 1473, 1484, 1503, 1543, 1547, 1561, 1574, |
| | 1589, 1598, 1611, 1667, 1676, 1694, 1713, 1716, 1742, 1777, 1796, 1841, 1896, |
| | 1906, 1921-1922, 1994, 2021, 2054, 2059, 2075, 2089-2090, 2156, 2179, 2195- |
| | 2196, 2225, 2269, 2284, 2321, 2368, 2428-2429, 2444, 2465, 2472, 2479, 2486- |
| | 2487, 2502-2503, 2519, 2522, 2547, 2561, 2564, 2573, 2577, 2664, 2738, 2828, |
| | 2867, 2891, 2900, 2936, 3002, 3086, 3200, 3277, 3281, 3341, 3361, 3410, 3427, |
| | 3440, 3478, 3531, 3533, 3550, 3651, 3705, 3763, 3808, 3819 |
| South East Asia | 75, 87, 230, 243, 262, 264, 405, 478, 485, 490, 493, 500, 529, 542, 546, 551, 560, |
| | 564, 574, 638, 645, 665, 684, 698, 709, 741, 743, 761, 770, 778, 818, 851, 859, |
| | 865, 871, 910, 915, 927, 950, 1005, 1016, 1041, 1098, 1118, 1123, 1126, 1141, |
| | 1188, 1193, 1222, 1225, 1230, 1240, 1251, 1293, 1305, 1309, 1322, 1348, 1355, |
| | 1384, 1411, 1424, 1447, 1473, 1484, 1542-1543, 1561, 1574, 1598, 1611, 1667, |
| | 1694, 1713, 1716, 1767, 1838, 1841, 1896, 1906, 1921-1922, 1994, 2009, 2021, |
| | 2054, 2059, 2075, 2089-2090, 2120, 2124, 2156, 2179, 2185, 2196, 2225, 2269, |
| | 2284, 2321, 2418, 2428, 2444, 2465, 2472, 2479, 2486, 2501-2502, 2514, 2519, |
| | 2522, 2561, 2573, 2577, 2633, 2637, 2681, 2701, 2738, 2794, 2828, 2841, 2867, |
| | 2891, 2900, 2936, 2944, 2983, 2998, 3124, 3270, 3274, 3281, 3410, 3427, 3450, |
| | 3467, 3533, 3550, 3558, 3568, 3575, 3600, 3727, 3814, 3827 |
| Sub-Saharan Africa | 7, 75, 87, 230, 243, 262, 264, 405, 478, 490, 493, 500, 529, 542, 546, 551, 560, |
| | 564, 574, 638, 645, 665, 684, 709, 743, 761, 818, 851, 859, 865, 871, 910, 915, |
| | 927, 1005, 1041, 1118, 1123, 1126, 1188, 1193, 1222, 1230, 1240, 1305, 1355, |
| | 1384, 1424, 1447, 1484, 1543, 1589, 1598, 1667, 1676, 1716, 1796, 1838, 1841, |
| | 1921-1922, 1994, 2021, 2054, 2059, 2090, 2120, 2124, 2156, 2179, 2196, 2225, |
| | 2321, 2428-2429, 2465, 2472, 2479, 2486-2487, 2502-2503, 2519, 2535, 2573, |
| | 2637, 2664, 2681, 2701, 2738, 2841, 2867, 2936, 3052, 3177, 3200, 3270, 3274, |
| | 3281-3282, 3361, 3440, 3467, 3506, 3549, 3626, 3650-3651, 3696, 3766, 3780 |
| Western Asia | 7, 75, 87, 230, 243, 262, 264, 333, 405, 478, 485, 490, 493, 500, 529, 542, 546, |
| | 551, 560, 564, 574, 638, 645, 665, 684, 698, 709, 743, 761, 770, 778, 818, 824- |
| | 825, 851, 859, 865, 871, 910, 915, 927, 950, 1005, 1041, 1098, 1118, 1123, 1126, |
| | 1141, 1188, 1193, 1222, 1225, 1230, 1234, 1240, 1251, 1305, 1309, 1322, 1348, |
| | 1355, 1384, 1411, 1424, 1447, 1456, 1473, 1484, 1542-1543, 1561, 1574, 1589, |
| | 1598, 1611, 1667, 1694, 1713, 1716, 1742, 1777, 1796, 1838, 1841, 1921-1922, |
| | 1994, 2009, 2021, 2054, 2075, 2089-2090, 2120, 2156, 2179, 2196, 2225, 2269, |
| | 2284, 2321, 2368, 2428, 2444, 2465, 2472, 2479, 2486-2487, 2502-2503, 2514, |
| | 2519, 2522, 2547, 2561, 2564, 2573, 2637, 2701, 2738, 2794, 2828, 2841, 2867, |
| | 2900, 2936, 2983, 2998, 3052, 3179, 3281, 3333, 3361, 3410, 3440, 3535, 3552, |
| | 3626, 3651, 3664, 3705, 3763, 3808, 3819 |

TABLE 21
Region-specific peptide pools derived from NSP16 protein for CD8 T cell assays.

| Region | Seq. IDs of peptides |
|---|---|
| Australia | 8, 176-177, 208, 211, 376, 413, 422, 424, 469, 583, 651, 670, 841, 843, 864, 879, |
| | 888, 922, 940, 943, 961, 1025, 1031, 1070, 1090, 1150, 1173, 1194, 1254, 1278, |
| | 1313, 1323, 1346, 1351, 1415, 1430, 1454, 1499, 1545, 1650, 1653, 1779, |
| | 1789-1790, 1806, 1881, 1883, 1889, 1931, 2058, 2121, 2250, 2288, 2351, 2391, |
| | 2415, 2417, 2437, 2460, 2488, 2494, 2569, 2581, 2617, 2646, 2648, 2660, 2680, |
| | 2682, 2696, 2747, 2774, 2784, 2789, 2836, 2870, 2902, 2922, 2963, 2974, 2980, |
| | 2982, 3035, 3048, 3063, 3140, 3152, 3232, 3388, 3527, 3538, 3634, 3648, 3735, |
| | 3757 |
| Europe | 8, 177, 208, 211, 376, 413, 422, 424, 469, 501, 583, 670, 841, 843, 864, 879, 888, |
| | 940, 943, 1025, 1031, 1070, 1090, 1150, 1170, 1173, 1194, 1254, 1278, 1323, |
| | 1346, 1351, 1366, 1415, 1430, 1454, 1499, 1653, 1779, 1789, 1881, 1883, 1889, |
| | 1931, 2121, 2163, 2233, 2250, 2253, 2391, 2415, 2460, 2494, 2507, 2520, 2569, |
| | 2617, 2646, 2660, 2696, 2789, 2902, 2922, 3035, 3152, 3191, 3388, 3403, 3476, |
| | 3512, 3538, 3735 |
| North Africa | 8, 176-177, 208, 376, 413, 422, 424, 469, 651, 670, 841, 843, 864, 879, 888, 922, |
| | 940, 943, 961, 1031, 1070, 1090, 1150, 1173, 1194, 1254, 1278, 1313, 1323, |
| | 1346, 1366, 1415, 1430, 1454, 1499, 1653, 1779, 1789, 1806, 1881, 1883, 1889, |
| | 1931, 2036, 2058, 2121, 2250, 2351, 2391, 2415, 2437, 2439, 2460, 2488, 2569, |
| | 2581, 2617, 2646, 2648, 2660, 2774, 2784, 2789, 2870, 2902, 2965, 2974, 2982, |
| | 3035, 3140, 3152, 3232, 3310, 3359, 3388, 3495, 3538, 3541, 3634, 3735, 3737, |
| | 3757 |
| North America | 8, 176-177, 208, 211, 376, 413, 422, 424, 469, 501, 510, 583, 670, 841, 843, 864, |
| | 879, 888, 940, 943, 961, 1031, 1070, 1090, 1150, 1170, 1173, 1194, 1254, 1278, |
| | 1313, 1323, 1351, 1415, 1430, 1454, 1779, 1789, 1881, 1883, 1889, 1931, 2121, |
| | 2163, 2250, 2253, 2288, 2348, 2351, 2391, 2415, 2507, 2569, 2581, 2617, 2646, |
| | 2660, 2680, 2747, 2774, 2789, 2902, 2922, 2974, 2980, 2982, 3035, 3048, 3140, |
| | 3152, 3191, 3334, 3388, 3482, 3512, 3538, 3563, 3634, 3648, 3724, 3735, 3737, |
| | 3793 |
| North East Asia | 176-177, 208, 211, 376, 413, 469, 501, 583, 651, 670, 841, 843, 864, 879, 888, |
| | 922, 940, 943, 961, 1031, 1070, 1090, 1150, 1170, 1173, 1194, 1254, 1278, 1313, |
| | 1323, 1346, 1351, 1366, 1415, 1430, 1454, 1499, 1653, 1779, 1789-1790, 1881, |
| | 1883, 1889, 1931, 1995, 2058, 2121, 2163, 2250, 2253, 2351, 2391, 2415, 2417, |
| | 2437, 2439, 2460, 2488, 2507, 2557, 2569, 2581, 2617, 2646, 2660, 2680, 2682, |
| | 2696, 2774, 2784, 2789, 2870, 2902, 2922, 2974, 2980, 2982, 3035, 3048, 3140, |
| | 3191, 3359, 3388, 3495, 3512, 3538, 3737 |
| Oceania | 176-177, 208, 376, 413, 424, 469, 501, 510, 583, 651, 670, 841, 843, 864, 879, |
| | 888, 922, 940, 943, 961, 1025, 1031, 1070, 1090, 1150, 1170, 1173, 1194, 1254, |
| | 1278, 1313, 1323, 1346, 1351, 1366, 1415, 1430, 1454, 1499, 1545, 1650, 1653, |
| | 1769, 1779, 1789-1790, 1806, 1881, 1883, 1889, 1931, 1995, 2058, 2121, 2163, |
| | 2250, 2253, 2288, 2348, 2351, 2391, 2415, 2417, 2437, 2439, 2460, 2488, 2494, |
| | 2507, 2557, 2569, 2581, 2617, 2646, 2648, 2660, 2680, 2682, 2696, 2747, 2774, |
| | 2784, 2789, 2870, 2902, 2963, 2974, 2980, 2982, 3035, 3048, 3063, 3140, 3152, |
| | 3191, 3322, 3334, 3359, 3384, 3388, 3482, 3512, 3538, 3563, 3634, 3648, 3681, |
| | 3724, 3757 |
| South and Central America | 8, 176-177, 208, 376, 413, 422, 424, 469, 501, 510, 583, 651, 670, 841, 843, 864, |
| | 879, 888, 922, 940, 943, 961, 1031, 1070, 1090, 1150, 1170, 1173, 1194, 1254, |
| | 1278, 1313, 1323, 1346, 1351, 1415, 1430, 1454, 1499, 1545, 1779, 1789, 1806, |
| | 1881, 1883, 1889, 1931, 2058, 2121, 2163, 2250, 2253, 2288, 2351, 2391, 2415, |
| | 2417, 2437, 2439, 2460, 2488, 2494, 2507, 2569, 2581, 2617, 2646, 2648, 2660, |
| | 2680, 2682, 2747, 2784, 2789, 2870, 2902, 2922, 2963, 2974, 2980, 2982, 3035, |
| | 3048, 3063, 3140, 3191, 3232, 3388, 3495, 3512, 3527, 3538, 3594, 3648, 3676, |
| | 3735, 3737, 3757, 3793 |
| South Asia | 8, 176-177, 208, 376, 389-390, 413, 422, 424, 469, 583, 651, 670, 841, 843, 864, |
| | 879, 888, 922, 940, 943, 961, 1025, 1031, 1070, 1090, 1150, 1170, 1173, 1194, |
| | 1254, 1278, 1313, 1323, 1346, 1351, 1366, 1415, 1430, 1454, 1499, 1545, 1653, |
| | 1779, 1789-1790, 1806, 1881, 1883, 1889, 1931, 1995, 2036, 2058, 2121, 2163, |
| | 2233, 2250, 2253, 2351, 2391, 2415, 2417, 2437, 2439, 2460, 2488, 2494, 2507, |
| | 2557, 2569, 2581, 2617, 2646, 2648, 2660, 2680, 2682, 2690, 2774, 2784, 2789, |
| | 2836, 2870, 2902, 2922, 2965, 2974, 2982, 3035, 3063, 3140, 3152, 3232, 3322, |
| | 3359, 3384, 3388, 3446, 3476, 3495, 3512, 3538, 3541, 3676, 3735, 3737, 3757, |
| | 3828 |
| South East Asia | 176-177, 208, 376, 413, 424, 469, 501, 510, 583, 651, 670, 841, 843, 864, 879, |
| | 888, 922, 940, 943, 961, 1025, 1031, 1070, 1090, 1150, 1170, 1173, 1194, 1254, |
| | 1278, 1313, 1323, 1346, 1351, 1366, 1415, 1430, 1454, 1499, 1650, 1769, 1779, |
| | 1789-1790, 1806, 1881, 1883, 1889, 1931, 2058, 2121, 2163, 2250, 2253, 2288, |
| | 2348, 2351, 2391, 2415, 2439, 2460, 2494, 2507, 2569, 2581, 2617, 2646, 2660, |
| | 2680, 2682, 2690, 2696, 2747, 2774, 2784, 2789, 2836, 2902, 2922, 2963, 2980, |
| | 3035, 3048, 3063, 3140, 3152, 3191, 3194, 3322, 3334, 3359, 3385, 3388, 3482, |
| | 3495, 3512, 3527, 3538, 3563, 3648, 3737, 3782, 3840 |
| Sub-Saharan Africa | 8, 176-177, 208, 211, 376, 413, 422, 424, 469, 501, 510, 583, 651, 670, 841, 843, |
| | 864, 879, 888, 922, 940, 943, 961, 1031, 1070, 1090, 1150, 1170, 1173, 1194, |
| | 1254, 1278, 1313, 1323, 1351, 1415, 1430, 1454, 1545, 1650, 1653, 1789-1790, |
| | 1806, 1881, 1883, 1889, 1931, 2058, 2121, 2163, 2250, 2253, 2351, 2391, 2415, |
| | 2507, 2520, 2569, 2581, 2617, 2646, 2660, 2747, 2774, 2789, 2836, 2870, 2902, |
| | 2922, 2965, 2974, 2980, 2982, 3035, 3152, 3191, 3232, 3403, 3512, 3538, 3634, |
| | 3724, 3735, 3850 |
| Western Asia | 8, 176-177, 208, 376, 413, 422, 424, 469, 501, 510, 583, 651, 670, 841, 843, 864, |
| | 879, 888, 922, 940, 943, 961, 1025, 1031, 1070, 1090, 1150, 1170, 1173, 1194, |
| | 1254, 1278, 1323, 1346, 1351, 1366, 1415, 1430, 1454, 1499, 1545, 1653, 1769, |
| | 1779, 1789, 1806, 1881, 1883, 1889, 1931, 2058, 2121, 2163, 2233, 2250, 2253, |
| | 2288, 2348, 2351, 2391, 2415, 2439, 2460, 2494, 2507, 2520, 2569, 2581, 2617, |
| | 2646, 2648, 2660, 2690, 2696, 2747, 2789, 2836, 2870, 2902, 2922, 2963, 2965, |
| | 2974, 2982, 3035, 3063, 3140, 3152, 3191, 3194, 3322, 3334, 3359, 3384, 3388, |
| | 3403, 3446, 3476, 3482, 3495, 3512, 3538, 3563, 3594, 3648, 3676, 3735, 3737, |
| | 3757 |

TABLE 22
Region-specific peptide pools derived from N protein for CD8 T cell assays.

| Region | Seq. IDs of peptides |
|---|---|
| Australia | 11, 26-27, 30-32, 64, 74, 122-123, 214, 216, 220, 263, 272-273, 276, 280, 310, |
| | 336, 374, 427-428, 585, 628, 632, 652, 1010, 1050, 1058, 1082, 1120, 1134, 1174, |
| | 1206, 1375, 1392, 1410, 1689, 1746, 1772, 1872, 1907, 2165, 2169, 2214, 2217, |
| | 2243, 2390, 2397, 2400, 2432, 2482, 2497, 2568, 2570, 2580, 2592, 2605, 2726, |
| | 2734, 2750, 3001, 3016, 3031, 3037, 3126, 3268, 3299, 3316, 3437, 3448, 3484, |
| | 3487, 3645, 3740, 3858 |
| Europe | 11, 26-27, 30-32, 64, 74, 122-123, 214, 216, 220, 263, 266, 272-273, 276, 280, |
| | 310, 336, 361, 374, 427-428, 585, 628,632, 1010, 1050, 1058, 1082, 1120, 1134, |
| | 1174, 1206, 1375, 1392, 1655, 1689, 1746, 1862, 2217, 2337, 2390, 2397, 2432, |
| | 2497, 2605, 2734, 2917, 3106, 3112, 3234, 3387, 3454, 3487, 3714, 3719, 3721, |
| | 3797 |
| North Africa | 11, 26-27, 30-32, 64, 153-154, 214, 263, 272-273, 276, 280, 310, 336, 361, 374, |
| | 427-428, 585, 628, 632, 652, 1010, 1050, 1058, 1082, 1120, 1134, 1174, 1375, |
| | 1392, 1410, 1689, 1872, 1907, 2165, 2169, 2214, 2361, 2397, 2400, 2432, 2482, |
| | 2570, 2605, 2726, 2734, 2750, 2777, 3001, 3153, 3316, 3340, 3342, 3437, 3487, |
| | 3675, 3734, 3775 |
| North America | 11, 26-27, 30-32, 64, 74, 214, 216, 220, 263, 272-273, 276, 280, 310, 361, 374, |
| | 427-428, 585, 628, 632, 1010, 1058, 1082, 1120, 1134, 1174, 1206, 1375, 1392, |
| | 1655, 1689, 1746, 1772, 1862, 1907, 2217, 2307, 2397, 2400, 2432, 2482, 2497, |
| | 2568, 2570, 2580, 2605, 2726, 2728, 2734, 2750, 2917, 3016, 3037, 3096, 3106, |
| | 3112, 3234, 3268, 3299, 3316, 3387, 3437, 3448, 3484, 3487, 3610, 3645, 3740 |
| North East Asia | 26-27, 30-32, 64, 74, 122-123, 214, 216, 220, 263, 272-273, 276, 280, 336, 361, |
| | 585, 628, 632, 1010, 1050, 1058, 1082, 1120, 1134, 1174, 1206, 1375, 1392, 1410, |
| | 1655, 1689, 1746, 1772, 1862, 1872, 1907, 2169, 2214, 2217, 2243, 2307, 2342, |
| | 2397, 2400, 2432, 2482, 2497, 2533, 2568, 2570, 2580, 2592, 2605, 2726, 2734, |
| | 2750, 2777, 2917, 3001, 3016, 3018, 3021, 3031, 3106, 3112, 3126, 3153, 3234, |
| | 3268, 3299, 3301, 3387, 3437, 3448, 3484, 3487, 3645, 3714, 3721, 3774 |
| Oceania | 26-27, 30-32, 64, 74, 122-123, 154, 214, 263, 266, 272-273, 276, 280, 336, 374, |
| | 585, 628, 632, 652, 1010, 1050, 1058, 1082, 1120, 1134, 1174, 1206, 1375, 1392, |
| | 1410, 1655, 1689, 1746, 1772, 1862, 1872, 1907, 2165, 2214, 2217, 2243, 2342, |
| | 2390, 2397, 2400, 2432, 2482, 2497, 2533, 2568, 2570, 2580, 2592, 2605, 2643, |
| | 2726, 2728, 2732, 2734, 2750, 2777, 2906, 2917, 3001, 3016, 3018, 3021, 3031, |
| | 3037, 3096, 3126-3127, 3135, 3153, 3255, 3268, 3299, 3301, 3316, 3373, 3437, |
| | 3448, 3484, 3487, 3645, 3721, 3740, 3774, 3858 |
| South and Central America | 11, 26-27, 30-32, 64, 122, 214, 263, 272-273, 276, 280, 336, 361, 374, 427-428, |
| | 585, 628, 632, 1010, 1050, 1058, 1082, 1120, 1134, 1174, 1206, 1375, 1392, 1410, |
| | 1655, 1689, 1746, 1772, 1862, 1872, 2165, 2169, 2214, 2217, 2243, 2397, 2400, |
| | 2432, 2482, 2497, 2533, 2568, 2580, 2605, 2726, 2734, 2750, 2906, 2917, 3016, |
| | 3021, 3031, 3106, 3112, 3126, 3234, 3387, 3437, 3448, 3487, 3645, 3714, 3721, |
| | 3774 |
| South Asia | 11, 26-27, 30-32, 64, 74, 214, 263, 266, 272-273, 276, 280, 336, 361, 374, 427- |
| | 428, 585, 628, 632, 1010, 1050, 1058, 1082, 1120, 1134, 1174, 1206, 1375, 1392, |
| | 1410, 1655, 1689, 1746, 1772, 1862, 1872, 2165, 2169, 2214, 2217, 2243, 2342, |
| | 2361, 2397, 2400, 2432, 2482, 2497, 2533, 2570, 2580, 2592, 2605, 2643, 2732, |
| | 2734, 2750, 2777, 2917, 3000, 3018, 3021, 3026, 3031, 3126-3127, 3153, 3268, |
| | 3301, 3315, 3373, 3448, 3487, 3663, 3721, 3774, 3798 |
| South East Asia | 26-27, 30-32, 64, 74, 122, 154, 214, 263, 266, 272-273, 280, 336, 361, 374, 427, |
| | 585, 628, 632, 1010, 1050, 1058, 1082, 1120, 1134, 1174, 1206, 1375, 1392, 1655, |
| | 1689, 1746, 1772, 1862, 1872, 1907, 2072, 2165, 2169, 2214, 2217, 2243, 2342, |
| | 2361, 2390, 2397, 2400, 2432, 2482, 2497, 2533, 2568, 2570, 2580, 2592, 2605, |
| | 2726, 2728, 2734, 2750, 2777, 2906, 2917, 3016, 3021, 3031, 3096, 3126, 3135, |
| | 3255, 3268, 3299, 3301, 3437, 3448-3449, 3484, 3487, 3645, 3721, 3771, 3774 |
| Sub-Saharan Africa | 11, 26-27, 30-32, 64, 74, 153-154, 162, 214, 216, 220, 263, 272-273, 276, 280, |
| | 374, 427-428, 585, 628, 632, 652, 1010, 1050, 1058, 1082, 1120, 1134, 1174, |
| | 1206, 1375, 1392, 1689, 1746, 1772, 1862, 1872, 1907, 2165, 2214, 2217, 2307, |
| | 2397, 2400, 2432, 2482, 2497, 2533, 2570, 2605, 2726, 2734, 2750, 2917, 3001, |
| | 3016, 3037, 3153, 3234, 3285, 3316, 3350, 3437, 3487, 3599 |
| Western Asia | 11, 26-27, 30-32, 64, 74, 122-123, 154, 214, 263, 266, 272-273, 276, 280, 336, |
| | 374, 427-428, 585, 628, 632, 1010, 1050, 1058, 1082, 1120, 1134, 1174, 1206, |
| | 1375, 1392, 1410, 1655, 1689, 1746, 1772, 1862, 1872, 2072, 2165, 2169, 2214, |
| | 2217, 2397, 2400, 2432, 2482, 2497, 2580, 2605, 2643, 2728, 2732, 2734, 2750, |
| | 2777, 2917, 3000, 3026, 3096, 3112, 3135, 3153, 3234, 3255, 3268, 3285, 3340, |
| | 3342, 3350, 3373, 3387, 3437, 3448, 3487, 3610, 3663, 3721, 3740, 3774, 3797 |

TABLE 23
Region-specific peptide pools derived from M protein for CD8 T cell assays.

| Region | Seq. IDs of peptides |
|---|---|
| Australia | 12, 37, 53, 147, 157, 173, 187, 194, 198, 200, 204-205, 209, 212, 219, 229, 236, |
| | 240, 312, 431, 492, 522, 530, 562, 604, 767-768, 868, 875, 937, 982, 1032, 1167, |
| | 1192, 1195, 1201, 1287, 1378-1379, 1434, 1601, 1656, 1700, 1793, 1878, 1901, |
| | 1903, 1942, 2015, 2053, 2078, 2122, 2128, 2143, 2180, 2199, 2268, 2274, 2293, |
| | 2375, 2405, 2448, 2527, 2597, 2604, 2754, 2829, 2889, 3051, 3065, 3098, 3138, |
| | 3189, 3303, 3327, 3346, 3358, 3447, 3534, 3561, 3759 |
| Europe | 12, 37, 53, 147, 152, 187, 194, 198, 200, 204-205, 209, 212, 219, 229, 236, 240, |
| | 258, 312, 431, 492, 522, 530, 562, 604, 683, 749-750, 767-768, 858, 860, 868, 875, |
| | 937, 982, 1032, 1073, 1124, 1167, 1192, 1195, 1287, 1378, 1434, 1507, 1599, |
| | 1601, 1627, 1656, 1700, 1793, 1878, 1901, 1903, 1942, 2078, 2128, 2143, 2180, |
| | 2199, 2268, 2293, 2330, 2366, 2375, 2384, 2405, 2426, 2448, 2604, 2754, 2873, |
| | 2889, 2992, 3051, 3065, 3103, 3189, 3303, 3346, 3351, 3733, 3754, 3759, 3851, |
| | 3859 |
| North Africa | 12, 37, 53, 147, 151, 157, 160, 204-205, 209, 229, 236, 240, 312, 431, 492, 522, |
| | 530, 562, 767-768, 858, 860, 868, 875, 937, 982, 1032, 1167, 1192, 1195, 1201, |
| | 1287, 1378-1379, 1434, 1601, 1656, 1700, 1793, 1878, 1901, 1942, 1944, 2015, |
| | 2053, 2078, 2128, 2180, 2199, 2268, 2276, 2293, 2375, 2405, 2448, 2452, 2527, |
| | 2597, 2604, 2694, 2754, 2829, 2850, 2889, 2895, 2994, 3051, 3065, 3098, 3189, |
| | 3246, 3271, 3303, 3327, 3346, 3349, 3358, 3534, 3561, 3718, 3759, 3810 |
| North America | 12, 37, 53, 147, 152, 157, 173, 187, 194, 198, 204-205, 209, 212, 219, 229, 236, |
| | 240, 258, 312, 431, 522, 530, 562, 604, 750, 767-768, 868, 875, 937, 1032, 1124, |
| | 1167, 1192, 1195, 1378-1379, 1434, 1507, 1599, 1601, 1656, 1700, 1793, 1878, |
| | 1901, 1903, 1942, 2015, 2053, 2078, 2122, 2143, 2199, 2268, 2274, 2276, 2293, |
| | 2366, 2375, 2384, 2405, 2426, 2448, 2597, 2604, 2754, 2889, 3051, 3138, 3303, |
| | 3327, 3346, 3358, 3447, 3561, 3668, 3733 |
| North East Asia | 12, 37, 53, 147, 152, 157, 173, 187, 194, 198, 200, 204-205, 209, 212, 219, 229, |
| | 240, 258, 312, 492, 522, 530, 562, 604, 767-768, 858, 868, 875, 937, 982, 1032, |
| | 1124, 1167, 1195, 1201, 1287, 1378-1379, 1434, 1507, 1599, 1601, 1656, 1700, |
| | 1793, 1878, 1901, 1903, 1942, 1944, 2053, 2078, 2122, 2128, 2143, 2199, 2268, |
| | 2274, 2276, 2330, 2366, 2375, 2384, 2405, 2426, 2452, 2527, 2597, 2604, 2694, |
| | 2740, 2754, 2829, 2850, 2889, 2895, 2994, 3051, 3065, 3098, 3138, 3189, 3246, |
| | 3271, 3303, 3327, 3346, 3349, 3358, 3362, 3447, 3485, 3534, 3569, 3668, 3733, |
| | 3825 |
| Oceania | 12, 37, 53, 147, 157, 173, 187, 194, 198, 200, 204-205, 209, 229, 240, 312, 492, |
| | 522, 530, 562, 604, 683, 750, 767-768, 858, 860, 868, 875, 937, 982, 1032, 1073, |
| | 1124, 1167, 1192, 1195, 1201, 1287, 1378-1379, 1434, 1507, 1599, 1601, 1627, |
| | 1656, 1700, 1793, 1878, 1901, 1903, 1942, 1944, 2015, 2053, 2078, 2122, 2128, |
| | 2143, 2180, 2199, 2268, 2274, 2276, 2293, 2330, 2366, 2375, 2384, 2405, 2422, |
| | 2426, 2448, 2527, 2597, 2604, 2740, 2754, 2829, 2889, 2895, 3051, 3065, 3138, |
| | 3189, 3249, 3271, 3303, 3327, 3346, 3349, 3358, 3447, 3485, 3534, 3561, 3569, |
| | 3580, 3712 |
| South and Central America | 12, 37, 53, 147, 152, 157, 173, 187, 194, 198, 200, 204-205, 209, 229, 240, 312, |
| | 431, 492, 522, 530, 562, 604, 767-768, 858, 868, 875, 937, 982, 1032, 1124, 1167, |
| | 1192, 1195, 1201, 1287, 1378-1379, 1434, 1507, 1599, 1601, 1656, 1700, 1793, |
| | 1878, 1901, 1903, 1942, 1944, 2015, 2053, 2078, 2128, 2143, 2180, 2199, 2268, |
| | 2274, 2276, 2293, 2366, 2375, 2384, 2405, 2426, 2448, 2527, 2597, 2604, 2740, |
| | 2754, 2829, 2850, 2873, 2889, 3051, 3065, 3138, 3271, 3303, 3346, 3349, 3358, |
| | 3447, 3534, 3561, 3825 |
| South Asia | 12, 37, 53, 147, 157, 173, 187, 194, 198, 200, 204-205, 209, 229, 240, 312, 431, |
| | 492, 522, 530, 562, 604, 683, 750, 767-768, 858, 860, 868, 875, 937, 982, 1032, |
| | 1073, 1124, 1167, 1192, 1195, 1201, 1287, 1378-1379, 1434, 1507, 1599, 1601, |
| | 1627, 1656, 1700, 1793, 1878, 1901, 1903, 1942, 1944, 2015, 2053, 2078, 2122, |
| | 2128, 2143, 2180, 2199, 2268, 2274, 2276, 2293, 2330, 2366, 2375, 2384, 2405, |
| | 2422, 2426, 2448, 2452, 2527, 2597, 2604, 2694, 2740, 2754, 2829, 2850, 2889, |
| | 2994, 3051, 3065, 3098, 3138, 3189, 3271, 3303, 3327, 3346, 3349, 3358, 3362, |
| | 3447, 3485, 3534, 3561 |
| South East Asia | 12, 37, 53, 147, 157, 173, 187, 194, 198, 200, 204-205, 209, 229, 258, 312, 492, |
| | 522, 530, 562, 604, 683, 750, 767-768, 858, 860, 868, 875, 937, 982, 1032, 1073, |
| | 1124, 1167, 1192, 1195, 1201, 1378-1379, 1434, 1507, 1599, 1601, 1627, 1656, |
| | 1699-1700, 1793, 1878, 1901, 1903, 1942, 1944, 2053, 2078, 2122, 2128, 2143, |
| | 2180, 2199, 2268, 2274, 2276, 2293, 2366, 2375, 2384, 2405, 2422, 2426, 2597, |
| | 2604, 2740, 2754, 2829, 2850, 2873, 2889, 2895, 2994, 3051, 3103, 3138, 3249, |
| | 3271-3272, 3303, 3327, 3346, 3349, 3351, 3362, 3569, 3580, 3668, 3784 |
| Sub-Saharan Africa | 12, 37, 53, 147, 151, 157, 160, 173, 187, 194, 198, 204-205, 209, 212, 219, 229, |
| | 236, 240, 431, 492, 522, 530, 562, 604, 767-768, 860, 868, 875, 937, 1032, 1167, |
| | 1192, 1195, 1378-1379, 1434, 1507, 1599, 1601, 1656, 1700, 1793, 1878, 1901, |
| | 1903, 1942, 2015, 2053, 2078, 2143, 2180, 2199, 2268, 2274, 2276, 2293, 2330, |
| | 2375, 2405, 2426, 2448, 2527, 2597, 2604, 2754, 2829, 2889, 2895, 3051, 3065, |
| | 3098, 3246, 3303, 3327, 3346, 3358, 3445, 3447, 3561, 3693, 3784 |
| Western Asia | 12, 37, 53, 147, 152, 157, 173, 187, 194, 198, 204-205, 209, 229, 236, 240, 312, |
| | 431, 492, 522, 530, 562, 604, 683, 750, 767-768, 858, 868, 875, 937, 982, 1032, |
| | 1073, 1124, 1167, 1192, 1195, 1201, 1287, 1378-1379, 1434, 1507, 1599, 1601, |
| | 1627, 1656, 1699-1700, 1793, 1878, 1901, 1903, 1942, 1944, 2053, 2078, 2128, |
| | 2143, 2180, 2199, 2268, 2274, 2276, 2293, 2366, 2375, 2384, 2405, 2422, 2426, |
| | 2448, 2452, 2527, 2597, 2604, 2694, 2740, 2754, 2829, 2850, 2873, 2889, 2895, |
| | 3051, 3065, 3098, 3189, 3249, 3271, 3303, 3327, 3346, 3349, 3351, 3358, 3561, |
| | 3718, 3733, 3754, 3759 |

TABLE 24
Region-specific peptide pools derived from NSP5 protein for CD8 T cell assays.

| Region | Seq. IDs of peptides |
|---|---|
| Australia | 13, 222, 227, 412, 426, 468, 474, 476, 579, 694, 830, 838, 853, 881, 958, 1064, |
| | 1104, 1122, 1155, 1204, 1227, 1271, 1281-1282, 1338, 1342, 1374, 1442, 1490, |
| | 1631, 1637, 1687, 1719, 1747, 1819, 1844, 1866, 2074, 2095, 2099, 2103, 2125, |
| | 2130, 2211, 2329, 2333, 2339, 2349, 2454, 2476, 2510, 2512, 2550, 2567, 2629, |
| | 2638, 2736, 2746, 2773, 2793, 2808, 2820, 2826, 2864, 2869, 2884, 2953, 2956, |
| | 2987, 3033, 3056, 3143, 3150, 3252, 3260, 3276, 3365, 3397, 3500, 3588, 3657, |
| | 3803 |
| Europe | 13, 227, 412, 426, 468, 474, 476, 579, 589, 815, 830, 838, 881, 952, 958, 1064, |
| | 1122, 1155, 1227, 1281-1282, 1338, 1374, 1442, 1568, 1631, 1637, 1719, 1844, |
| | 2091, 2099, 2103, 2125, 2130, 2167, 2304, 2329, 2339, 2411, 2454, 2476, 2512, |
| | 2550, 2574, 2629, 2638, 2746, 2773, 2793, 2808, 2862, 2864, 2869, 2884, 2953, |
| | 2956, 2987, 3073, 3170, 3273, 3307, 3339, 3365, 3397, 3500, 3627, 3803 |
| North Africa | 13, 222, 227, 412, 426, 468, 474, 476, 694, 830, 838, 853, 881, 958, 1064, 1104, |
| | 1122, 1204, 1227, 1271, 1281-1282, 1374, 1442, 1490, 1631, 1637, 1687, 1719, |
| | 1747, 1819, 1844, 1866, 1904, 1972, 2074, 2095, 2099, 2125, 2329, 2339, 2349, |
| | 2411, 2476, 2512, 2550, 2567, 2638, 2746, 2773, 2793, 2808, 2820, 2826, 2849, |
| | 2864, 2869, 2879, 2884, 2953, 2956, 2987, 3033, 3073, 3143, 3150, 3252, 3260, |
| | 3273, 3276, 3397, 3457, 3500, 3588, 3627, 3755, 3760, 3855 |
| North America | 13, 222, 227, 426, 468, 474, 476, 579, 589, 838, 853, 881, 952, 958, 1122, 1155, |
| | 1204, 1227, 1271, 1281-1282, 1338, 1442, 1490, 1582, 1631, 1819, 1844, 1939, |
| | 2074, 2099, 2103, 2125, 2329, 2339, 2349, 2454, 2476, 2510, 2512, 2550, 2567, |
| | 2629, 2638, 2736, 2746, 2773, 2793, 2808, 2820, 2862, 2864, 2884, 2956, 2987, |
| | 3056, 3073, 3307, 3339, 3365, 3397, 3500, 3588, 3764 |
| North East Asia | 412, 426, 468, 474, 476, 579, 589, 694, 830, 838, 853, 881, 952, 958, 1064, 1122, |
| | 1155, 1204, 1227, 1271, 1281-1282, 1338, 1342, 1374, 1442, 1490, 1568, 1582, |
| | 1631, 1719, 1819, 1844, 1866, 1904, 2074, 2095, 2099, 2103, 2125, 2130, 2167, |
| | 2211, 2329, 2339, 2349, 2411, 2454, 2476, 2510, 2512, 2550, 2567, 2629, 2638, |
| | 2736, 2746, 2773, 2793, 2808, 2820, 2849, 2862, 2864, 2869, 2879, 2884, 2953, |
| | 2956, 2987, 3033, 3056, 3073, 3150, 3276, 3307, 3339, 3365, 3397, 3426, 3500, |
| | 3627, 3680, 3803 |
| Oceania | 222, 227, 412, 426, 468, 474, 476, 579, 589, 694, 830, 838, 853, 881, 952, 958, |
| | 1064, 1122, 1155, 1204, 1227, 1271, 1281-1282, 1338, 1342, 1374, 1442, 1490, |
| | 1498, 1568, 1582, 1619, 1631, 1637, 1687, 1719, 1747, 1819, 1844, 1866, 1904, |
| | 1939, 2074, 2091, 2095, 2099, 2103, 2125, 2130, 2167, 2211, 2304, 2329, 2333, |
| | 2339, 2349, 2411, 2454, 2476, 2510, 2512, 2550, 2567, 2629, 2638, 2736, 2746, |
| | 2773, 2793, 2808, 2820, 2826, 2849, 2862, 2864, 2869, 2879, 2884, 2953, 2956, |
| | 2987, 3033, 3056, 3150, 3170, 3252, 3276, 3307, 3339, 3365, 3390, 3397, 3426, |
| | 3500, 3565, 3588, 3657, 3680, 3682, 3764, 3803 |
| South and Central America | 13, 222, 426, 468, 474, 476, 579, 589, 830, 838, 853, 881, 952, 958, 1064, 1104, |
| | 1122, 1155, 1204, 1227, 1281-1282, 1338, 1342, 1374, 1442, 1490, 1582, 1619, |
| | 1631, 1637, 1719, 1747, 1819, 1844, 1866, 1904, 2074, 2095, 2099, 2103, 2125, |
| | 2130, 2211, 2329, 2339, 2349, 2454, 2476, 2510, 2512, 2550, 2567, 2629, 2638, |
| | 2736, 2746, 2773, 2793, 2808, 2820, 2826, 2862, 2864, 2869, 2884, 2953, 2956, |
| | 2987, 3033, 3056, 3143, 3150, 3170, 3252, 3260, 3307, 3339, 3365, 3397, 3500, |
| | 3803, 3849 |
| South Asia | 13, 222, 227, 412, 426, 468, 474, 476, 579, 589, 694, 815, 830, 838, 853, 881, 958, |
| | 1064, 1104, 1122, 1155, 1204, 1227, 1281-1282, 1338, 1342, 1374, 1442, 1498, |
| | 1568, 1582, 1619, 1631, 1637, 1687, 1719, 1747, 1819, 1844, 1866, 1904, 2091, |
| | 2095, 2099, 2103, 2125, 2130, 2167, 2211, 2304, 2329, 2339, 2411, 2454, 2476, |
| | 2512, 2550, 2567, 2629, 2638, 2736, 2746, 2773, 2793, 2808, 2820, 2826, 2864, |
| | 2869, 2884, 2956, 2987, 3056, 3073, 3143, 3150, 3215, 3252, 3260, 3273, 3307, |
| | 3339, 3365, 3390, 3397, 3426, 3457, 3500, 3577, 3588, 3627, 3680, 3787, 3848 |
| South East Asia | 412, 426, 468, 474, 476, 579, 589, 694, 838, 853, 881, 952, 958, 1064, 1122, 1155, |
| | 1204, 1227, 1271, 1281-1282, 1338, 1342, 1442, 1490, 1498, 1568, 1582, 1619, |
| | 1631, 1637, 1687, 1819, 1844, 1866, 1904, 1939, 1972, 2074, 2091, 2099, 2125, |
| | 2130, 2167, 2211, 2304, 2329, 2339, 2349, 2411, 2454, 2476, 2510, 2512, 2550, |
| | 2629, 2638, 2736, 2746, 2773, 2793, 2808, 2820, 2862, 2864, 2869, 2884, 2953, |
| | 2956, 2987, 3033, 3056, 3073, 3143, 3170, 3307, 3339, 3365, 3390, 3397, 3500, |
| | 3627, 3657, 3682, 3834 |
| Sub-Saharan Africa | 13, 222, 227, 426, 468, 474, 476, 579, 589, 838, 853, 881, 952, 958, 1064, 1104, |
| | 1122, 1204, 1227, 1271, 1281-1282, 1338, 1442, 1490, 1631, 1637, 1747, 1844, |
| | 1866, 1939, 1972, 2074, 2099, 2103, 2125, 2130, 2329, 2333, 2339, 2349, 2454, |
| | 2476, 2512, 2550, 2567, 2629, 2638, 2746, 2773, 2793, 2820, 2849, 2862, 2864, |
| | 2879, 2884, 2953, 2956, 2987, 3033, 3073, 3260, 3276, 3307, 3365, 3397, 3500, |
| | 3588, 3627, 3680, 3755, 3760 |
| Western Asia | 13, 222, 227, 412, 426, 468, 474, 476, 579, 589, 694, 815, 830, 838, 853, 881, 952, |
| | 958, 1064, 1104, 1122, 1155, 1204, 1227, 1281-1282, 1338, 1374, 1442, 1498, |
| | 1619, 1631, 1637, 1719, 1747, 1819, 1844, 1866, 1904, 1939, 1972, 2091, 2099, |
| | 2103, 2125, 2130, 2167, 2304, 2329, 2339, 2454, 2476, 2512, 2550, 2567, 2629, |
| | 2638, 2746, 2773, 2793, 2808, 2820, 2826, 2862, 2864, 2869, 2884, 2956, 2987, |
| | 3073, 3143, 3170, 3215, 3252, 3273, 3307, 3339, 3390, 3397, 3588, 3627, 3682, |
| | 3803 |

TABLE 25
Region-specific peptide pools derived from NSP9 protein for CD8 T cell assays.

| Region | Seq. IDs of peptides |
|---|---|
| Australia | 14, 448, 649, 732, 855, 1202, 1256, 1437-1438, 1731, 1738, 1831, 1997, 2002, |
| | 2158, 2191, 2496, 2785, 2909, 2940, 3007, 3111, 3134, 3197, 3226, 3519 |
| Europe | 14, 448, 649, 732, 855, 1038, 1202, 1256, 1438, 1604, 1731, 1738, 1831, 1997, |
| | 2002, 2158, 2345, 2496, 2821, 2939-2940, 3111, 3226 |
| North Africa | 14, 448, 649, 732, 855, 1202, 1235, 1256, 1438, 1731, 1738, 1971, 1997, 2158, |
| | 2191, 2496, 2785, 2875, 2909, 3007, 3111, 3197, 3226, 3519, 3621 |
| North America | 14, 448, 649, 855, 1146, 1202, 1438, 1604, 1738, 1831, 1997, 2002, 2158, 2198, |
| | 2496, 2785, 2909, 2939-2940, 3111, 3134, 3214, 3226, 3545 |
| North East Asia | 448, 649, 732, 1202, 1235, 1256, 1437-1438, 1604, 1738, 1831, 1997, 2002, 2158, |
| | 2191, 2496, 2785, 2909, 2939-2940, 3007, 3111, 3134, 3226, 3545 |
| Oceania | 448, 649, 695, 732, 855, 1038, 1146, 1202, 1235, 1256, 1437-1438, 1604, 1731, |
| | 1738, 1831, 1997, 2002, 2158, 2191, 2198, 2496, 2785, 2909, 2939-2940, 3007, |
| | 3111, 3134, 3197, 3226, 3511, 3519, 3621, 3786 |
| South and Central America | 14, 448, 649, 732, 855, 1202, 1235, 1256, 1437-1438, 1604, 1731, 1738, 1997, |
| | 2002, 2158, 2191, 2198, 2496, 2909, 2939-2940, 3007, 3111, 3226, 3519 |
| South Asia | 14, 448, 649, 695, 732, 855, 1038, 1202, 1235, 1256, 1437-1438, 1731, 1738, |
| | 1997, 2002, 2158, 2191, 2496, 2939-2940, 3007, 3111, 3197, 3226, 3519, 3545 |
| South East Asia | 2496, 2785, 2909, 2939-2940, 3007, 3111, 3134, 3221, 3226, 3511 |
| Sub-Saharan Africa | 14, 448, 649, 695, 1146, 1202, 1256, 1438, 1604, 1731, 1738, 1831, 1971, 1997, |
| | 2002, 2158, 2198, 2496, 2785, 2909, 2940, 3111, 3134, 3197, 3214, 3226, 3511, |
| | 3519, 3621, 3786 |
| Western Asia | 14, 448, 649, 695, 732, 855, 1038, 1146, 1202, 1235, 1256, 1438, 1604, 1731, |
| | 1738, 1831, 1997, 2002, 2158, 2191, 2198, 2496, 2785, 2940, 3007, 3111, 3197, |
| | 3226, 3519, 3621, 3786 |

TABLE 26
Region-specific peptide pools derived from NSP12 protein for CD8 T cell assays.

| Region | Seq. IDs of peptides |
|---|---|
| Australia | 15, 19-20, 50, 63, 66-70, 72-73, 126, 179-180, 185, 189-191, 206, 235, 248, 253, |
| | 260, 300, 319-322, 327, 362, 364, 379, 398, 402, 404, 407, 414, 419, 430, 434, |
| | 441, 446, 454, 462, 465, 479, 481, 488, 491, 497, 516, 518, 528, 537, 553, 588, |
| | 591, 600, 614, 622, 641, 646, 654, 660, 662, 676, 697, 717, 724, 739, 765, 799, |
| | 812, 845, 848, 876, 887, 894-895, 903, 906-907, 917, 921, 932, 946, 975, 979, 998, |
| | 1000, 1012, 1033, 1043, 1047, 1074, 1085, 1087, 1093, 1107, 1125, 1143, 1184, |
| | 1215, 1232-1233, 1260, 1283, 1291, 1300, 1308, 1353, 1359, 1369-1370, 1391, |
| | 1399, 1418-1419, 1435, 1481, 1496, 1502, 1529, 1544, 1553, 1563, 1593, 1597, |
| | 1609, 1654, 1662, 1677, 1720, 1748, 1763, 1765, 1815, 1829-1830, 1851, 1857, |
| | 1873, 1926, 1936, 1947, 1960, 1967-1968, 1982, 1985, 1988, 2004, 2020, 2034, |
| | 2052, 2070, 2073, 2082, 2087, 2116, 2129, 2145, 2149, 2153, 2160, 2173, 2256, |
| | 2291, 2340, 2350, 2373, 2406, 2427, 2483, 2540, 2544, 2548, 2662, 2706, 2768, |
| | 2798, 2823, 2865, 2880, 2894, 2904, 2946, 3066, 3088, 3094, 3121, 3225, 3302, |
| | 3348, 3429, 3441, 3524, 3560, 3629 |
| Europe | 15, 19-20, 50, 63, 66-70, 72-73, 126, 179-180, 189-191, 235, 248, 253, 255, 260, |
| | 300, 319-322, 327, 344, 350-351, 364, 379, 398, 402, 404, 407, 414, 419, 430, 434, |
| | 441, 446, 454, 465, 479, 481, 488, 491, 497, 502, 508, 516, 518, 528, 537, 553, |
| | 571, 588, 591, 600, 627, 654, 662, 676, 697, 717, 724, 735, 739, 751, 765, 799, |
| | 812, 876, 887, 894-895, 903, 907, 917, 921, 946, 975, 979, 1012, 1033, 1043, |
| | 1047, 1074, 1085, 1093, 1099, 1107, 1125, 1143, 1166, 1179, 1184, 1215, 1233, |
| | 1245, 1260, 1264, 1283, 1291, 1300, 1308, 1316, 1353, 1370, 1399, 1419, 1481, |
| | 1496, 1544, 1553, 1609, 1636, 1662, 1671, 1780, 1830, 1847, 1857, 1873, 1926- |
| | 1927, 1936, 1947, 1960, 1988, 2004, 2020, 2023, 2034, 2045, 2052, 2070, 2116, |
| | 2129, 2133, 2145, 2207, 2373, 2540, 2630, 2742, 2798, 2811, 2865, 2908, 3109, |
| | 3302, 3421, 3429, 3434, 3480, 3501, 3524, 3526, 3554 |
| North Africa | 15, 19-20, 50, 63, 66, 126, 179-180, 206, 235, 260, 300, 319-322, 327, 350-351, |
| | 362, 364, 379, 398, 402, 404, 407, 414, 419, 430, 434, 441, 446, 454, 462, 465, |
| | 479, 481, 488, 491, 516, 518, 528, 537, 553, 588, 591, 614, 622, 641, 646, 654, |
| | 662, 676, 697, 724, 735, 739, 765, 799, 812, 845, 848, 876, 887, 894-895, 903, |
| | 906-907, 917, 921, 932, 946, 975, 979, 998, 1033, 1043, 1047, 1074, 1087, 1093, |
| | 1107, 1125, 1131, 1166, 1184, 1215, 1233, 1252, 1260, 1283, 1291, 1353, 1359, |
| | 1369-1370, 1391, 1399, 1418-1419, 1435, 1502, 1529-1530, 1544, 1553, 1577, |
| | 1593, 1597, 1654, 1662, 1677, 1720, 1748, 1765, 1830, 1851, 1857, 1873, 1926, |
| | 1932, 1947, 1949, 1967-1968, 1982, 1985, 1988, 2004, 2020, 2034, 2071, 2073, |
| | 2082, 2087, 2108, 2116, 2129, 2133, 2145, 2149, 2161, 2173, 2237, 2239, 2256, |
| | 2292, 2350, 2360, 2406, 2427, 2430, 2434, 2458, 2531, 2540, 2544, 2572, 2768, |
| | 2778, 2798, 2823, 2890, 2894, 2904, 2910, 2946, 2966, 2969, 2971, 3094, 3102, |
| | 3209, 3302, 3313, 3459, 3462, 3547, 3560, 3629, 3685, 3691, 3726, 3839 |
| North America | 15, 19-20, 50, 63, 66-70, 72-73, 126, 180, 185, 206, 235, 248, 253, 255, 260, 300, |
| | 319-322, 327, 344, 350-351, 362, 364, 379, 398, 402, 404, 407, 414, 419, 430, 434, |
| | 441, 446, 454, 465, 479, 481, 488, 491, 497, 502, 507-508, 516, 518, 528, 537, |
| | 553, 571, 588, 591, 627, 641, 646, 654, 697, 717, 739, 765, 845, 848, 876, 887, |
| | 894-895, 903, 906-907, 917, 932, 946, 975, 979, 998, 1043, 1047, 1093, 1107, |
| | 1143, 1165, 1179, 1184, 1215, 1233, 1245, 1260, 1264, 1283, 1291, 1300, 1308, |
| | 1370, 1399, 1419, 1435, 1481, 1496, 1529, 1553, 1563, 1593, 1597, 1636, 1638, |
| | 1662, 1671, 1718, 1780, 1815, 1829, 1847, 1856-1857, 1868, 1927, 1960, 1982, |
| | 1988, 2004, 2023, 2034-2035, 2051-2052, 2116, 2129, 2145, 2149, 2153, 2160, |
| | 2262, 2291, 2350, 2373, 2382, 2540, 2586, 2609, 2706, 2798, 2865, 2894, 2946, |
| | 2957, 3066, 3088, 3094, 3107, 3121, 3257, 3297, 3302, 3312, 3348, 3441, 3629, |
| | 3692 |
| North East Asia | 15, 19, 42, 50, 66-70, 72-73, 126, 179-180, 185, 248, 253, 255, 260, 300, 327, 344, |
| | 350-351, 362, 364, 379, 419, 434, 441, 446, 454, 462, 465, 479, 481, 488, 491, |
| | 497, 502, 508, 516, 518, 528, 537, 553, 571, 588, 591, 600, 614, 622, 627, 641, |
| | 646, 654, 660, 662, 676, 697, 717, 724, 739, 765, 799, 812, 845, 848, 876, 887, |
| | 894-895, 903, 906-907, 917, 921, 932, 946, 975, 979, 998, 1000, 1012, 1033, 1043, |
| | 1047, 1074, 1087, 1093, 1107, 1125, 1131, 1143, 1166, 1179, 1184, 1215, 1233, |
| | 1245, 1252, 1260, 1264, 1283, 1291, 1300, 1308, 1353, 1359, 1369-1370, 1391, |
| | 1399, 1418-1419, 1435, 1472, 1481, 1496, 1502, 1529-1530, 1544, 1553, 1563, |
| | 1577, 1593, 1597, 1636, 1638, 1662, 1671, 1763, 1765, 1780, 1815, 1829-1830, |
| | 1835, 1847, 1851, 1856-1857, 1868, 1873, 1897, 1926-1927, 1932, 1936, 1947, |
| | 1949, 1960, 1968, 1980, 1982, 1985, 1988, 2004, 2020, 2023, 2034, 2051-2052, |
| | 2073, 2082, 2087, 2108, 2116, 2129, 2145, 2160-2161, 2237, 2256, 2291, 2340, |
| | 2350, 2373, 2427, 2430, 2483, 2540, 2548, 2662, 2706, 2778, 2798, 2872, 2880, |
| | 2892, 2934, 2946, 3066, 3088, 3094, 3107, 3121, 3297, 3348, 3421, 3429, 3441, |
| | 3459, 3462, 3524, 3547, 3560, 3692 |
| Oceania | 15, 42, 50, 63, 66-70, 72-73, 126, 179-180, 185, 206, 248, 253, 260, 300, 327, 362, |
| | 364, 379, 419, 434, 441, 446, 454, 462, 465, 479, 481, 488, 491, 497, 502, 507- |
| | 508, 516, 518, 528, 537, 553, 571, 588, 591, 600, 614, 622, 627, 641, 646, 654, |
| | 660, 662, 676, 697, 717, 724, 735, 739, 751, 765, 771, 799, 812, 845, 848, 876, |
| | 887, 894-895, 903, 906-907, 917, 921, 932, 946, 975, 979, 998, 1000, 1012, 1033, |
| | 1043, 1047, 1053, 1074, 1087, 1093, 1107, 1125, 1131, 1143, 1165-1166, 1179, |
| | 1184, 1215, 1232-1233, 1245, 1252, 1260, 1264, 1283, 1291, 1300, 1308, 1316, |
| | 1353, 1359, 1369-1370, 1383, 1391, 1397, 1399, 1418-1419, 1435, 1472, 1481, |
| | 1486, 1496, 1502, 1505, 1529-1530, 1544, 1553, 1563, 1593, 1597, 1636, 1638, |
| | 1654, 1662, 1677, 1693, 1720, 1748, 1763, 1765, 1815, 1829-1830, 1835, 1847, |
| | 1856-1857, 1868, 1873, 1897, 1927, 1932, 1936, 1947, 1949, 1967-1968, 1980, |
| | 1982, 1988, 2004, 2020, 2023, 2034-2035, 2045, 2051-2052, 2073, 2082, 2087, |
| | 2116, 2129, 2145, 2149, 2153, 2160, 2173, 2207, 2256, 2262, 2291-2292, 2340, |
| | 2350, 2373, 2382, 2406, 2427, 2483, 2537, 2540, 2548, 2551, 2601, 2630, 2662, |
| | 2679, 2706, 2768, 2797-2798, 2823, 2865, 2880, 2890, 2894, 2904, 2934, 2945- |
| | 2946, 3012, 3066, 3088, 3094, 3107, 3121, 3225, 3304, 3348, 3429, 3441, 3524, |
| | 3560, 3629 |
| South and Central America | 15, 19-20, 50, 63, 66-70, 126, 179-180, 185, 189-191, 206, 248, 260, 300, 327, |
| | 344, 350-351, 362, 364, 379, 398, 402, 404, 407, 414, 419, 430, 434, 441, 446, |
| | 454, 462, 465, 479, 481, 488, 491, 497, 502, 508, 516, 518, 528, 537, 553, 571, |
| | 588, 591, 600, 614, 627, 641, 646, 654, 662, 676, 697, 717, 724, 735, 739, 765, |
| | 771, 799, 812, 845, 848, 876, 887, 894-895, 903, 906-907, 917, 921, 946, 975, 979, |
| | 998, 1000, 1033, 1047, 1087, 1093, 1107, 1125, 1131, 1143, 1179, 1184, 1215, |
| | 1233, 1245, 1252, 1260, 1264, 1283, 1291, 1300, 1308, 1353, 1359, 1369-1370, |
| | 1391, 1399, 1418-1419, 1435, 1481, 1486, 1496, 1502, 1529-1530, 1544, 1553, |
| | 1563, 1577, 1593, 1597, 1636, 1654, 1662, 1671, 1677, 1720, 1748, 1763, 1765, |
| | 1815, 1829-1830, 1835, 1847, 1851, 1857, 1873, 1926-1927, 1932, 1936, 1947, |
| | 1949, 1960, 1967-1968, 1982, 1985, 1988, 2004, 2020, 2023, 2034, 2051-2052, |
| | 2073, 2082, 2087, 2108, 2116, 2129, 2145, 2149, 2160-2161, 2173, 2239, 2256, |
| | 2291-2292, 2340, 2350, 2373, 2406, 2427, 2430, 2483, 2540, 2544, 2548, 2572, |
| | 2630, 2662, 2768, 2798, 2823, 2865, 2880, 2894, 2904, 2937, 2946, 2971, 3088, |
| | 3094, 3121, 3302, 3421, 3429, 3524, 3560 |
| South Asia | 15, 19-20, 50, 63, 66, 68-70, 72-73, 126, 179-180, 185, 189-191, 206, 248, 260, |
| | 300, 327, 350-351, 362, 364, 379, 387-388, 398, 402, 404, 407, 414, 419, 430, 434, |
| | 441, 446, 454, 462, 465, 479, 481, 488, 491, 497, 516, 518, 528, 537, 553, 571, |
| | 588, 591, 600, 614, 622, 627, 641, 646, 654, 662, 676, 697, 717, 724, 735, 739, |
| | 751, 765, 771, 799, 812, 845, 848, 876, 887, 894-895, 903, 906-907, 917, 921, 932, |
| | 946, 975, 979, 998, 1000, 1012, 1033, 1043, 1047, 1053, 1074, 1087, 1093, 1107, |
| | 1125, 1131, 1143, 1166, 1179, 1184, 1215, 1233, 1245, 1252, 1260, 1264, 1283, |
| | 1291, 1300, 1308, 1316, 1353, 1359, 1369-1370, 1399, 1418-1419, 1435, 1472, |
| | 1481, 1496, 1502, 1505, 1530, 1544, 1553, 1577, 1593, 1597, 1609, 1636, 1654, |
| | 1662, 1677, 1693, 1718, 1720, 1748, 1763, 1765, 1780, 1815, 1830, 1835, 1847, |
| | 1851, 1856-1857, 1868, 1873, 1897, 1926-1927, 1932, 1936, 1947, 1949, 1960, |
| | 1967-1968, 1982, 1985, 1988, 2004, 2020, 2023, 2034, 2045, 2052, 2082, 2087, |
| | 2116, 2129, 2133, 2145, 2173, 2207, 2237, 2239, 2256, 2340, 2350, 2373, 2406, |
| | 2427, 2449, 2483, 2531, 2540, 2551, 2662, 2742, 2778, 2798, 2811, 2823, 2865, |
| | 2916, 2934, 2946, 2957, 2971, 2976, 3094, 3107, 3116, 3256, 3302, 3369, 3374, |
| | 3415, 3429, 3459, 3462, 3547, 3551, 3560, 3687, 3691, 3739, 3773, 3792, 3796, |
| | 3837, 3845 |
| South East Asia | 15, 42, 50, 63, 66-70, 72-73, 126, 179-180, 185, 248, 253, 255, 260, 300, 327, 350- |
| | 351, 362, 364, 379, 398, 402, 404, 407, 414, 419, 434, 441, 446, 454, 462, 465, |
| | 479, 481, 488, 491, 497, 502, 507-508, 516, 518, 528, 537, 553, 571, 591, 600, |
| | 614, 627, 641, 646, 654, 660, 662, 676, 697, 717, 724, 739, 751, 765, 771, 812, |
| | 845, 848, 876, 887, 894-895, 903, 906-907, 917, 921, 932, 946, 975, 979, 1000, |
| | 1012, 1033, 1043, 1047, 1053, 1074, 1085, 1087, 1093, 1099, 1107, 1125, 1131, |
| | 1143, 1165-1166, 1179, 1184, 1215, 1232-1233, 1245, 1252, 1260, 1264, 1283, |
| | 1291, 1300, 1308, 1316, 1353, 1369-1370, 1383, 1391, 1397, 1399, 1418-1419, |
| | 1435, 1481, 1496, 1505, 1517, 1525, 1529-1530, 1544, 1553, 1563, 1577, 1593, |
| | 1597, 1609, 1636, 1654, 1662, 1693, 1763, 1815, 1829, 1835, 1847, 1851, 1857, |
| | 1897, 1926-1927, 1932, 1936, 1949, 1967-1968, 1982, 1985, 1988, 2004, 2023, |
| | 2034-2035, 2045, 2051-2052, 2116, 2126, 2129, 2133, 2145, 2160, 2227, 2262, |
| | 2291-2292, 2340, 2350, 2373, 2382, 2427, 2483, 2540, 2544, 2601, 2679, 2706, |
| | 2797-2798, 2800, 2946, 3066, 3088, 3094, 3107, 3121, 3297, 3343, 3348, 3441, |
| | 3692, 3769, 3785, 3807 |
| Sub-Saharan Africa | 15, 19-20, 50, 63, 66-70, 72-73, 126, 179-180, 206, 235, 248, 253, 260, 362, 364, |
| | 379, 398, 402, 404, 407, 414, 419, 430, 434, 441, 446, 454, 462, 465, 479, 481, |
| | 488, 491, 497, 502, 507-508, 516, 518, 528, 537, 553, 571, 588, 591, 600, 614, |
| | 622, 627, 641, 646, 654, 660, 662, 697, 717, 735, 739, 765, 845, 848, 876, 887, |
| | 894-895, 903, 906-907, 917, 921, 932, 946, 975, 979, 998, 1043, 1047, 1087, 1093, |
| | 1107, 1125, 1143, 1165, 1179, 1184, 1215, 1232-1233, 1245, 1260, 1264, 1283, |
| | 1291, 1300, 1308, 1359, 1370, 1399, 1419, 1435, 1481, 1496, 1517, 1529, 1544, |
| | 1553, 1593, 1597, 1638, 1654, 1662, 1677, 1718, 1720, 1748, 1829, 1847, 1856- |
| | 1857, 1897, 1927, 1960, 1967-1968, 1982, 1988, 2004, 2020, 2034, 2051-2052, |
| | 2070-2071, 2108, 2116, 2126, 2129, 2133, 2145, 2149, 2161, 2173, 2262, 2292, |
| | 2350, 2360, 2373, 2430, 2434, 2458, 2540, 2544, 2548, 2572, 2768, 2798, 2823, |
| | 2894, 2904, 2946, 2969, 3094, 3187, 3209, 3302, 3313, 3515, 3656 |
| Western Asia | 15, 19-20, 50, 63, 66-70, 72-73, 126, 179-180, 185, 235, 248, 260, 300, 327, 344, |
| | 362, 364, 398, 402, 404, 407, 414, 419, 430, 434, 441, 446, 454, 462, 465, 479, |
| | 481, 488, 491, 497, 502, 507-508, 516, 518, 528, 537, 553, 571, 588, 591, 600, |
| | 614, 622, 627, 641, 646, 654, 662, 676, 697, 717, 724, 735, 739, 751, 765, 771, |
| | 799, 812, 845, 876, 887, 894-895, 903, 906-907, 917, 921, 932, 946, 975, 979, 998, |
| | 1012, 1033, 1043, 1047, 1053, 1074, 1087, 1093, 1107, 1125, 1131, 1143, 1165, |
| | 1179, 1184, 1215, 1232-1233, 1245, 1252, 1260, 1264, 1283, 1291, 1300, 1308, |
| | 1316, 1353, 1359, 1369-1370, 1383, 1391, 1397, 1399, 1418-1419, 1435, 1481, |
| | 1486, 1496, 1505, 1517, 1525, 1530, 1544, 1553, 1577, 1593, 1597, 1609, 1636, |
| | 1654, 1662, 1671, 1693, 1718, 1720, 1763, 1780, 1830, 1847, 1851, 1856-1857, |
| | 1873, 1897, 1926-1927, 1932, 1947, 1949, 1960, 1967-1968, 1985, 1988, 2004, |
| | 2020, 2034-2035, 2052, 2116, 2129, 2133, 2145, 2153, 2160, 2173, 2207, 2237, |
| | 2239, 2262, 2282, 2292, 2350, 2373, 2406, 2427, 2540, 2548, 2551, 2572, 2609, |
| | 2778, 2782, 2797-2798, 2811, 2823, 2865, 2971, 3302, 3429, 3434, 3459, 3462, |
| | 3471, 3508, 3524, 3526, 3547 |

TABLE 27
Region-specific peptide pools derived from NSP6 protein for CD8 T cell assays.

| Region | Seq. IDs of peptides |
|---|---|
| Australia | 21-22, 35-36, 82, 105, 210, 394, 432, 439, 452, 472, 480, 484, 494, 517, 584, 610, |
| | 636, 653, 656-657, 666, 699, 705, 708, 756, 781, 795, 840, 846, 874, 891, 911, |
| | 928, 930, 954, 1013, 1026, 1049, 1078, 1089, 1106, 1116, 1127, 1145, 1164, 1191, |
| | 1253, 1275-1276, 1292, 1315, 1320, 1388, 1396, 1398, 1425, 1461, 1475, 1488, |
| | 1550, 1554, 1562, 1603, 1753, 1762, 1800, 1874, 1894, 1937, 1943, 1966, 2003, |
| | 2032, 2044, 2047, 2146, 2171, 2181, 2222, 2280, 2306, 2341, 2404, 2410, 2447, |
| | 2478, 2521, 2528, 2575, 2582, 2587, 2593, 2791, 2810, 2827, 2897, 2985, 3129- |
| | 3130, 3218, 3250, 3345, 3582, 3843 |
| Europe | 21-22, 35-36, 82, 105, 210, 394, 432, 439, 452, 472, 480, 484, 494, 517, 584, 610, |
| | 653, 656-657, 666, 699, 705, 708, 710, 747, 752, 756, 781, 795, 840, 874, 891, |
| | 911, 928, 930, 954, 1026, 1078, 1089, 1106, 1116, 1127, 1145, 1164, 1191, 1253, |
| | 1275-1276, 1292, 1315, 1320, 1388, 1396, 1398, 1425, 1461, 1603, 1725, 1753, |
| | 1762, 1800, 1937, 1966, 2003, 2032, 2047, 2146, 2222, 2280, 2295, 2306, 2451, |
| | 2478, 2528, 2559, 2593, 2733, 2791, 2810, 2990, 3008, 3045, 3130, 3147, 3258, |
| | 3321, 3345, 3375, 3407, 3843 |
| North Africa | 21-22, 35-36, 105, 394, 432, 439, 452, 472, 480, 484, 494, 517, 584, 610, 636, 653, |
| | 656-657, 666, 699, 705, 708, 710, 756, 774, 781, 795, 840, 846, 874, 891, 911, |
| | 928, 930, 954, 984, 1049, 1078, 1089, 1106, 1116, 1127, 1145, 1164, 1191, 1210, |
| | 1253, 1275-1276, 1292, 1315, 1320, 1388, 1396, 1398, 1461, 1475, 1501, 1550- |
| | 1551, 1554, 1725, 1737, 1753, 1762, 1800, 1874, 1894, 1937, 1943, 2032, 2040, |
| | 2047, 2146, 2222, 2280, 2306, 2404, 2414, 2447, 2478, 2521, 2528, 2582, 2587, |
| | 2593, 2735, 2791, 2810, 2827, 2897, 2924, 3105, 3130, 3212, 3243, 3250, 3345, |
| | 3371, 3407, 3470, 3749, 3815 |
| North America | 21-22, 35-36, 82, 105, 210, 394, 432, 439, 452, 494, 517, 584, 610, 636, 653, 656- |
| | 657, 666, 699, 705, 708, 756, 781, 846, 874, 891, 911, 928, 930, 954, 1013, 1026, |
| | 1049, 1078, 1089, 1106, 1116, 1127, 1145, 1164, 1191, 1253, 1276, 1292, 1304, |
| | 1320, 1388, 1396, 1398, 1461, 1488, 1562, 1603, 1617, 1753, 1762, 1800, 1879, |
| | 1894, 1937, 1943, 1966, 2003, 2032, 2044, 2092, 2146, 2171, 2181, 2222, 2280, |
| | 2306, 2410, 2478, 2528, 2559, 2575, 2582, 2593, 2733, 2810, 2885, 2985, 3045, |
| | 3105, 3128-3130, 3180, 3218, 3250, 3258, 3345, 3582, 3640, 3748, 3843 |
| North East Asia | 21-22, 35-36, 82, 105, 210, 394, 432, 439, 452, 472, 480, 484, 494, 517, 584, 610, |
| | 636, 653, 656-657, 666, 699, 705, 708, 710, 756, 774, 781, 795, 840, 846, 874, |
| | 891, 911, 928, 930, 954, 984, 1013, 1078, 1089, 1106, 1116, 1127, 1145, 1164, |
| | 1191, 1210, 1253, 1276, 1292, 1304, 1315, 1320, 1388, 1396, 1398, 1425, 1461, |
| | 1475, 1488, 1501, 1550-1551, 1554, 1562, 1603, 1617, 1753, 1762, 1800, 1874, |
| | 1894, 1943, 1966, 2003, 2032, 2040, 2044, 2047, 2092, 2171, 2181, 2222, 2280, |
| | 2295, 2306, 2404, 2410, 2447, 2478, 2521, 2528, 2559, 2575, 2582, 2587, 2593, |
| | 2733, 2735, 2810, 2897, 2924, 2985, 3045, 3105, 3129-3130, 3147, 3180, 3212, |
| | 3218, 3258, 3345, 3407, 3582, 3640, 3748 |
| Oceania | 21-22, 35-36, 82, 105, 394, 432, 439, 452, 472, 480, 484, 494, 517, 584, 610, 636, |
| | 653, 656-657, 666, 699, 705, 708, 710, 747, 752, 756, 774, 781, 795, 840, 846, |
| | 874, 891, 911, 928, 930, 954, 984, 990, 1013, 1026, 1049, 1078, 1089, 1106, 1116, |
| | 1127, 1145, 1164, 1191, 1210, 1253, 1276, 1290, 1292, 1315, 1318, 1320, 1388, |
| | 1396, 1398, 1425, 1461, 1475, 1488, 1501, 1550-1551, 1554, 1562, 1603, 1725, |
| | 1737, 1753, 1762, 1800, 1874, 1879, 1894, 1937, 1943, 1966, 2003, 2032, 2044, |
| | 2047, 2092, 2146, 2171, 2181, 2222, 2280, 2295, 2306, 2341, 2404, 2410, 2447, |
| | 2478, 2528, 2559, 2582, 2587, 2593, 2733, 2810, 2827, 2897, 2985, 3008, 3045, |
| | 3105, 3128-3130, 3204, 3212, 3218, 3250, 3258, 3292, 3345, 3371, 3407, 3582 |
| South and Central America | 21-22, 35-36, 82, 105, 394, 432, 439, 452, 472, 480, 484, 494, 517, 584, 610, 636, |
| | 653, 656-657, 666, 699, 705, 708, 710, 756, 774, 781, 795, 840, 846, 874, 891, |
| | 911, 928, 930, 954, 984, 990, 1013, 1026, 1049, 1078, 1089, 1106, 1116, 1127, |
| | 1145, 1164, 1191, 1210, 1253, 1275-1276, 1292, 1315, 1318, 1320, 1388, 1396, |
| | 1398, 1425, 1461, 1475, 1488, 1551, 1554, 1603, 1753, 1762, 1800, 1894, 1937, |
| | 1943, 1966, 2003, 2032, 2040, 2044, 2047, 2146, 2222, 2280, 2295, 2306, 2447, |
| | 2478, 2521, 2559, 2582, 2587, 2593, 2733, 2810, 2827, 3045, 3130, 3147, 3250, |
| | 3258, 3345, 3371, 3582, 3843 |
| South Asia | 21-22, 35-36, 82, 105, 394, 432, 439, 452, 472, 480, 484, 494, 517, 584, 610, 636, |
| | 653, 656-657, 666, 699, 705, 708, 710, 747, 752, 756, 774, 781, 795, 840, 846, |
| | 874, 891, 911, 928, 930, 954, 984, 990, 1013, 1026, 1049, 1078, 1089, 1106, 1116, |
| | 1127, 1145, 1164, 1191, 1210, 1253, 1276, 1290, 1292, 1304, 1315, 1318, 1320, |
| | 1388, 1396, 1398, 1425, 1461, 1475, 1488, 1501, 1550-1551, 1554, 1562, 1603, |
| | 1725, 1737, 1753, 1762, 1800, 1874, 1894, 1937, 1966, 2003, 2032, 2044, 2047, |
| | 2146, 2222, 2280, 2295, 2306, 2478, 2521, 2528, 2559, 2582, 2587, 2593, 2733, |
| | 2735, 2758, 2810, 2827, 2897, 2924, 2990, 3008, 3045, 3105, 3130, 3204, 3218, |
| | 3258, 3292, 3371, 3666, 3815, 3843 |
| South East Asia | 21-22, 35-36, 82, 105, 394, 432, 439, 452, 472, 480, 484, 494, 517, 584, 610, 636, |
| | 653, 656-657, 666, 699, 705, 708, 710, 747, 756, 774, 781, 840, 846, 874, 891, |
| | 911, 928, 930, 954, 984, 990, 1013, 1026, 1078, 1089, 1106, 1116, 1127, 1145, |
| | 1191, 1210, 1253, 1275-1276, 1292, 1315, 1318, 1320, 1388, 1396, 1398, 1425, |
| | 1461, 1475, 1488, 1501, 1550-1551, 1554, 1562, 1603, 1725, 1753, 1762, 1800, |
| | 1879, 1937, 1943, 1966, 2003, 2032, 2040, 2044, 2092, 2171, 2181, 2222, 2280, |
| | 2295, 2306, 2341, 2410, 2447, 2478, 2521, 2528, 2559, 2582, 2587, 2593, 2733, |
| | 2810, 2897, 2985, 3008, 3045, 3105, 3128-3130, 3204, 3218, 3258, 3292, 3328, |
| | 3345, 3407, 3543, 3582, 3636, 3640, 3748 |
| Sub-Saharan Africa | 21-22, 35-36, 82, 105, 210, 394, 432, 439, 452, 480, 484, 494, 517, 584, 610, 636, |
| | 653, 656-657, 666, 699, 705, 708, 710, 756, 781, 846, 874, 891, 911, 928, 930, |
| | 954, 984, 1013, 1049, 1078, 1089, 1106, 1116, 1127, 1145, 1164, 1191, 1253, |
| | 1275-1276, 1290, 1292, 1304, 1315, 1320, 1388, 1396, 1398, 1461, 1475, 1488, |
| | 1550, 1554, 1562, 1603, 1617, 1737, 1753, 1762, 1800, 1874, 1879, 1894, 1937, |
| | 1966, 2003, 2032, 2040, 2047, 2092, 2146, 2171, 2181, 2222, 2280, 2295, 2306, |
| | 2404, 2447, 2478, 2521, 2528, 2575, 2582, 2587, 2593, 2733, 2791, 2810, 2885, |
| | 2924, 2985, 3105, 3130, 3212, 3243, 3250, 3258, 3292, 3345, 3407, 3470, 3749 |
| Western Asia | 21-22, 35-36, 82, 105, 394, 432, 439, 452, 472, 480, 484, 494, 517, 584, 610, 653, |
| | 656-657, 666, 699, 705, 708, 710, 747, 756, 774, 781, 795, 840, 846, 874, 891, |
| | 911, 928, 930, 954, 984, 990, 1013, 1026, 1049, 1078, 1089, 1106, 1116, 1127, |
| | 1145, 1164, 1191, 1210, 1253, 1275-1276, 1290, 1292, 1304, 1315, 1318, 1320, |
| | 1388, 1396, 1398, 1425, 1461, 1475, 1488, 1501, 1550-1551, 1554, 1562, 1603, |
| | 1617, 1737, 1753, 1762, 1800, 1874, 1879, 1894, 1937, 1943, 1966, 2003, 2032, |
| | 2040, 2044, 2047, 2092, 2146, 2222, 2280, 2295, 2306, 2447, 2478, 2521, 2528, |
| | 2559, 2582, 2587, 2593, 2733, 2735, 2791, 2810, 2827, 2897, 2990, 3008, 3045, |
| | 3105, 3128, 3130, 3147, 3204, 3258, 3292, 3321, 3371, 3543, 3843 |

TABLE 28
Region-specific peptide pools derived from E protein for CD8 T cell assays.

| Region | Seq. IDs of peptides |
|---|---|
| Australia | 29, 396, 444, 578, 948, 1081, 1196, 1345, 1441, 1629, 1690, 1740, 1744, 1848, |
| | 1902, 1969, 2022, 2064, 2294, 2347, 2442, 2468, 2583, 2611, 2852, 2955, 3188, |
| | 3265, 3298, 3566, 3605 |
| Europe | 29, 396, 444, 578, 948, 1081, 1196, 1345, 1441, 1690, 1740, 1744, 1848, 1902, |
| | 1969, 2064, 2139, 2294, 2347, 2442, 2468, 2491, 2583, 2611, 3188, 3287, 3605, |
| | 3644 |
| North Africa | 29, 396, 444, 948, 1081, 1196, 1345, 1629, 1690, 1740, 1744, 1848, 1902, 1969, |
| | 2022, 2064, 2294, 2347, 2468, 2491, 2583, 2611, 3188, 3265, 3298, 3378, 3566, |
| | 3605, 3678 |
| North America | 29, 396, 444, 578, 948, 1081, 1196, 1441, 1690, 1740, 1744, 1848, 1902, 1969, |
| | 2064, 2139, 2294, 2347, 2468, 2611, 2852, 3188, 3265, 3287, 3298, 3605, 3768 |
| North East Asia | 29, 396, 444, 578, 948, 1081, 1196, 1345, 1441, 1629, 1690, 1740, 1744, 1848, |
| | 1902, 1969, 2022, 2064, 2139, 2294, 2347, 2442, 2468, 2491, 2583, 2611, 2955, |
| | 3188, 3265, 3287, 3662, 3678, 3768 |
| Oceania | 29, 396, 444, 578, 948, 1081, 1196, 1345, 1441, 1629, 1690, 1740, 1744, 1848, |
| | 1902, 1969, 2022, 2064, 2139, 2294, 2347, 2442, 2468, 2491, 2611, 2852, 2955, |
| | 3188, 3265, 3298, 3566, 3605, 3662 |
| South and Central America | 29, 396, 444, 578, 948, 1081, 1196, 1345, 1441, 1629, 1690, 1740, 1744, 1848, |
| | 1902, 1969, 2022, 2064, 2139, 2294, 2442, 2468, 2491, 2583, 2611, 2852, 2955, |
| | 3188, 3265, 3287, 3298, 3566, 3605 |
| South Asia | 29, 396, 444, 578, 948, 1081, 1196, 1345, 1441, 1629, 1690, 1740, 1744, 1848, |
| | 1902, 1969, 2022, 2064, 2139, 2294, 2347, 2442, 2468, 2491, 2583, 2611, 2955, |
| | 3188, 3265, 3378, 3566, 3605, 3662, 3678 |
| South East Asia | 29, 396, 444, 578, 948, 1081, 1196, 1441, 1629, 1690, 1740, 1744, 1848, 1902, |
| | 1969, 2064, 2139, 2294, 2347, 2442, 2468, 2491, 2583, 2611, 2852, 2955, 3188, |
| | 3768, 3805 |
| Sub-Saharan Africa | 29, 396, 444, 578, 948, 1081, 1196, 1345, 1441, 1629, 1690, 1740, 1744, 1848, |
| | 1902, 1969, 2064, 2294, 2347, 2468, 2611, 3188, 3265, 3287, 3298, 3378, 3566, |
| | 3605 |
| Western Asia | 29, 396, 444, 578, 948, 1081, 1196, 1345, 1441, 1629, 1690, 1740, 1744, 1848, |
| | 1902, 1969, 2064, 2294, 2347, 2468, 2491, 2583, 2611, 2852, 2955, 3188, 3265, |
| | 3287, 3566, 3605, 3644, 3678 |

TABLE 29
Region-specific peptide pools derived from NSP8 protein for CD8 T cell assays.

| Region | Seq. IDs of peptides |
|---|---|
| Australia | 33, 335, 459, 513, 534, 548, 602, 671, 731, 822, 897, 974, 1042, 1103, 1105, |
| | 1108, 1139, 1205, 1306, 1321, 1371, 1380, 1493, 1514, 1614, 1632, 1641, 1646, |
| | 1749, 1827, 1839, 1946, 1959, 2081, 2107, 2110, 2127, 2201, 2220, 2235, 2265, |
| | 2281, 2296, 2433, 2466, 2473, 2578, 2622, 2709, 2741, 2831, 2878, 2970, 3011, |
| | 3053, 3158, 3222, 3356, 3400, 3402, 3420, 3489, 3673, 3746 |
| Europe | 33, 335, 459, 513, 534, 548, 602, 671, 731, 897, 974, 1042, 1103, 1108, 1139, |
| | 1205, 1306, 1321, 1371, 1493, 1614, 1632, 1641, 1749, 1827, 1839, 1946, 2081, |
| | 2107, 2110, 2220, 2235, 2265, 2296, 2385, 2433, 2466, 2473, 2709, 2878, 2970, |
| | 3011, 3053, 3136, 3158, 3222, 3356, 3400, 3489 |
| North Africa | 33, 335, 459, 534, 548, 671, 731, 822, 897, 919, 974, 1042, 1103, 1105, 1108, |
| | 1139, 1205, 1306, 1321, 1371, 1380, 1493, 1514, 1614, 1632, 1646, 1685, 1749, |
| | 1827, 1839, 1946, 1959, 1989, 2081, 2107, 2110, 2127, 2201, 2220, 2265, 2296, |
| | 2401, 2466, 2473, 2878, 2970, 3011, 3053, 3115, 3158, 3172, 3222, 3356, 3400, |
| | 3489, 3751, 3804, 3821 |
| North America | 33, 335, 459, 504, 513, 534, 548, 602, 671, 897, 974, 1042, 1103, 1105, 1108, |
| | 1139, 1306, 1321, 1371, 1380, 1493, 1514, 1614, 1632, 1641, 1646, 1749, 1827, |
| | 1946, 1987, 2081, 2107, 2110, 2189, 2201, 2265, 2281, 2296, 2466, 2473, 2578, |
| | 2816, 2878, 2970, 3011, 3053, 3136, 3158, 3222, 3356, 3400, 3402, 3674, 3842 |
| North East Asia | 33, 335, 459, 513, 534, 548, 602, 671, 731, 822, 897, 919, 974, 1042, 1103, 1105, |
| | 1108, 1139, 1205, 1306, 1321, 1371, 1380, 1493, 1514, 1614, 1632, 1641, 1646, |
| | 1749, 1827, 1839, 1946, 1987, 2081, 2107, 2110, 2201, 2220, 2235, 2265, 2296, |
| | 2433, 2466, 2473, 2498, 2578, 2709, 2741, 2970, 3011, 3053, 3136, 3158, 3172, |
| | 3356, 3400, 3443, 3502, 3576, 3674 |
| Oceania | 33, 335, 459, 504, 513, 534, 548, 602, 671, 731, 822, 897, 919, 974, 1042, 1103, |
| | 1105, 1108, 1139, 1205, 1306, 1321, 1371, 1380, 1493, 1514, 1614, 1632, 1641, |
| | 1646, 1685, 1749, 1827, 1839, 1946, 1959, 2081, 2107, 2110, 2127, 2201, 2220, |
| | 2235, 2265, 2281, 2296, 2385, 2433, 2466, 2473, 2498, 2578, 2622, 2702, 2709, |
| | 2741, 2816, 2970, 3011, 3053, 3136, 3158, 3172, 3222, 3356, 3400, 3402, 3420, |
| | 3443, 3489, 3576, 3673 |
| South and Central America | 33, 335, 459, 513, 534, 548, 602, 671, 731, 822, 897, 919, 974, 1042, 1103, 1105, |
| | 1108, 1139, 1205, 1306, 1321, 1371, 1380, 1493, 1514, 1614, 1632, 1646, 1749, |
| | 1827, 1946, 1959, 2081, 2107, 2110, 2127, 2201, 2220, 2265, 2281, 2296, 2466, |
| | 2473, 2498, 2578, 2622, 2741, 2831, 2878, 2970, 3011, 3053, 3136, 3158, 3222, |
| | 3356, 3400, 3402, 3420, 3443, 3489, 3730 |
| South Asia | 33, 335, 459, 513, 534, 548, 602, 671, 731, 822, 897, 919, 974, 1042, 1103, 1105, |
| | 1108, 1139, 1205, 1306, 1321, 1371, 1380, 1493, 1514, 1614, 1632, 1641, 1646, |
| | 1685, 1749, 1827, 1839, 1946, 1959, 1987, 1989, 2081, 2107, 2110, 2127, 2220, |
| | 2235, 2265, 2296, 2385, 2466, 2473, 2498, 2578, 2686, 2702, 2878, 2970, 3011, |
| | 3053, 3136, 3158, 3172, 3222, 3356, 3400, 3443, 3489, 3576 |
| South East Asia | 33, 335, 459, 504, 513, 534, 548, 602, 671, 731, 897, 919, 974, 1042, 1103, 1105, |
| | 1108, 1139, 1306, 1321, 1371, 1380, 1493, 1514, 1614, 1632, 1641, 1646, 1685, |
| | 1749, 1827, 1839, 1946, 2081, 2107, 2110, 2201, 2235, 2265, 2281, 2296, 2385, |
| | 2433, 2466, 2473, 2498, 2578, 2702, 2709, 2816, 2831, 2878, 2970, 3011, 3053, |
| | 3136, 3158, 3202, 3356, 3400, 3402, 3420, 3443, 3674, 3746 |
| Sub-Saharan Africa | 33, 335, 459, 504, 513, 534, 548, 602, 671, 731, 897, 974, 1042, 1103, 1105, |
| | 1108, 1139, 1306, 1321, 1371, 1380, 1493, 1514, 1614, 1632, 1641, 1646, 1749, |
| | 1827, 1839, 1946, 1959, 2081, 2107, 2110, 2127, 2189, 2201, 2265, 2281, 2296, |
| | 2401, 2466, 2473, 2970, 3011, 3053, 3158, 3356, 3400, 3489, 3615, 3751, 3804 |
| Western Asia | 33, 335, 459, 504, 513, 534, 548, 602, 671, 731, 897, 919, 974, 1042, 1103, 1105, |
| | 1108, 1139, 1205, 1306, 1321, 1380, 1493, 1514, 1614, 1632, 1641, 1646, 1749, |
| | 1827, 1839, 1946, 1959, 2081, 2107, 2110, 2127, 2189, 2201, 2220, 2235, 2265, |
| | 2281, 2296, 2385, 2433, 2466, 2473, 2686, 2702, 2709, 2816, 2831, 2878, 2970, |
| | 3011, 3053, 3136, 3158, 3172, 3222, 3356, 3400, 3402, 3489, 3746, 3854 |

TABLE 30
Region-specific peptide pools derived from ORF3a protein for CD8 T cell assays.

| Region | Seq. IDs of peptides |
|---|---|
| Australia | 38, 40, 80, 92, 128-129, 131, 184, 186, 228, 275, 323, 375, 399, 435, 442-443, |
| | 451, 461, 464, 466, 524, 526, 552, 619, 642, 721, 810, 1003, 1062, 1140, 1223, |
| | 1265, 1279, 1327, 1340, 1445, 1476, 1539, 1549, 1585-1586, 1643, 1730, 1792, |
| | 1820, 1824-1825, 1840, 1898, 1986, 2068, 2083, 2136, 2138, 2164, 2190, 2200, |
| | 2242, 2301, 2309, 2314, 2374, 2408, 2504, 2513, 2590, 2672, 2685, 2739, 2801, |
| | 2807, 2815, 2860, 2929, 3043, 3054, 3095, 3110, 3154, 3161, 3171, 3220, 3231, |
| | 3323, 3494, 3496, 3578, 3638, 3654, 3683, 3756, 3799, 3823, 3841 |
| Europe | 38, 40, 80, 92, 128-129, 131, 184, 186, 228, 275, 323, 352-353, 366, 375, 399, |
| | 435, 442-443, 451, 461, 464, 466, 524, 526, 552, 619, 642, 658, 721, 740, 810, |
| | 1003, 1062, 1140, 1198, 1223, 1279, 1340, 1387, 1445, 1476, 1539, 1549, 1585, |
| | 1590, 1643, 1792, 1799, 1820, 1822, 1824-1825, 1898, 1965, 1986, 2068, 2083, |
| | 2136, 2164, 2190, 2209, 2278, 2309, 2314, 2374, 2408, 2504, 2513, 2590, 2672, |
| | 2739, 2801, 2807, 2815, 2929, 3110, 3154, 3220, 3494, 3578, 3715, 3731, 3799, |
| | 3841 |
| North Africa | 38, 40, 80, 92, 128-129, 131, 228, 275, 323, 352-353, 366, 375, 399, 435, 442- |
| | 443, 451, 461, 464, 466, 524, 526, 552, 619, 642, 658, 721, 779, 810, 1003, 1062, |
| | 1140, 1223, 1265, 1279, 1327, 1340, 1445, 1476, 1539, 1549, 1569, 1586, 1643, |
| | 1730, 1792, 1820, 1824-1825, 1840, 1986, 2025, 2068, 2083, 2136, 2138, 2190, |
| | 2200, 2242, 2278, 2314, 2374, 2408, 2504, 2513, 2590, 2666, 2672, 2685, 2739, |
| | 2801, 2807, 2815, 2859-2860, 2888, 2923, 2929, 3054, 3110, 3154, 3220, 3231, |
| | 3269, 3323, 3332, 3494, 3496, 3578, 3638, 3654, 3736, 3799 |
| North America | 38, 40, 80, 92, 128-129, 131, 184, 186, 228, 275, 323, 352-353, 375, 399, 435, |
| | 442-443, 451, 461, 464, 466, 524, 526, 552, 619, 642, 721, 810, 1003, 1062, 1140, |
| | 1198, 1223, 1279, 1327, 1340, 1387, 1445, 1539, 1549, 1585, 1643, 1730, 1792, |
| | 1799, 1820, 1824, 1840, 1898, 2025, 2083, 2138, 2164, 2190, 2200, 2209, 2242, |
| | 2309, 2314, 2374, 2408, 2504, 2513, 2546, 2739, 2815, 2860, 2923, 2929, 3043, |
| | 3110, 3154, 3171, 3220, 3494, 3578, 3654, 3711, 3722, 3799, 3823, 3838, 3841 |
| North East Asia | 38, 40, 80, 92, 128-129, 131, 184, 186, 275, 352-353, 375, 399, 435, 442-443, |
| | 451, 461, 464, 466, 524, 526, 552, 619, 642, 658, 721, 779, 810, 1003, 1062, |
| | 1140, 1198, 1223, 1265, 1279, 1327, 1340, 1387, 1445, 1476, 1539, 1549, 1569, |
| | 1585-1586, 1643, 1730, 1792, 1799, 1824, 1840, 1898, 1986, 2068, 2083, 2136, |
| | 2138, 2164, 2190, 2209, 2242, 2254, 2301, 2309, 2314, 2374, 2408, 2504, 2513, |
| | 2546, 2590, 2672, 2685, 2739, 2801, 2807, 2815, 2859-2860, 2923, 2929, 3043, |
| | 3095, 3110, 3154, 3157, 3171, 3269, 3323, 3332, 3494, 3573, 3641, 3683, 3699, |
| | 3711, 3823 |
| Oceania | 38, 40, 80, 92, 128-129, 184, 186, 275, 366, 375, 435, 442-443, 451, 461, 464, |
| | 466, 524, 526, 552, 619, 642, 658, 721, 779, 810, 1003, 1062, 1140, 1198, 1223, |
| | 1265, 1279, 1327, 1340, 1387, 1445, 1474, 1476, 1539, 1549, 1569, 1585-1586, |
| | 1590, 1643, 1730, 1792, 1799, 1820, 1824-1825, 1840, 1898, 1965, 1986, 2025, |
| | 2068, 2083, 2136, 2138, 2164, 2190, 2200, 2209, 2242, 2254, 2266, 2301, 2309, |
| | 2314, 2374, 2408, 2504, 2513, 2590, 2672, 2685, 2739, 2801, 2807, 2815, 2859- |
| | 2860, 2923, 2929, 3043, 3095, 3110, 3154, 3161, 3171, 3220, 3231, 3269, 3317, |
| | 3323, 3332, 3494, 3496, 3573, 3596, 3638, 3641, 3683, 3699, 3794, 3823 |
| South and Central America | 38, 40, 80, 92, 128-129, 184, 186, 275, 352-353, 366, 375, 399, 435, 442-443, |
| | 451, 461, 464, 466, 524, 526, 552, 619, 642, 658, 721, 779, 810, 1003, 1062, |
| | 1140, 1198, 1223, 1265, 1279, 1327, 1340, 1387, 1445, 1476, 1539, 1549, 1569, |
| | 1585-1586, 1643, 1730, 1792, 1799, 1820, 1824-1825, 1840, 1898, 1965, 1986, |
| | 2068, 2083, 2138, 2190, 2200, 2209, 2242, 2266, 2301, 2309, 2314, 2374, 2408, |
| | 2504, 2513, 2590, 2672, 2685, 2739, 2801, 2807, 2815, 2859-2860, 2929, 3020, |
| | 3043, 3054, 3095, 3110, 3154, 3161, 3220, 3231, 3323, 3332, 3494, 3496, 3556, |
| | 3578, 3638, 3683, 3699, 3799, 3823, 3841 |
| South Asia | 38, 40, 80, 92, 128-129, 131, 184, 186, 275, 352-353, 366, 375, 399, 435, 442- |
| | 443, 451, 461, 464, 466, 524, 526, 552, 619, 642, 658, 721, 779, 810, 1003, 1062, |
| | 1140, 1198, 1223, 1265, 1279, 1327, 1340, 1387, 1445, 1474, 1476, 1539, 1549, |
| | 1569, 1585-1586, 1590, 1643, 1730, 1792, 1820, 1822, 1824-1825, 1840, 1898, |
| | 1965, 1986, 2025, 2068, 2083, 2136, 2138, 2164, 2190, 2209, 2242, 2254, 2266, |
| | 2278, 2301, 2309, 2314, 2374, 2408, 2504, 2513, 2590, 2672, 2685, 2739, 2801, |
| | 2807, 2815, 2859-2860, 2888, 2929, 3043, 3054, 3110, 3154, 3220, 3231, 3269, |
| | 3323, 3332, 3392, 3496, 3555-3556, 3573, 3578, 3592, 3635, 3641, 3683, 3762, |
| | 3795, 3799, 3841 |
| South East Asia | 38, 40, 80, 92, 128-129, 131, 184, 186, 275, 352-353, 366, 375, 399, 435, 442- |
| | 443, 451, 461, 464, 466, 524, 526, 552, 619, 642, 658, 721, 779, 810, 1003, 1062, |
| | 1140, 1198, 1223, 1265, 1279, 1327, 1340, 1387, 1445, 1476, 1539, 1549, 1569, |
| | 1585-1586, 1590, 1643, 1730, 1792, 1799, 1820, 1822, 1824-1825, 1840, 1898, |
| | 1965, 2083, 2136, 2138, 2164, 2190, 2209, 2242, 2266, 2301, 2309, 2314, 2374, |
| | 2408, 2504, 2513, 2672, 2739, 2801, 2815, 2859, 2923, 2929, 2993, 3020, 3043, |
| | 3054, 3095, 3110, 3154, 3161, 3171, 3269, 3317, 3323, 3332, 3494, 3596, 3711, |
| | 3794, 3823 |
| Sub-Saharan Africa | 38, 40, 80, 92, 128-129, 131, 184, 186, 228, 275, 375, 399, 435, 442-443, 451, |
| | 461, 464, 466, 524, 526, 552, 619, 642, 658, 721, 810, 1003, 1062, 1140, 1198, |
| | 1223, 1279, 1340, 1387, 1445, 1474, 1539, 1549, 1585, 1643, 1730, 1792, 1799, |
| | 1820, 1824-1825, 1840, 2068, 2083, 2164, 2190, 2200, 2209, 2309, 2314, 2374, |
| | 2408, 2504, 2513, 2546, 2666, 2672, 2739, 2801, 2807, 2815, 2860, 2923, 2929, |
| | 3110, 3154, 3220, 3231, 3269, 3494, 3496, 3555, 3578, 3638, 3654, 3722, 3794, |
| | 3799 |
| Western Asia | 38, 40, 80, 92, 128-129, 131, 184, 186, 228, 275, 366, 375, 399, 435, 442-443, |
| | 451, 461, 464, 466, 524, 526, 552, 619, 642, 658, 721, 779, 810, 1003, 1062, |
| | 1140, 1198, 1223, 1265, 1279, 1327, 1340, 1387, 1445, 1474, 1476, 1539, 1549, |
| | 1569, 1585-1586, 1590, 1643, 1792, 1799, 1820, 1822, 1824-1825, 1898, 1965, |
| | 1986, 2068, 2083, 2138, 2164, 2190, 2209, 2242, 2254, 2266, 2278, 2309, 2314, |
| | 2374, 2408, 2504, 2513, 2590, 2672, 2739, 2801, 2807, 2815, 2859-2860, 2929, |
| | 3054, 3095, 3110, 3154, 3220, 3231, 3323, 3496, 3556, 3578, 3596, 3715, 3799, |
| | 3817, 3841, 3857 |

TABLE 31
Region-specific peptide pools derived from NSP1 protein for CD8 T cell assays.

| Region | Seq. IDs of peptides |
|---|---|
| Australia | 39, 231, 634, 820, 870, 883, 964, 980, 1001-1002, 1019, 1048, 1310-1311, 1332, |
| | 1343, 1376, 1385, 1443, 1613, 1623, 1724, 1892, 2028, 2267, 2311, 2378, 2591, |
| | 2654, 2725, 2868, 2913, 3024, 3080, 3182, 3518, 3536, 3553, 3571, 3632, 3690, |
| | 3770 |
| Europe | 39, 231, 870, 883, 964, 1001-1002, 1019, 1310-1311, 1332, 1385, 1613, 1892, |
| | 2028, 2654, 2803, 2817, 2825, 2913, 3024, 3125, 3182, 3186, 3195, 3474, 3571, |
| | 3649, 3690, 3770 |
| North Africa | 39, 231, 634, 820, 870, 883, 964, 1001-1002, 1019, 1048, 1310-1311, 1332, 1341, |
| | 1343, 1385, 1443, 1613, 2028, 2228, 2267, 2378, 2591, 2654, 2725, 2913, 2979, |
| | 3024, 3080, 3182, 3192, 3198, 3380, 3412, 3474, 3518, 3546, 3571, 3633, 3690 |
| North America | 39, 231, 634, 870, 883, 964, 1001-1002, 1019, 1048, 1310-1311, 1332, 1385, |
| | 1443, 1613, 1724, 1892, 2267, 2370, 2378, 2654, 2725, 2803, 2817, 2868, 2913, |
| | 3024, 3080, 3125, 3182, 3195, 3522, 3553, 3571, 3601, 3632, 3690, 3770 |
| North East Asia | 39, 231, 634, 820, 870, 883, 964, 1001-1002, 1019, 1048, 1310-1311, 1332, 1341, |
| | 1343, 1376, 1385, 1443, 1613, 1623, 1892, 2028, 2206, 2267, 2311, 2378, 2591, |
| | 2654, 2803, 2817, 2868, 2913, 3024, 3080, 3125, 3182, 3195, 3198, 3546, 3553, |
| | 3632 |
| Oceania | 39, 231, 634, 820, 870, 883, 964, 980, 1001-1002, 1019, 1048, 1310-1311, 1332, |
| | 1341, 1343, 1376, 1385, 1443, 1613, 1623, 1724, 1892, 2028, 2206, 2267, 2311, |
| | 2378, 2591, 2654, 2725, 2817, 2868, 2913, 2915, 3024, 3080, 3125, 3182, 3195, |
| | 3522, 3553, 3632, 3770 |
| South and Central America | 39, 231, 634, 820, 870, 883, 964, 980, 1001-1002, 1019, 1048, 1310-1311, 1332, |
| | 1341, 1343, 1376, 1385, 1443, 1613, 1623, 1724, 1892, 2028, 2228, 2267, 2311, |
| | 2378, 2591, 2654, 2725, 2803, 2817, 2843, 2868, 2913, 3024, 3080, 3125, 3182, |
| | 3192, 3195, 3198, 3571, 3690, 3770 |
| South Asia | 39, 634, 820, 870, 883, 964, 980, 1001-1002, 1019, 1048, 1310-1311, 1332, 1341, |
| | 1343, 1376, 1385, 1443, 1613, 1623, 1892, 2028, 2206, 2228, 2267, 2311, 2378, |
| | 2591, 2654, 2817, 2843, 2868, 2913, 2915, 3024, 3080, 3125, 3182, 3192, 3195, |
| | 3464, 3571, 3633, 3690, 3729, 3770 |
| South East Asia | 39, 231, 870, 883, 964, 980, 1001-1002, 1019, 1048, 1310-1311, 1332, 1341, |
| | 1376, 1385, 1443, 1613, 1623, 1724, 1892, 2028, 2206, 2228, 2267, 2311, 2378, |
| | 2654, 2817, 2825, 2843, 2868, 2913, 2915, 3093, 3125, 3182, 3195, 3518, 3522, |
| | 3553, 3632 |
| Sub-Saharan Africa | 39, 231, 634, 870, 883, 964, 1001-1002, 1019, 1048, 1310-1311, 1332, 1385, |
| | 1443, 1613, 1724, 1892, 2267, 2370, 2378, 2654, 2725, 2913, 3024, 3080, 3125, |
| | 3182, 3198, 3380, 3518, 3546, 3571, 3631, 3690 |
| Western Asia | 39, 231, 634, 870, 883, 964, 980, 1001-1002, 1019, 1048, 1310-1311, 1332, 1341, |
| | 1385, 1443, 1613, 1724, 1892, 2028, 2228, 2267, 2370, 2654, 2803, 2817, 2825, |
| | 2843, 2868, 2913, 2915, 3024, 3093, 3125, 3182, 3195, 3474, 3518, 3522, 3571, |
| | 3649, 3690, 3770 |

TABLE 32
Region-specific peptide pools derived from ORF7a protein for CD8 T cell assays.

| Region | Seq. IDs of peptides |
|---|---|
| Australia | 54, 207, 225, 237, 535, 701, 738, 938, 1034, 1147, 1258, 1331, 1352, 1448, 1500, |
| | 1642, 1645, 1659, 1675, 1913, 2069, 2086, 2098, 2229, 2534, 2541, 2689, 2771, |
| | 2950, 3014, 3068, 3168, 3185, 3236, 3239, 3254, 3352, 3395 |
| Europe | 54, 207, 225, 237, 535, 701, 738, 938, 1034, 1147, 1258, 1352, 1448, 1500, 1645, |
| | 1672, 1675, 1682, 1913, 1999, 2069, 2086, 2229, 2367, 2541, 2689, 2692, 2771, |
| | 2950, 3014, 3090-3091, 3168, 3185, 3239, 3267, 3352, 3395 |
| North Africa | 54, 207, 225, 237, 535, 701, 738, 938, 1034, 1147, 1212, 1258, 1331, 1352, 1500, |
| | 1642, 1645, 1659, 1675, 1682, 1913, 2069, 2086, 2098, 2229, 2541, 2576, 2689, |
| | 2806, 3014, 3131, 3168, 3236, 3239, 3352, 3395, 3431, 3628, 3652, 3689, 3824 |
| North America | 54, 207, 225, 237, 535, 738, 938, 1034, 1147, 1258, 1352, 1448, 1672, 1675, |
| | 1913, 1999, 2069, 2098, 2229, 2534, 2771, 3014, 3068, 3168, 3199, 3239, 3254, |
| | 3267, 3352, 3791 |
| North East Asia | 207, 225, 535, 701, 738, 938, 1034, 1147, 1212, 1258, 1331, 1352, 1448, 1500, |
| | 1642, 1645, 1659, 1672, 1675, 1913, 1999, 2069, 2086, 2098, 2229, 2534, 2541, |
| | 2576, 2689, 2771, 2806, 3014, 3068, 3168, 3236, 3239, 3254, 3267, 3352, 3395, |
| | 3725, 3791 |
| Oceania | 207, 225, 535, 701, 738, 938, 1034, 1147, 1212, 1258, 1331, 1352, 1448, 1500, |
| | 1642, 1645, 1659, 1672, 1675, 1913, 1999, 2069, 2086, 2098, 2148, 2229, 2534, |
| | 2541, 2576, 2689, 2771, 2806, 2950, 3014, 3068, 3168, 3185, 3199, 3236, 3239, |
| | 3254, 3267, 3352, 3395, 3431, 3725, 3743 |
| South and Central America | 54, 207, 225, 535, 701, 738, 938, 1034, 1147, 1212, 1258, 1352, 1448, 1500, |
| | 1645, 1659, 1672, 1675, 1913, 1999, 2069, 2086, 2098, 2229, 2534, 2541, 2689, |
| | 2771, 2806, 3014, 3068, 3168, 3236, 3239, 3254, 3267, 3352, 3395, 3698, 3743 |
| South Asia | 54, 207, 225, 535, 701, 738, 938, 1034, 1147, 1212, 1258, 1331, 1352, 1448, |
| | 1500, 1642, 1645, 1659, 1672, 1675, 1682, 1913, 1999, 2069, 2086, 2098, 2148, |
| | 2229, 2534, 2541, 2576, 2689, 2771, 2806, 3014, 3027, 3068, 3090, 3131, 3168, |
| | 3236, 3239, 3267, 3395, 3431, 3684, 3723, 3743 |
| South East Asia | 207, 225, 535, 701, 738, 938, 1034, 1147, 1212, 1258, 1352, 1448, 1500, 1642, |
| | 1645, 1672, 1675, 1913, 1999, 2069, 2086, 2098, 2148, 2229, 2534, 2539, 2541, |
| | 2576, 2689, 2771, 2806, 2950, 3014, 3068, 3091, 3168, 3199, 3239, 3254, 3267, |
| | 3352, 3725, 3743, 3765 |
| Sub-Saharan Africa | 54, 207, 225, 237, 535, 701, 738, 938, 1034, 1147, 1212, 1258, 1331, 1352, 1448, |
| | 1642, 1672, 1675, 1913, 1999, 2069, 2098, 2541, 2576, 2689, 2771, 3014, 3027, |
| | 3068, 3090, 3168, 3199, 3239, 3395, 3431, 3652, 3689 |
| Western Asia | 207, 225, 237, 535, 701, 738, 938, 1034, 1147, 1212, 1258, 1352, 1448, 1500, |
| | 1642, 1645, 1659, 1672, 1675, 1682, 1913, 1999, 2069, 2086, 2098, 2148, 2229, |
| | 2539, 2541, 2576, 2689, 2771, 2806, 2950, 3014, 3027, 3068, 3090, 3168, 3199, |
| | 3239, 3352, 3395, 3431, 3765 |

TABLE 33
Region-specific peptide pools derived from NSP2 protein for CD8 T cell assays.

| Region | Seq. IDs of peptides |
|---|---|
| Australia | 65, 81, 250, 302, 395, 514, 547, 576, 580, 592-593, 606, 612, 618, 640, 667, 669, |
| | 675, 714, 718, 727-728, 762, 790, 798, 801, 829, 839, 912, 941, 956, 963, 983, |
| | 1014, 1076, 1083, 1091, 1096, 1130, 1142, 1161, 1178, 1183, 1213, 1220, 1284, |
| | 1336, 1344, 1350, 1354, 1361-1362, 1364, 1381, 1421, 1423, 1431, 1449, 1453, |
| | 1455, 1489, 1510, 1526, 1541, 1558, 1584, 1621, 1624, 1652, 1668, 1679, 1709, |
| | 1727, 1739, 1751, 1761, 1770, 1776, 1811, 1816, 1850, 1876, 1911, 1919, 1925, |
| | 1930, 1938, 1974, 2000, 2030, 2076, 2085, 2097, 2113, 2137, 2150, 2197, 2232, |
| | 2238, 2277, 2289, 2300, 2303, 2305, 2312, 2353, 2419, 2435, 2440, 2446, 2492, |
| | 2529, 2558, 2596, 2603, 2620, 2626, 2636, 2670, 2700, 2757, 2759, 2812, 2824, |
| | 2846, 2920, 2938, 2978, 3005, 3017, 3044, 3064, 3070, 3087, 3149, 3207, 3295, |
| | 3319, 3326, 3353, 3357, 3372, 3383, 3401, 3404, 3430, 3456, 3458, 3491, 3499, |
| | 3503, 3514, 3544, 3617, 3643, 3677, 3679, 3709 |
| Europe | 65, 81, 250, 254, 332, 392, 395, 514, 576, 580, 592-593, 606, 612, 618, 667, 669, |
| | 675, 714, 718, 727-728, 748, 790, 829, 839, 912, 941, 956, 963, 983, 1083, 1096, |
| | 1130, 1142, 1161, 1178, 1183, 1213, 1220, 1336, 1344, 1350, 1361-1362, 1364, |
| | 1381, 1421, 1423, 1431, 1440, 1449, 1453, 1455, 1489, 1579, 1584, 1679, 1709, |
| | 1727, 1739, 1761, 1776, 1850, 1876, 1911-1912, 1919, 1925, 1930, 1974, 2030, |
| | 2038, 2085, 2097, 2137, 2197, 2232, 2238, 2244, 2289, 2300, 2305, 2312, 2320, |
| | 2353, 2419, 2435, 2446, 2492, 2565, 2603, 2620, 2824, 2838, 2978, 3070, 3087, |
| | 3117, 3207, 3230, 3295, 3326, 3357, 3383, 3401, 3430, 3456, 3514, 3677, 3679 |
| North Africa | 65, 250, 302, 395, 592-593, 612, 618, 640, 667, 669, 675, 714, 718, 727-728, 762, |
| | 790, 798, 801, 829, 839, 912, 941, 956, 963, 983, 1014, 1017, 1076, 1083, 1091, |
| | 1096, 1128, 1161, 1178, 1220, 1284, 1336, 1344, 1350, 1354, 1361-1362, 1364, |
| | 1381, 1423, 1440, 1453, 1455, 1489, 1510, 1526, 1534, 1558, 1579, 1584, 1602, |
| | 1607, 1624, 1647, 1652, 1668, 1679, 1703, 1709, 1727, 1739, 1761, 1770, 1776, |
| | 1816, 1850, 1876, 1882, 1888, 1911, 1919, 1925, 1930, 1938, 1974, 2000, 2030, |
| | 2061, 2076, 2085, 2097, 2137, 2150, 2154, 2226, 2230, 2232, 2251, 2277, 2289, |
| | 2305, 2312, 2353, 2386, 2419, 2435, 2446, 2450, 2492, 2529, 2558, 2565, 2596, |
| | 2620, 2626, 2636, 2699-2700, 2737, 2757, 2792, 2824, 2846, 2920, 2938, 2978, |
| | 3017, 3023, 3044, 3087, 3149, 3207, 3235, 3295, 3319, 3353, 3357, 3383, 3401, |
| | 3430, 3456, 3491, 3497, 3503, 3514, 3528, 3613, 3677 |
| North America | 65, 81, 250, 254, 332, 395, 514, 576, 580, 592-593, 606, 612, 618, 640, 669, 714, |
| | 718, 727-728, 762, 829, 839, 912, 941, 956, 963, 983, 1014, 1083, 1142, 1161, |
| | 1213, 1220, 1284, 1350, 1361-1362, 1364, 1381, 1423, 1431, 1453, 1455, 1489, |
| | 1510, 1526, 1531, 1691, 1709, 1751, 1761, 1776, 1876, 1912, 1974, 2030, 2097, |
| | 2150, 2197, 2232, 2238, 2289, 2303, 2320, 2353, 2419, 2435, 2440, 2446, 2457, |
| | 2492, 2603, 2620, 2626, 2700, 2757, 2759, 2824, 2834, 2938, 2958, 2978, 3005, |
| | 3025, 3029, 3044, 3064, 3070, 3087, 3117, 3149, 3151, 3207, 3295, 3319, 3326, |
| | 3353, 3357, 3372, 3383, 3404, 3430, 3458, 3499, 3503, 3514, 3608, 3613, 3617, |
| | 3677, 3679, 3818 |
| North East Asia | 65, 81, 250, 254, 302, 332, 395, 514, 547, 576, 580, 592-593, 606, 612, 618, 640, |
| | 667, 669, 675, 714, 718, 727-728, 762, 790, 798, 801, 829, 839, 912, 941, 956, |
| | 963, 983, 1014, 1017, 1076, 1083, 1091, 1096, 1128, 1130, 1142, 1161, 1178, |
| | 1183, 1213, 1220, 1284, 1336, 1344, 1350, 1354, 1361-1362, 1364, 1381, 1393, |
| | 1421, 1423, 1431, 1449, 1453, 1455, 1489, 1510, 1526, 1534, 1541, 1558, 1584, |
| | 1602, 1607, 1621, 1624, 1652, 1668, 1679, 1691, 1709, 1727, 1739, 1751, 1761, |
| | 1770, 1776, 1811, 1816, 1850, 1876, 1888, 1911-1912, 1919, 1925, 1928, 1930, |
| | 1938, 1974, 2000, 2030, 2061, 2076, 2085, 2097, 2137, 2150, 2154, 2197, 2232, |
| | 2238, 2244, 2261, 2289, 2300, 2303, 2305, 2320, 2353, 2364, 2419, 2435, 2440, |
| | 2446, 2492, 2529, 2565, 2596, 2603, 2620, 2626, 2636, 2674, 2697, 2699-2700, |
| | 2757, 2759, 2812, 2824, 2846, 2863, 2938, 2978, 3005, 3017, 3044, 3064, 3070, |
| | 3117, 3149, 3207, 3295, 3326, 3372, 3404, 3430, 3458, 3497, 3514, 3521, 3544, |
| | 3548, 3608, 3613, 3777 |
| Oceania | 65, 81, 250, 302, 392, 395, 514, 547, 576, 580, 592-593, 606, 612, 618, 640, 667, |
| | 669, 675, 714, 718, 727-728, 748, 762, 790, 798, 801, 829, 839, 912, 941, 956, |
| | 963, 983, 989, 1014, 1017, 1076, 1083, 1091, 1096, 1128, 1130, 1142, 1161, 1178, |
| | 1183, 1213, 1220, 1284, 1336, 1344, 1350, 1354, 1361-1362, 1364, 1381, 1393, |
| | 1421, 1423, 1431, 1449, 1453, 1455, 1489, 1510, 1526, 1531, 1534, 1541, 1558, |
| | 1584, 1602, 1607, 1621, 1624, 1652, 1668, 1679, 1691, 1709, 1727, 1739, 1751, |
| | 1761, 1770, 1776, 1811, 1816, 1850, 1876, 1888, 1911-1912, 1925, 1928, 1930, |
| | 1938, 1974, 2000, 2030, 2061, 2076, 2085, 2097, 2113, 2137, 2144, 2150, 2197, |
| | 2232, 2238, 2261, 2277, 2289, 2300, 2303, 2305, 2312, 2320, 2353, 2364, 2419, |
| | 2435, 2440, 2446, 2457, 2492, 2529, 2558, 2565, 2596, 2603, 2620, 2626, 2631, |
| | 2636, 2670, 2674, 2697, 2700, 2757, 2759, 2812, 2824, 2834, 2838, 2846, 2863, |
| | 2920, 2938, 2958, 2978, 2991, 3005, 3017, 3025, 3029, 3044, 3062, 3064, 3070, |
| | 3087, 3149, 3207, 3264, 3295, 3319, 3326, 3353, 3357, 3372, 3404, 3456, 3458, |
| | 3466, 3491, 3499, 3503, 3514, 3521, 3544, 3548, 3613, 3617, 3643, 3709, 3777, |
| | 3779 |
| South and Central America | 65, 81, 250, 302, 332, 395, 514, 547, 576, 580, 592-593, 606, 612, 618, 640, 667, |
| | 669, 675, 714, 718, 727-728, 762, 790, 798, 801, 829, 839, 912, 941, 956, 963, |
| | 983, 1014, 1017, 1076, 1083, 1096, 1128, 1130, 1142, 1161, 1178, 1183, 1213, |
| | 1220, 1284, 1336, 1344, 1350, 1361-1362, 1364, 1381, 1421, 1423, 1431, 1453, |
| | 1455, 1489, 1510, 1526, 1534, 1541, 1558, 1584, 1602, 1607, 1621, 1624, 1652, |
| | 1668, 1679, 1709, 1727, 1739, 1751, 1761, 1770, 1776, 1811, 1816, 1850, 1876, |
| | 1888, 1911-1912, 1925, 1930, 1938, 1974, 2000, 2030, 2076, 2085, 2097, 2137, |
| | 2144, 2150, 2197, 2226, 2232, 2238, 2244, 2277, 2289, 2303, 2312, 2320, 2353, |
| | 2386, 2419, 2435, 2446, 2457, 2492, 2529, 2558, 2596, 2603, 2620, 2626, 2636, |
| | 2670, 2674, 2700, 2737, 2757, 2759, 2824, 2920, 2978, 2996, 3005, 3017, 3044, |
| | 3062, 3070, 3087, 3117, 3144, 3149, 3207, 3264, 3295, 3326, 3357, 3372, 3383, |
| | 3404, 3456, 3458, 3466, 3503, 3514, 3521, 3525, 3544, 3643, 3677, 3679, 3709, |
| | 3831, 3853 |
| South Asia | 65, 81, 250, 302, 392, 395, 514, 547, 576, 580, 592-593, 606, 612, 618, 640, 667, |
| | 669, 675, 714, 718, 727-728, 748, 762, 790, 798, 801, 829, 839, 912, 941, 956, |
| | 963, 983, 989, 1014, 1017, 1076, 1083, 1096, 1128, 1130, 1142, 1161, 1178, 1183, |
| | 1220, 1284, 1336, 1344, 1350, 1354, 1361-1362, 1364, 1381, 1421, 1423, 1431, |
| | 1440, 1449, 1453, 1455, 1489, 1510, 1534, 1541, 1558, 1579, 1584, 1602, 1607, |
| | 1621, 1624, 1647, 1652, 1668, 1679, 1691, 1703, 1709, 1727, 1739, 1751, 1761, |
| | 1776, 1811, 1816, 1850, 1876, 1882, 1888, 1911-1912, 1919, 1925, 1928, 1930, |
| | 1938, 1940, 1974, 2000, 2030, 2076, 2085, 2097, 2137, 2144, 2150, 2154, 2197, |
| | 2226, 2232, 2238, 2251, 2261, 2277, 2289, 2305, 2312, 2320, 2353, 2419, 2435, |
| | 2446, 2492, 2529, 2558, 2565, 2596, 2620, 2626, 2636, 2674, 2697, 2699, 2737, |
| | 2757, 2824, 2838, 2846, 2863, 2920, 2978, 2996, 3017, 3023, 3049, 3064, 3087, |
| | 3144, 3149, 3207, 3331, 3357, 3383, 3401, 3456, 3463, 3486, 3497, 3514, 3521, |
| | 3548, 3677, 3679, 3777 |
| South East Asia | 65, 81, 250, 254, 392, 395, 514, 547, 576, 580, 592-593, 606, 612, 618, 640, 667, |
| | 669, 675, 714, 718, 727-728, 748, 762, 790, 829, 839, 912, 941, 956, 963, 983, |
| | 989, 1014, 1017, 1083, 1096, 1128, 1142, 1161, 1178, 1183, 1213, 1220, 1284, |
| | 1344, 1350, 1354, 1361-1362, 1364, 1381, 1421, 1423, 1431, 1449, 1453, 1455, |
| | 1489, 1510, 1526, 1531, 1534, 1541, 1584, 1602, 1607, 1621, 1679, 1709, 1727, |
| | 1751, 1761, 1776, 1850, 1876, 1888, 1912, 1938, 1974, 2000, 2030, 2038, 2076, |
| | 2085, 2097, 2113, 2137, 2150, 2197, 2226, 2232, 2238, 2289, 2300, 2303, 2305, |
| | 2312, 2320, 2353, 2419, 2435, 2440, 2446, 2457, 2471, 2492, 2558, 2565, 2620, |
| | 2631, 2670, 2674, 2700, 2737, 2759, 2812, 2824, 2834, 2846, 2863, 2938, 2958, |
| | 2968, 2978, 2991, 2996, 3005, 3025, 3029, 3044, 3062, 3064, 3070, 3117, 3207, |
| | 3230, 3264, 3295, 3326, 3372, 3404, 3411, 3430, 3456, 3458, 3466, 3486, 3491, |
| | 3514, 3521, 3613, 3777 |
| Sub-Saharan Africa | 65, 81, 250, 395, 514, 576, 580, 592-593, 606, 612, 618, 640, 667, 669, 714, 718, |
| | 728, 762, 829, 839, 912, 941, 956, 963, 983, 1014, 1017, 1083, 1091, 1096, 1142, |
| | 1161, 1213, 1220, 1336, 1344, 1350, 1354, 1361-1362, 1364, 1381, 1423, 1431, |
| | 1453, 1455, 1489, 1510, 1526, 1531, 1607, 1679, 1691, 1709, 1751, 1761, 1776, |
| | 1811, 1850, 1876, 1912, 1919, 1938, 1940, 1974, 2030, 2061, 2076, 2097, 2113, |
| | 2137, 2144, 2150, 2197, 2230, 2232, 2238, 2244, 2277, 2289, 2312, 2353, 2419, |
| | 2435, 2446, 2492, 2603, 2620, 2626, 2636, 2700, 2757, 2824, 2846, 2863, 2920, |
| | 2938, 2978, 3044, 3070, 3087, 3117, 3149, 3207, 3235, 3295, 3319, 3326, 3353, |
| | 3357, 3383, 3456, 3458, 3473, 3491, 3499, 3503, 3514, 3528, 3659, 3671, 3677, |
| | 3700 |
| Western Asia | 65, 81, 250, 332, 392, 395, 514, 576, 580, 592-593, 606, 612, 618, 640, 667, 669, |
| | 675, 714, 718, 727-728, 748, 762, 790, 829, 839, 912, 941, 956, 963, 983, 989, |
| | 1014, 1017, 1083, 1096, 1128, 1130, 1142, 1161, 1178, 1183, 1213, 1220, 1284, |
| | 1336, 1344, 1350, 1361-1362, 1364, 1381, 1423, 1431, 1440, 1449, 1453, 1455, |
| | 1489, 1510, 1531, 1534, 1579, 1584, 1602, 1607, 1621, 1624, 1668, 1679, 1691, |
| | 1709, 1727, 1739, 1751, 1761, 1776, 1811, 1850, 1876, 1888, 1911-1912, 1919, |
| | 1925, 1930, 1938, 1974, 2000, 2030, 2038, 2076, 2085, 2097, 2137, 2144, 2150, |
| | 2154, 2197, 2226, 2232, 2238, 2244, 2277, 2289, 2300, 2305, 2312, 2320, 2353, |
| | 2419, 2435, 2446, 2457, 2492, 2558, 2620, 2626, 2631, 2699, 2737, 2757, 2824, |
| | 2838, 2846, 2920, 2958, 2968, 2978, 2991, 2996, 3025, 3029, 3062, 3064, 3087, |
| | 3117, 3149, 3151, 3207, 3230, 3264, 3295, 3357, 3360, 3383, 3401, 3430, 3456, |
| | 3466, 3497, 3514, 3593, 3607, 3677, 3679, 3753 |

TABLE 34
Region-specific peptide pools derived from NSP13 protein for CD8 T cell assays.

| Region | Seq. IDs of peptides |
|---|---|
| Australia | 76, 102, 223, 245, 393, 475, 519, 540, 543, 545, 557, 559, 566, 598, 616-617, 620, |
| | 629, 633, 648, 673, 681, 700, 704, 729, 793, 802, 807-808, 823, 832-833, 836, 862, |
| | 869, 873, 880, 892, 901, 918, 924, 929, 935, 939, 987, 991, 997, 1037, 1039, 1067, |
| | 1080, 1119, 1137, 1156, 1187, 1203, 1209, 1247-1248, 1255, 1280, 1296, 1356, |
| | 1367, 1389, 1403, 1413, 1433, 1436, 1519-1521, 1537, 1559, 1592, 1594, 1658, |
| | 1686, 1705, 1752, 1755, 1786, 1803, 1855, 1899, 1916-1917, 1920, 1964, 1976, |
| | 1996, 1998, 2010, 2037, 2057, 2109, 2114, 2155, 2162, 2188, 2212, 2215, 2236, |
| | 2257, 2263, 2323, 2354, 2369, 2381, 2423, 2484, 2499, 2506, 2673, 2704, 2707, |
| | 2724, 2744, 2752, 2796, 2853, 2871, 2911, 2919, 2930, 2943, 2975, 2988, 3072, |
| | 3079, 3133, 3139, 3142, 3173, 3184, 3210-3211, 3219, 3418, 3483, 3589, 3614, |
| | 3653 |
| Europe | 76, 102, 223, 245, 256, 393, 475, 499, 506, 519, 540, 543, 545, 557, 559, 566, 598, |
| | 616-617, 620, 629, 673, 700, 704, 722, 729, 746, 793, 802, 808, 823, 832, 836, |
| | 862, 873, 880, 892, 901, 918, 924, 929, 935, 939, 987, 991, 997, 1037, 1039, 1067, |
| | 1137, 1156, 1187, 1209, 1247-1248, 1255, 1280, 1296, 1389, 1401, 1403, 1413, |
| | 1433, 1436, 1509, 1520-1521, 1559, 1592, 1686, 1705, 1714, 1755, 1786, 1803, |
| | 1855, 1899, 1914, 1964, 2037, 2042, 2057, 2106, 2109, 2114-2115, 2159, 2162, |
| | 2177, 2188, 2192, 2212, 2215, 2352, 2381, 2423, 2470, 2571, 2595, 2705, 2752, |
| | 2866, 2871, 2886, 2919, 2930, 2975, 2988, 3009, 3022, 3058, 3083, 3100, 3123, |
| | 3133, 3142, 3164, 3184, 3219, 3262, 3293, 3477, 3516, 3614, 3639, 3708 |
| North Africa | 102, 245, 393, 475, 519, 540, 543, 545, 557, 559, 566, 616-617, 620, 629, 633, |
| | 648, 673, 681, 700, 729, 793, 802, 807-808, 823, 832-833, 836, 862, 873, 880, 892, |
| | 901, 918, 924, 929, 935, 939, 987, 991, 997, 1037, 1039, 1067, 1119, 1137, 1154, |
| | 1168, 1187, 1203, 1209, 1247, 1255, 1296, 1389, 1403, 1413, 1433, 1436, |
| | 1519-1520, 1537, 1559, 1592, 1596, 1648, 1658, 1680, 1686, 1705, 1755, 1786, |
| | 1803, 1855, 1917, 1920, 1976, 1996, 1998, 2010, 2037, 2057, 2067, 2109, 2155, |
| | 2188, 2236, 2257, 2263, 2352, 2354, 2369, 2381, 2409, 2423, 2470, 2484, 2506, |
| | 2553, 2673, 2704, 2707, 2752, 2766, 2804, 2818, 2871, 2919, 2930, 2943, 2975, |
| | 3055, 3072, 3100, 3142, 3160, 3173, 3184, 3219, 3305, 3417, 3428, 3436, 3461, |
| | 3490, 3591, 3609, 3614, 3653, 3772 |
| North America | 76, 102, 223, 245, 256, 393, 499, 506, 519, 540, 543, 545, 557, 559, 566, 598, 617, |
| | 620, 629, 633, 704, 722, 729, 808, 832-833, 836, 862, 869, 873, 880, 892, 901, |
| | 918, 924, 929, 935, 939, 987, 991, 997, 1037, 1039, 1067, 1080, 1137, 1156, 1187, |
| | 1247-1248, 1255, 1280, 1356, 1367, 1389, 1401, 1403, 1413, 1436, 1509, 1519-1521, |
| | 1592, 1658, 1686, 1705, 1735, 1752, 1755, 1786, 1803, 1855, 1899, 1914, |
| | 1916-1917, 1920, 1964, 1996, 1998, 2010, 2109, 2132, 2155, 2162, 2177, 2188, |
| | 2212, 2215, 2263, 2323, 2354, 2362, 2369, 2381, 2423, 2506, 2563, 2571, 2595, |
| | 2673, 2707, 2752, 2853, 2866, 2871, 2911, 2919, 2925, 2975, 2988, 3009, 3022, |
| | 3133, 3139, 3211, 3219, 3238, 3262, 3418, 3606, 3614, 3653, 3747, 3761 |
| North East Asia | 76, 102, 223, 245, 256, 475, 499, 506, 519, 540, 543, 545, 557, 559, 566, 598, |
| | 616-617, 620, 629, 633, 673, 681, 700, 704, 722, 729, 746, 793, 802, 807-808, |
| | 823, 832-833, 836, 862, 869, 873, 880, 892, 901, 918, 924, 929, 935, 939, 987, |
| | 991, 997, 1037, 1039, 1067, 1080, 1119, 1137, 1154, 1156, 1168, 1187, 1203, |
| | 1209, 1247-1248, 1255, 1280, 1296, 1356, 1367, 1389, 1401, 1403, 1413, 1433, |
| | 1436, 1509, 1519-1521, 1537, 1559, 1592, 1594, 1596, 1648, 1658, 1680, 1686, |
| | 1705, 1735, 1752, 1755, 1786, 1803, 1855, 1899, 1914, 1916-1917, 1920, 1964, |
| | 1996, 1998, 2010, 2057, 2067, 2109, 2155, 2162, 2177, 2188, 2192, 2212, 2215, |
| | 2236, 2257, 2323, 2352, 2354, 2362, 2369, 2423, 2470, 2484, 2499, 2506, 2571, |
| | 2595, 2673, 2707, 2744, 2752, 2818, 2837, 2853, 2866, 2871, 2911, 2925, 2943, |
| | 2975, 2988, 3009, 3022, 3055, 3072, 3079, 3100, 3133, 3139, 3142, 3164, 3173, |
| | 3184, 3210-3211, 3227, 3262, 3305, 3483, 3498, 3517, 3822 |
| Oceania | 76, 102, 245, 393, 475, 499, 506, 519, 540, 543, 545, 557, 559, 566, 598, 616-617, |
| | 620, 629, 633, 648, 673, 685, 700, 704, 722, 729, 746, 793, 802, 807-808, 823, |
| | 832-833, 836, 862, 869, 873, 880, 892, 901, 918, 924, 929, 935, 939, 987, 991, |
| | 997, 1037, 1039, 1067, 1080, 1119, 1137, 1154, 1156, 1168, 1187, 1203, 1209, |
| | 1247-1248, 1255, 1280, 1296, 1356, 1367, 1389, 1401, 1403, 1413, 1429, 1433, |
| | 1436, 1506, 1509, 1519-1521, 1537, 1546, 1559, 1592, 1594, 1596, 1640, 1648, |
| | 1658, 1680, 1686, 1705, 1752, 1755, 1786, 1803, 1855, 1899, 1914, 1916-1917, |
| | 1920, 1964, 1976, 1998, 2010, 2037, 2042, 2057, 2067, 2109, 2114, 2132, 2155, |
| | 2162, 2177, 2188, 2192, 2212, 2215, 2236, 2257, 2263, 2323, 2354, 2369, 2381, |
| | 2413, 2423, 2484, 2499, 2506, 2563, 2571, 2595, 2667, 2673, 2703-2704, 2707, |
| | 2724, 2744, 2752, 2796, 2818, 2837, 2853, 2866, 2871, 2911, 2919, 2925, 2943, |
| | 2975, 2988, 3009, 3022, 3072, 3079, 3083, 3100, 3133, 3139, 3142, 3164, 3173, |
| | 3184, 3210-3211, 3227, 3238, 3278, 3293, 3417-3418, 3438-3439, 3483, 3498, |
| | 3589, 3609, 3653, 3655, 3822 |
| South and Central America | 76, 102, 245, 393, 475, 499, 506, 519, 540, 543, 545, 557, 559, 566, 598, 617, 620, |
| | 629, 633, 648, 673, 681, 700, 704, 722, 729, 746, 793, 802, 807-808, 823, 832-833, |
| | 836, 862, 869, 873, 880, 892, 901, 918, 924, 929, 935, 939, 987, 991, 997, 1037, |
| | 1039, 1067, 1119, 1137, 1168, 1187, 1203, 1209, 1247, 1255, 1280, 1296, 1389, |
| | 1401, 1403, 1413, 1433, 1436, 1506, 1509, 1519, 1521, 1537, 1559, 1594, 1596, |
| | 1648, 1658, 1680, 1686, 1705, 1752, 1755, 1786, 1803, 1855, 1899, 1914, |
| | 1916-1917, 1920, 1964, 1976, 1996, 2010, 2057, 2109, 2155, 2162, 2177, 2188, |
| | 2192, 2212, 2215, 2236, 2257, 2263, 2352, 2354, 2369, 2381, 2409, 2413, 2423, |
| | 2484, 2499, 2506, 2571, 2595, 2673, 2704, 2707, 2724, 2744, 2796, 2818, 2853, |
| | 2866, 2871, 2883, 2919, 2943, 2975, 2988, 3009, 3022, 3055, 3072, 3079, 3100, |
| | 3133, 3139, 3142, 3164, 3190, 3219, 3253, 3262, 3278, 3417, 3483, 3498, 3589, |
| | 3614, 3717, 3846 |
| South Asia | 76, 102, 245, 393, 475, 519, 540, 543, 545, 557, 559, 566, 598, 616-617, 620, 629, |
| | 633, 648, 673, 681, 685, 700, 704, 722, 729, 746, 793, 802, 807-808, 823, 832-833, |
| | 836, 862, 873, 880, 892, 901, 918, 924, 929, 935, 939, 987, 991, 997, 1037, 1039, |
| | 1067, 1080, 1119, 1137, 1154, 1156, 1168, 1187, 1203, 1209, 1247, 1255, 1280, |
| | 1296, 1356, 1367, 1389, 1401, 1403, 1413, 1429, 1433, 1436, 1506, 1519-1521, |
| | 1537, 1546, 1559, 1594, 1596, 1640, 1648, 1680, 1705, 1752, 1755, 1786, 1803, |
| | 1855, 1899, 1914, 1916-1917, 1920, 1964, 1976, 1996, 2010, 2037, 2042, 2057, |
| | 2109, 2155, 2162, 2177, 2188, 2192, 2212, 2215, 2236, 2257, 2263, 2323, 2352, |
| | 2354, 2381, 2423, 2470, 2484, 2499, 2571, 2595, 2667, 2704, 2707, 2752, 2766, |
| | 2804, 2818, 2837, 2866, 2871, 2919, 2925, 2930, 2943, 2975, 3009, 3022, 3036, |
| | 3055, 3072, 3083, 3123, 3133, 3139, 3142, 3164, 3184, 3219, 3227, 3293, 3305, |
| | 3405, 3417, 3428, 3438, 3483, 3498, 3532, 3609, 3614, 3639, 3783, 3809, 3822, |
| | 3833 |
| South East Asia | 76, 102, 245, 256, 393, 475, 499, 506, 519, 540, 543, 545, 557, 559, 566, 598, |
| | 616-617, 620, 629, 633, 673, 681, 685, 700, 704, 722, 729, 746, 808, 832-833, |
| | 836, 862, 869, 873, 880, 892, 901, 918, 924, 929, 935, 939, 987, 991, 997, 1037, |
| | 1039, 1067, 1080, 1119, 1137, 1154, 1156, 1187, 1203, 1247-1248, 1255, 1280, |
| | 1296, 1324, 1356, 1367, 1389, 1401, 1403, 1413, 1436, 1506, 1509, 1519-1521, |
| | 1537, 1546, 1559, 1592, 1594, 1596, 1640, 1648, 1658, 1680, 1686, 1705, 1714, |
| | 1752, 1755, 1786, 1803, 1855, 1899, 1914, 1916-1917, 1920, 1964, 1996, 1998, |
| | 2010, 2037, 2042, 2057, 2067, 2109, 2132, 2155, 2159, 2162, 2177, 2188, 2192, |
| | 2212, 2215, 2236, 2352, 2354, 2369, 2413, 2423, 2499, 2506, 2563, 2571, 2595, |
| | 2673, 2703, 2705, 2752, 2818, 2853, 2866, 2871, 2883, 2911, 2975, 2988, 3009, |
| | 3022, 3055, 3100, 3133, 3139, 3164, 3173, 3210-3211, 3238, 3278, 3289, 3300, |
| | 3417, 3439, 3498, 3517, 3537, 3609, 3655, 3822 |
| Sub-Saharan Africa | 76, 223, 245, 393, 499, 506, 519, 540, 543, 545, 557, 559, 566, 598, 616-617, 620, |
| | 629, 633, 648, 704, 832-833, 836, 862, 873, 880, 892, 901, 918, 924, 929, 935, |
| | 939, 987, 991, 1037, 1039, 1067, 1080, 1137, 1154, 1156, 1187, 1203, 1247-1248, |
| | 1255, 1280, 1296, 1356, 1389, 1401, 1403, 1413, 1429, 1433, 1436, 1509, 1519, |
| | 1521, 1592, 1594, 1596, 1648, 1658, 1686, 1705, 1735, 1755, 1786, 1803, 1855, |
| | 1899, 1916-1917, 1920, 1964, 1996, 1998, 2010, 2037, 2057, 2067, 2109, 2114, |
| | 2132, 2155, 2162, 2188, 2212, 2263, 2354, 2362, 2369, 2381, 2409, 2423, 2470, |
| | 2506, 2553, 2563, 2571, 2595, 2673, 2705, 2707, 2744, 2871, 2919, 2925, 2930, |
| | 2975, 2988, 3009, 3022, 3072, 3100, 3133, 3139, 3160, 3210, 3219, 3241, 3262, |
| | 3428, 3490, 3507, 3517, 3562, 3591, 3614, 3642 |
| Western Asia | 76, 102, 245, 393, 475, 499, 506, 519, 540, 543, 545, 557, 559, 566, 598, 616-617, |
| | 620, 629, 633, 648, 673, 681, 685, 700, 704, 722, 729, 746, 793, 802, 808, 823, |
| | 832-833, 836, 862, 873, 880, 892, 901, 918, 924, 929, 935, 939, 987, 991, 997, |
| | 1037, 1039, 1067, 1080, 1119, 1137, 1154, 1156, 1168, 1187, 1203, 1209, 1247, |
| | 1255, 1280, 1296, 1324, 1356, 1367, 1389, 1401, 1403, 1413, 1429, 1433, 1436, |
| | 1506, 1509, 1519-1521, 1546, 1559, 1592, 1596, 1640, 1648, 1658, 1680, 1705, |
| | 1714, 1752, 1755, 1786, 1803, 1855, 1899, 1914, 1916, 1920, 1964, 1976, 1996, |
| | 2010, 2037, 2042, 2057, 2067, 2106, 2109, 2114, 2132, 2155, 2159, 2162, 2177, |
| | 2188, 2192, 2212, 2215, 2236, 2263, 2352, 2354, 2381, 2413, 2423, 2470, 2563, |
| | 2571, 2595, 2703-2704, 2707, 2752, 2818, 2866, 2871, 2883, 2919, 2930, 2975, |
| | 3009, 3022, 3036, 3055, 3072, 3083, 3123, 3133, 3139, 3142, 3173, 3184, 3219, |
| | 3238, 3262, 3278, 3289, 3300, 3305, 3417, 3537, 3584, 3606, 3614, 3639, 3706 |

TABLE 35
Region-specific peptide pools derived from ORF8 protein for CD8 T cell assays.

| Region | Seq. IDs of peptides |
|---|---|
| Australia | 104, 301, 556, 780, 896, 933, 978, 1238, 1307, 1326, 1363, 1444, 1535, 1564, |
| | 1578, 1669, 1981, 2588, 2627, 2695, 2712, 2833, 2842, 2914, 2926, 2999, 3099, |
| | 3137, 3251, 3296, 3330, 3583, 3595, 3619, 3702 |
| Europe | 104, 301, 556, 572, 896, 933, 978, 1238, 1444, 1463, 1535, 1564, 1578, 1669, |
| | 1812, 1981, 2588, 2712, 2833, 2842, 2926, 3099, 3251, 3259, 3619, 3702, 3832 |
| North Africa | 104, 556, 780, 816, 896, 933, 978, 1238, 1326, 1363, 1444, 1482, 1535, 1564, |
| | 1578, 1669, 1981, 2407, 2588, 2712, 2833, 2842, 2926, 2999, 3099, 3137, 3251, |
| | 3259, 3330, 3391, 3419, 3583, 3602, 3619, 3661, 3702, 3835 |
| North America | 104, 301, 556, 572, 780, 896, 933, 1307, 1326, 1444, 1463, 1535, 1564, 1578, |
| | 1669, 2407, 2588, 2627, 2695, 2712, 2842, 2914, 2926, 3099, 3263, 3296, 3391, |
| | 3595, 3612, 3619, 3844 |
| North East Asia | 104, 301, 556, 572, 780, 816, 896, 933, 978, 1238, 1307, 1326, 1363, 1444, 1463, |
| | 1482, 1535, 1564, 1578, 1669, 1981, 2407, 2588, 2627, 2695, 2712, 2729, 2842, |
| | 2914, 2926, 2960, 2999, 3099, 3137, 3263, 3296, 3391, 3595, 3602, 3612, 3619 |
| Oceania | 104, 301, 556, 572, 780, 816, 896, 933, 978, 1238, 1307, 1326, 1363, 1444, 1463, |
| | 1482, 1535, 1564, 1578, 1669, 1812, 1981, 2588, 2627, 2695, 2712, 2729, 2833, |
| | 2842, 2914, 2926, 2960, 2999, 3099, 3137, 3251, 3263, 3296, 3330, 3391, 3595, |
| | 3612, 3619 |
| South and Central America | 104, 556, 572, 780, 816, 896, 933, 978, 1238, 1307, 1326, 1363, 1444, 1463, 1535, |
| | 1578, 1669, 1812, 1981, 2407, 2588, 2627, 2695, 2712, 2833, 2842, 2914, 2926, |
| | 3099, 3137, 3330, 3391, 3595, 3602, 3619 |
| South Asia | 104, 556, 572, 780, 816, 896, 933, 978, 1238, 1307, 1326, 1363, 1444, 1463, 1535, |
| | 1564, 1578, 1669, 1981, 2407, 2588, 2712, 2729, 2833, 2842, 2914, 2926, 2960, |
| | 3099, 3137, 3251, 3259, 3263, 3330, 3391, 3583, 3602, 3619, 3661, 3702 |
| South East Asia | 104, 301, 556, 572, 780, 816, 896, 933, 1238, 1307, 1326, 1363, 1444, 1463, 1535, |
| | 1564, 1578, 1669, 1812, 1981, 2407, 2588, 2627, 2695, 2712, 2729, 2842, 2914, |
| | 2926, 2960, 2999, 3099, 3251, 3263, 3296, 3376, 3391, 3469, 3595, 3612, 3619, |
| | 3829 |
| Sub-Saharan Africa | 104, 556, 572, 780, 816, 896, 933, 1238, 1307, 1326, 1444, 1482, 1535, 1564, |
| | 1578, 1669, 1981, 2588, 2627, 2833, 2842, 2926, 2999, 3099, 3251, 3391, 3583, |
| | 3587, 3619 |
| Western Asia | 104, 556, 572, 780, 816, 896, 933, 978, 1238, 1307, 1363, 1444, 1463, 1535, 1564, |
| | 1578, 1669, 1812, 1981, 2407, 2588, 2712, 2833, 2842, 2914, 2926, 3099, 3259, |
| | 3330, 3391, 3583, 3602, 3702, 3829, 3832 |

TABLE 36
Region-specific peptide pools derived from ORF6 protein for CD8 T cell assays.

| Region | Seq. IDs of peptides |
|---|---|
| Australia | 232, 244, 457, 806, 1175, 1301, 1626, 1634, 1756, 1990, 2066, 2168, 2324, 2431, |
| | 2770, 2912, 3074, 3163, 3379, 3513 |
| Europe | 232, 244, 457, 806, 1175, 1301, 1626, 1634, 1756, 1990, 2066, 2770, 2912, 3074, |
| | 3163, 3386, 3513 |
| North Africa | 232, 244, 457, 806, 1626, 1634, 1756, 1990, 2066, 2431, 2671, 2770, 2912, 3074, |
| | 3163, 3181, 3379, 3513 |
| North America | 232, 244, 457, 806, 1175, 1301, 1756, 1990, 2770, 2912, 3163, 3286, 3379, 3513 |
| North East Asia | 232, 244, 457, 806, 1175, 1301, 1626, 1634, 1756, 1990, 2066, 2094, 2168, 2431, |
| | 2770, 2912, 3163, 3181, 3379, 3660 |
| Oceania | 232, 244, 457, 806, 1175, 1301, 1626, 1634, 1756, 1990, 2066, 2094, 2168, 2431, |
| | 2770, 2912, 3074, 3163, 3286, 3379, 3481, 3630, 3660 |
| South and Central America | 232, 244, 457, 806, 1301, 1626, 1634, 1756, 1990, 2066, 2168, 2431, 2671, 2770, |
| | 2912, 3074, 3163, 3181, 3379, 3513, 3660 |
| South Asia | 232, 244, 457, 806, 1175, 1301, 1626, 1634, 1756, 1990, 2066, 2094, 2431, 2770, |
| | 2912, 3074, 3163, 3181, 3379, 3513, 3660, 3745 |
| South East Asia | 244, 457, 806, 1175, 1301, 1626, 1634, 1756, 1990, 2066, 2094, 2168, 2324, 2770, |
| | 2912, 3163, 3286, 3386, 3481, 3630, 3660 |
| Sub-Saharan Africa | 232, 244, 457, 806, 1175, 1301, 1634, 1756, 1990, 2066, 2671, 2770, 2912, 3074, |
| | 3163, 3181, 3379, 3386, 3513 |
| Western Asia | 232, 244, 457, 806, 1175, 1301, 1626, 1634, 1756, 1990, 2066, 2168, 2431, 2671, |
| | 2770, 2912, 3074, 3163, 3286, 3379, 3386, 3513, 3630, 3660 |

TABLE 37
Region-specific peptide pools derived from NSP15 protein for CD8 T cell assays.

| Region | Seq. IDs of peptides |
|---|---|
| Australia | 265, 425, 460, 487, 495, 565, 605, 661, 663, 692, 723, 753, 788, 797, 831, 834, |
| | 842, 889, 949, 955, 969, 986, 1008, 1084, 1092, 1102, 1135, 1162, 1270, 1288, |
| | 1339, 1360, 1417, 1480, 1483, 1560, 1622, 1688, 1708, 1750, 1768, 1773, 1783, |
| | 1801, 1853, 1875, 1909, 1955, 1963, 1991, 1993, 2093, 2119, 2142, 2175, 2205, |
| | 2210, 2231, 2286-2287, 2299, 2359, 2363, 2474, 2585, 2612, 2623, 2634, 2683, |
| | 2715, 2760, 2763, 2765, 2813, 2857, 2874, 2901, 2933, 2959, 2973, 2984, 2989, |
| | 2995, 3010, 3069, 3092, 3132, 3288, 3336, 3368, 3416, 3451, 3488, 3529, |
| | 3603-3604, 3672, 3688, 3710, 3716, 3741, 3801, 3852 |
| Europe | 265, 425, 460, 487, 495, 565, 605, 661, 663, 692, 723, 753, 788, 797, 831, 842, |
| | 889, 949, 955, 969, 986, 1008, 1075, 1084, 1102, 1135, 1162, 1189, 1288, 1339, |
| | 1360, 1417, 1480, 1483, 1556, 1622, 1688, 1708, 1750, 1773, 1783, 1875, 1909, |
| | 1991, 1993, 2093, 2142, 2205, 2286, 2299, 2359, 2363, 2474, 2542, 2612, 2623, |
| | 2683, 2715, 2760, 2763, 2765, 2813, 2857, 2901, 2932-2933, 2942, 2973, 2989, |
| | 3092, 3119, 3320, 3366, 3416, 3672, 3716 |
| North Africa | 265, 425, 460, 487, 495, 565, 661, 663, 692, 723, 753, 788, 797, 831, 834, 842, |
| | 889, 949, 951, 955, 969, 986, 1008, 1092, 1102, 1135, 1162, 1270, 1274, 1288, |
| | 1339, 1360, 1417, 1480, 1483, 1560, 1622, 1688, 1708, 1750, 1768, 1773, 1783, |
| | 1801, 1875, 1955, 1991, 1993, 2093, 2142, 2175, 2205, 2286-2287, 2299, 2363, |
| | 2474, 2585, 2623, 2683, 2715, 2760, 2763, 2765, 2813, 2830, 2857, 2874, 2896, |
| | 2933, 2973, 2984, 2989, 3010, 3060, 3069, 3078, 3092, 3132, 3201, 3216, 3314, |
| | 3336-3337, 3368, 3416, 3603, 3620, 3672, 3688, 3716, 3738, 3778, 3801, 3860 |
| North America | 265, 425, 460, 487, 495, 565, 605, 661, 663, 692, 723, 753, 831, 834, 889, 949, |
| | 955, 969, 986, 1008, 1075, 1084, 1092, 1135, 1189, 1270, 1288, 1339, 1417, 1483, |
| | 1560, 1622, 1688, 1708, 1750, 1773, 1783, 1801, 1853, 1875, 1909, 1991, 1993, |
| | 2119, 2131, 2205, 2231, 2287, 2299, 2359, 2363, 2474, 2542, 2612, 2715, 2760, |
| | 2813, 2932-2933, 2942, 2949, 2959, 2973, 2989, 3010, 3092, 3336, 3366, 3416, |
| | 3472, 3488, 3604, 3672, 3695 |
| North East Asia | 265, 460, 487, 495, 565, 605, 661, 663, 692, 723, 753, 788, 797, 831, 834, 842, |
| | 889, 949, 951, 955, 969, 986, 1008, 1075, 1084, 1092, 1102, 1135, 1162, 1189, |
| | 1270, 1274, 1288, 1339, 1360, 1417, 1480, 1483, 1622, 1688, 1708, 1750, 1768, |
| | 1773, 1783, 1801, 1853, 1875, 1909, 1955, 1963, 1991, 1993, 2093, 2142, 2175, |
| | 2205, 2210, 2231, 2286-2287, 2299, 2359, 2363, 2474, 2542, 2612, 2683, 2715, |
| | 2745, 2760, 2763, 2765, 2813, 2857, 2874, 2901, 2932-2933, 2942, 2959, 2989, |
| | 3069, 3078, 3132, 3288, 3366, 3396, 3416, 3488, 3510, 3529, 3603-3604, 3695, |
| | 3741, 3752, 3806 |
| Oceania | 265, 425, 460, 487, 495, 565, 605, 661, 663, 692, 723, 753, 788, 797, 831, 834, |
| | 842, 889, 949, 951, 955, 969, 986, 1008, 1075, 1084, 1092, 1102, 1135, 1162, |
| | 1189, 1270, 1274, 1288, 1339, 1360, 1417, 1480, 1483, 1560, 1622, 1688, 1708, |
| | 1750, 1773, 1783, 1801, 1853, 1875, 1909, 1955, 1963, 1991, 1993, 2093, 2119, |
| | 2131, 2142, 2175, 2205, 2210, 2231, 2286-2287, 2299, 2359, 2363, 2474, 2585, |
| | 2612, 2623, 2634, 2683, 2715-2716, 2745, 2760, 2763, 2765, 2813, 2857, 2874, |
| | 2901, 2932-2933, 2942, 2949, 2959, 2973, 2984, 2989, 2995, 3010, 3069, 3092, |
| | 3132, 3288, 3336, 3366, 3368, 3396, 3416, 3451, 3472, 3488, 3510, 3529, |
| | 3603-3604, 3672, 3688, 3703, 3741, 3750, 3752, 3778, 3801, 3856 |
| South and Central America | 265, 425, 460, 487, 495, 565, 605, 661, 663, 692, 723, 753, 788, 797, 831, 834, |
| | 842, 889, 949, 955, 969, 986, 1008, 1075, 1084, 1092, 1102, 1135, 1162, 1189, |
| | 1270, 1288, 1339, 1360, 1417, 1480, 1483, 1560, 1622, 1688, 1708, 1750, 1768, |
| | 1773, 1783, 1801, 1875, 1909, 1955, 1963, 1991, 1993, 2093, 2119, 2142, 2175, |
| | 2205, 2210, 2231, 2286, 2299, 2363, 2474, 2542, 2585, 2612, 2623, 2634, 2683, |
| | 2715, 2745, 2760, 2763, 2765, 2813, 2857, 2874, 2901, 2932-2933, 2942, 2973, |
| | 2989, 2995, 3010, 3069, 3092, 3132, 3336, 3366, 3368, 3416, 3510, 3529, 3559, |
| | 3603-3604, 3672, 3688, 3741, 3778 |
| South Asia | 265, 425, 460, 487, 495, 565, 605, 661, 663, 692, 723, 753, 788, 797, 831, 834, |
| | 842, 889, 949, 951, 955, 969, 986, 1008, 1084, 1092, 1102, 1135, 1162, 1270, |
| | 1274, 1288, 1339, 1360, 1417, 1480, 1483, 1560, 1622, 1688, 1708, 1750, 1768, |
| | 1773, 1783, 1801, 1853, 1875, 1909, 1955, 1963, 1991, 1993, 2093, 2142, 2175, |
| | 2205, 2210, 2231, 2286, 2299, 2363, 2474, 2585, 2612, 2623, 2683, 2745, 2760, |
| | 2763, 2765, 2813, 2857, 2874, 2901, 2932-2933, 2942, 2973, 2989, 3069, 3078, |
| | 3092, 3132, 3201, 3216, 3314, 3320, 3337, 3368, 3396, 3416, 3510, 3559, |
| | 3603-3604, 3667, 3672, 3688, 3752, 3801 |
| South East Asia | 265, 425, 460, 487, 495, 565, 605, 661, 663, 692, 723, 753, 797, 831, 834, 842, |
| | 889, 949, 951, 955, 969, 986, 1008, 1075, 1084, 1092, 1135, 1189, 1270, 1288, |
| | 1339, 1417, 1480, 1483, 1556, 1622, 1688, 1708, 1750, 1768, 1773, 1783, 1801, |
| | 1853, 1875, 1909, 1963, 1991, 1993, 2093, 2119, 2131, 2142, 2205, 2210, 2231, |
| | 2287, 2299, 2359, 2363, 2474, 2612, 2623, 2634, 2715-2716, 2745, 2760, 2765, |
| | 2783, 2813, 2857, 2874, 2901, 2932-2933, 2942, 2949, 2959, 2989, 3010, 3201, |
| | 3266, 3288, 3366, 3416, 3472, 3488, 3510, 3604, 3672, 3703, 3710, 3816 |
| Sub-Saharan Africa | 265, 425, 460, 487, 495, 565, 605, 661, 663, 692, 723, 753, 834, 889, 949, 955, |
| | 969, 986, 1008, 1075, 1084, 1092, 1135, 1162, 1189, 1270, 1288, 1339, 1417, |
| | 1480, 1483, 1560, 1622, 1688, 1708, 1750, 1773, 1783, 1801, 1853, 1875, 1909, |
| | 1963, 1991, 2119, 2131, 2142, 2205, 2299, 2363, 2474, 2585, 2612, 2715, 2760, |
| | 2763, 2765, 2813, 2857, 2932-2933, 2973, 2984, 2989, 3010, 3060, 3078, 3092, |
| | 3336, 3416, 3472, 3665, 3672, 3716, 3801 |
| Western Asia | 265, 425, 460, 487, 495, 565, 605, 661, 663, 692, 723, 753, 788, 797, 831, 834, |
| | 842, 889, 949, 951, 955, 969, 986, 1008, 1075, 1084, 1092, 1102, 1135, 1162, |
| | 1189, 1270, 1288, 1339, 1360, 1417, 1480, 1483, 1560, 1622, 1688, 1708, 1750, |
| | 1768, 1773, 1783, 1801, 1853, 1875, 1909, 1955, 1963, 1991, 1993, 2093, 2119, |
| | 2131, 2142, 2205, 2210, 2286, 2299, 2359, 2363, 2474, 2542, 2585, 2612, 2623, |
| | 2683, 2715-2716, 2748, 2760, 2763, 2765, 2783, 2813, 2857, 2874, 2901, |
| | 2932-2933, 2942, 2949, 2973, 2989, 3010, 3069, 3078, 3092, 3119, 3320, 3366, |
| | 3368, 3416, 3472, 3559, 3603, 3672, 3688, 3716, 3778, 3801 |

TABLE 38
Region-specific peptide pools derived from ORF3c protein for CD8 T cell assays.

| Region | Seq. IDs of peptides |
|---|---|
| Australia | 498, 908, 981, 1045, 1412, 1606, 1710, 1721, 1887, 1934, 1941, 2298, 2495, 2560 |
| Europe | 498, 581, 590, 908, 981, 1045, 1286, 1412, 1606, 1941, 2298, 2495, 2560, 3570 |
| North Africa | 908, 981, 1045, 1412, 1606, 1710, 1721, 1887, 1910, 1934, 1941, 2152, 2298, |
| | 2560, 3019, 3616 |
| North America | 498, 581, 590, 908, 981, 1045, 1286, 1606, 1721, 1887, 1941, 2298, 2495, 2560 |
| North East Asia | 498, 581, 590, 908, 981, 1045, 1286, 1412, 1606, 1710, 1721, 1910, 1934, 1941, |
| | 2152, 2298, 2495, 2560, 3019 |
| Oceania | 498, 581, 590, 908, 981, 1045, 1286, 1412, 1606, 1710, 1721, 1910, 1934, 1941, |
| | 2152, 2298, 2495, 2560, 3493, 3570 |
| South and Central America | 498, 581, 590, 908, 981, 1045, 1286, 1412, 1606, 1710, 1721, 1887, 1910, 1941, |
| | 2152, 2298, 2495, 2560, 3019 |
| South Asia | 498, 581, 590, 908, 981, 1045, 1412, 1606, 1710, 1721, 1887, 1910, 1934, 1941, |
| | 2152, 2495, 2560, 3019, 3493, 3570, 3616 |
| South East Asia | 498, 581, 590, 908, 981, 1045, 1286, 1412, 1606, 1710, 1721, 1910, 1934, 1941, |
| | 2152, 2298, 2495, 2560, 3019, 3104, .3570 |
| Sub-Saharan Africa | 498, 581, 590, 908, 981, 1045, 1286, 1606, 1721, 1887, 1934, 1941, 2298, 2495, |
| | 2560, 3019, 3616 |
| Western Asia | 498, 581, 590, 908, 981, 1045, 1286, 1412, 1606, 1710, 1721, 1887, 1910, 1934, |
| | 1941, 2152, 2495, 2560, 3019, 3104,3493 |

TABLE 39
Region-specific peptide pools derived from
NSP11 protein for CD8 T cell assays.

| Region | Seq. IDs of peptides |
|---|---|
| Australia | 549, 615 |
| Europe | 549, 615 |
| North Africa | 549, 615, 3145 |
| North America | 615 |
| North East Asia | 549, 615 |
| Oceania | 549, 615 |
| South and Central America | 549, 615 |
| South Asia | 549, 615, 3145 |
| South East Asia | 549, 615 |
| Sub-Saharan Africa | 549, 615 |
| Western Asia | 549, 615, 3145 |

TABLE 40
Region-specific peptide pools derived from ORF3d protein for CD8 T cell assays.

| Region | Seq. IDs of peptides |
|---|---|
| Australia | 577, 1766, 2166, 2641, 3038, 3114, 3208, 3335, 3505, 3637, 3758 |
| Europe | 577, 1766, 2166, 2641, 2650, 3038, 3335 |
| North Africa | 2166, 2463, 2641, 2762, 3038, 3114, 3208, 3335, 3637, 3647 |
| North America | 577, 1766, 2166, 2641, 2650, 3038, 3208, 3335, 3505 |
| North East Asia | 577, 1766, 2166, 2463, 2641, 2650, 3038, 3114, 3208, 3318, 3335, 3505, 3637, |
| | 3767 |
| Oceania | 577, 1766, 2166, 2463, 2641, 3038, 3114, 3208, 3318, 3335, 3398, 3505, 3624, |
| | 3637, 3758 |
| South and Central America | 577, 1766, 2166, 2463, 2641, 2650, 3038, 3114, 3208, 3335, 3398, 3637 |
| South Asia | 577, 1766, 2166, 2463, 2641, 2762, 3038, 3114, 3208, 3335, 3398, 3637, 3647 |
| South East Asia | 577, 1766, 2166, 2279, 2463, 2641, 3038, 3114, 3318, 3335, 3398, 3505, 3624, |
| | 3637, 3767 |
| Sub-Saharan Africa | 577, 1766, 2166, 2641, 2650, 3038, 3208, 3335, 3637 |
| Western Asia | 577, 1766, 2166, 2279, 2463, 2641, 2650, 3038, 3114, 3208, 3335, 3398, 3637 |

TABLE 41
Region-specific peptide pools derived from ORF10 protein for CD8 T cell assays.

| Region | Seq. IDs of peptides |
|---|---|
| Australia | 595, 754, 1007, 1077, 1186, 1214, 1328, 1764, 1782, 2013, 2117, 2272, 2336, 2469, 3205 |
| Europe | 595, 754, 1007, 1077, 1186, 1214, 1328, 1764, 1782, 2117, 2272, 2336, 2898, 3442 |
| North Africa | 595, 754, 1007, 1077, 1186, 1214, 1328, 1764, 1782, 2013, 2272, 2336, 2469, 3205, 3479 |
| North America | 595, 754, 1007, 1077, 1186, 1214, 1328, 1764, 1782, 2013, 2117, 2272, 2469, 2898, 3205 |
| North East Asia | 595, 754, 1007, 1077, 1186, 1214, 1328, 1764, 1782, 2013, 2117, 2272, 2336, 2469, 2898, |
| | 3479 |
| Oceania | 595, 754, 1007, 1077, 1186, 1214, 1328, 1764, 1782, 2013, 2117, 2272, 2336, 2469, 2898, |
| | 3205 |
| South and | 595, 754, 1007, 1077, 1186, 1328, 1764, 1782, 2013, 2117, 2272, 2336, 2469, 2898, 3205, |
| Central | 3479 |
| America | |
| South Asia | 595, 754, 1007, 1077, 1186, 1214, 1328, 1764, 1782, 2013, 2117, 2272, 2336, 2469, 2898, |
| | 3479 |
| South East Asia | 595, 754, 1007, 1077, 1186, 1214, 1328, 1764, 1782, 2013, 2117, 2272, 2469, 2898 |
| Sub-Saharan | 595, 754, 1007, 1077, 1186, 1214, 1328, 1764, 1782, 2013, 2117, 2272, 2336, 2469, 2898, |
| Africa | 3205, 3442 |
| Western Asia | 595, 754, 1007, 1077, 1186, 1214, 1328, 1764, 1782, 2117, 2272, 2336, 2469, 2898, 3442, |
| | 3479 |

TABLE 42
Region-specific peptide pools derived from ORF9b protein for CD8 T cell assays.

| Region | Seq. IDs of peptides |
|---|---|
| Australia | 596, 637, 668, 814, 1023, 1333, 1458-1460, 1915, 1975, 2012, 2545, 2653, 2669, 2730, |
| | 2835, 2961, 2981, 3077, 3176, 3291, 3433, 3669, 3728 |
| Europe | 596, 637, 668, 814, 1023, 1063, 1458-1460, 1975, 2545, 2713, 2730, 2961, 2981, 3040, |
| | 3077, 3176, 3433, 3669 |
| North Africa | 596, 637, 668, 814, 1023, 1333, 1458-1460, 1915, 1975, 2012, 2545, 2669, 2713, 2835, |
| | 2961, 2981, 3034, 3077, 3165, 3176, 3183, 3291, 3433, 3646, 3669 |
| North America | 596, 637, 668, 1023, 1333, 1458-1459, 1915, 1975, 2012, 2524, 2545, 2653, 2669, 2730, |
| | 2961, 3077, 3176, 3291, 3433, 3669-3670, 3728 |
| North East Asia | 596, 637, 668, 814, 1023, 1063, 1333, 1458-1460, 1975, 2012, 2545, 2632, 2653, 2669, |
| | 2730, 2981, 3034, 3077, 3176, 3291, 3433, 3646, 3669-3670 |
| Oceania | 596, 637, 668, 814, 1023, 1063, 1333, 1458-1460, 1915, 1975, 1983, 2012, 2524, 2545, |
| | 2632, 2653, 2669, 2730, 2835, 2961, 2981, 3034, 3059, 3077, 3176, 3291, 3433, 3646, |
| | 3728 |
| South and Central | 596, 637, 668, 814, 1023, 1063, 1333, 1458-1460, 1915, 1975, 2012, 2545, 2669, 2713, |
| America | 2730, 2961, 2981, 3059, 3077, 3176, 3291, 3812 |
| South Asia | 596, 637, 668, 814, 1023, 1063, 1333, 1458-1460, 1975, 1983, 2012, 2545, 2632, 2669, |
| | 2730, 2835, 2961, 2981, 3034, 3077, 3165, 3176, 3291, 3433 |
| South East Asia | 596, 637, 668, 814, 1023, 1063, 1333, 1458-1460, 1975, 2012, 2524, 2545, 2632, 2653, |
| | 2669, 2730, 2835, 3034, 3059, 3077, 3176, 3291, 3433, 3646, 3669-3670 |
| Sub-Saharan Africa | 596, 637, 668, 1023, 1333, 1458-1459, 1915, 1975, 2524, 2545, 2632, 2713, 2835, 2961, |
| | 3034, 3077, 3176, 3291, 3433 |
| Western Asia | 596, 637, 668, 814, 1023, 1063, 1333, 1458-1460, 1975, 1983, 2012, 2524, 2545, 2669, |
| | 2713, 2730, 2835, 2961, 2972, 2981, 3034, 3059, 3077, 3176, 3291, 3433, 3646, 3669 |

TABLE 43
Region-specific peptide pools derived from NSP10 protein for CD8 T cell assays.

| Region | Seq. IDs of peptides |
|---|---|
| Australia | 702, 898, 966, 1040, 1114, 1246, 1446, 1570, 1833, 1900, 2014, 2343, 2467, 2743, 2769, |
| | 2787, 2887, 2967, 3047, 3067, 3178, 3229, 3306 |
| Europe | 702, 898, 966, 1040, 1114, 1160, 1246, 1570, 1833, 2764, 2769, 2787, 2967, 3047, 3067, |
| | 3084, 3178, 3422 |
| North Africa | 702, 898, 966, 1040, 1114, 1160, 1246, 1570, 1833, 1900, 2014, 2455, 2467, 2743, 2764, |
| | 2769, 2787, 2967, 3047, 3067, 3178, 3422, 3598 |
| North America | 702, 898, 1114, 1246, 1402, 1446, 1833, 2343, 2467, 2556, 2769, 2787, 2967, 3067, |
| | 3084, 3122, 3178, 3229, 3306, 3623, 3802 |
| North East Asia | 702, 898, 966, 1040, 1114, 1160, 1246, 1446, 1570, 1833, 2014, 2343, 2402, 2455, 2467, |
| | 2556, 2743, 2764, 2769, 2787, 2887, 2967, 3046-3047, 3067, 3084, 3122, 3167, 3178, |
| | 3229, 3306, 3422, 3623, 3802 |
| Oceania | 702, 777, 898, 966, 1040, 1114, 1160, 1246, 1402, 1446, 1570, 1833, 1900, 2014, 2343, |
| | 2402, 2467, 2556, 2743, 2764, 2769, 2787, 2887, 2967, 3046, 3067, 3084, 3167, 3178, |
| | 3229, 3306, 3422 |
| South and Central | 702, 898, 966, 1040, 1114, 1160, 1246, 1446, 1570, 1833, 1900, 2014, 2343, 2467, 2743, |
| America | 2769, 2787, 2887, 2967, 3046-3047, 3067, 3084, 3178, 3229, 3422 |
| South Asia | 702, 777, 898, 966, 1040, 1114, 1160, 1246, 1446, 1570, 1833, 1900, 2014, 2343, 2402, |
| | 2455, 2467, 2556, 2743, 2764, 2769, 2787, 2967, 3046-3047, 3067, 3084, 3167, 3178, |
| | 3422, 3598 |
| South East Asia | 702, 777, 898, 966, 1040, 1114, 1160, 1246, 1402, 1446, 1833, 2343, 2402, 2467, 2556, |
| | 2764, 2769, 2787, 2967, 3046, 3067, 3084, 3178, 3229, 3306, 3422, 3623, 3802 |
| Sub-Saharan Africa | 702, 898, 966, 1040, 1114, 1160, 1246, 1402, 1833, 1900, 2017, 2343, 2467, 2769, 2787, |
| | 2967, 3047, 3067, 3084, 3122, 3178, 3229, 3306, 3422 |
| Western Asia | 702, 777, 898, 966, 1040, 1114, 1160, 1246, 1402, 1570, 1833, 1900, 2014, 2017, 2343, |
| | 2402, 2455, 2467, 2556, 2769, 2787, 2967, 3046-3047, 3067, 3084, 3178, 3422 |

TABLE 44
Region-specific peptide pools derived from ORF9c protein for CD8 T cell assays.

| Region | Seq. IDs of peptides |
|---|---|
| Australia | 725, 787, 804, 1044, 1060, 1069, 1272, 1395, 1580, 1683, 1757, 1785, 1794, 2105, 2562, |
| | 2756, 2856, 2918, 2986, 3166, 3217, 3244, 3465, 3658 |
| Europe | 725, 787, 804, 1044, 1069, 1272, 1395, 1580, 1628, 1757, 1785, 1794, 2562, 2756, 2856, |
| | 2918, 3465 |
| North Africa | 725, 787, 804, 1011, 1044, 1060, 1069, 1272, 1395, 1580, 1683, 1757, 1785, 1794, 2105, |
| | 2319, 2562, 2756, 2856, 2918, 2964, 3166, 3423, 3465, 3658, 3713 |
| North America | 804, 1044, 1060, 1069, 1272, 1580, 1628, 1683, 1757, 1785, 1794, 2756, 2856, 2918, |
| | 2986, 3217, 3244, 3465, 3658, 3836 |
| North East Asia | 725, 787, 804, 1011, 1044, 1060, 1069, 1272, 1395, 1580, 1628, 1683, 1757, 1785, 1794, |
| | 2105, 2562, 2756, 2856, 2918, 2964, 2986, 3217, 3465, 3836 |
| Oceania | 725, 787, 804, 1011, 1044, 1060, 1069, 1272, 1395, 1580, 1628, 1683, 1757, 1785, 1794, |
| | 2105, 2562, 2756, 2856, 2918, 2986, 3166, 3217, 3244, 3465, 3658 |
| South and Central | 725, 787, 804, 1011, 1044, 1060, 1069, 1272, 1395, 1580, 1628, 1683, 1757, 1785, 1794, |
| America | 2105, 2562, 2756, 2856, 2918, 3118, 3166, 3217, 3455, 3465, 3658, 3826 |
| South Asia | 725, 787, 804, 1011, 1044, 1060, 1069, 1272, 1395, 1580, 1683, 1757, 1785, 1794, 2105, |
| | 2562, 2756, 2856, 2918 |
| South East Asia | 725, 787, 804, 1011, 1044, 1060, 1069, 1272, 1395, 1580, 1628, 1683, 1757, 1785, 1794, |
| | 2105, 2562, 2756, 2856, 2918, 2964, 2986, 3166, 3217, 3399, 3465, 3836 |
| Sub-Saharan Africa | 804, 1044, 1060, 1069, 1272, 1580, 1628, 1683, 1757, 1785, 1794, 2319, 2756, 2856, |
| | 2918, 2964, 3244, 3465, 3658 |
| Western Asia | 725, 787, 804, 1011, 1044, 1060, 1069, 1272, 1395, 1580, 1628, 1757, 1785, 1794, 2562, |
| | 2756, 2856, 2918, 2964, 3118, 3166, 3244, 3455, 3465 |

TABLE 45
Region-specific peptide pools derived from NSP7 protein for CD8 T cell assays.

| Region | Seq. IDs of peptides |
|---|---|
| Australia | 769, 920, 960, 967, 1028, 1101, 1250, 1467, 1566, 1630, 1723, 1788, 2245, 2456, |
| | 2720, 2928, 3101, 3148 |
| Europe | 769, 920, 960, 967, 1028, 1101, 1467, 1566, 1630, 1723, 1977, 2245, 2731, 2847, |
| | 2928, 3101, 3148 |
| North Africa | 769, 920, 960, 967, 1101, 1250, 1467, 1566, 1630, 1723, 1726, 1788, 1951, 1977, |
| | 2123, 2245, 2456, 2462, 2477, 2658, 2845, 2928, 3101, 3148, 3175 |
| North America | 769, 920, 960, 967, 1101, 1467, 1566, 1630, 1723, 2720, 2847, 2928, 3101, 3141 |
| North East Asia | 769, 920, 960, 967, 1101, 1250, 1467, 1566, 1630, 1723, 1726, 1788, 1951, 2245, |
| | 2456, 2477, 2720, 2847, 2928, 3101, 3280, 3742 |
| Oceania | 769, 920, 960, 967, 1028, 1101, 1250, 1467, 1566, 1630, 1723, 1726, 1788, 1951, |
| | 2245, 2456, 2720, 2731, 2847, 2928, 3101, 3148, 3280, 3284, 3742 |
| South and Central America | 769, 920, 960, 967, 1101, 1250, 1467, 1566, 1630, 1723, 1788, 2245, 2456, 2462, |
| | 2477, 2658, 2720, 2847, 2928, 3101, 3148, 3622 |
| South Asia | 769, 920, 960, 967, 1028, 1101, 1250, 1467, 1566, 1630, 1723, 1788, 1951, 1977, |
| | 2123, 2245, 2456, 2462, 2477, 2720, 2731, 2845, 2847, 2928, 3101, 3148, 3175, |
| | 3280 |
| South East Asia | 769, 920, 960, 967, 1028, 1101, 1250, 1467, 1566, 1630, 1723, 1951, 2456, 2462, |
| | 2477, 2658, 2720, 2731, 2847, 2928, 3101, 3284, 3622, 3742, 3776 |
| Sub-Saharan Africa | 769, 920, 960, 967, 1101, 1467, 1566, 1630, 1723, 1726, 1788, 2456, 2720, 2847, |
| | 2928, 3101, 3148 |
| Western Asia | 769, 920, 960, 967, 1101, 1250, 1467, 1566, 1630, 1723, 1788, 1951, 1977, 2245, |
| | 2456, 2462, 2477, 2720, 2731, 2847, 2928, 3101, 3141, 3148, 3280, 3284, 3622 |

TABLE 46
Region-specific peptide pools derived from ORF3b protein for CD8 T cell assays.

| Region | Seq. IDs of peptides |
|---|---|
| Australia | 835, 1199, 1386, 2221, 2335, 3228 |
| Europe | 835, 1199, 1386, 2221, 2335, 2948 |
| North Africa | 835, 1199, 2221, 2255, 2335, 2505, 2948, 3228, 3813 |
| North America | 835, 1386, 2221, 3228, 3789 |
| North East Asia | 835, 1199, 1386, 2221, 2255, 2335, 2505, 2948, 3228, 3789 |
| Oceania | 835, 1199, 1386, 2221, 2335, 2505, 3228 |
| South and Central America | 835, 1199, 1386, 2221, 2255, 2335, 2505 |
| South Asia | 835, 1199, 1386, 2221, 2255, 2335, 2948, 3228 |
| South East Asia | 835, 1199, 1386, 2221, 2335, 2505, 3789 |
| Sub-Saharan Africa | 835, 1199, 1386, 2221, 2335, 2505, 2948, 3228, 3813 |
| Western Asia | 835, 1199, 1386, 2221, 2255, 2335, 2505, 2948, 3228 |

TABLE 47
Region-specific peptide pools derived from ORF7b protein for CD8 T cell assays.

| Region | Seq. IDs of peptides |
|---|---|
| Australia | 1027, 1046, 1148, 1368, 1978, 2252, 2271, 2453, 2554, 2947, 3261, 3581, 3586 |
| Europe | 1027, 1046, 1148, 1239, 1368, 1978, 2271, 2453, 2947, 3581, 3625, 3704 |
| North Africa | 996, 1027, 1046, 1148, 1368, 1978, 2252, 2271, 2453, 2554, 2947, 3015, 3203, |
| | 3261, 3581, 3586 |
| North America | 1027, 1046, 1148, 1239, 1368, 1978, 2453, 2947, 3581 |
| North East Asia | 996, 1027, 1046, 1148, 1239, 1368, 1978, 2252, 2271, 2453, 2554, 2947, 3015, |
| | 3261 |
| Oceania | 996, 1027, 1046, 1148, 1239, 1368, 1978, 2252, 2271, 2453, 2554, 2947, 3261, |
| | 3581, 3586, 3625 |
| South and Central America | 996, 1027, 1046, 1148, 1368, 1978, 2252, 2271, 2453, 2554, 2947, 3581, 3586 |
| South Asia | 996, 1027, 1046, 1148, 1239, 1368, 1978, 2252, 2271, 2453, 2554, 2947, 3015, |
| | 3203, 3261, 3581 |
| South East Asia | 996, 1027, 1046, 1148, 1239, 1368, 1978, 2252, 2271, 2453, 3015, 3261, 3581, |
| | 3625 |
| Sub-Saharan Africa | 996, 1027, 1046, 1148, 1239, 1368, 1978, 2252, 2271, 2453, 2947, 3581, 3586 |
| Western Asia | 996, 1027, 1046, 1148, 1239, 1368, 1978, 2252, 2271, 2453, 2554, 2947, 3261, |
| | 3581 |

### Discussion

SARS2TPools is a first-of-its-kind software platform that provides optimized SARS-CoV-2 peptide pools for assessing vaccine-induced T cell responses in a geographical region. These pools can be used to explore key questions related to COVID-19 vaccines and the role of T cell immunity. For example, characterizing effects of mutations within T cell epitopes in emerging SARS-CoV-2 variants on vaccine-induced T cell responses, investigating association between targeting specific T cell epitopes and protection from severe disease, characterizing the breadth, diversity, and durability of vaccine-induced T cell responses, and contrasting the T cell responses elicited by different vaccines. These questions are important and relevant for both academic and industry research focused on development and assessment of COVID-19 vaccines. Answering them can help provide pre-emptive indicators of the potential for T-cell escape (e.g., due to variants) for specific vaccines.

At present, HLA alleles commonly found in various regions (e.g., South-east Asia, South Asia, and Oceania) are underrepresented in the available experimental data (FIG. 6B). T cell responses against SARS-CoV-2 proteins other than S are also understudied (FIG. 6C). Characterizing these responses is particularly important for inactivated whole-virion based vaccines (already in use in more than 48 countries (Shrotri et al. 2021)) that have been shown to elicit T cell responses against not only S but also against other SARS-CoV-2 proteins (Bueno et al. 2021). This characterization will also be important for emerging SARS-CoV-2 vaccines (Hwang et al. 2021; Sohail et al. 2021) that incorporate domains or peptides derived from S as well as other SARS-CoV-2 proteins (e.g., EpiVacCorona involves peptides derived from both S and nucleocapsid (Aleksandr B. Ryzhikov et al. 2021; A. B. Ryzhikov et al. 2021)). The peptide pools provided by the developed platform can help with assessing T cell responses in above scenarios.

Analysis of the predictions of our in silico approach revealed that some of the experimentally-determined epitopes may be highly promiscuous and thus may cover a large percentage of the global population. Specifically, we identified 12 epitopes which have been experimentally-determined to be associated with at least 2 HLA alleles, while they are predicted to be associated with at least eight or more additional HLA alleles (Table 14). For example, in silico predictions suggest that the epitope YLQPRTFLL—the most immunoprevalent SARS-CoV-2 epitope determined so far (Quadeer et al. 2021)—is associated with a total of 22 HLA alleles, two of which (HLA-A*02:01 and HLA-B*08:01) have been determined experimentally thus far. While further experiments are required to validate these predicted associations, such promiscuous epitopes, recognized potentially by a large population, can be of interest in the context of designing robust next generation COVID-19 vaccines.

TABLE 14
List of epitopes having experimentally-determined association
with at least 2 HLA alleles and in silico
predicted association with at least
8 additional HLA alleles.

| | | | | | Experimentally- | |
|---|---|---|---|---|---|---|
| Sr. | | | | | determined | Predicted HLA |
| no. | Peptide | Protein | Start | Stop | HLA associations | associations |
| 1 | AEAELAKN | NSP3 | 1798 | 1806 | B*44:02, B*44:03 | B*40:01, B*40:02, |
| | V | | | | | B*40:06, B*40:10, |
| | | | | | | B*41:01, B*44:02, |
| | | | | | | B*44:03, B*44:04, |
| | | | | | | B*45:01, B*50:01 |
| 2 | ASMPTTI | NSP3 | 1374 | 1382 | A*11:01, A*30:01 | A*03:01, A*03:02, |
| | AK | | | | | A*03:27, A*11:01, |
| | | | | | | A*11:02, A*11:06, |
| | | | | | | A*30:01, A*31:01, |
| | | | | | | A*31:03, A*68:01, |
| | | | | | | A*74:01 |
| 3 | FLAHIQWM | NSP4 | 359 | 367 | A*02:01, A*02:06 | A*02:01, A*02:02, |
| | V | | | | | A*02:03, A*02:04, |
| | | | | | | A*02:05, A*02:06, |
| | | | | | | A*02:07, A*02:11, |
| | | | | | | A*02:12, A*02:19, |
| | | | | | | A*02:24, A*02:26, |
| | | | | | | A*02:52 |
| 4 | GTHWFVTQ | S | 1099 | 1107 | A*11:01, A*31:01 | A*03:01, A*03:02, |
| | R | | | | | A*03:27, A*11:01, |
| | | | | | | A*11:02, A*11:06, |
| | | | | | | A*31:01, A*31:03, |
| | | | | | | A*31:29, A*33:03, |
| | | | | | | A*34:01, A*68:01, |
| | | | | | | A*68:03, A*74:01 |
| 5 | IPTNFTI | S | 714 | 722 | B*07:02, B*51:01 | B*07:02, B*07:07, |
| | SV | | | | | B*07:08, B*07:12, |
| | | | | | | B*35:03, B*42:01, |
| | | | | | | B*51:01, B*51:10, |
| | | | | | | B*54:01, B*54:18, |
| | | | | | | B*55:02, B*56:01, |
| | | | | | | B*56:02, B*56:43 |
| 6 | KAYNVTQA | N | 266 | 274 | B*35:01, B*57:01 | A*31:08, A*32:01, |
| | F | | | | | B*07:17, B*15:01, |
| | | | | | | B*15:02, B*15:06, |
| | | | | | | B*15:13, B*15:21, |
| | | | | | | B*15:25, B*35:01, |
| | | | | | | B*35:05, B*35:08, |
| | | | | | | B*35:19, B*35:27, |
| | | | | | | B*35:43, B*46:01, |
| | | | | | | B*53:01, B*57:01, |
| | | | | | | B*58:01, C*02:02, |
| | | | | | | C*02:03, C*02:09, |
| | | | | | | C*02:10, C*03:02, |
| | | | | | | C*03:03, C*03:04, |
| | | | | | | C*03:05, C*03:13, |
| | | | | | | C*06:03, C*07:01, |
| | | | | | | C*12:02, C*12:03, |
| | | | | | | C*16:01 |
| 7 | KTFPPTE | N | 361 | 369 | A*03:01, A*11:01, | A*03:01, A*03:02, |
| | PK | | | | A *68:01 | A*03:27, A*11:01, |
| | | | | | | A*11:02, A*11:06, |
| | | | | | | A*30:01, A*31:01, |
| | | | | | | A*31:03, A*31:29, |
| | | | | | | A*34:01, A*68:01, |
| | | | | | | A*74:01 |
| 8 | KTIQPRV | NSP2 | 102 | 110 | A*03:01, A*11:01 | A*03:01, A*03:02, |
| | EK | | | | | A*03:27, A*11:01, |
| | | | | | | A*11:02, A*11:06, |
| | | | | | | A*30:01, A*31:01, |
| | | | | | | A*31:03, A*31:29, |
| | | | | | | A*74:01 |
| 9 | SASKIIT | ORF3a | 58 | 66 | A*03:01, A*11:01 | A*03:01, A*03:02, |
| | LK | | | | | A*03:27, A*11:01, |
| | | | | | | A*11:02, A*11:06, |
| | | | | | | A*30:01, A*31:03, |
| | | | | | | A*34:01, A*68:01, |
| | | | | | | A*68:03, A*74:01 |
| 10 | VTNNTFT | NSP2 | 628 | 636 | A*03:01, A*11:01 | A*03:01, A*03:02, |
| | LK | | | | | A*03:27, A*11:01, |
| | | | | | | A*11:02, A*11:06, |
| | | | | | | A*30:01, A*31:01, |
| | | | | | | A*31:03, A*31:29, |
| | | | | | | A*34:01, A*68:01, |
| | | | | | | A*68:03, A*74:01 |
| 11 | YLQPRTF | S | 269 | 277 | A*02:01, B*08:01 | A*02:01, A*02:02, |
| | LL | | | | | A*02:03, A*02:04, |
| | | | | | | A*02:05, A*02:06, |
| | | | | | | A*02:07, A*02:11, |
| | | | | | | A*02:12, A*02:19, |
| | | | | | | A*02:24, A*02:26, |
| | | | | | | A*02:52, B*08:01, |
| | | | | | | B*08:02, B*08:03, |
| | | | | | | C*17:01 |
| 12 | YYQLYSTQ | ORF3a | 211 | 219 | A*24:02, C*07:02 | A*23:01, A*24:02, |
| | L | | | | | A*24:06, A*24:07, |
| | | | | | | C*04:01, C*07:02, |
| | | | | | | C*07:03, C*07:17, |
| | | | | | | C*14:02, C*14:03 |

Compared to T cell response assessment using overlapping peptide pools, peptide pools optimized for a specific region comprise of a limited set of peptides in the context of cognate HLA alleles, and would thus be less susceptible to peptide competition (Pala et al. 1988). This is of practical importance particularly for a virus like SARS-CoV-2 which has a large (˜10k residues) proteome. In addition to overlapping peptide pools, generalized peptide pools have also been proposed to assess SARS-CoV-2 T cell responses. Such pools comprise of peptides associated with a few globally prevalent HLA alleles (e.g., the pool proposed in (Grifoni et al. 2020) comprises of peptides associated with 12 most-prevalent HLA-A and -B alleles). Compared to such generalized peptide pools, a peptide pool optimized for the HLA alleles prevalent in a specific region would be expected to measure T cell responses in a population more comprehensively.

Recent studies have demonstrated that COVID-19 vaccines elicit strong CD4⁺ T cell responses, along with CD8⁺ responses (Sahin et al. 2020; Tauzin et al. 2021). The pools for measuring CD4⁺ T cell responses, provided on the platform at present, comprise only of experimentally-determined CD4⁺ T cell epitopes. In future when sufficient experimentally-determined HLA-resolved CD4⁺ T cell epitope data becomes available, CD4⁺ pools provided by SARS2TPools may then be supplemented by in silico predictions following an approach similar to the one employed for CD8⁺ T cell epitopes. SARS2TPools will be periodically updated to incorporate more experimental CD4⁺ and CD8⁺ T cell epitope data as it becomes available.

### Methods and Materials

Data collection. We downloaded experimentally-determined HLA class I and class II restricted SARS-CoV-2 T cell epitope data (CD8⁺ and CD4⁺, respectively) from the immune epitope database (IEDB) (Vita et al. 2019) on Mar. 10, 2021. We included all epitopes that were reported in positive T cell assays with associated HLA information available. The data consisted of 768 and 445 unique class I and class II epitope-HLA pairs, respectively. Majority of the HLA class I restricted epitopes (474/768) were nine residues long, which is the canonical length of epitopes restricted by HLA class I alleles. The epitope data was found to be biased towards a handful of HLA alleles, with only 10 HLA class I alleles (HLA-A*02:01, HLA-A*03:01, HLA-A*11:01, HLA-A*24:02, HLA-A*29:02, HLA-A*68:01, HLA-B*07:02, HLA-B*35:01, HLA-B*51:01, HLA-B*57:01) having 20 or more nine-residue-long epitopes. Collectively, the epitopes restricted by these 10 HLA alleles corresponded to ˜62% (295/474) of nine-residue-long epitopes in the data. In the case of HLA class II restricted epitopes, all the available epitopes were 15 resides long, and only 3 HLA alleles had more than 20 epitopes in the data.

In silico prediction methods. Performance of several in silico epitope prediction methods were benchmarked against the set of experimentally-determined SARS-CoV-2 epitopes associated with the 10 HLA class I alleles having the most data. The considered methods included the current state-of-the-art methods such as MHCflurry (O'Donnell et al. 2020), NetMHCpan4.1 (Reynisson et al. 2020), HLAthena (Sarkizova et al. 2020), NetMHCpan4.0 (Jurtz et al. 2017), NetMHC4.0 (Andreatta and Nielsen 2016), along with other common prediction methods that have been employed for predicting SARS-CoV-2 epitopes (Sohail et al. 2021) such as NetMHCpan3.0 (Nielsen and Andreatta 2016), SMM (Peters and Sette 2005), SMMPMBEC (Kim et al. 2009), and IEDB consensus (Moutaftsi et al. 2006). We considered the eluted ligand and binding affinity predictions (denoted by suffix BA and EL respectively) of NetMHCpan4.1 and NetMHCpan4.0 as separate methods, as was done for the latter method in (Sarkizova et al. 2020) and (Paul et al. 2020). Similarly, we considered the binding affinity and the presentation score predictions of MHCflurry as two separate methods, referred to as MHCflurry2.0BA and MHCflurry2.0P. In cases where a method required an input other than the protein sequence, HLA allele, and length of the predicted peptides, we used the default parameter settings for that method.

Union approach. In this work, we have proposed a union approach based on combining the top-ranked predictions of MHCflurry2.0P and NetMHCpan4.1BA to obtain a set of peptides restricted by a given HLA. This approach was motivated by performance comparison analysis of the 12 in silico epitope prediction methods listed above. Briefly, we ranked peptides in ascending order of their predicted score using each method and compared the histograms of ranks of experimentally-determined SARS-CoV-2 CD8⁺ T cell epitopes associated with the 10 HLA class I alleles with the most data. We found that these histograms were bi-modal for all 12 methods. That is, while the top predictions of each method contained a large number of experimentally-determined epitopes, a good number of epitopes were also ranked quite low by each method (FIG. 10). Exploring the relationships among the set of top 20 ranked peptides per HLA allele predicted by these methods revealed that the predictions of MHCflurry2.0P were most distinct from those of other methods (FIGS. 11A-D). Consistent results were obtained when this set was constructed by pooling the top 10 to top 25 ranked peptides restricted by each HLA allele (FIGS. 11A-D). Predictions of MHCflurry2.0P also contained a large number of experimentally-determined SARS-CoV-2 epitopes that were not present in the set of top-ranked peptides predicted by any other method (FIG. 7A). Given the uniqueness of the predictions of MHCflurry2.0P, we asked if a strategy that combines the predictions of MHCflurry2.0P with any of the other 11 methods would work better than any individual method. The union strategy combines the top x predictions of any two methods and provides a set of peptides whose size can vary between x and 2x depending on the number of common peptides predicted by each method. We fixed one of the methods as MHCflurry2.0P and predicted 11 peptide pools by combining predictions of MHCflurry2.0P with those of the other 11 methods. Our analysis showed that the pool predicted by the union approach always had a higher hit-rate (the fraction of experimentally-determined SARS-CoV-2 epitopes present in the set of top-ranked peptides) than those predicted by the individual methods (FIG. 12). While comparison among the various union approaches did not readily reveal a clear winner, the unions of MHCflurry2.0P with the in silico methods NetMHC4.0, NetMHCpan4.0BA, NetMHCpan4.1BA, and NetMHCpan4.1EL ranked among the top (FIG. 13 Table 13.)

TABLE 13
Performance comparison of the MHCflurry2.0P-based union methods based on the rank-sum metric, Rᵢ.

| | Union | | | | | | | | | | | | |
|---|---|---|---|---|---|---|---|---|---|---|---|---|---|
| i | method | rᵢ(10) | rᵢ(11) | rᵢ(12) | rᵢ(13) | rᵢ(14) | rᵢ(15) | rᵢ(16) | rᵢ(17) | rᵢ(18) | rᵢ(19) | rᵢ(20) | Rᵢ* |
| 1 | MHCflurry2.0P | 1 | 1 | 2 | 1 | 3 | 2 | 2 | 2 | 5 | 4 | 4 | 27 |
| | and NetMHC4.0 | | | | | | | | | | | | |
| 2 | MHCflurry2.0P and | 3 | 4 | 6 | 7 | 2 | 1 | 1 | 1 | 1 | 2 | 2 | 30 |
| | NetMHCpan4.0BA | | | | | | | | | | | | |
| 3 | MHCflurry2.0P and | 2 | 2 | 1 | 3 | 6 | 3 | 5 | 5 | 3 | 3 | 1 | 34 |
| | NetMHCpan4.1BA | | | | | | | | | | | | |
| 4 | MHCflurry2.0P and | 7 | 5 | 4 | 2 | 4 | 6 | 3 | 4 | 2 | 1 | 3 | 41 |
| | NetMHCpan4.1EL | | | | | | | | | | | | |
| 5 | MHCflurry2.0P | 10 | 10 | 11 | 5 | 1 | 4 | 6 | 3 | 4 | 5 | 6 | 65 |
| | and HLAthena | | | | | | | | | | | | |
| 6 | MHCflurry2.0P | 5 | 3 | 5 | 4 | 5 | 5 | 8 | 9 | 10 | 11 | 7 | 72 |
| | and NetMHCpan3.0 | | | | | | | | | | | | |
| 7 | MHCflurry2.0P and | 6 | 9 | 7 | 6 | 7 | 8 | 4 | 6 | 8 | 7 | 9 | 77 |
| | SMMPMBECv2.24 | | | | | | | | | | | | |
| 8 | MHCflurry2.0P and | 4 | 6 | 3 | 9 | 8 | 7 | 9 | 8 | 7 | 6 | 10 | 77 |
| | MHCflurry2.0BA | | | | | | | | | | | | |
| 9 | MHCflurry2.0P and | 9 | 7 | 8 | 8 | 9 | 9 | 7 | 7 | 6 | 8 | 5 | 83 |
| | NetMHCpan4.0EL | | | | | | | | | | | | |
| 10 | MHCflurry2.0P and | 8 | 8 | 9 | 10 | 10 | 10 | 10 | 11 | 9 | 9 | 8 | 102 |
| | IEDBConsv2.24 | | | | | | | | | | | | |
| 11 | MHCflurry2.0P | 11 | 11 | 10 | 11 | 11 | 11 | 11 | 10 | 11 | 10 | 11 | 118 |
| | and SMMv2.24 | | | | | | | | | | | | |

*Rank-sum metric Rᵢ is defined as Rᵢ = Σₓ₌₁₀²⁰ rᵢ(x), where rᵢ(x) is the i-th union method's rank (assigned based on hit-rate) among the 11 union methods for the set of top x ranked predicted peptides (FIG. 13). Hit-rate represents the fraction of experimentally known epitopes present in the set of top x ranked predicted peptides.

The union method implemented in SARS2TPools combines the predictions of MHCflurry2.0P with NetMHCpan4.1BA. SARS2TPools provides optimized peptide pools by supplementing experimentally-determined epitopes with small or large sized group of in silico predicted epitopes corresponding respectively to top 10 and top 20 predictions of each method being combined. We used the default thresholds of MHCflurry2.0P and NetM1HCpan4.1BA to assess whether or not a peptide is predicted to be an epitope. However, the platform also provides a relaxed threshold which can be particularly useful for specific proteins with very limited number of predicted epitopes.

Statistical analysis. Statistical analyses were performed using the R language (version 3.6) on the RStudio server (version 1.3). The software platform was developed using the open source R Shiny (version 1.5) development framework.

### Example 3—COVIDEP: A Web-Based Platform for Real-Time Reporting of Vaccine Target Recommendations for SARS-COV-2

### Introduction

The COVID-19 pandemic, caused by the novel coronavirus SARS-CoV-2, has brought much of the world to a virtual lockdown. As the virus continues to spread rapidly and the pandemic intensifies, the need for an effective vaccine is becoming increasingly apparent. A critical part of vaccine design is to identify targets, or epitopes, that can induce an effective immune response against SARS-CoV-2. This problem is challenged by our limited understanding of this novel coronavirus and of its interplay with the human immune system.

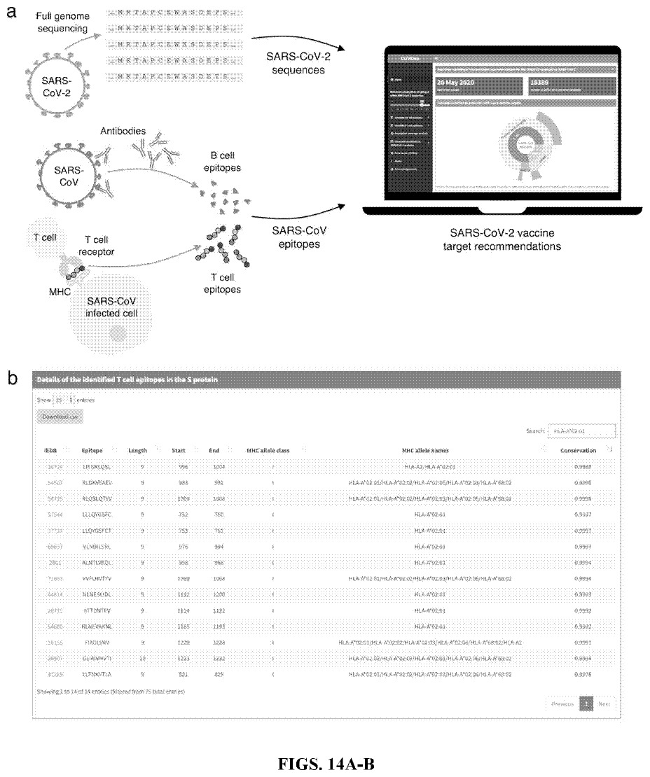

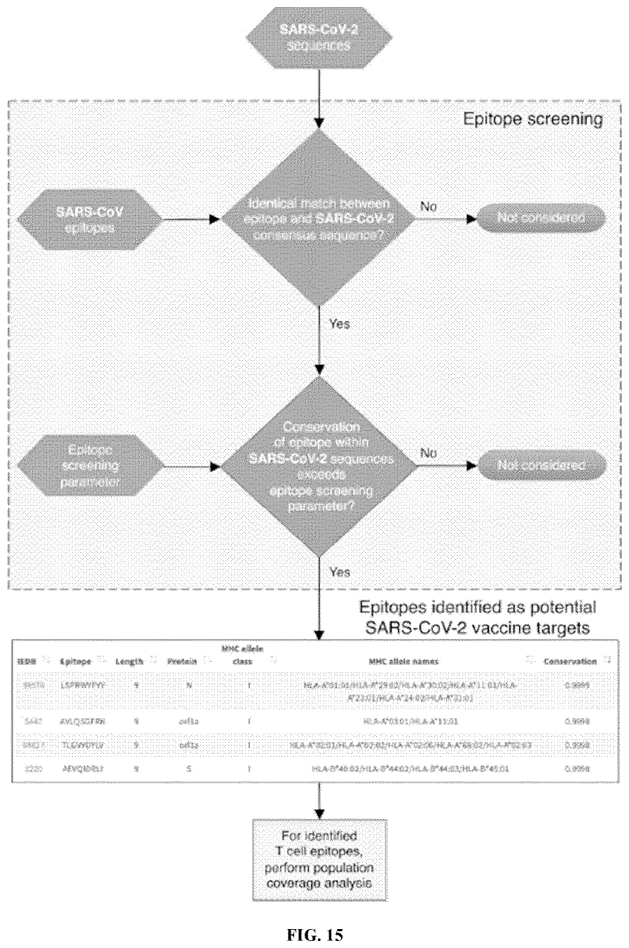

In response to this challenge, we have developed COVIDep (COVIDep.ust.hk), a first-of-its-kind web-based platform that pools genetic data for SARS-CoV-2 and immunological data for the 2003 SARS virus, SARS-CoV, to identify B-cell and T-cell epitopes to serve as vaccine target recommendations for SARS-CoV-2 (FIG. 14A). The identified epitopes are experimentally-derived from SARS-CoV and have a close genetic match with the available SARS-CoV-2 sequences (see FIG. 15 for a detailed protocol description). Briefly, COVIDep periodically pools SARS-CoV-2 sequence data from the GISAID database (gisaid.org) and compares with experimentally-determined T cell and B cell epitopes of SARS-CoV, obtained from the ViPR database (www.viprbrc.org). The T cell epitopes were determined based on either positive T cell assays or positive MHC binding assays for SARS-CoV. For the B cell epitopes, both linear and discontinuous epitopes were considered. The system outputs those epitopes that are genetically similar in SARS-CoV-2, based on an epitope screening parameter. This user-defined parameter allows the user to select epitopes based on their conservation in the SARS-CoV-2 sequence data, where conservation is defined as the fraction of SARS-CoV-2 sequences with the exact epitope sequence. The value of this parameter is set to 0.95 as default; however, the user may change this value to adjust the stringency of the screening criterion. For example, reducing the value of the parameter will allow for the consideration of epitopes with greater genetic variation, potentially increasing the set of recommended SARS-CoV-2 vaccine targets. For the identified T cell epitopes, the population coverage analysis tool available at IEDB (www.iedb.org) is used to estimate the percentage of a specified population that can elicit a response against them. For T-cell epitopes, it provides estimates of population coverage, globally and for specific regions. COVIDep is flexible and user-friendly, comprising an intuitive graphical interface and interactive visualizations. In addition to producing formatted, exportable lists of the identified B-cell and T-cell epitopes and their basic characteristics, COVIDep includes displays for each of the SARS-CoV-2 proteins, showing the locations of the identified epitopes on the primary structure. Further graphical displays are provided to aid interpretation of the data, including a temporal and geographical breakdown of the analysed sequences, and a display of the observed genetic variation (amino acid mutation frequencies) for each of the SARS-CoV-2 proteins. The platform is updated daily, based on the latest SARS-CoV-2 sequence data in the GISAID database (gisaid.org). Periodic updates are important since SARS-CoV-2 sequences are being made available at an increasing rate through international data sharing efforts, and the identification of vaccine targets is influenced by newly observed genetic variation.

The vaccine targets recommended by COVIDep exploit the genetic similarities between SARS-CoV-2 and SARS-CoV, along with known immune targets for SARS-CoV that have been determined experimentally (available at the ViPR database; viprbrc.org). The system implements a protocol that identifies, from among the SARS epitopes that can induce a human immune response, those that are genetically similar in SARS-CoV-2. This approach, proposed and tested in Example 1 [see also 56] based on limited early data, identified known SARS-CoV epitopes that had an identical genetic match in SARS-CoV-2. These epitopes presented initial vaccine target recommendations for potentially eliciting a protective, cross-reactive immune response against SARS-CoV-2. Similar results were reported in a subsequent independent study [57], where a related approach exploiting genetic similarity between SARS-CoV and SARS-CoV-2 was used to identify potential SARS-CoV-2 vaccine targets.

The use of SARS-CoV immunological data to inform vaccine targets for SARS-CoV-2 is being supported by experimental results. There is evidence of cross-neutralization by SARS-CoV-derived antibodies binding to genetically similar regions of SARS-CoV-2's spike protein [58-60]. Conversely, studies have demonstrated that specific SARS-CoV-derived antibodies binding to the spike's receptor binding domain, which has significant genetic differences in SARS-CoV-2, have limited cross-reactivity [61]. T cell responses against spike protein epitopes that are genetically similar in SARS-CoV and SARS-CoV-2 have also been reported in COVID-19 infected patients[62, 63], and in a preclinical vaccine trial [64](FIG. 14B). For instance, FIG. 14B illustrates the T-cell epitopes reported by COVIDep (as of 20 May 2020) for the spike protein of SARS-CoV-2. Here, the Search box (in the top right) was used to select only the HLA-A*02:01-restricted epitopes. (An explanation of all interactive COVIDep visualizations is incorporated in the “How to use COVIDep page” of the platform.). Of the 14 epitopes listed in the display, 9 of them (IEDB IDs 36724, 54507, 54725, 69657, 71663, 2801, 54680, 16156, and 37289) overlap with epitopes against which cytotoxic CD8+ T cell responses have been observed in peripheral blood mononuclear cells isolated from COVID-19 patients [62,63]. T cell responses were also recorded against protein regions overlapping with the epitope with IEDB ID 71663 in a pre-clinical trial of a DNA vaccine candidate [64]. Epitopes recommended by COVIDep have notable overlap with the findings in these and other [65, 66] experimental studies [67] (see FIGS. 2 and 3 in [67]).

The recommendations provided by COVIDep may be used to broadly guide vaccine designs and associated experimental studies, and may help to expedite the discovery of an effective vaccine for COVID-19.

### Methods and Materials

Data availability. The SARS-CoV-2 full genome sequence data was periodically downloaded from the Global Initiative on Sharing Avian Influenza Database (GISAID; www.gisaid.org). The SARS-CoV epitope sequence data was downloaded from the Virus Pathogen Database and Analysis Resource (ViPR; viprbrc.org). The population coverage statistics of HLA alleles were obtained from the Immune Epitope Database and Analysis Resource (IEDB; iedb.org).

Code availability. The source code for the developed platform is available at the COVIDep GitHub repository github.com/COVIDep).

All patents, patent applications, and other publications, including GenBank Accession Numbers and equivalents, cited in this application are incorporated by reference in the entirety for all purposes.

### REFERENCES

1. Wang, C.; Horby, P. W.; Hayden, F. G.; Gao, G. F. A novel coronavirus outbreak of global health concern. Lancet 2020, 395, 470-473.
2. Centers-of-Disease-Control-and-Prevention Confirmed 2019-nCoV cases globally. Available online: https://www.ede.gov/coronavirus/2019-ncov/locations-confirmed-cases.html (accessed on Feb. 24, 2020).
3. World-Health-Organization Statement on the second meeting of the International Health Regulations (2005) Emergency Committee regarding the outbreak of novel coronavirus (2019-nCoV). Available online: https://www.who.int/news-room/detail/30-01-2020-statement-on-the-second-meeting-of-the-international-health-regulations-(2005)-emergency-committee-regarding-the-outbreak-of-novel-coronavirus-(2019-ncov) (accessed on Feb. 24, 2020).
4. World-Health-Organization Coronavirus disease (COVID-19) outbreak. Available online: https://www.who.int/emergencies/diseases/novel-coronavirus-2019 (accessed on Feb. 23, 2020).
5. World-Health-Organization Statement on the meeting of the International Health Regulations (2005) Emergency Committee regarding the outbreak of novel coronavirus (2019-nCoV). Available online: https://www.who.int/news-room/detail/23-01-2020-statement-on-the-meeting-of-the-international-health-regulations-(2005)-emergency-committee-regarding-the-outbreak-of-novel-coronavirus-(2019-ncov) (accessed on Feb. 24, 2020).
6. Huang, C.; Wang, Y.; Li, X.; Ren, L.; Zhao, J.; Hu, Y.; Zhang, L.; Fan, G.; Xu, J.; Gu, X.; et al. Clinical features of patients infected with 2019 novel coronavirus in Wuhan, China. Lancet 2020, 395, 497-506.
7. Heymann, D. L. Data sharing and outbreaks: best practice exemplified. Lancet 2020, 395, 469-470.
8. Liu, X.; Wang, X.-J. Potential inhibitors for 2019-nCoV coronavirus M protease from clinically approved medicines. bioRxiv 2020.01.29.924100 2020.
9. Zhou, P.; Yang, X.-L.; Wang, X.-G.; Hu, B.; Zhang, L.; Zhang, W.; Si, H.-R.; Zhu, Y.; Li, B.; Huang, C.-L.; et al. A pneumonia outbreak associated with a new coronavirus of probable bat origin. Nature 2020.
10. World-Health-Organization Update 49—SARS case fatality ratio, incubation period. Available online: https://www.who.int/csr/sars/archive/2003_05_07a/en/ (accessed on Feb. 23, 2020).
11. World-Health-Organization Middle East respiratory syndrome coronavirus (MERS-CoV). Available online: https://www.who.int/emergencies/mers-cov/en/ (accessed on Feb. 23, 2020).
12. Lu, R.; Zhao, X.; Li, J.; Niu, P.; Yang, B.; Wu, H.; Wang, W.; Song, H.; Huang, B.; Zhu, N.; et al. Genomic characterisation and epidemiology of 2019 novel coronavirus: implications for virus origins and receptor binding. Lancet 2020, 6736, 1-10.
13. Letko, M.; Munster, V. Functional assessment of cell entry and receptor usage for lineage B 3-coronaviruses, including 2019-nCoV. bioRxiv 2020.01.22.915660 2020.
14. Hoffmann, M.; Kleine-Weber, H.; Kruger, N.; Muller, M.; Drosten, C.; Pohlmann, S. The novel coronavirus 2019 (2019-nCoV) uses the SARS-coronavirus receptor ACE2 and the cellular protease TMPRSS2 for entry into target cells. bioRxiv 2020.01.31.929042 2020.
15. Yang, Z.-Y.; Kong, W.-P.; Huang, Y.; Roberts, A.; Murphy, B. R.; Subbarao, K.; Nabel, G. J. A DNA vaccine induces SARS coronavirus neutralization and protective immunity in mice. Nature 2004, 428, 561-564.
16. Deming, D.; Sheahan, T.; Heise, M.; Yount, B.; Davis, N.; Sims, A.; Suthar, M.; Harkema, J.; Whitmore, A.; Pickles, R.; et al. Vaccine efficacy in senescent mice challenged with recombinant SARS-CoV bearing epidemic and zoonotic spike variants. PLoS Med. 2006, 3, e525.
17. Graham, R. L.; Becker, M. M.; Eckerle, L. D.; Bolles, M.; Denison, M. R.; Baric, R. S. A live, impaired-fidelity coronavirus vaccine protects in an aged, immunocompromised mouse model of lethal disease. Nat. Med. 2012, 18, 1820-1826.
18. Lin, Y.; Shen, X.; Yang, R. F.; Li, Y. X.; Ji, Y. Y.; He, Y. Y.; Shi, M. De; Lu, W.; Shi, T. L.; Wang, J.; et al. Identification of an epitope of SARS-coronavirus nucleocapsid protein. Cell Res. 2003, 13, 141-145.
19. Wang, J.; Wen, J.; Li, J.; Yin, J.; Zhu, Q.; Wang, H.; Yang, Y.; Qin, E.; You, B.; Li, W.; et al. Assessment of immunoreactive synthetic peptides from the structural proteins of severe acute respiratory syndrome coronavirus. Clin. Chem. 2003, 49, 1989-1996.
20. Liu, X.; Shi, Y.; Li, P.; Li, L.; Yi, Y.; Ma, Q.; Cao, C. Profile of antibodies to the nucleocapsid protein of the severe acute respiratory syndrome (SARS)-associated coronavirus in probable SARS patients. Clin. Vaccine Immunol. 2004, 11, 227-228.
21. Tang, F.; Quan, Y.; Xin, Z.-T.; Wrammert, J.; Ma, M.-J.; Lv, H.; Wang, T.-B.; Yang, H.; Richardus, J. H.; Liu, W.; et al. Lack of peripheral memory B cell responses in recovered patients with severe acute respiratory syndrome: A six-year follow-up study. J. Immunol. 2011, 186, 7264-7268.
22. Peng, H.; Yang, L.-T.; Wang, L.-Y.; Li, J.; Huang, J.; Lu, Z.-Q.; Koup, R. A.; Bailer, R. T.; Wu, C.-Y. Long-lived memory T lymphocyte responses against SARS coronavirus nucleocapsid protein in SARS-recovered patients. Virology 2006, 351, 466-475.
23. Fan, Y.-Y.; Huang, Z.-T.; Li, L.; Wu, M.-H.; Yu, T.; Koup, R. A.; Bailer, R. T.; Wu, C.-Y. Characterization of SARS-CoV-specific memory T cells from recovered individuals 4 years after infection. Arch. Virol. 2009, 154, 1093-1099.
24. Ng, O.-W.; Chia, A.; Tan, A. T.; Jadi, R. S.; Leong, H. N.; Bertoletti, A.; Tan, Y.-J. Memory T cell responses targeting the SARS coronavirus persist up to 11 years post-infection. Vaccine 2016, 34, 2008-2014.
25. Liu, W. J.; Zhao, M.; Liu, K.; Xu, K.; Wong, G.; Tan, W.; Gao, G. F. T-cell immunity of SARS-CoV: Implications for vaccine development against MERS-CoV. Antiviral Res. 2017, 137, 82-92.
26. Li, C. K.-F.; Wu, H.; Yan, H.; Ma, S.; Wang, L.; Zhang, M.; Tang, X.; Temperton, N. J.; Weiss, R. A.; Brenchley, J. M.; et al. T cell responses to whole SARS coronavirus in humans. J Immunol. 2008, 181, 5490-5500.
27. Channappanavar, R.; Fett, C.; Zhao, J.; Meyerholz, D. K.; Perlman, S. Virus-specific memory CD8 T cells provide substantial protection from lethal severe acute respiratory syndrome coronavirus infection. J Virol. 2014, 88, 11034-11044.
28. Katoh, K.; Standley, D. M. MAFFT multiple sequence alignment software version 7: Improvements in performance and usability. Mol. Biol. Evol. 2013, 30, 772-780.
29. Pickett, B. E.; Sadat, E. L.; Zhang, Y.; Noronha, J. M.; Squires, R. B.; Hunt, V.; Liu, M.; Kumar, S.; Zaremba, S.; Gu, Z.; et al. ViPR: An open bioinformatics database and analysis resource for virology research. Nucleic Acids Res. 2012, 40, D593-D598.
30. Vita, R.; Mahajan, S.; Overton, J. A.; Dhanda, S. K.; Martini, S.; Cantrell, J R.; Wheeler, D. K.; Sette, A.; Peters, B. The immune epitope database (IEDB): 2018 update. Nucleic Acids Res. 2019, 47, D339-D343.
31. Mirarab, S.; Nguyen, N.; Guo, S.; Wang, L.-S.; Kim, J.; Warnow, T. PASTA: Ultra-large multiple sequence alignment for nucleotide and amino-acid sequences. J. Comput. Biol. 2015, 22, 377-386.
32. Huson, D. H.; Scomavacca, C. Dendroscope 3: An interactive tool for rooted phylogenetic trees and networks. Syst. Biol. 2012, 61, 1061-1067.
33. Ahmed, S. F. Data and software code for reproducing results of this paper. Available online: https://github.com/faraz107/2019-nCoV-T-Cell-Vaccine-Candidates (accessed on Feb. 25, 2020).
34. Li, F. Structure of SARS coronavirus spike receptor-binding domain complexed with receptor. Science. 2005, 309, 1864-1868.
35. Dahirel, V.; Shekhar, K.; Pereyra, F.; Miura, T.; Artyomov, M.; Talsania, S.; Allen, T. M.; Altfeld, M.; Carrington, M.; Irvine, D. J.; et al. Coordinate linkage of HIV evolution reveals regions of immunological vulnerability. Proc. Natl. Acad. Sci. 2011, 108, 11530-11535.
36. Quadeer, A. A.; Louie, R. H. Y.; Shekhar, K.; Chakraborty, A. K.; Hsing, I.-M.; McKay, M. R. Statistical linkage analysis of substitutions in patient-derived sequences of genotype 1a hepatitis C virus nonstructural protein 3 exposes targets for immunogen design. J. Virol. 2014, 88, 7628-7644.
37. Ahmed, S. F.; Quadeer, A. A.; Morales-Jimenez, D.; McKay, M. R. Sub-dominant principal components inform new vaccine targets for HIV Gag. Bioinformatics 2019, 35, 3884-3889.
38. Quadeer, A. A.; Morales-Jimenez, D.; McKay, M. R. Co-evolution networks of HIV/HCV are modular with direct association to structure and function. PLOS Comput. Biol. 2018, 14, e1006409.
39. Prabakaran, P.; Gan, J.; Feng, Y.; Zhu, Z.; Choudhry, V.; Xiao, X.; Ji, X.; Dimitrov, D. S. Structure of severe acute respiratory syndrome coronavirus receptor-binding domain complexed with neutralizing antibody. J. Biol. Chem. 2006, 281, 15829-15836.
40. Zhu, Z.; Chakraborti, S.; He, Y.; Roberts, A.; Sheahan, T.; Xiao, X.; Hensley, L. E.; Prabakaran, P.; Rockx, B.; Sidorov, I. A.; et al. Potent cross-reactive neutralization of SARS coronavirus isolates by human monoclonal antibodies. Proc. Natl. Acad. Sci. 2007, 104, 12123-12128.
41. Hwang, W. C.; Lin, Y.; Santelli, E.; Sui, J.; Jaroszewski, L.; Stec, B.; Farzan, M.; Marasco, W. A.; Liddington, R. C. Structural basis of neutralization by a human anti-severe acute respiratory syndrome spike protein antibody, 80R. J. Biol. Chem. 2006, 281, 34610-34616.
42. UniProt UniProtKB-P59594 (SPIKE_CVHSA). Available online: https://www.uniprot.org/uniprot/P59594 (accessed on Feb. 23, 2020).
43. Walls, A. C.; Park, Y.-J.; Tortorici, M. A.; Wall, A.; McGuire, A. T.; Veesler, D. Structure, function and antigenicity of the SARS-CoV-2 spike glycoprotein. bioRxiv 2020, 2020.02.19.956581.
44. Walls, A. C.; Xiong, X.; Park, Y.-J.; Tortorici, M. A.; Snijder, J.; Quispe, J.; Cameroni, E.; Gopal, R.; Dai, M.; Lanzavecchia, A.; et al. Unexpected receptor functional mimicry elucidates activation of coronavirus fusion. Cell 2019, 176, 1026-1039.e15.
45. Walls, A. C.; Tortorici, M. A.; Snijder, J.; Xiong, X.; Bosch, B.-J.; Rey, F. A.; Veesler, D. Tectonic conformational changes of a coronavirus spike glycoprotein promote membrane fusion. Proc. Natl. Acad. Sci. 2017, 114, 11157-11162.
46. Song, W.; Gui, M.; Wang, X.; Xiang, Y. Cryo-E M structure of the SARS coronavirus spike glycoprotein in complex with its host cell receptor ACE2. PLOS Pathog. 2018, 14, e1007236.
47. Wrapp, D.; Wang, N.; Corbett, K. S.; Goldsmith, J. A.; Hsieh, C.-L.; Abiona, O.; Graham, B. S.; McLellan, J. S. Cryo-E M structure of the 2019-nCoV spike in the prefusion conformation. Science. 2020, 2011, eabb2507.
48. Tian, X.; Li, C.; Huang, A.; Xia, S.; Lu, S.; Shi, Z.; Lu, L.; Jiang, S.; Yang, Z.; Wu, Y.; et al. Potent binding of 2019 novel coronavirus spike protein by a SARS coronavirus-specific human monoclonal antibody. Emerg. Microbes Infect. 2020, 9, 382-385.
49. Ferguson, A. L.; Mann, J. K.; Omarjee, S.; Ndung'u, T.; Walker, B. D.; Chakraborty, A. K. Translating HIV sequences into quantitative fitness landscapes predicts viral vulnerabilities for rational immunogen design. Immunity 2013, 38, 606-617.
50. Chakraborty, A. K.; Barton, J. P. Rational design of vaccine targets and strategies for HIV: a crossroad of statistical physics, biology, and medicine. Reports Prog. Phys. 2017, 80, 032601.
51. Quadeer, A. A.; Louie, R. H. Y.; McKay, M. R. Identifying immunologically-vulnerable regions of the HCV E2 glycoprotein and broadly neutralizing antibodies that target them. Nat. Commun. 2019, 10, 2073.
52. Louie, R. H. Y.; Kaczorowski, K. J.; Barton, J. P.; Chakraborty, A. K.; McKay, M. R. Fitness landscape of the human immunodeficiency virus envelope protein that is targeted by antibodies. Proc. Natl. Acad. Sci. 2018, 115, E564-E573.
53. Quadeer, A. A.; Barton, J. P.; Chakraborty, A. K.; McKay, M. R. Deconvolving mutational patterns of poliovirus outbreaks reveals its intrinsic fitness landscape. Nat. Commun. 2020, 11, 377.
54. Mann, J. K.; Barton, J. P.; Ferguson, A. L.; Omarjee, S.; Walker, B. D.; Chakraborty, A.; Ndung'u, T. The fitness landscape of HIV-1 Gag: Advanced modeling approaches and validation of model predictions by in vitro testing. PLoS Comput. Biol. 2014, 10, e1003776.
55. Ramaiah, A.; Arumugaswami, V. Insights into cross-species evolution of novel human coronavirus 2019-nCoV and defining immune determinants for vaccine development. bioRxiv 2020.01.29.925867 2020.
56. Ahmed, S. F., Quadeer, A. A. & McKay, M. R. Preliminary identification of potential vaccine targets for the COVID-19 coronavirus (SARS-CoV-2) based on SARS-CoV immunological studies. Viruses 12, 254 (2020).
57. Grifoni, A. et al. A sequence homology and bioinformatic approach can predict candidate targets for immune responses to SARS-CoV-2. Cell Host Microbe 27, 1-10 (2020).
58. Walls, A. C. et al. Structure, function, and antigenicity of the SARS-CoV-2 spike glycoprotein. Cell 180, 1-12 (2020).
59. Wang, C. et al. A human monoclonal antibody blocking SARS-CoV-2 infection. Nat. Commun. 11, 2251 (2020).
60. Pinto, D. et al. Cross-neutralization of SARS-CoV-2 by a human monoclonal SARS-CoV antibody. Nature (2020). doi:10.1038/s41586-020-2349-y
61. Wrapp, D. et al. Cryo-EM structure of the 2019-nCoV spike in the prefusion conformation. Science 367, 1260-1263 (2020).
62. Chour, W. et al. Shared antigen-specific CD8+ T cell responses against the SARS-COV-2 spike protein in HLA A*02:01 COVID-19 participants. medRxiv 2020.05.04.20085779 (2020). doi:10.1101/2020.05.04.20085779
63. Shomuradova, A. S. et al. SARS-CoV-2 epitopes are recognized by a public and diverse repertoire of human T-cell receptors. bioRxiv 2020.05.20.20107813 (2020). doi: 10.1101/2020.05.20.20107813
64. Smith, T. R. F. et al. Immunogenicity of a DNA vaccine candidate for COVID-19. Nat. Commun. 11, 2601 (2020).
65. Poh, C. M. et al. Two linear epitopes on the SARS-CoV-2 spike protein that elicit neutralising antibodies in COVID-19 patients. Nat. Commun. 11, 2806 (2020).
66. Yin, D. et al. A single dose SARS-CoV-2 simulating particle vaccine induces potent neutralizing activities. bioRxiv 2020.05.14.093054 (2020). doi:10.1101/2020.05.14.093054
67. Ahmed, S. F., Quadeer, A. A. & McKay, M. R. COVIDep platform for real-time reporting of vaccine target recommendations for SARS-CoV-2: Description and connections with COVID-19 immune responses and preclinical vaccine trials. bioRxiv 2020.05.23.111385 (2020). doi:10.1101/2020.05.23.111385.
68. Abdool Karim S S, de Oliveira T. 2021. New SARS-CoV-2 variants—clinical, public health, and vaccine implications. N. Engl. J. Med. [Internet]:NEJMc2100362. Available from: www.nejm.org/doi/10.1056/NEJMc2100362.
69. Abu-Raddad L J, Chemaitelly H, Butt A A. 2021. Effectiveness of the BNT 162b2 COVID-19 vaccine against the B.1.1.7 and B.1.351 variants. N. Engl. J. Med. [Internet]:NEJMc2104974. Available from: www.nejm.org/doi/10.1056/NEJMc2104974.
70. Altmann D M, Boyton R J. 2020. SARS-CoV-2 T cell immunity: Specificity, function, durability, and role in protection. Sci. Immunol. [Internet] 5:2-7. Available from: 10.0.4.102/sciimmunol.abd6160.
71. Anderson E J, Rouphael N G, Widge A T, Jackson L A, Roberts P C, Makhene M, Chappell J D, Denison M R, Stevens L J, Pruijssers A J, et al. 2020. Safety and immunogenicity of SARS-CoV-2 mRNA-1273 vaccine in older adults. N. Engl. J. Med. 383:2427-2438.
72. Andreatta M, Nielsen M. 2016. Gapped sequence alignment using artificial neural networks: Application to the MHC class I system. Bioinformatics [Internet] 32:511-517. Available from: https://academic.oup.com/bioinformatics/article/32/4/511/1744469.
73. Bergamaschi L, Mescia F, Turner L, Hanson A, Kotagiri P, Dunmore B J, Ruffieux H, De Sa A, Huhn O, Morgan M D, et al. 2021. Delayed bystander CD8+ T cell activation, early immune pathology and persistent dysregulation characterise severe COVID-19. medRxiv [Internet]:2021.01.11.20248765. Available from: medrxiv.org/content/early/2021/03/26/2021.01.11.20248765.abstract.
74. Bertoletti A, Tan A T, Le Bert N. 2021. The T-cell response to SARS-CoV-2: Kinetic and quantitative aspects and the case for their protective role. Oxford Open Immunol. [Internet] 2:1-9. Available from: https://academic.oup.com/ooim/article/doi/10.1093/oxfimm/iqab006/6146940.
75. Bueno S M, Abarca K, González P A, Gálvez NMS, Soto J A, Duarte L F, Schultz B M, Pacheco G A, González L A, Rios M, et al. 2021. Interim report: Safety and immunogenicity of an inactivated vaccine against SARS-CoV-2 in healthy Chilean adults in a phase 3 clinical trial. medRxiv [Internet]:2021.03.31.21254494. Available from: medrxiv.org/content/early/2021/04/01/2021.03.31.21254494.abstract.
76. Chen Z, John Wherry E. 2020. T cell responses in patients with COVID-19. Nat. Rev. Immunol. [Internet]20:529-536. Available from: www.nature.com/articles/s41577-020-0402-6.
77. Cohen K W, Linderman S L, Moodie Z, Czartoski J, Lai L, Mantus G, Norwood C, Nyhoff L E, Edara V V, Floyd K, et al. 2021. Longitudinal analysis shows durable and broad immune memory after SARS-CoV-2 infection with persisting antibody responses and memory B and T cells. medRxiv [Internet]:2021.04.19.21255739. Available from: medrxiv.org/content/early/2021/04/27/2021.04.19.21255739.abstract.
78. Collier D A, De Marco A, Ferreira I A T M, Meng B, Datir R, Walls A C, Kemp S S A, Bassi J, Pinto D, Fregni C S, et al. 2021. Sensitivity of SARS-CoV-2 B.1.1.7 to mRNA vaccine-elicited antibodies. Nature.
79. Ella R, Vadrevu K M, Jogdand H, Prasad S, Reddy S, Sarangi V, Ganneru B, Sapkal G, Yadav P, Abraham P, et al. 2021. Safety and immunogenicity of an inactivated SARS-CoV-2 vaccine, BBV152: A double-blind, randomised, phase 1 trial. Lancet Infect. Dis. [Internet] 3099:2020.12.21.20248643. Available from: https://doi.org/10.1016/S1473-3099(20)30942-7.
80. Emary K R W, Golubchik T, Aley P K, Ariani C V., Angus B, Bibi S, Blane B, Bonsall D, Cicconi P, Charlton S, et al. 2021. Efficacy of ChAdOx1 nCoV-19 (AZD1222) vaccine against SARS-CoV-2 variant of concern 202012/01 (B.1.1.7): An exploratory analysis of a randomised controlled trial. Lancet 397:1351-1362.
81. Fedry J, Hurdiss D L, Wang C, Li W, Obal G, Drulyte I, Du W, Howes S C, van Kuppeveld F J M, Forster F, et al. 2021. Structural insights into the cross-neutralization of SARS-CoV and SARS-CoV-2 by the human monoclonal antibody 47D11. Sci. Adv. [Internet] 7:eabf5632. Available from: https://advances.sciencemag.org/lookup/doi/10.1126/sciadv.abf5632.
82. Folegatti P M, Ewer K J, Aley P K, Angus B, Becker S, Belij-Rammerstorfer S, Bellamy D, Bibi S, Bittaye M, Clutterbuck E A, et al. 2020. Safety and immunogenicity of the ChAd0x1 nCoV-19 vaccine against SARS-CoV-2: A preliminary report of a phase 1/2, single-blind, randomised controlled trial. Lancet [Internet] 396:467-478. Available from: https://linkinghub.elsevier.com/retrieve/pii/SO140673620316044.
83. Gonzalez-Galarza F F, McCabe A, Santos E J M dos, Jones J, Takeshita L, Ortega-Rivera N D, Cid-Pavon G M Del, Ramsbottom K, Ghattaoraya G, Alfirevic A, et al. 2019. Allele frequency net database (AFND) 2020 update: Gold-standard data classification, open access genotype data and new query tools. Nucleic Acids Res. [Internet]. Available from: https://academic.oup.com/nar/advance-article/doi/10.1093/nar/gkz1029/5624967.
84. Grifoni A, Weiskopf D, Ramirez S I, Mateus J, Dan J M, Moderbacher C R, Rawlings S A, Sutherland A, Premkumar L, Jadi R S, et al. 2020. Targets of T cell responses to SARS-CoV-2 coronavirus in humans with COVID-19 disease and unexposed individuals. Cell [Internet] 181:1489-1501.e15. Available from: https://doi.org/10.1016/j.cell.2020.05.015.
85. Hall V J, Foulkes S, Saei A, Andrews N, Oguti B, Charlett A, Wellington E, Stowe J, Gillson N, Atti A, et al. 2021. COVID-19 vaccine coverage in health-care workers in England and effectiveness of BNT 162b2 mRNA vaccine against infection (SIREN): A prospective, multicentre, cohort study. Lancet [Internet]6736:1-11. Available from: https://linkinghub.elsevier.com/retrieve/pii/S014067362100790X.
86. Hwang W, Lei W, Katritsis N M, MacMahon M, Chapman K, Han N. 2021. Current and prospective computational approaches and challenges for developing COVID-19 vaccines. Adv. Drug Deliv. Rev. [Internet] 172:249-274. Available from: https://linkinghub.elsevier.com/retrieve/pii/S0169409X21000387
87. Jin Y, Wang J, Bachtiar M, Chong S S, Lee C G L. 2018. Architecture of polymorphisms in the human genome reveals functionally important and positively selected variants in immune response and drug transporter genes. Hum. Genomics [Internet] 12:43. Available from: https://humgenomics.biomedeentral.com/articles/10.1186/s40246-018-0175-1
88. Jurtz V, Paul S, Andreatta M, Marcatili P, Peters B, Nielsen M. 2017. NetMHCpan-4.0: Improved peptide-MHC class I interaction predictions integrating eluted ligand and peptide binding affinity data. J Immunol. [Internet] 199:3360-3368. Available from: www.jimmunol.org/lookup/doi/10.4049/jimmunol.1700893
89. Keech C, Albert G, Cho I, Robertson A, Reed P, Neal S, Plested J S, Zhu M, Cloney-Clark S, Zhou H, et al. 2020. Phase 1-2 trial of a SARS-CoV-2 recombinant spike protein nanoparticle vaccine. N. Engl. J. Med. [Internet]:NEJMoa2026920. Available from: www.nejm.org/doi/10.1056/NEJMoa2026920
90. Kim Y, Sidney J, Pinilla C, Sette A, Peters B. 2009. Derivation of an amino acid similarity matrix for peptide: MHC binding and its application as a Bayesian prior. BMC Bioinformatics [Internet] 10:394. Available from: www.nebi.nlm.nih.gov/pubmed/19948066
91. Klasse P J, Nixon D F, Moore J P. 2021. Immunogenicity of clinically relevant SARS-CoV-2 vaccines in nonhuman primates and humans. Sci. Adv. 7:1-23.
92. Liao M, Liu Y, Yuan J, Wen Y, Xu G, Zhao J, Cheng L, Li J, Wang X, Wang F, et al. 2020. Single-cell landscape of bronchoalveolar immune cells in patients with COVID-19. Nat. Med. [Internet] 26:842-844. Available from: dx.doi.org/10.1038/s41591-020-0901-9.
93. Liu Y, Liu J, Xia H, Zhang X, Fontes-Garfias C R, Swanson K A, Cai H, Sarkar R, Chen W, Cutler M, et al. 2021. Neutralizing activity of BNT 162b2-elicited serum. N. Engl. J. Med. [Internet] 384:1466-1468. Available from: www.nejm.org/doi/10.1056/NEJMc2102017.
94. Logunov D Y, Dolzhikova I V., Zubkova O V., Tukhvatullin A I, Shcheblyakov D V., Dzharullaeva A S, Grousova D M, Erokhova A S, Kovyrshina A V., Botikov A G, et al. 2020. Safety and immunogenicity of an rAd26 and rAd5 vector-based heterologous prime-boost COVID-19 vaccine in two formulations: Two open, non-randomised phase 1/2 studies from Russia. Lancet 396:887-897.
95. Madhi S A, Baillie V, Cutland C L, Voysey M, Koen A L, Fairlie L, Padayachee S D, Dheda K, Barnabas S L, Bhorat Q E, et al. 2021. Efficacy of the ChAd0x1 nCoV-19 COVID-19 vaccine against the B.1.351 variant. N. Engl. J. Med. [Internet]:NEJMoa2102214. Available from: www.nejm.org/doi/10.1056/NEJMoa2102214.
96. Mazzoni A, Maggi L, Capone M, Spinicci M, Salvati L, Colao M G, Vanni A, Kiros S T, Mencarini J, Zammarchi L, et al. 2020. Cell-mediated and humoral adaptive immune responses to SARS-CoV-2 are lower in asymptomatic than symptomatic COVID-19 patients. Eur. J. Immunol. 50:2013-2024.
97. Mor V, Gutman R, Yang X, White E M, McConeghy K W, Feifer R A, Blackman C R, Kosar C M, Bardenheier B H, Gravenstein S A. 2021. Short-term impact of nursing home SARS-CoV-2 vaccinations on new infections, hospitalizations, and deaths. J. Am. Geriatr. Soc. [Internet]:jgs.17176. Available from: https://onlinelibrary.wiley.com/doi/10.1111/jgs.17176
98. Moutaftsi M, Peters B, Pasquetto V, Tscharke D C, Sidney J, Bui H H, Grey H, Sette A. 2006. A consensus epitope prediction approach identifies the breadth of murine TCD8+-cell responses to vaccinia virus. Nat. Biotechnol. 24:817-819.
99. Nielsen M, Andreatta M. 2016. NetMHCpan-3.0; improved prediction of binding to MHC class I molecules integrating information from multiple receptor and peptide length datasets. Genome Med. [Internet] 8:33. Available from: https://genomemedicine.biomedeentral.com/articles/10.1186/s13073-016-0288-x
100. O'Donnell T J, Rubinsteyn A, Laserson U. 2020. MHCflurry 2.0: Improved pan-allele prediction of MHC class I-presented peptides by incorporating antigen processing. Cell Syst. [Internet] 11:42-48.e7. Available from: https://doi.org/10.1016/j.cels.2020.06.010
101. Pala P, Bodmer H C, Pemberton R M, Cerottini J-C, Maryanski J L, Askonas B A. 1988. Competition between unrelated peptides recognized by H-2-Kd restricted T cells. J. Immunol. 141:2289-2294.
102. Paul S, Croft N P, Purcell A W, Tscharke D C, Sette A, Nielsen M, Peters B. 2020. Benchmarking predictions of MHC class I restricted T cell epitopes in a comprehensively studied model system. PLoS Comput. Biol. [Internet] 16:1-18. Available from: dx.doi.org/10.1371/journal.pcbi.1007757
103. Peters B, Sette A. 2005. Generating quantitative models describing the sequence specificity of biological processes with the stabilized matrix method. BMC Bioinformatics [Internet] 6. Available from: www.nebi.nlm.nih.gov/pubmed/15927070
104. Piccoli L, Park Y-J, Tortorici M A, Czudnochowski N, Walls A C, Beltramello M, Silacci-Fregni C, Pinto D, Rosen L E, Bowen J E, et al. 2020. Mapping neutralizing and immunodominant sites on the SARS-CoV-2 spike receptor-binding domain by structure-guided high-resolution serology. Cell [Internet] 183:1024-1042.e21. Available from: https://linkinghub.elsevier.com/retrieve/pii/S0092867420312344
105. Pinto D, Park Y, Beltramello M, Walls A C, Tortorici M A, Bianchi S, Jaconi S, Culap K, Zatta F, De Marco A, et al. 2020. Cross-neutralization of SARS-CoV-2 by a human monoclonal SARS-CoV antibody. Nature [Internet] 583:290-295. Available from: dx.doi.org/10.1038/s41586-020-2349-y
106. Planas D, Bruel T, Grzelak L, Guivel-Benhassine F, Staropoli I, Porrot F, Planchais C, Buchrieser J, Rajah M M, Bishop E, et al. 2021. Sensitivity of infectious SARS-CoV-2 B.1.1.7 and B.1.351 variants to neutralizing antibodies. Nat. Med. [Internet]. Available from: www.nature.com/articles/s41591-021-01318-5
107. Quadeer A A, Ahmed S F, McKay M R. 2021. Landscape of epitopes targeted by T cells in 852 convalescent COVID-19 patients: Meta-analysis, immunoprevalence and web platform. Cell Reports Med. [Internet]:100312. Available from: https://linkinghub.elsevier.com/retrieve/pii/S2666379121001555
108. Ramasamy M N, Minassian A M, Ewer K J, Flaxman A L, Folegatti P M, Owens D R, Voysey M, Aley P K,
Angus B, Babbage G, et al. 2020. Safety and immunogenicity of ChAdOx1 nCoV-19 vaccine administered in a prime-boost regimen in young and old adults (COV002): A single-blind, randomised, controlled, phase 2/3 trial. Lancet 396:1979-1993.
109. Reynisson B, Alvarez B, Paul S, Peters B, Nielsen M. 2020. NetMHCpan-4.1 and NetMHCIIpan-4.0: Improved predictions of MHC antigen presentation by concurrent motif deconvolution and integration of M S MHC eluted ligand data. Nucleic Acids Res. 48:W449-W454.
110. Reynolds C J, Swadling L, Gibbons J M, Pade C, Jensen M P, Diniz M O, Schmidt N M, Butler D K, Amin O E, Bailey S N L, et al. 2020. Discordant neutralizing antibody and T cell responses in asymptomatic and mild SARS-CoV-2 infection. Sci. Immunol. [Internet] 5:eabf3698. Available from: https://immunology.sciencemag.org/lookup/doi/10.1126/sciimmunol.abf3698
111. Robinson J, Halliwell J A, Hayhurst J D, Flicek P, Parham P, Marsh SGE. 2015. The IPD and IMGT/HLA database: Allele variant databases. Nucleic Acids Res. [Internet] 43:D423-D431. Available from: academic.oup.com/nar/article/43/DI/D423/2438496/The-IPD-and-IMGTHLA-database-allele-variant
112. Rydyznski Moderbacher C, Ramirez S I, Dan J M, Grifoni A, Hastie K M, Weiskopf D, Belanger S, Abbott R K, Kim Christina, Choi J, et al. 2020. Antigen-specific adaptive immunity to SARS-CoV-2 in acute COVID-19 and associations with age and disease severity. Cell [Internet]:1-17. Available from: https://doi.org/10.1016/j.cell.2020.09.038
113. Ryzhikov Aleksandr B., Ryzhikov E A, Bogryantseva M P, Danilenko E D, Imatdinov I R, Nechaeva E A,
Pyankov O V., Pyankova O G, Susloparov I M, Taranov O S, et al. 2021. Immunogenicity and protectivity of the peptide candidate vaccine against SARS-CoV-2. Ann. Russ. Acad. Med. Sci. [Internet] 76:5-19. Available from: https://vestnikramn.spr-journal.ru/jour/article/view/1528
114. Ryzhikov A. B., Ryzhikov E A, Bogryantseva M P, Usova S V., Danilenko E D, Nechaeva E A, Pyankov O V., Pyankova O G, Gudymo A S, Bodnev S A, et al. 2021. A single blind, placebo-controlled randomized study of the safety, reactogenicity and immunogenicity of the “EpiVacCorona” vaccine for the prevention of COVID-19, in volunteers aged 18-60 years (phase I-II). Russ. J. Infect. Immun. [Internet] 11:283-296. Available from: https://www.iimmun.ru/iimm/article/view/1699
115. Sadoff J, Le Gars M, Shukarev G, Heerwegh D, Truyers C, de Groot A M, Stoop J, Tete S, Van Damme W, Leroux-Roels I, et al. 2021. Interim results of a phase 1-2a trial of Ad26.COV2.S COVID-19 vaccine. N. Engl. J. Med.:1-12.
116. Sahin U, Muik A, Derhovanessian E, Vogler I, Kranz L M, Vormehr M, Baum A, Pascal K, Quandt J, Maurus D, et al. 2020. COVID-19 vaccine BNT162b1 elicits human antibody and TH1 T cell responses. Nature [Internet] 586:594-599. Available from: dx.doi.org/10.1038/s41586-020-2814-7
117. Sahin U, Muik A, Vogler I, Derhovanessian E, Kranz L M, Vormehr M, Quandt J, Bidmon N, Ulges A, Baum A, et al. 2021. BNT162b2 vaccine induces neutralizing antibodies and poly-specific T cells in humans. Nature [Internet]. Available from: www.nature.com/articles/s41586-021-03653-6
118. Sarkizova S, Klaeger S, Le P M, Li L W, Oliveira G, Keshishian H, Hartigan C R, Zhang W, Braun D A, Ligon K L, et al. 2020. A large peptidome dataset improves HLA class I epitope prediction across most of the human population. Nat. Biotechnol. [Internet] 38:199-209. Available from: dx.doi.org/10.1038/s41587-019-0322-9
119. Shrotri M, Swinnen T, Kampmann B, Parker E P K. 2021. An interactive website tracking COVID-19 vaccine development. Lancet Glob. Heal. [Internet] 9:e590-e592. Available from: dx.doi.org/10.1016/S2214-109X(21)00043-7
120. Sohail M S, Ahmed S F, Quadeer A A, McKay M R. 2021. In silico T cell epitope identification for SARS-CoV-2: Progress and perspectives. Adv. Drug Deliv. Rev. [Internet] 171:29-47. Available from: https://doi.org/10.1016/j.addr.2021.01.007
121. Tarke A, Sidney J, Methot N, Zhang Y, Dan J M, Goodwin B, Rubiro P, Sutherland A, da Silva Antunes R, Frazier A, et al. 2021. Negligible impact of SARS-CoV-2 variants on CD4+ and CD8+ T cell reactivity in COVID-19 exposed donors and vaccinees. bioRxiv [Internet]:2021.02.27.433180. Available from: www.nebi.nlm.nih.gov/pubmed/33688655% 0Awww.pubmedcentral.nih.gov/articlerender.fcgi?artid=PMC7941626
122. Tauzin A, Nayrac M, Benlarbi M, Gong S Y, Gasser R, Beaudoin-Bussieres G, Brassard N, Laumaea A, Vézina D, Prevost J, et al. 2021. A single dose of the SARS-CoV-2 vaccine BNT 162b2 elicits Fe-mediated antibody effector functions and T-cell responses. Cell Host Microbe [Internet]. Available from: https://doi.org/10.1016/j.chom.2021.06.001
123. Vita R, Mahajan S, Overton J A, Dhanda S K, Martini S, Cantrell J R, Wheeler D K, Sette A, Peters B. 2019. The immune epitope database (IEDB): 2018 update. Nucleic Acids Res. [Internet] 47:D339-D343. Available from: https://doi.org/10.1093/nar/gky1006
124. Wall E C, Wu M, Harvey R, Kelly G, Warchal S, Sawyer C, Daniels R, Hobson P, Hatipoglu E, Ngai Y, et al. 2021. Neutralising antibody activity against SARS-CoV-2 VOCs B.1.617.2 and B.1.351 by BNT 162b2 vaccination. Lancet [Internet] 6736:3-5. Available from: dx.doi.org/10.1016/S0140-6736(21)01290-3
125. Woldemeskel B A, Garliss C C, Blankson J N. 2021. SARS-CoV-2 mRNA vaccines induce broad CD4+ T cell responses that recognize SARS-CoV-2 variants and HCoV-NL63. J Clin. Invest. 131.
128. Wyllie D, Mulchandani R, Jones H E, Taylor-phillips S, Brooks T, Charlett A, Ades A E, Makin A, Oliver I. 2020. SARS-CoV-2 responsive T cell numbers are associated with protection from COVID-19: A prospective cohort study in keyworkers. medRxiv.
127. Zhou D, Dejnirattisai W, Supasa P, Liu C, Mentzer A J, Ginn H M, Zhao Y, Duyvesteyn H M E, Tuekprakhon A, Nutalai R, et al. 2021. Evidence of escape of SARS-CoV-2 variant B.1.351 from natural and vaccine-induced sera. Cell [Internet] 184:2348-2361.e6. Available from: https://doi.org/10.1016/j.cell.2021.02.037
128. Zhu F C, Guan X H, Li Y H, Huang J Y, Jiang T, Hou L H, Li J X, Yang B F, Wang L, Wang W J, et al. 2020. Immunogenicity and safety of a recombinant adenovirus type-5-vectored COVID-19 vaccine in healthy adults aged 18 years or older: A randomised, double-blind, placebo-controlled, phase 2 trial. Lancet [Internet] 396:479-488. Available from: dx.doi.org/10.1016/S0140-6736(20)31605-6
129. Jim Boonyaratanakomkit and Justin J. Taylor. 2019. Techniques to Study Antigen-Specific B Cell Responses. Front. Immunol., 24 July 2019. doi.org/10.3389/fimmu.2019.01694
130. David D. Chaplin, 2010. Overview of the immune response. J Allergy Clin Immunol. 2010 February; 125(2 Suppl 2): S3-23. doi: 10.1016/j.jaci.2009.12.980
131. Bercovici et al., 2000. New Methods for Assessing T-Cell Responses. Clin Diagn Lab Immunol. 2000 November;
7(6): 859-864. doi: 10.1128/cdli.7.6.859-864.2000
132. Powell & Newman, eds., Vaccine Design (the subunit and adjuvant approach) (1995).
133. Rolland, Crit. Rev. Therap. Drug Carrier Systems 15:143-198 (1998)
134. Fisher-Hoch et al., Proc. Natl. Acad. Sci. USA 86:317-321 (1989)
135. Flexner et al., Ann. N. Y. Acad. Sci. 569:86-103 (1989)
136. Flexner et al., Vaccine 8:17-21 (1990)
137. Berkner, Biotechniques 6:616-627 (1988)
138. Rosenfeld et al., Science 252:431-434 (1991)
139. Kolls et al., Proc. Natl. Acad. Sci. USA 91:215-219 (1994)
140. Kass-Eisler et al., Proc. Natl. Acad. Sci. USA 90:11498-11502 (1993)
141. Guzman et al., Circulation 88:2838-2848 (1993)
142. Guzman et al., Cir. Res. 73:1202-1207 (1993)
143. Ulmer et al., Science 259:1745-1749 (1993) and reviewed by Cohen, Science 259:1691-1692 (1993)
144. Kobiyama, et al Vaccines, 2013, 1(3), 278-292
145. Mosmann & Coffman, Ann. Rev. Immunol. 7:145-173 (1989)
146. Sato et al., Science 273:352 (1996).
147. Pharmaceutical Dosage Forms (vols. 1-3, 1992)
148. Lloyd, The Art, Science and Technology of Pharmaceutical Compounding (1999)
149. Pickar, Dosage Calculations (1999)
150. Bray, B. L., 2003. Large-scale manufacture of peptide therapeutics by chemical synthesis. Nature Reviews Drug Discovery, 2(7), pp. 587-593.

1. A peptide of no more than 500 amino acids, comprising (1) at least one T cell epitope set forth in Table 3 or Tables 15-47 or (2) at least one B cell epitope set forth in Table 4, the peptide optionally further comprising at least one heterologous amino acid sequence.

2. The peptide of claim 1, comprising or consisting of at least one of the T cell epitopes.

3. The peptide of claim 1, comprising or consisting of at least one of the B cell epitopes.

4. The peptide of claim 1, comprising or consisting of at least one of the T cell epitopes and at least one heterologous amino acid sequence.

5. The peptide of claim 1, comprising or consisting of at least one of the B cell epitopes and at least one heterologous amino acid sequence.

6. A nucleic acid comprising a polynucleotide sequence encoding the peptide of claim 1.

7. An expression cassette comprising a polynucleotide sequence encoding the peptide of claim 1, operably linked to a promoter.

8. A vector comprising the expression cassette of claim 7.

9. A host cell comprising the vector of claim 8.

10. A composition comprising (1) the peptide of claim 1; and (2) a pharmaceutically acceptable excipient.

11. The composition of claim 10, further comprising an adjuvant.

12. The composition of claim 10, comprising a plurality of peptides each comprising a T cell epitope set forth in Table 3 or Tables 15-47.

13. A method of eliciting an immune response in a subject in need thereof, the method comprising administering to the subject an effective amount of a composition comprising (1) the peptide of claim 1.

14. The method of claim 13, wherein the composition is administered to the subject by a route selected from the group consisting of, subcutaneous, intramuscular, and oral.

15. The method of claim 13, wherein the subject is at risk of exposure to SARS-CoV or SARS-CoV-2 infection.

16. A kit for eliciting an immune response in a subject in need thereof, the kit comprising a first container containing the composition comprising (1) the peptide of claim 1, optionally an additional container containing a therapeutic agent against SARS-CoV-2.

17. The kit of claim 16, further comprising at least a second container each containing at least one different composition.

18. A method for detecting T cell immunity against SARS-CoV-2 in a subject, comprising:
(1) contacting T cells obtained from the subject with a T cell epitope set forth in Table 3 or Tables 15-47 and antigen-presenting cells having an HLA allele associated with the epitope; and
(2) detecting activation of the T cells, thereby detecting presence of T cell immunity against SARS-CoV-2 in the subject.

19. The method of claim 18, wherein step (2) comprises detection of T cell proliferation or T cell secretion of one or more cytokines.

20. The method of claim 18, wherein step (2) comprises T cell proliferation assay, flow cytometry, ELISPOT, or ELISA.

21. The method of claim 18, wherein step (1) comprises contacting T cells obtained from the subject with a composition comprising a plurality of peptides each comprising a T cell epitope set forth in Table 3 or Tables 15-47 and antigen-presenting cells having HLA alleles associated with each of the plurality of epitopes.
